(19) United States
(12) Patent Application Publication
(10) Pub. No.: US 2020/0255823 A1
(43) Pub. Date: Aug. 13, 2020

(54) GUIDE STRAND LIBRARY CONSTRUCTION AND METHODS OF USE THEREOF

(71) Applicant: PIONEER BIOLABS, LLC, Provo, UT (US)

(72) Inventors: Joshua D. Yates, Spanish Fork, UT (US); Jonathon T. Hill, Provo, UT (US)

(21) Appl. No.: 16/794,137

(22) Filed: Feb. 18, 2020

Related U.S. Application Data
(63) Continuation-in-part of application No. 15/727,279, filed on Oct. 6, 2017, now Pat. No. 10,669,539.
(60) Provisional application No. 62/807,679, filed on Feb. 19, 2019.
(60) Provisional application No. 62/902,601, filed on Sep. 19, 2019.
(60) Provisional application No. 62/405,070, filed on Oct. 6, 2016.

(51) Int. Cl. C12N 15/10; C12N 15/11

(52) U.S. Cl. CPC C12N 15/1093; C12N 2310/20; C12N 15/11

(57) ABSTRACT

An improved method for the rapid and efficient production of guide strand libraries is disclosed. Also included are kits comprising reagents suitable for practicing the method.

### INCORPORATION-BY-REFERENCE OF MATERIAL SUBMITTED IN ELECTRONIC FORM

Applicant hereby incorporates by reference the Sequence Listing material filed in electronic form herewith. This file is labeled “PIO100_20190905_SequenceListing_ST25.txt”, created Sep. 5, 2019, and having 280,745 bytes in size.

### FIELD

The disclosure generally relates to compositions, polynucleotides, kits, methods, and systems for enzymatic construction of clustered regularly interspaced short palindromic repeats (CRISPR) guide strand libraries and methods of use for the same.

### BACKGROUND

Several publications and patent documents are cited throughout the specification in order to describe the state of the art to which this invention pertains. Each of these citations is incorporated by reference herein as though set forth in full.

Many of the cutting-edge innovations to the CRISPR/Cas9 system involve the use of complex sgRNA libraries. Such libraries have recently been used in functional genomics to screen for specific genes in both cell lines and animals, in subcellular imaging to fluorescently label chromatin in living cells, and several other applications. However, chemically synthesizing customized libraries can be cost prohibitive, requires precise genomic information on gene structure and nucleotide polymorphisms between the reference and the sample, and can take several weeks.

In view of the foregoing, methods which reduce library production time while maintaining accuracy are clearly needed.

### SUMMARY

Compositions and methods for creating guide strand libraries are disclosed. In one aspect, a first polynucleotide which encodes an RNA bound by an enzyme which functions in the CRISPR system is provided. An exemplary first polynucleotide comprises a constant region encoding sequence for a CRISPR single guide RNA (sgRNA) or CRISPR targeting RNA (crRNA) recognized by a cas enzyme for example, having a non-palindromic recognition site recognized by a type II restriction enzyme oriented in said sequence such that a second operably linked polynucleotide is cleaved 17 to 27 base pairs from said recognition site, when said second polynucleotide is present. The type II restriction enzyme has methylase activity and cleavage activity and the recognition site comprises a nucleotide which is methylated by said type II restriction enzyme upon cleavage, methylation of said nucleotide altering said recognition site such that the type II enzyme no longer binds said site. In other aspects the constant region encoding sequence is operably linked to the second polynucleotide which encodes a variable or targeting region which hybridizes to a sequence of interest.

In preferred aspects, the non-palindromic recognition site when transcribed is capable of being incorporated within a stem-loop structure of the CRISPR sgRNA or CRISPR crRNA without disrupting Cas9 binding at the constant region in said polynucleotide. In another aspect, the first polynucleotide is a plurality of first polynucleotides; at least a portion of the plurality of first polynucleotides is operably linked to a second polynucleotides to form a plurality of linked second polynucleotides; wherein the plurality of linked second polynucleotides are digested with the type II restriction enzyme having methylase activity to form a plurality of third polynucleotides encoding a plurality of CRISPR sgRNAs or CRISPR crRNAs, wherein at least one of the plurality of CRISPR sgRNAs or CRISPR crRNAs has a variable region different from the other CRISPR sgRNAs or CRISPR crRNAs. In certain embodiments the olynucleotides described above are optionally operably linked to promoter sequence(s) and the type II RE site has been methylated. Type II restriction enzyme sites useful in the compositions and methods of the invention include, without limitation, one or more of NmeAIII, MmeI, CstMI, EcoP15I, ApyPI, AquII, AquIII, AquIV, CdpI, CstMI, DraRI, DrdIV, EsaSSI, MaqI, NhaXI, NlaCI, PlaDI, PspOMII, PspPRI, Rcel, RpaB5I, SdeAI, SpoDI, and Bsb.

In certain embodiments, the polynucleotides described above comprise one or more adapter sequences. The polynucleotides can comprise a single stranded overhang of between 1 and 100 nucleotides on one or both ends in certain embodiments.

In another aspect, the constant region sequence contains a portion of the stem loop sequence and said second constant region polynucleotide encodes the remaining portion of a functional stem loop structure, wherein operable linkage via an adapter reforms a functional stem loop structure bound by a Cas enzyme. In other aspects, the constant region sequence encodes elongated stem loops or additional sequences at a 5′ end, a 3′ end or both, while maintaining a CRISPR enzyme binding site.

Also disclosed is a method for generating DNA templates for production of a CRISPR/Cas guide strand library. An exemplary method comprises providing a polynucleotide sample; digesting said polynucleotide sample at protospacer adjacent motifs (PAM) to form target region containing fragments with a first restriction enzyme (RE) in the presence of a ligase and a first adapter sequence. The first adapter sequence comprises a constant region having a CRISPR enzyme binding site, and the ligase operably links the first adapter sequence to one or both ends of the fragments, thereby forming an intermediate product which lacks a binding site recognized by said first RE, said intermediate product comprising at least one binding site for a second Type II RE. The intermediate product is then digested with the second Type II RE in the presence of a ligase and a second adapter sequence, wherein the Type II RE having methylation and nuclease activity which methylates said product at said recognition site and cleaves said targeting region between 17 to 27 base pairs away from said recognition site to form methylated fragments comprising a CRISPR constant region and targeting region, the presence of said methyl group preventing further digestion of said fragments by the second Type II RE, wherein the second adapter optionally comprises one or more of a cloning sequence, an adapter sequence, and a vector backbone. In certain aspects, the first digestion and ligation are carried out in two steps. In other aspects, the second digestion and ligation are carried out in two steps. In other aspects, the second two steps are combined.

The polynucleotide sample to be digested at PAM motifs can be obtained from any source, e.g., from one or more of an organism of interest, an organism at a selected stage of development, a tissue of interest, a cell at a selected stage of differentiation, a cell at a particular stage of the cell cycle, a tissue or cell having a selected pathology. The polynucleotide sample can comprise a nucleic acids obtained from any source of interest, including without limitation, cDNA synthesized from RNA, genomic DNA, mitochondrial DNA, human DNA, animal DNA, plant DNA, fungal DNA, archaeal DNA, or bacterial DNA. The polynucleotides can be isolated using any method useful in the art for this purpose, including, but not limited to precipitation, hybridization, antibody isolation, and co-precipitation.

In another approach, all of the steps described above are performed in a single reaction vessel. In another embodiment of the method, the first polynucleotide is a plurality of first polynucleotides; at least a portion of the plurality of first polynucleotides is operably linked to DNA to form a plurality of second linked polynucleotides; and the plurality of second linked polynucleotides are digested with the type II restriction enzyme having methylase activity to form a plurality of third polynucleotides encoding a plurality of CRISPR sgRNAs or CRISPR crRNAs, wherein at least one of the plurality of CRISPR sgRNAs or CRISPR crRNAs is methylated and has a variable region different from the other CRISPR sgRNAs or CRISPR crRNAs. The starting sample (e.g., a cDNA sample) may optionally be normalized to remove repeated transcripts from said cDNA sample, thereby increasing equal representation of transcripts in said library. In certain aspects of the method, the adapters lack a 5′ phosphate. In other aspects, the adapters contain at least six consecutive phosphorothioates at the 5′ end to render then resistant to nuclease digestion.

The nucleic acid to be obtained can be digested with enzymes, which include, but are not limited to, HpaII, MspI, ScrFI, BfaI, and PacI. In certain approaches, the ligated CRISPR sgRNA or CRISPR crRNA product comprises at least one nick. In other aspects, the second adapter comprises a promoter sequence, e.g., a T7 RNA polymerase promoter sequence. In some embodiments, the ligated product does not maintain a G-U hydrogen bond at position 1 of the scaffold in the constant region sequence.

The inventive method can also comprise purifying the ligated CRISPR sgRNA or CRISPR crRNA product. In another aspect, the first adapter sequence comprises a 5′ single stranded overhang, in certain cases to facilitate purification. In one embodiment, the ligated CRISPR sgRNA or CRISPR crRNA product is purified using a solid support operably linked to a capture oligonucleotide at a 3′ end, said oligonucleotide hybridizing to a 5′ overhang present in the first adapter sequence. In some embodiments, the solid support is a magnetic bead and the purification step includes magnetic separation.

In another embodiment of the method, the separated beads are suspended in a buffer comprising Bst 3.0 polymerase and nucleotide triphosphates (NTPs) at about 45° C. for about 15 minutes, thereby repairing and extending the nicked CRISPR sgRNA or CRISPR crRNA product, said extension causing displacement of repaired CRISPR sgRNA or CRISPR crRNA product from the bead. The method can also comprise transcribing the sgRNA template libraries in the presence of DNase I. Another embodiment of the method comprises PCR amplification of said ligated CRISPR sgRNA or CRISPR crRNA product. In certain aspects of the foregoing method, the digestion and ligation steps are performed essentially simultaneously.

In another aspect, a method for generating DNA templates for production of a CRISPR/Cas guide strand library is disclosed. An exemplary method comprises digesting a polynucleotide sample from a source of interest at protospacer adjacent motifs (PAM) with a first restriction enzyme (RE) to form target region containing fragments in the presence of a ligase and a first adapter sequence, said first adapter sequence comprising a constant region having a CRISPR enzyme binding site, said ligase operably linking said first adapter sequence to one or both ends of said fragments, thereby forming an intermediate product which lacks a binding site recognized by said first RE, said intermediate product comprising at least one binding site for a second Type II RE. The intermediate product is then digested with the second Type II RE in the presence of a ligase and a second adapter sequence, said Type II RE having methylation and nuclease activity which methylates said product at said recognition site and cleaves said targeting region between 17 to 27 base pairs away from said recognition site to form one or more methylated fragments. The resulting methylated polynucleotide fragments comprising operably linked CRISPR constant region and targeting region sequences, the presence of said methyl group preventing further digestion of said polynucleotide fragments by the second Type II RE, said second adapter optionally comprising one or more of a cloning sequence, one or more adapter sequences, and a vector backbone. The method further comprises immobilizing said polynucleotides to a solid support by immobilizing one or more first single stranded polynucleotide(s) to a solid support, said first single stranded polynucleotide having 5′ and 3′ ends and having binding affinity for at least a portion of a second polynucleotide(s), said 5′ end of the first single stranded polynucleotide protruding from the solid support. The immobilized one or more first polynucleotide(s) are then contacted with one or more second polynucleotide(s) of sufficient complementarity such that a polynucleotide duplex forms, thereby immobilizing the one or more second polynucleotide(s) on the solid support. The resulting polynucleotide duplexes are then contacted with a polymerase having strand displacement activity in the presence of dNTPs, the second polynucleotide serving as a template for extension by said polymerase, wherein the first polynucleotide is displaced by the extension of the second polynucleotide, and said extension preventing rehybridization between the first and second polynucleotides, said second polynucleotide encoding a CRISPR sgRNA or CRISPR crRNA.

In another aspect, a method for eluting polynucleotides from solid supports is disclosed. An exemplary method comprises immobilizing one or more first single stranded polynucleotide(s) to a solid support, the first single stranded polynucleotide having 5′ and 3′ ends and having binding affinity for at least a portion of a second polynucleotide(s), said 5′ end of the first single stranded polynucleotide protruding from the solid support; contacting the one or more first polynucleotide(s) with one or more second polynucleotide(s) of sufficient complementarity such that a polynucleotide duplex forms, thereby immobilizing the one or more second polynucleotide(s) on the solid support; and contacting the polynucleotide duplex(s) so formed with a polymerase having strand displacement activity in the presence of dNTPs, the second polynucleotide serving as a template for extension by said polymerase, wherein the first polynucleotide is displaced by the extension of the second polynucleotide, and said extension preventing rehybridization between the first and second polynucleotides, said second polynucleotide encoding a CRISPR sgRNA or CRISPR crRNA. In certain embodiments, the support is a bead or a column. In other embodiments, the support is a magnetic bead. In other embodiments, the attached polynucleotide is a plurality of polynucleotides, preferably, a plurality of polynucleotides with different sequences. The method can further comprise purifying the second polynucleotide from a mixture. In one approach, the length of the hybridizing sequences is adjusted to increase or decrease the temperature at which hybridization will occur. In other approaches, polynucleotides of interest are purified at different temperatures. In certain aspects, the polynucleotides are purified via a process selected from the group consisting of magnetic separation, precipitation, hybridization, antibody isolation, and co-precipitation. Preferably,

one or more second polynucleotides encode for one or more CRISPR/Cas9 guide RNAs.

In certain aspects, certain intermediate or ligated products comprise at least five phosphorothioate linked polynucleotides at the 5′ end, and any unwanted polynucleotides present in the reaction are degraded by contacting the reaction with first exonuclease and second exonucleases which cleave double stranded and single stranded DNA respectively, said phosphorothioate containing polynucleotides being resistant to exonuclease cleavage.

In certain aspects, the aforementioned phosphorothioates are incorporated by ligating adapters or via PCR.

Also provided is a kit for production of an sgRNA guide strand library comprising: a first polynucleotide which encodes an RNA bound by a Cas enzyme, comprising a constant region encoding sequence for a CRISPR single guide RNA (sgRNA) or CRISPR targeting RNA (crRNA) having a non-palindromic recognition site recognized by a type II restriction enzyme oriented in said sequence such that a second operably linked polynucleotide is cleaved 17 to 27 base pairs from said recognition site, when operably linked, said type II restriction enzyme having methylase activity and cleavage activity, said recognition site comprising a nucleotide which is methylated by said type II restriction enzyme upon cleavage, methylation of said nucleotide altering said recognition site such that said type II enzyme no longer binds said site. In certain aspects the kit can comprise second polynucleotides encoding a variable or targeting sequence. The kit can also contain one or more ligases for operably linking the constant region and targeting polynucleotides or one or more adapters. The kit may also contain one or more Type II restriction enzymes. In certain aspects, the kit includes a solid support. The kit can comprise a strand displacing polymerase. The kit may also include one or more adapter sequences encoding a promoter and/or cloning site and/or phosphorothioate linked polynucleotides. The kit can further comprise buffers suitable for simultaneous digestion and ligation of a polynucleotide; and optionally, reagents suitable for PCR amplification. The kit can also contain reagents suitable for normalization of input nucleic acid sample.

### BRIEF DESCRIPTION OF THE DRAWINGS

FIG. 1. A DNA substrate is cleaved by a first type II restriction endonuclease (RE) containing a PAM motif in its recognition sequence and ligation of a first adapter containing the CRISPR guide RNA scaffold sequence is carried out in a single reaction. A modification to the scaffold sequence prevents the first adapter from being cleaved from the DNA substrate after being ligated. The adapter ligation is therefore irreversible and forces the reaction to proceed toward the formation of the intermediate product. In the embodiment shown, the first adapter was synthesized without a phosphate group on the end to be ligated. This prevents the first adapter from ligating to other first adapters, which could result in the final library having truncated products. Because the first adapter is not phosphorylated, the intermediate ligation product will contain a nick.

FIG. 2. Digestion of the intermediate product (from the first reaction) by a second type II RE and ligation of a second adapter containing a promoter sequence is carried out in a single reaction. The addition of a methyl moiety to the RE binding site in the first adapter by the second RE prevents cleavage of the second adapter after it has been ligated. Despite the nick in the intermediate product, the second RE can still bind to the recognition sequence and cut across the nick. The second adapter was also synthesized without a phosphate group on the end to be ligated. This prevents the second adapter from ligating to other second adapters, increasing the final yield of the library. In order to maintain the sequence modification shown in FIG. 1, an engineered version of MmeI was used that would recognize a sequence which maintained this first modification. The asterisk (*) indicates that the reaction is not strictly reversible because the methyl group is incorporated during digestion. Excess adapter drives the reaction to the final product.

FIG. 3. The CRISPR Scaffold adapter is synthesized with a long, 5′, single-stranded overhang that is capable of hybridizing to a single stranded oligo immobilized on a solid support, such as a magnetic. Incubation of the library with a surface coated with a capture oligo, with the 5′ end protruding, immobilized the library on the surface and can be purified from buffers, protein, and nucleic acid by physical separation. A strand displacing polymerase can be added to permanently remove the fragments from the solid support. This is due to the directionality of the oligo attached to the beads and extension direction of the polymerase. The captured oligos are used as a template by the polymerase and extension by the polymerase prevents re-hybridization. Double stranded DNA can then be preferentially isolated using standard silica-based separation techniques. Immobilization of the library to the solid support can occur at any point in the library construction process.

FIG. 4. Phosphorothioate linkages (shown in black) are synthesized into the adapters on the outside ends of the 5′ strand. After the library has been constructed, there could be many byproducts, but only the final product has both of the 5′ ends protected from degradation. Addition of a double stranded, 5′ to 3′ DNA endonuclease that is blocked by the protecting groups as well as a single stranded DNA endonuclease will cause the degradation of any remaining adapters, DNA substrates, ligation products, and intermediate products, but the final library will be protected. PCR primers could also be used to incorporate the linkages into a DNA product that could then be subjected to the endonucleases. Additionally, a 3′ to 5′ endonuclease could also be used if protecting groups are incorporated into the outside ends of the 3′ strand.

FIGS. 5A to 5D provide an example embodiment of the method wherein the first adaptor only contains a portion of the sgRNA or crRNA sequence. FIG. 5A shows a restriction digestion map of a representative template for an sgRNA modified to contain an MmeI site (SeqID 454). Type II enzymes that cut once in the region are shown. FIG. 5B provides a first adapter created to contain an MmeI site, an overhang for ligation with a second DNA fragment, and the portion of the sgRNA template sequence up to the SetI restriction site. FIG. 5C shows a final product generated using the first adapter in FIG. 5B and a second adapter containing a T7 promoter and a second restriction site (BamHI). FIG. 5C shows that the final product can be digested using BamHI and SetI and ligated into a plasmid containing the remainder of the sgRNA template and a cloning site digested with the above enzymes or an isoschizomer thereof, thus creating a complete sgRNA template. Ellipses indicate that the two ends are connected.

FIG. 6A shows the hairpin structure of a wildtype crRNA (SEQ ID NO: 423) for CRISPR Cas9.

FIG. 6B shows polynucleotides of various embodiments encoding for an sgRNA or crRNA recognized by a CRIPSR Cas9 protein and having non-palindromic recognition site for MmeI, NmeAIII, HpaII or MspI, ScrFI, and BfaI. S. pyogenes crRNA sequence was modified to insert binding sites for restriction enzymes while maintaining secondary structure. WT crRNA indicates the previously published CRISPR sgRNA sequence for Cas9 binding. Areas surrounded by dotted lines indicate the hairpin region. Binding sites are shown surrounded by a solid line. Underlined letters indicate bases that do not match the original sequence. SEQ ID NOs: 424 to 439 are shown in FIG. 6B in descending order.

FIG. 7A shows the hairpin structure of a wildtype crRNA (SEQ ID NO: 440) for CRISPR Cpf1.

FIG. 7B shows polynucleotides of various embodiments encoding for an sgRNA or crRNA recognized by a CRIPSR Cpf1 protein and having non-palindromic recognition site for MmeI and NmeAIII. F. novicida crRNA sequence was modified to insert an MmeI binding site while maintaining secondary structure. WT crRNA indicates the previously published CRISPR sgRNA sequence for Cpf1 binding. Areas surrounded by dotted lines indicate the hairpin region. Cpf1-based systems lack a tracrRNA. MmeI and NmeAIII binding sites are shown surrounded by a solid line. Underlined letters indicate bases that do not match the original sequence. O.H. indicates the presence of an overhang added to the crRNA. SEQ ID NOs: 441 to 453 are shown in FIG. 7B in descending order.

### DETAILED DESCRIPTION

A rapid and efficient enzymatic method to generate sgRNA libraries is described herein, which is applicable to a large variety of DNA substrates. The methods disclosed significantly reduce the cost of custom library generation while maintaining fidelity in the guide strands produced. Input DNA is obtained using one of several different methods, and optionally normalized. A series of digestion and ligation steps are performed to create DNA templates that are transcribed into sgRNAs. By altering the sequence of the first stem loop of the sgRNA and by taking advantage of the methyltransferase activity of type IIS restriction enzymes, we were able to develop a method that combines restriction digestion and ligation steps into single reactions. We also developed methods for purification and handling of the library during the synthesis process. Together, these steps result in a method that delivers high yield, fidelity, efficiency, and ease of use.

### Terms Commonly Used in the Art of Library Construction

The terms “polynucleotide”, “nucleotide”, “nucleotide sequence”, “nucleic acid”, and “oligonucleotide” are used interchangeably in this disclosure. They refer to a polymeric form of nucleotides of any length, either deoxyribonucleotides or ribonucleotides, or analogs thereof. Polynucleotides may have any three-dimensional structure, and may perform any function, known or unknown. The following are non-limiting examples of polynucleotides: single-, double-, or multi-stranded DNA or RNA, genomic DNA, cDNA, DNA-RNA hybrids, or a polymer comprising purine and pyrimidine bases or other natural, chemically or biochemically modified, non-natural, or derivatized nucleotide bases. The terms “polynucleotide” and “nucleic acid” should be understood to include, as applicable to the embodiment being described, single-stranded (such as sense or antisense) and double-stranded polynucleotides. A polynucleotide may comprise one or more modified nucleotides, such as methylated nucleotides and nucleotide analogs. If present, modifications to the nucleotide structure may be imparted before or after assembly of the polymer. The sequence of nucleotides may be interrupted by non-nucleotide components. A polynucleotide may be further modified after polymerization, such as by conjugation with a labeling component.

The term “exogenous” nucleic acid can refer to a nucleic acid that is not normally or naturally found in or produced by a given bacterium, organism, or cell in nature. The term “endogenous” nucleic acid can refer to a nucleic acid that is normally found in or produced by a given bacterium, organism, or cell in nature.

The term “recombinant” is understood to mean that a particular nucleic acid (DNA or RNA) or protein is the product of various combinations of cloning, restriction, or ligation steps resulting in a construct having a structural coding or non-coding sequence distinguishable from endogenous nucleic acids found in natural systems.

The terms “construct”, “cassette”, “expression cassette”, “plasmid”, “vector”, or “expression vector” is understood to mean a recombinant nucleic acid, generally recombinant DNA, which has been generated for the purpose of the expression or propagation of a nucleotide sequence(s) of interest, or is to be used in the construction of other recombinant nucleotide sequences.

The term “promoter” or “promoter polynucleotide” is understood to mean a regulatory sequence/element or control sequence/element that is capable of binding/recruiting an RNA polymerase and initiating transcription of sequence downstream or in a 3′ direction from the promoter. A promoter can be, for example, constitutively active, or always on, or inducible in which the promoter is active or inactive in the presence of an external stimulus. Example of promoters include T7 promoters or U6 promoters.

An “adapter or adaptor”, or a “linker” for use in the compositions and methods described herein is a short, chemically synthesized, single-stranded or double-stranded oligonucleotide that can be ligated to the ends of other DNA or RNA molecules. Double stranded adapters can be synthesized to have blunt ends to both terminals or to have sticky end at one end and blunt end at the other, or sticky ends at both ends. For instance, a double stranded DNA adapter can be used to link the ends of two other DNA molecules (i.e., ends that do not have “sticky ends”, that is complementary protruding single strands by themselves). It may be used to add sticky ends to cDNA allowing it to be ligated into the plasmid much more efficiently. Two adapters could base pair to each other to form dimers. A conversion adapter is used to join a DNA insert cut with one restriction enzyme, say EcoRl, with a vector opened with another enzyme, Bam Hl. This adapter can be used to convert the cohesive end produced by Bam Hl to one produced by Eco Rl or vice versa. One of its applications is ligating cDNA into a plasmid or other vectors instead of using Terminal Deoxynucleotide Transferase enzyme to add poly A to the cDNA fragment.

The term “operably linked” can mean the positioning of components in a relationship which permits them to function in their intended manner. For example, a promoter can be linked to a polynucleotide sequence to induce transcription of the polynucleotide sequence.

The terms “sequence identity” or “identity” refers to a specified percentage of residues in two nucleic acid or amino acid sequences that are identical when aligned for maximum correspondence over a specified comparison window, as measured by sequence comparison algorithms or by visual inspection. When sequences differ in conservative substitutions, the percent sequence identity may be adjusted upwards to correct for the conservative nature of the substitution. Sequences that differ by such conservative substitutions are said to have “sequence similarity” or “similarity.” Means for making this adjustment are well known to those of skill in the art. Typically this involves scoring a conservative substitution as a partial rather than a full mismatch, thereby increasing the percentage sequence identity.

The term “comparison window” refers to a segment of at least about 20 contiguous positions in which a sequence may be compared to a reference sequence of the same number of contiguous positions after the two sequences are aligned optimally. In a refinement, the comparison window is from 15 to 30 contiguous positions in which a sequence may be compared to a reference sequence of the same number of contiguous positions after the two sequences are aligned optimally. In another refinement, the comparison window is usually from about 50 to about 200 contiguous positions in which a sequence may be compared to a reference sequence of the same number of contiguous positions after the two sequences are aligned optimally.

“Normalization” is the process of ensuring equal representation of different polynucleotides. For example, the number of RNA copies per gene in a sample will depend on the expression level of that gene. These levels can vary over several orders of magnitude. However, it is often desirable to identify or include genes with very low expression level during data analysis or library generation. In order to improve representation of low expression genes, a number of methods can be used to flatten the distribution of polynucleotides in the sample to ensure all polynucleotide species are represented at roughly equal levels.

The terms “complementarity” or “complement” refer to the ability of a nucleic acid to form hydrogen bond(s) with another nucleic acid sequence by either traditional Watson-Crick or other non-traditional types. A percent complementarity indicates the percentage of residues in a nucleic acid molecule which can form hydrogen bonds (e.g., Watson-Crick base pairing) with a second nucleic acid sequence (e.g., 4, 5, and 6 out of 6 being 66.67%, 83.33%, and 100% complementary). “Perfectly complementary” means that all the contiguous residues of a nucleic acid sequence will hydrogen bond with the same number of contiguous residues in a second nucleic acid sequence. “Substantially complementary” as used herein refers to a degree of complementarity that is at least 40%, 50%, 60%, 62.5%, 70%, 75%, 80%, 85%, 90%, 95%, 97%, 98%, 99%, or 100%, or percentages in between over a region of 4, 5, 6, 7, and 8 nucleotides, or refers to two nucleic acids that hybridize under stringent conditions.

A “selected phenotype” refers to any phenotype, e.g., any observable characteristic or functional effect that can be measured in an assay such as changes in cell growth, proliferation, morphology, enzyme function, signal transduction, expression patterns, downstream expression patterns, reporter gene activation, hormone release, growth factor release, neurotransmitter release, ligand binding, apoptosis, and product formation. Such assays include, e.g., transformation assays, e.g., changes in proliferation, anchorage dependence, growth factor dependence, foci formation, growth in soft agar, tumor proliferation in nude mice, and tumor vascularization in nude mice; apoptosis assays, e.g., DNA laddering and cell death, expression of genes involved in apoptosis; signal transduction assays, e.g., changes in intracellular calcium, cAMP, cGMP, IP3, changes in hormone and neurotransmitter release; receptor assays, e.g., estrogen receptor and cell growth; growth factor assays, e.g., EPO, hypoxia and erythrocyte colony forming units assays; enzyme product assays, e.g., FAD-2 induced oil desaturation; transcription assays, e.g., reporter gene assays; and protein production assays, e.g., VEGF ELISAs. A candidate gene is “associated with” a selected phenotype if modulation of gene expression of the candidate gene causes a change in the selected phenotype

In general, a CRISPR system is characterized by elements that promote the formation of a CRISPR complex at the site of a target sequence (also referred to as a protospacer in the context of an endogenous CRISPR system). In the context of formation of a CRISPR complex, “target sequence” refers to a sequence to which a guide sequence is designed to have complementarity, where hybridization between a target sequence and a guide sequence promotes the formation of a CRISPR complex. Full complementarity is not necessarily required, provided there is sufficient complementarity to cause hybridization and promote formation of a CRISPR complex. A target sequence may comprise any polynucleotide, such as DNA or RNA polynucleotides. In some embodiments, a target sequence is located in the nucleus or cytoplasm of a cell. In some embodiments, the target sequence may be within an organelle of a eukaryotic cell, for example, mitochondrion or chloroplast. A sequence or template that may be used for recombination into the targeted locus comprising the target sequences is referred to as an “editing template” or “editing polynucleotide” or “editing sequence”. In aspects of the invention, an exogenous template polynucleotide may be referred to as an editing template. In an aspect of the invention the recombination is homologous recombination.

In general, a guide sequence is any polynucleotide sequence having sufficient complementarity with a target polynucleotide sequence to hybridize with the target sequence and direct sequence-specific binding of a CRISPR complex to the target sequence. In some embodiments, the degree of complementarity between a guide sequence and its corresponding target sequence, when optimally aligned using a suitable alignment algorithm, is about or more than about 85%, 90%, 95%, 97.5%, 99%, or more. Optimal alignment may be determined with the use of any suitable algorithm for aligning sequences, non-limiting example of which include the Smith-Waterman algorithm, the Needleman-Wunsch algorithm, algorithms based on the Burrows-Wheeler Transform (e.g. the Burrows Wheeler Aligner), ClustalW, Clustal X, BLAT, Novoalign (Novocraft Technologies, ELAND (Illumina, San Diego, Calif.), SOAP (available at soap.genomics.org.cn), and Maq (available at maq.sourceforge.net).

In various embodiments are disclosed polynucleotide or polynucleotides encoding for a constant region of a CRISPR single guide RNA (sgRNA) or CRISPR targeting RNA (crRNA) comprising a non-palindromic recognition site for a type II restriction enzyme, the non-palindromic recognition site being oriented in a manner recognized by the type II restriction enzyme for cutting a site that is 17 to 27 base pairs past an end of the constant region in an operably linked target region polynucleotide, if present. In various embodiments, the polynucleotides encode for a plurality of sgRNAs or crRNAs. The polynucleotide of various embodiments is double-stranded with sense and antisense strands and the non-palindromic recognition site of various embodiments is oriented in a manner recognized by the type II restriction enzyme for cutting a site that is 17 to 27 base pairs upstream from a 5′ end of the sense strand or downstream from a 3′ end of the antisense strand. In other embodiments, the polynucleotide is double-stranded with sense and antisense strands and the non-palindromic recognition site of various embodiments is oriented in a manner recognized by the type II restriction enzyme for cutting a site that is 17 to 27 base pairs downstream from a 3′ end of the sense strand or upstream from a 5′ end of the antisense strand.

CRISPR protein forms a complex with a guide RNA and is capable of binding or modifying by, for example, cleaving, nicking, methylating, or demethylating a target nucleic acid or a polypeptide associated with the target nucleic acid. One example of a CRISPR protein, CRISPR/Cas9, is described in PCT Patent Application Publication No. WO 2016/196805 and references referred in WO 2016/196805, which are also incorporated in its entirety by reference herein. The Cas9 protein utilizes variable regions to bind specific sequences of DNA in a genome. Examples of Cas9 proteins are from Streptococcus pyogenes or Staphylococcus aureus. The Cas9 protein utilizes guide RNAs to bind specific regions of a DNA sequence. Cpf1 is another protein, which uses a guide RNA in order to bind a specific sequence in genomic DNA. Cpf1 is from Francisella novicida and also cuts DNA making a staggered cut. U.S. patent application Ser. No. 15/727,279 is also incorporated by reference herein.

CRISPR proteins such as Cas9 and Cpf1 utilize variable regions to bind specific sequences of DNA in a genome. Particularly, CRISPR proteins such as Cpf1 and Cas9 use a guide RNA. The guide RNA provides target specificity to the complex by having a nucleotide sequence that is complementary to a sequence of a target nucleic acid. A number of methods have been employed to create guide RNAs. In one example, two RNA segments known as CRISPR RNA (crRNA) and trans-activating crRNA (tracrRNA) have complementary portions allowing them to combine to form an RNA duplex known as a crRNA/tracrRNA complex. The crRNA/tracrRNA complex has structures such as or similar to hairpin/stem-loop structures and are recognized by CRISPR proteins. Another method is using single RNA segments (e.g. single guide RNA or sgRNA) that can form hairpin or stem-loop structures and are recognized by CRISPR proteins. The guide RNA such as crRNA and sgRNA includes two segments: a variable region, which is also known as a targeting region; and a constant region, which is also known as a scaffold region to which the CRISPR protein binds.

The term “region” is understood to mean a segment/section/region of a molecule, e.g., a contiguous stretch of nucleotides in a nucleic acid molecule. A “region” can also mean a region/section of a complex such that a segment may comprise regions of more than one molecule. The guide RNA includes hairpin regions, which are conserved regions which bind to the CRISPR proteins such as Cas9 and Cpf1. These hairpin regions are located in the constant region of the guide RNA.

Non-limiting examples of Cas proteins include those listed below. In some embodiments, a vector comprises a regulatory element operably linked to an enzyme-coding sequence encoding a CRISPR enzyme, such as a Cas protein. Non-limiting examples of Cas proteins include Cas1, Cas1B, Cas2, Cas3, Cas4, Cas5, Cas6, Cas7, Cas8, Cas9 (also known as Csn1 and Csx12), Cas10, Csy1, Csy2, Csy3, Cse1, Cse2, Csc1, Csc2, Csa5, Csn2, Csm2, Csm3, Csm4, Csm5, Csm6, Cmr1, Cmr3, Cmr4, Cmr5, Cmr6, Csb1, Csb2, Csb3, Csx17, Csx14, Csx10, Csx16, CsaX, Csx3, Csx1, Csx15, Csf1, Csf2, Csf3, Csf4, homologs thereof, or modified versions thereof. These enzymes are known; for example, the amino acid sequence of S. pyogenes Cas9 protein may be found in the SwissProt database under accession number Q99ZW2. In some embodiments, the unmodified CRISPR enzyme has DNA cleavage activity, such as Cas9. In some embodiments the CRISPR enzyme is Cas9, and may be Cas9 from S. pyogenes or S. pneumoniae. In some embodiments, the CRISPR enzyme directs cleavage of one or both strands at the location of a target sequence, such as within the target sequence and/or within the complement of the target sequence. In some embodiments, the CRISPR enzyme directs cleavage of one or both strands within about 1, 2, 3, 4, 5, 6, 7, 8, 9, 10, 15, 20, 25, 50, 100, 200, 500, or more base pairs from the first or last nucleotide of a target sequence. In some embodiments, a vector encodes a CRISPR enzyme that is mutated with respect to a corresponding wild-type enzyme such that the mutated CRISPR enzyme lacks the ability to cleave one or both strands of a target polynucleotide containing a target sequence. For example, an aspartate-to-alanine substitution (D10A) in the RuvC I catalytic domain of Cas9 from S. pyogenes converts Cas9 from a nuclease that cleaves both strands to a nickase (cleaves a single strand). Other examples of mutations that render Cas9 a nickase include, without limitation, H840A, N854A, and N863A. In aspects of the invention, nickases may be used for genome editing via homologous recombination.

There are a variety of principles related to rapid and efficient construction of CRISPR/Cas9 guide RNA libraries taught herein. These principles may be implemented in a wide range of systems, methods and compositions. The descriptions given herein describe several illustrative examples of the application of these principles. The examples are only illustrative and are not limiting. The examples may be added to, simplified, or combined while still applying the principles described. For example, when an illustrative method describes steps, the steps may be combined, one or more of the steps may be removed, the steps may be reordered, and additional steps may be added.

Further, the various examples and specific implementations may be combined with other examples and implementations while still applying the principles described. For example, a first example may apply the principles in a first series of steps and/or compositions. A second example may apply the principles in a second series of steps and/or compositions. These two examples or portions thereof may be merged or combined in a variety of ways to accomplish the principles described. Additionally, techniques, methods, and compositions that are not specifically described may be applied and still be within the scope of the principles described herein.

In one embodiment, systems, methods, and compositions for the rapid and efficient construction of CRISPR/Cas9 guide RNA libraries may include a methyl moiety composition. For example, a polynucleotide encoding for a constant region of a CRISPR single guide RNA (sgRNA) or CRISPR targeting RNA (crRNA) may include a methyl moiety that prevents digestion of the polynucleotide by a restriction endonuclease. The methyl moiety may be added by an enzyme containing methyltransferase activity. The polynucleotide may further include a non-palindromic recognition site for a Type IIS restriction enzyme binding site.

In one example, there may be one or more sequence modifications to a scaffold composition. In one illustrative embodiment, a polynucleotide encoding for a constant region of a CRISPR single guide RNA (sgRNA) or CRISPR targeting RNA (crRNA) may include a sequence that when ligated to digested fragments does not restore the restriction enzyme binding site sequence used to digest the fragments. The modifications may maintain the stem loop structure of the CRISPR guide RNA molecule in order for the specificity and endonuclease activity of CRISPR RNA complexes to function. The polynucleotide may include a non-palindromic recognition site for a type II restriction enzyme and the non-palindromic recognition site may be oriented in a manner recognized by the type II restriction enzyme.

A kit for generating CRISPR guide RNA (gRNA) libraries may result in a polynucleotide containing a methyl moiety and/or encoding for a constant region of a CRISPR sgRNA or CRISPR crRNA and having a sequence that does not restore a restriction enzyme binding site. The kit may include one or more of the following: a type II restriction enzyme, a solid support that is configured to immobilize the polynucleotide on the solid support, a strand displacing polymerase, a promoter polynucleotide recognized by an RNA polymerase and buffers that allow/facilitate digestion and ligation to occur in the same reaction vessel.

In one embodiment, a kit for generating CRISPR guide RNA (gRNA) libraries may include at least one of the following: a type II restriction enzyme, a solid support, wherein the polynucleotide is capable of being immobilized on the solid supports or is immobilized on the solid support.

A first polynucleotide may include a sequence encoding for a constant region of one of a CRISPR single guide RNA (sgRNA) and CRISPR targeting RNA (crRNA), the sequence may include a non-palindromic recognition site recognized by a type II restriction enzyme, wherein the non-palindromic recognition site is configured such that a second polynucleotide operably linked to the first polynucleotide is cleaved 17 to 27 base pairs from the recognition site by the type II restriction enzyme, where the type II restriction enzyme is configured to add a methyl moiety to the recognition site such that methylation of the recognition site prevents subsequent cleavage of the second polynucleotide.

Additionally or alternatively, a first polynucleotide may include a sequence encoding for a constant region of one of a CRISPR single guide RNA (sgRNA) and CRISPR targeting RNA (crRNA). The sequence may include a non-palindromic recognition site configured to be recognized by a type II restriction enzyme, wherein the non-palindromic recognition site is oriented such that a second polynucleotide operably linked to an end adjacent to the non-palindromic recognition site, the first polynucleotide configured to be cleaved 17 to 27 base pairs from the recognition site by the type II restriction enzyme. The type II restriction enzyme may add a methyl moiety to the recognition site such that methylation of the recognition site prevents subsequent cleavage of the second polynucleotide.

In one example, a first polynucleotide may include a sequence encoding for a constant region of one of a CRISPR single guide RNA (sgRNA) and CRISPR targeting RNA (crRNA), and an end configured to operably link a second polynucleotide. The sequence may include a non-palindromic recognition site recognized by a type II restriction enzyme, wherein the non-palindromic recognition site is configured to position and orient the type II restriction enzyme to cleave the second polynucleotide 17 to 27 base pairs from the recognition site. The type II restriction enzyme is configured to add a methyl moiety to the recognition site such that the methyl moiety prevents subsequent cleavage of the second polynucleotide.

In some embodiments, the non-palindromic recognition site is positioned and oriented such that the type II restriction enzyme is configured to cleave an operably linked second polynucleotide 17 to 27 base pairs from the recognition site.

In one embodiment, a polynucleotide may include a sequence encoding for at least part of protein binding segment of an RNA component of a CRISPR complex. The polynucleotide may include a non-palindromic recognition sequence for a type II restriction enzyme configured to cleave at least 17 nucleotides outside of the non-palindromic recognition sequence, the non-palindromic recognition sequence being oriented such that the type II restriction enzyme is configured such it cannot cleave within the sequence encoding for at least part of a protein binding segment of an RNA component of a CRISPR complex.

In one embodiment, a polynucleotide encoding at least part of a protein binding segment of an RNA component of a CRISPR complex may include: a non-palindromic recognition sequence for a type II restriction enzyme capable of cleaving at least 17 nucleotides outside of the non-palindromic recognition sequence, the recognition sequence being oriented such that, in the presence of the restriction enzyme, the DNA cleavage domain of the restriction enzyme is positioned outside of the sequence encoding for at least part of a protein binding segment of an RNA component of a CRISPR complex.

In some embodiments, a method for generating double stranded inputs for enzymatic CRISPR library generation, may include at least one of: selection of a DNA source and purification of polynucleotides from the source by physical or chemical separation. In one example, the DNA source is selected by one or more of an organism of interest, an organism at a selected stage of development, a tissue of interest, a cell at a selected stage of differentiation, a cell at a particular stage of the cell cycle, a tissue or cell having a selected pathology. The polynucleotides may be one or more of the following: cDNA created from RNA, genomic DNA, mitochondrial DNA, or other appropriate polynucleotides. The polynucleotides may be selected by at least one of: precipitation isolation, hybridization isolation, antibody isolation, and other co-precipitation isolation. The polynucleotides or one or more segments thereof may be amplified by PCR or other appropriate technique(s). The polynucleotides may be normalized as described above.

In one example, a method for generating DNA templates for production of a CRISPR/Cas9 guide RNA library may include providing a polynucleotide sample; digesting the polynucleotide sample at protospacer adjacent motifs (PAM) to form fragments with a first restriction enzyme (RE) in the presence of a ligase and first adapter, wherein the first adapter sequence may include a constant region containing a sequence CRISPR enzyme can bind to and/or cloning site sequence; attaching polynucleotide sequences to one or both ends of the fragments with a ligase, thereby forming an intermediate product which lacks binding sites recognized by the first RE; and digesting the second polynucleotide with the type II restriction enzyme to form a third polynucleotide encoding a CRISPR sgRNA or CRISPR crRNA, wherein the type II restriction enzyme cuts the DNA at a site that is 17 to 27 base pairs from the end of the first polynucleotide. The method may also include the step of ligating a polynucleotide to an end of the third polynucleotide. The ligated polynucleotide may include at least one of a promoter and a cloning adapter. The first adapter and second adapter may or may not contain a 5′ phosphate group.

In one example, a method for generating DNA templates for production of a CRISPR/Cas9 guide RNA library may include one or more of the following steps: providing a polynucleotide sample; digesting the polynucleotide sample at protospacer adjacent motifs (PAM) to form fragments with a first restriction enzyme (RE); ligation to a first adapter, wherein the first adapter sequence may include a constant region that may be a sequence a CRISPR enzyme can bind to. The method may further include connecting/linking polynucleotide sequences to one or both ends of the fragments with a ligase. The method may include contacting the intermediate product with a Type II S RE having methylase activity, in the presence of a ligase, the ligase operably linking a second adapter sequence to digested, methylated fragments formed from digestion of the intermediate product, thereby forming a ligated product having a variable or targeting region, and a scaffold region, wherein the second adapter sequence may include a promoter element and/or cloning site sequence.

In another example, a method for generating DNA templates for production of a CRISPR/Cas9 guide RNA library may include: providing a polynucleotide sample; digesting the polynucleotide sample at protospacer adjacent motifs (PAM) to form fragments with a first restriction enzyme (RE) in the presence of a ligase and first adapter, wherein the first adapter sequence may include a constant region containing a sequence a CRISPR enzyme can bind to and/or cloning site sequence, polynucleotide sequences to one or both ends of the fragments with a ligase, thereby forming an intermediate product which lacks binding sites recognized by the first RE. The method may also include contacting the intermediate product with a Type II S RE, in the presence of a ligase, the ligase operably linking a second adapter sequence to the digested fragments formed from digestion of the intermediate product, thereby forming a ligated product having a variable or targeting region, and a scaffold region, wherein the second adapter sequence may include a promoter element and/or cloning site sequence.

The method may include re-digestion that is blocked by simultaneous DNA modification of the DNA upon digestion. The modification in attachment of a methylation moiety. A cloning site sequence may be present, and the ligated product may be cloned into a vector which may include an operable promoter and/or scaffold sequence. When a promoter is present and the ligated product is cloned a vector may include a scaffold sequence.

One or more of these steps may be performed in a single reaction vessel. In one example, the first polynucleotide may be a plurality of first polynucleotides and at least a portion of the plurality of first polynucleotides are ligated with DNA to form a plurality of second polynucleotides. The plurality of second polynucleotides may be digested with the type II restriction enzyme having methylase activity to form a plurality of third polynucleotides encoding a plurality of CRISPR sgRNAs or CRISPR crRNAs. At least one of the plurality of CRISPR sgRNAs or CRISPR crRNAs may have a variable region different from the other CRISPR sgRNAs or CRISPR crRNAs.

The polynucleotide sample may be cDNA which is normalized to remove repeated transcripts from the cDNA, thereby increasing equal representation of transcripts in the library. The input polynucleotide sample may be obtained from a source selected from the group including: an organism of interest, an organism at a selected stage of development, a tissue of interest, a cell at a selected stage of differentiation, a cell at a particular stage of the cell cycle, a tissue or cell having a selected pathology, or other appropriate tissue or organism.

The components and intermediate products may have a number of different characteristics. For example, the adapters may lack a 5′ phosphate and the adapters may contain at least six consecutive phophorothioates at the 5′ end. The promoter may be a T7 RNA polymerase promoter. The polynucleotide sequence described above may be digested with an enzyme selected from the group consisting of HpaII, MspI, ScrFI, BfaI, and PacI. The ligated product of step may include at least one nick.

The method may include purifying the ligated product. For example, the ligated product may be purified using a capture oligonucleotide that may include a biotin at a 3′ end, which hybridizes to the scaffold portion of the ligated product operably linked to a solid support. In one example, the solid support is a magnetic bead and the purification step includes magnetic separation. Further the method may include suspending the separated beads in a buffer that may include Bst 3.0 polymerase and nucleotide triophosphates (NTPs) at about 45° C. for about 15 minutes, thereby repairing and extending the nicked strand, the extension causing displacement of the repaired product from the bead.

The method may also include transcribing the sgRNA template libraries in the presence of DNase I. The method may further include elution of the sgRNA template from the beads followed by PCR amplification of the sgRNA template. The first and second RE may be selected from the appropriate enzymes shown above or other appropriate enzymes. The digestion and ligation may be performed essentially simultaneously. A functional gRNA template produced by the method described herein may include one or more of the following: operably linked sequences, a promoter, a protospacer, an adapter, a RE II site, and a modified scaffold sequence. In one example, a genome wide library may include a plurality of unique CRISPR-Cas system guide sequences that are capable of targeting a plurality of target sequences in genomic loci, wherein the library is created using one of the methods described herein.

One method for eluting polynucleotides from solid supports, may include at least one of the following steps: attaching single stranded polynucleotide(s) to a solid support such that the 5′ end of the polynucleotide is protruding from the solid support and is capable of hybridizing with at least a portion of a second polynucleotide(s); contacting the attached polynucleotide with a second polynucleotide such that hybridization between the two polynucleotides can occur, forming a polynucleotide duplex, immobilizing the second polynucleotide; contacting the hybridized duplex with a polymerase exhibiting strand displacement activity in the presence of dNTPs, in which the second polynucleotide is used as a template for extension by the polymerase, wherein the first polynucleotide is displaced by the extension of the polymerase, and extension prevents rehybridization of the first and second polynucleotides. In one example, the second polynucleotide contains sequences encoding for CRISPR/Cas9 guide RNAs.

In one example, the support may be a bead which may or may not be magnetic or paramagnetic. The attached polynucleotide may be a plurality of polynucleotides which may or may not have different sequences. The method above may include a step of using the support to purify the second polynucleotide from a mixture. In some examples, hybridization may occur at different temperatures and the length of the hybridizing sequence can be adjusted to increase or decrease the temperature at which hybridization will occur. The method for eluting may also include a physical separation process.

Selectively degrading unwanted polynucleotide components of a mixture may provide enzymatic protection of a CRISPR guide RNA (gRNA) library. In one example, a method for selectively degrading unwanted polynucleotide components of a mixture, may include one or more of the following steps: incorporation of at least five nucleotides linked by phosphorothioates at the 5′ ends of the double stranded DNA fragment to be protected; contacting the double stranded DNA fragments with a first exonuclease that operates on double stranded DNA and is sensitive to phosphorothioate linkages and a second exonuclease that operates on single stranded DNA. In one example, polynucleotides with no protection or with a single end are degraded. The phosphorothioates may be incorporated by ligating adapters and/or incorporated by PCR.

In one example, a method for manipulation of DNA substrates through enzymatic processes to produce nucleotide sequences may include one or more of the following steps: simultaneously digesting DNA by targeting a restriction enzyme to sites containing protospacer adjacent motif (PAM) sequences in the DNA to produce DNA fragments; ligating adaptors to ends of DNA fragments to produce an intermediate product containing the scaffold ligated with a DNA fragment of an arbitrary length; and simultaneously digesting and ligating the intermediate product to produce guide RNA templates.

Simultaneously digesting DNA and ligating adaptors to ends of the DNA fragments may include digesting the DNA in presence of a ligase and a first adaptor. The digesting may include a type IIS restriction enzyme to digest the intermediate product to create a guide RNA that may include an 18 to 25 base pair protospacer connected to the engineered polynucleotide. In some examples, the type IIS restriction enzyme blocks re-digestion of the ligation product. For example, the type IIS restriction blocks its own function after digestion by chemically modifying its own binding site. Chemically modifying the binding site may include attaching a methyl group.

The ligation may include connection of the digestion product to an upstream adapter to produce a guide RNA template containing an upstream adapter, a proto spacer and the engineered polynucleotide. The upstream adapter may include at least one of a promoter, a cloning site, or other DNA integration site. In some examples, the upstream adapter does not contain a 5′ phosphate on the ligated end and the ligated product contains at least one nick.

The method may further include purifying the guide RNA templates. This purification may include attaching polynucleotides in a sequence dependent hybridization to a bead, washing to remove reagents and fragments that are not attached to the beads, and eluting the guide RNA templates from the bead. The bead may be a magnetic or paramagnetic bead. The purification may include elution and nick repair of the guide RNA templates. In some examples, the elution and nick repair occur in the same reaction and may both be performed by a single enzyme. This single enzyme may include a strand displacing polymerase to simultaneously elute and repair nicks in the attached polynucleotides. The strand displacing polymerase may use the previous captured polynucleotide as a template to displace and fill in a sequence, thereby permanently displacing the polynucleotide from the beads. A hybridized segment of the previously captured polynucleotide may be made double stranded by the polymerase, thereby preventing rehybridization to the polynucleotide attached to the beads.

The method may also include selecting a DNA source, which may include choosing a species of organism, selecting at least one of: a developmental stage of the organism, a tissue maturation stage, a state of cell differentiation and a stage of a cell cycle, selecting environmental conditions the organism is subject to, and selecting a tissue or cell type from the organism to be the DNA source. DNA or RNA may be extracted from the selected DNA source, where the extracting may include at least one of: a chemical separation of cellular components and a physical separation of cellular components. The extracting may isolate a nucleic acid species of interest. The chemical separation of DNA or RNA may include amplification of the nucleic acid species. For example, the chemical separation may include enzymatic amplification of at least one region of the nucleic acid species. This enzymatic amplification may include polymerase chain reaction (PCR). The physical separation of DNA or RNA may include isolation of DNA or RNA by at least one of: precipitation isolation, hybridization isolation, antibody isolation, and other co-precipitation isolation. The extracting may include extracting RNA from the selected DNA source and then converting the RNA into DNA. In one example, the extracting may include normalizing the DNA by enzymatic or chemical methods applied to the DNA, where the step of normalizing may include balancing quantities of various DNA components in the mixture to ensure more equal representation in an output library.

In one example, a method for generating DNA templates for production of a CRISPR/Cas9 guide RNA library may include digesting DNA into digested products in the presence of a ligase and an adaptor, wherein the digested products are chemically impeded/driven/prevented from recombining/undigesting/reversing the digestion and are ligated to the adaptor in a single reaction vessel. In some embodiments, the digesting and ligating may include digesting the polynucleotide sample at protospacer adjacent motifs (PAM) to form fragments with a first restriction enzyme (RE) in the presence of a ligase and first adapter, wherein the first adapter sequence may include a constant region containing a sequence CRISPR enzyme can bind to and/or cloning site sequence, polynucleotide sequences to one or both ends of the fragments with a ligase, thereby forming an intermediate product which lacks binding sites recognized by the first RE.

The method may include digesting the second polynucleotide with the type II restriction enzyme to form a third polynucleotide encoding a CRISPR sgRNA or CRISPR crRNA, wherein the type II restriction enzyme cuts the DNA at a site that is 17 to 27 base pairs from the end of the first polynucleotide.

In one example, a method for manipulation of DNA substrates through enzymatic processes to produce nucleotide sequences includes at least one of the following steps:

1. selecting a DNA source, where the selecting includes one or more of the following:
a. choosing a species of organism,
b. selecting at least one of: a developmental stage of the organism, a tissue maturation stage, a state of cell differentiation and a stage of a cell cycle,
c. selecting environmental conditions the organism is subject to, and
d. selecting a tissue or cell type from the organism to be the DNA source;
   2. extracting DNA or RNA from the selected DNA source, wherein extracting may include:
a. a chemical separation of cellular components and a physical separation of cellular components, wherein the extracting isolates a nucleic acid species of interest;
b. wherein chemical separation of DNA or RNA may include amplification of the nucleic acid species, wherein chemical separation may include enzymatic amplification of at least one region of the nucleic acid species, wherein the enzymatic amplification may include a polymerase chain reaction (PCR);
   i. wherein physical separation of DNA or RNA includes isolation of DNA or RNA by at least one of: precipitation isolation, hybridization isolation, antibody isolation, and other co-precipitation isolation;
   ii. wherein extracting may include extracting RNA from the selected DNA source and then converting the RNA into DNA;
   iii. wherein extracting may include normalizing the DNA using enzymatic or chemical methods applied to the DNA, wherein normalizing may include balancing quantities of various DNA components in the mixture to ensure more equal representation in an output library;
      3. simultaneously digesting the extracted DNA by targeting a restriction enzyme to sites containing PAM sequences to produce DNA fragments and ligating adaptors to ends of DNA fragments to produce an intermediate product containing the scaffold ligated with a DNA fragment of an arbitrary length;
a. wherein ligating may include introducing an engineered polynucleotide sequence that can associate with a CAS9 molecule (scaffold) such that the engineered polynucleotide sequence provides at least one of the following:
   i. prevents recreation of the restriction enzyme binding site upon ligation,
   ii. contains a type IIS binding site such that enzyme will cut between 18 and 25 base pairs upstream of the adapter,
   iii. maintains essential base pair interactions needed to maintain the endonuclease activity and binding specificity of the CAS9,
   iv. allows ligation of the digested DNA fragments by having a blunt end or containing an overhang compatible with the ends produced by enzymatic digestion;
   v. wherein the engineered polynucleotide does not contain a 5′ phosphate on the ligated end;
   vi. wherein the ligation product contains at least one nick;
b. wherein the engineered polynucleotide sequence does not maintain the G-U hydrogen bond of the G at position 1 of the scaffold sequence, wherein the G is replaced by another base to meet conditions described above;
c. wherein the engineered polynucleotide sequence may include a single gRNA scaffold or crRNA sequence or an adapter sequence for cloning into a vector containing either a single gRNA scaffold or crRNA sequence;
   4. simultaneously digesting and ligating the intermediate product to produce a ligated product;
a. wherein the digesting may include a type IIS restriction enzyme to digest the intermediate product to create a guide RNA may include an 18 to 25 base pair protospacer connected to the engineered polynucleotide;
   i. wherein the type IIS restriction enzyme blocks redigestion of the ligation product;
   ii. wherein the type IIS restriction blocks its own function after digestion by chemically modifying its own binding site; wherein the chemical modifying its own binding site may include attaching a methyl group;
b. wherein ligation may include connection of the digestion product to an upstream adapter to produce a guide RNA template containing an upstream adapter, a protospacer and the engineered polynucleotide; wherein the upstream adapter may include at least one of a promoter, a cloning site, or other DNA integration site;
   i. wherein the upstream adapter does not contain a 5′ phosphate on the ligated end;
   ii. wherein the ligated product contains at least one nick;
      5. purifying the guide RNA templates;
a. wherein purifying may include attaching polynucleotides in a sequence dependent hybridization to a bead, washing to remove reagents and fragments that are not attached to the beads, and eluting the guide RNA templates from the bead;
   i. wherein the bead may include a magnetic or paramagnetic bead;
b. wherein purifying may include elution and nick repair of the guide RNA templates;
   i. wherein the elution and nick repair occur in the same reaction;
   ii. wherein the elution and nick repair are both performed by a single enzyme;
   iii. wherein the single enzyme may include a strand displacing polymerase to simultaneously elute and repair nicks in the attached polynucleotides;
      1. wherein the strand displacing polymerase uses the previous captured polynucleotide as a template to displace and fill in a sequence, thereby permanently displacing the polynucleotide from the beads;
      2. wherein a hybridized segment of the previously captured polynucleotide is made double stranded by the polymerase thereby preventing rehybridization to the polynucleotide to the beads.

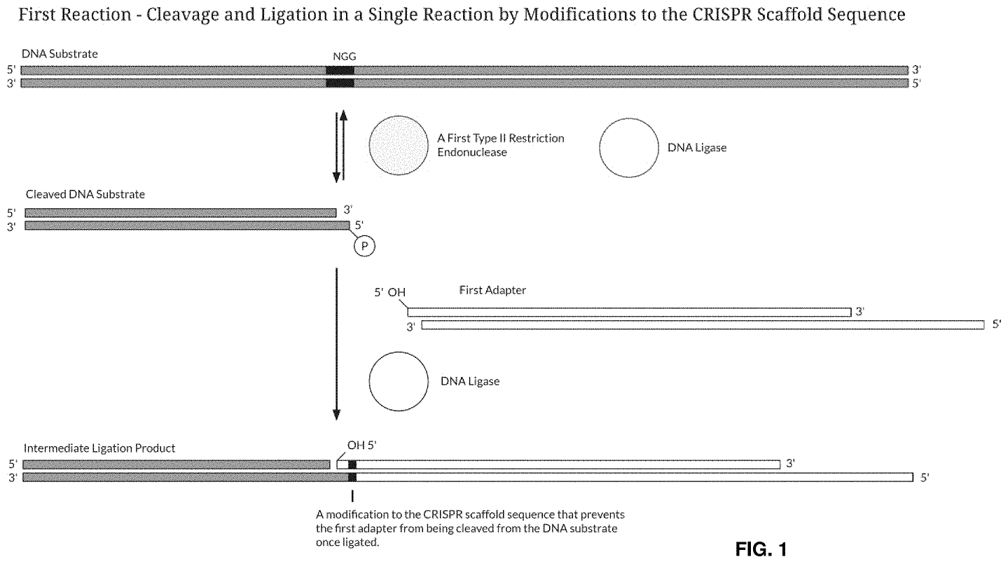

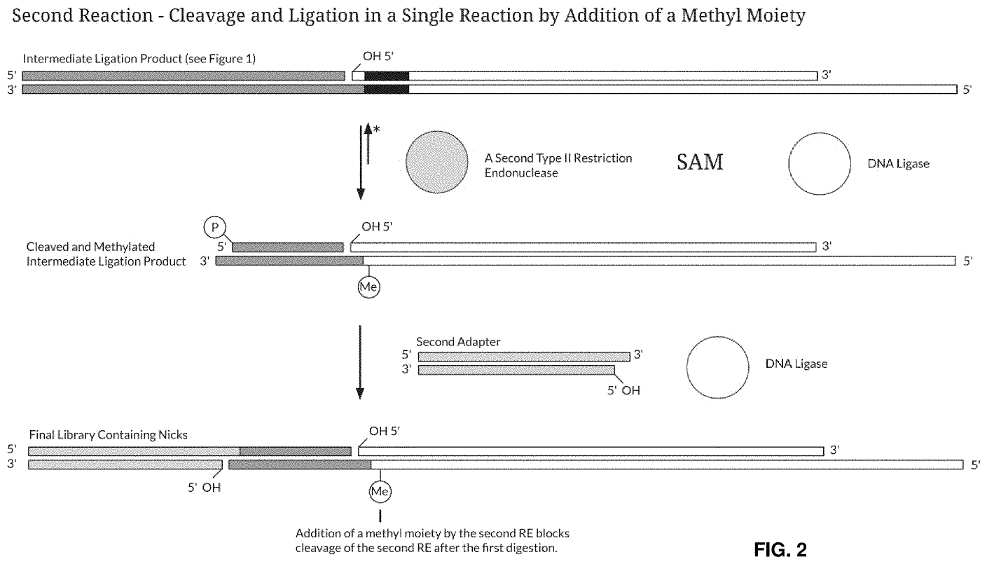

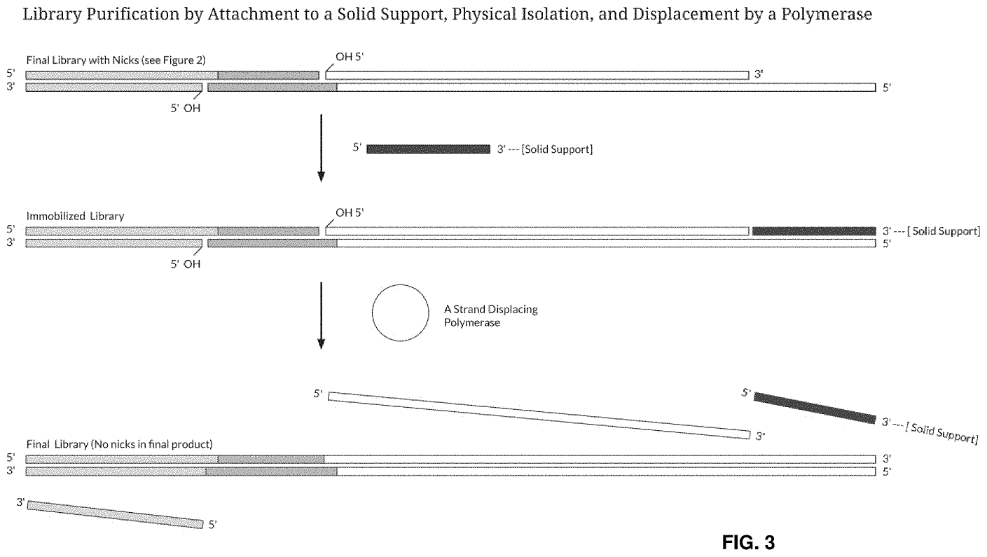

As shown in FIG. 1, a DNA substrate is cleaved by a first type II restriction endonuclease (RE) containing a PAM motif in its recognition sequence and ligation of a first adapter containing the CRISPR guide RNA scaffold sequence is carried out in a single reaction. A modification to the scaffold sequence prevents the first adapter from being cleaved from the DNA substrate after being ligated. The adapter ligation is therefore irreversible and forces the reaction to proceed toward the formation of the intermediate product. In the embodiment shown, the first adapter was synthesized without a phosphate group on the end to be ligated. This prevents the first adapter from ligating to other first adapters, which could result in the final library having truncated products. Because the first adapter is not phosphorylated, the intermediate ligation product will contain a nick. FIG. 2 depicts the digestion of the intermediate product (from the first reaction) by a second type II RE and ligation of a second adapter containing a promoter sequence is carried out in a single reaction. The addition of a methyl moiety to the RE binding site in the first adapter by the second RE prevents cleavage of the second adapter after it has been ligated. Despite the nick in the intermediate product, the second RE can still bind to the recognition sequence and cut across the nick. The second adapter was also synthesized without a phosphate group on the end to be ligated. This prevents the second adapter from ligating to other second adapters, increasing the final yield of the library. In order to maintain the sequence modification shown in FIG. 1, an engineered version of MmeI was used that would recognize a sequence which maintained this first modification. The asterisk (*) indicates that the reaction is not strictly reversible. Because the methyl group is incorporated during digestion, the product of the reverse reaction is a methylated version of the input and is, therefore, not exactly identical to the original intermediate ligation product. Excess second adapter outcompetes the reverse reaction and drives the reaction to the final product. The CRISPR Scaffold adapter is synthesized with a long, single-stranded, 5′ overhang capable of hybridizing to a single stranded oligo immobilized on a solid support, such as a magnetic or agarose bead as shown in FIG. 3. Immobilization of the library on a solid support by hybridization with a complementary capture oligo, with the 5′ end protruding, allows the library to be purified from buffers, protein, and nucleic acid by physical separation. A strand displacing polymerase can be added to permanently remove the fragments from the solid support. This is due to the directionality of the oligo attached to the beads and extension direction of the polymerase. The captured oligos are used as a template by the polymerase and extension by the polymerase prevents re-hybridization. Double stranded DNA can then be preferentially isolated using standard silica-based separation techniques. Immobilization of the library to the solid support can occur at any point in the library construction process.

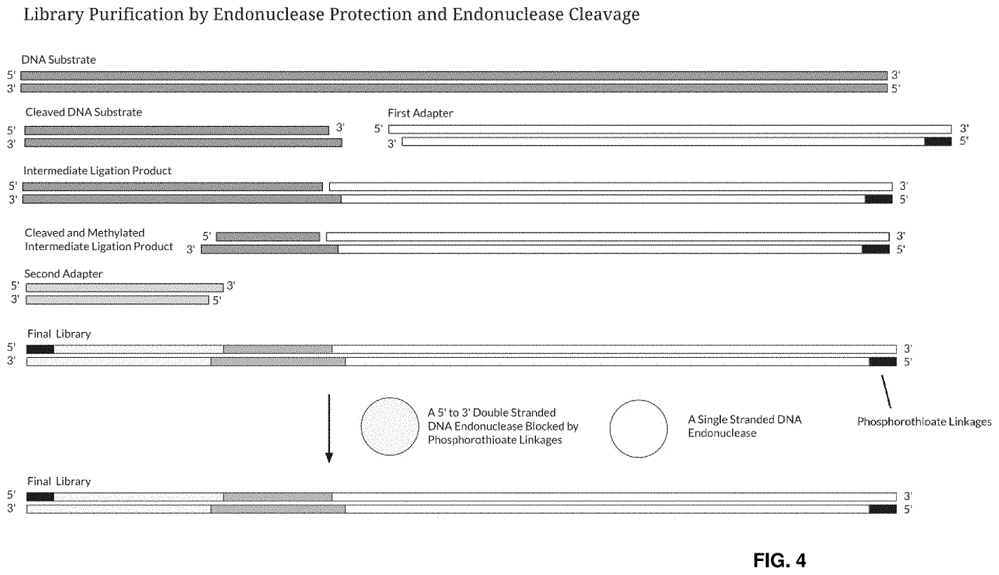

In certain approaches, phosphorothioate linkages (shown in black) are synthesized into the adapters on the outside ends of the 5′ strand as shown in FIG. 4. After the library has been constructed, there could be many byproducts, but only the final product has both of the 5′ ends protected from degradation. Addition of a double stranded, 5′ to 3′ DNA endonuclease that is blocked by the protecting groups as well as a single stranded DNA endonuclease will cause the degradation of any remaining adapters, DNA substrates, ligation products, and intermediate products, but the final library will be protected. PCR primers could also be used to incorporate the linkages into a DNA product that could then be subjected to the endonucleases. Additionally, a 3′ to 5′ endonuclease could also be used if protecting groups are incorporated into the outside ends of the 3′ strand.

For example, a constant region for sgRNA with nucleotide sequence of SEQ ID NO: 3 is recognized by CRISPR Cas9 protein. The sgRNA with nucleotide sequence of SEQ ID NO: 3 can be transcribed from a double stranded polynucleotide having sense strand with nucleotide sequence of SEQ ID NO: 1 and an antisense with nucleotide sequence of SEQ ID NO: 2.

In another example, a constant region for sgRNA with nucleotide sequence of SEQ ID NO: 151 is recognized by CRISPR Cpf1 protein. The sgRNA with nucleotide sequence of SEQ ID NO: 151 can be transcribed from a double stranded polynucleotide having sense strand with nucleotide sequence of SEQ ID NO: 149 and an antisense with nucleotide sequence of SEQ ID NO: 1.

The CRISPR sgRNA(s) or cRNA(s) encoded by the polynucleotide or polynucleotides of various embodiments has at least one hairpin/stem-loop structure. In various embodiments, the CRISPR sgRNA(s) or crRNA(s) encoded by the polynucleotide or polynucleotides has one hairpin/stem-loop structure that are recognized by a CRISPR protein such as Cpf1. In another embodiment, the CRISPR sgRNA(s) or crRNA(s) encoded by the polynucleotide or polynucleotides has a plurality of hairpin/stem-loop structures that are recognized by a CRISPR protein such as Cas9.

In various embodiments, the sequence of the polynucleotide or polynucleotides encoding for a constant region of a CRISPR sgRNA or crRNA excluding or including the non-palindromic recognition site, and optionally a substantially complimentary site to the non-palindromic recognition site, has a homology or percent identity similar to an endogenous sequence of a CRISPR guide RNA or the hairpin regions such that the hairpin regions transcribed from the polynucleotide form and are recognized by a CRISPR protein.

In various embodiments, the CRISPR sgRNA(s) or crRNA(s) encoded by the polynucleotide or polynucleotides of various embodiments are recognized by any CRISPR protein. The CRISPR protein of various embodiments can include, for example, Class 1 or Class 2 CRISPR systems. The CRISPR protein of various embodiments can include, for example, Type I, Type II, Type III, Type IV, or Type V CRISPR systems.

In various embodiments are disclosed polynucleotides encoding for a constant region of a CRISPR single guide RNA (sgRNA) or CRISPR targeting RNA (crRNA) having the following sequence:

5′-CR1N1-RSN2-CR2N3-3′

where:

CR1 is a first constant region with a nucleotide(s) or modified nucleotide(s) including Adenine (A or a), Guanine (G or g), Cytosine (C or c), Thymine (T or t), and/or Uracil (U or u);

N1 is 0, 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 105, 110, 115, 120 or more nucleotides;

RS is a non-palindromic recognition site for a type II restriction enzyme with a nucleotide(s) or modified nucleotide(s) including Adenine (A or a), Guanine (G or g), Cytosine (C or c), Thymine (T or t), or Uracil (U or u);

N2 is 4, 5, 6, 7, or 8;

CR2 is a second constant region with a nucleotide(s) or modified nucleotide(s) including Adenine (A or a), Guanine (G or g), Cytosine (C or c), Thymine (T or t), or Uracil (U or u); and

N3 is 0, 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 105, 110, 115, 120 or more nucleotides;

wherein RS is oriented in a manner recognized by the type II restriction enzyme for cutting a site that is 17 to 27 base pairs past a 5′ or 3′ end of the polynucleotide, in an operably linked targeting region sequence, if present.

In various embodiments are disclosed polynucleotides encoding for a constant region of a CRISPR single guide RNA (sgRNA) or CRISPR targeting RNA (crRNA) having the at least one of the following sequences:

5′-VRN4-CR1N1-RSN2-CR2N3-3′ or 5′-CR1N1-RSN2-CR2N3-VRN4-3′

where:

VR is a variable region (e.g. targeting region) with a nucleotide(s) or modified nucleotide(s) including Adenine (A or a), Guanine (G or g), Cytosine (C or c), Thymine (T or t), or Uracil (U or u);

N4 is 17, 18, 19, 20, 21, 22, 23, 24, 25, 26, or 27 nucleotides; and CR1, N1, RS, and N2 are as described above.

In various embodiments, N1 or N3 is range between any two number of nucleotides listed for N1 and N3 above.

In various embodiments, N2 is range between any two number of nucleotides listed for N2 above.

In various embodiments, N4 is range between any two number of nucleotides listed for N4 above.

The non-palindromic recognition site of the polynucleotide of various embodiments has a sequence recognized by a type IIS restriction enzyme. In various embodiments, the non-palindromic recognition site in a manner recognized by the type II restriction enzyme for cutting a site that is 17, 18, 19, 20, 21, 22, 23, 24, 25, 26, or 27 base pairs past an end of the polynucleotide, in an operably linked targeting region sequence, if present. In various embodiments, the cutting site is a range between any two base pair lengths past an end of the polynucleotide. In various embodiments, the non-palindromic recognition site is oriented in a manner recognized by the type II restriction enzyme for cutting a site that 17 to 27 base pairs past an end of the polynucleotide. In another embodiment, the non-palindromic recognition site is oriented in a manner recognized by the type II restriction enzyme for cutting a site that 18 to 24 base pairs past an end of the polynucleotide.

The type IIS restriction enzyme of various embodiments can include, for example, NmeAIII, MmeI, CstMI, EcoP15I, ApyPI, AquII, AquIII, AquIV, CdpI, CstMI, DraRI, DrdIV, EsaSSI, MaqI, NhaXI, NlaCI, PlaDI, PspOMII, PspPRI, Rcel, RpaB5I, SdeAI, SpoDI, BsbI, or combinations thereof. The recognition sites for listed restriction enzymes are listed below (where the recognition site for each is followed by the cleavage distance):

TABLE I

| | | |
|---|---|---|
| ApyPI: | EsaSSI: | PspPRI: |
| ATCGAC(20/18) | GACCAC(20/18) | CCYCAG(21/19) |
| AquII: | MaqI: | RceI: |
| GCCGNAC(20/18) | CRTTGAC(20/18) | CATCGAC(20/18) |
| AquIII: | MmeI: | RpaB5I: |
| GAGGAG(20/18) | TCCRAC(20/18) | CGRGGAC(20/18) |
| AquIV: | NhaXI: | SdeAI: |
| GRGGAAG(20/18) | CAAGRAG(20/18) | CAGRAG(21/19) |
| CdpI: | NlaCI: | SpoDI: |
| GCGGAG(20/18) | CATCAC(19/17) | GCGGRAG(20/18) |
| CstMI: | NmeAIII: | BsbI: |
| AAGGAG(20/18) | GCCGAG(21/19) | CAACAC(21/19) |
| DraRI: | PlaDI: | EcoP15I: |
| CAAGNAC(20/18) | CATCAG(21/19) | CAGCAG(25/27) |
| DrdIV: | PspOMII: | |
| TACGAC(20/18) | CGCCCAR(20/18) | |

In various embodiments, the polynucleotide further includes a region having a sequence substantially complementary the sequence of non-palindromic recognition site. The complementary region of various embodiments can be spaced either upstream of downstream from the non-palindromic recognition site. When transcribed, the complementary sequence of sgRNA is capable of forming bonds with the non-palindromic recognition site such that hairpins or stem-loop structures form.

In various embodiments, the CR1 and/or CR2 nucleotide can have a second restriction site or be prepared to have ends compatible with DNA digested with restriction enzymes that cut at protospacer adjacent motif (PAM) sites. Restriction enzymes that cut at PAM sites include, for example: HpaII, MspI, ScrFI, BfaI, and PacI. The recognition sites for the listed restriction enzymes are listed below.
HpaII: C/CGG
MspI: C/CGG
ScrFI: CC/NGG
BfaI: C/TAG
PactI: TTAAT/TAA

In various embodiments, the polynucleotide or polynucleotides encoding for a constant region of a CRISPR sgRNA or crRNA excluding or including the non-palindromic recognition site or the non-palindromic recognition site and the complimentary region has at least 80%, 85%, 90%, 95%, 99%, or 100% identity to at least one of SEQ ID NO: 4; SEQ ID NO: 9; SEQ ID NO: 14; SEQ ID NO: 19; SEQ ID NO: 24; SEQ ID NO: 29; SEQ ID NO: 34; SEQ ID NO: 39; SEQ ID NO: 44; SEQ ID NO: 49; SEQ ID NO: 54; SEQ ID NO: 59; SEQ ID NO: 64; SEQ ID NO: 69; SEQ ID NO: 74; SEQ ID NO: 79; SEQ ID NO: 84; SEQ ID NO: 89; SEQ ID NO: 94; SEQ ID NO: 99; SEQ ID NO: 104; SEQ ID NO: 109; SEQ ID NO: 114; SEQ ID NO: 119; SEQ ID NO: 124; SEQ ID NO: 129; SEQ ID NO: 134; SEQ ID NO: 139; SEQ ID NO: 144; SEQ ID NO: 152; SEQ ID NO: 157; SEQ ID NO: 162; SEQ ID NO: 167; SEQ ID NO: 172; SEQ ID NO: 177; SEQ ID NO: 182; SEQ ID NO: 187; SEQ ID NO: 192; SEQ ID NO: 197; SEQ ID NO: 202; SEQ ID NO: 207; SEQ ID NO: 212; SEQ ID NO: 217; SEQ ID NO: 222; SEQ ID NO: 227; SEQ ID NO: 232; SEQ ID NO: 237; SEQ ID NO: 242; SEQ ID NO: 247; SEQ ID NO: 252; SEQ ID NO: 257; SEQ ID NO: 262; SEQ ID NO: 267; SEQ ID NO: 272; SEQ ID NO: 277; SEQ ID NO: 282; SEQ ID NO: 287; SEQ ID NO: 292; SEQ ID NO: 297; SEQ ID NO: 302; SEQ ID NO: 307; SEQ ID NO: 312; SEQ ID NO: 317; SEQ ID NO: 322; SEQ ID NO: 327; SEQ ID NO: 332; SEQ ID NO: 337; SEQ ID NO: 342; SEQ ID NO: 347; SEQ ID NO: 352; SEQ ID NO: 357; SEQ ID NO: 362; SEQ ID NO: 367; SEQ ID NO: 372; SEQ ID NO: 377; SEQ ID NO: 382; SEQ ID NO: 387; SEQ ID NO: 392; SEQ ID NO: 397; or SEQ ID NO: 402.

In various embodiments, the polynucleotide or polynucleotides encoding for a constant region of a CRISPR sgRNA or crRNA comprise or are SEQ ID NO: 4; SEQ ID NO: 9; SEQ ID NO: 14; SEQ ID NO: 19; SEQ ID NO: 24; SEQ ID NO: 29; SEQ ID NO: 34; SEQ ID NO: 39; SEQ ID NO: 44; SEQ ID NO: 49; SEQ ID NO: 54; SEQ ID NO: 59; SEQ ID NO: 64; SEQ ID NO: 69; SEQ ID NO: 74; SEQ ID NO: 79; SEQ ID NO: 84; SEQ ID NO: 89; SEQ ID NO: 94; SEQ ID NO: 99; SEQ ID NO: 104; SEQ ID NO: 109; SEQ ID NO: 114; SEQ ID NO: 119; SEQ ID NO: 124; SEQ ID NO: 129; SEQ ID NO: 134; SEQ ID NO: 139; SEQ ID NO: 144; SEQ ID NO: 152; SEQ ID NO: 157; SEQ ID NO: 162; SEQ ID NO: 167; SEQ ID NO: 172; SEQ ID NO: 177; SEQ ID NO: 182; SEQ ID NO: 187; SEQ ID NO: 192; SEQ ID NO: 197; SEQ ID NO: 202; SEQ ID NO: 207; SEQ ID NO: 212; SEQ ID NO: 217; SEQ ID NO: 222; SEQ ID NO: 227; SEQ ID NO: 232; SEQ ID NO: 237; SEQ ID NO: 242; SEQ ID NO: 247; SEQ ID NO: 252; SEQ ID NO: 257; SEQ ID NO: 262; SEQ ID NO: 267; SEQ ID NO: 272; SEQ ID NO: 277; SEQ ID NO: 282; SEQ ID NO: 287; SEQ ID NO: 292; SEQ ID NO: 297; SEQ ID NO: 302; SEQ ID NO: 307; SEQ ID NO: 312; SEQ ID NO: 317; SEQ ID NO: 322; SEQ ID NO: 327; SEQ ID NO: 332; SEQ ID NO: 337; SEQ ID NO: 342; SEQ ID NO: 347; SEQ ID NO: 352; SEQ ID NO: 357; SEQ ID NO: 362; SEQ ID NO: 367; SEQ ID NO: 372; SEQ ID NO: 377; SEQ ID NO: 382; SEQ ID NO: 387; SEQ ID NO: 392; SEQ ID NO: 397; SEQ ID NO: 402; or SEQ ID NO: 407.

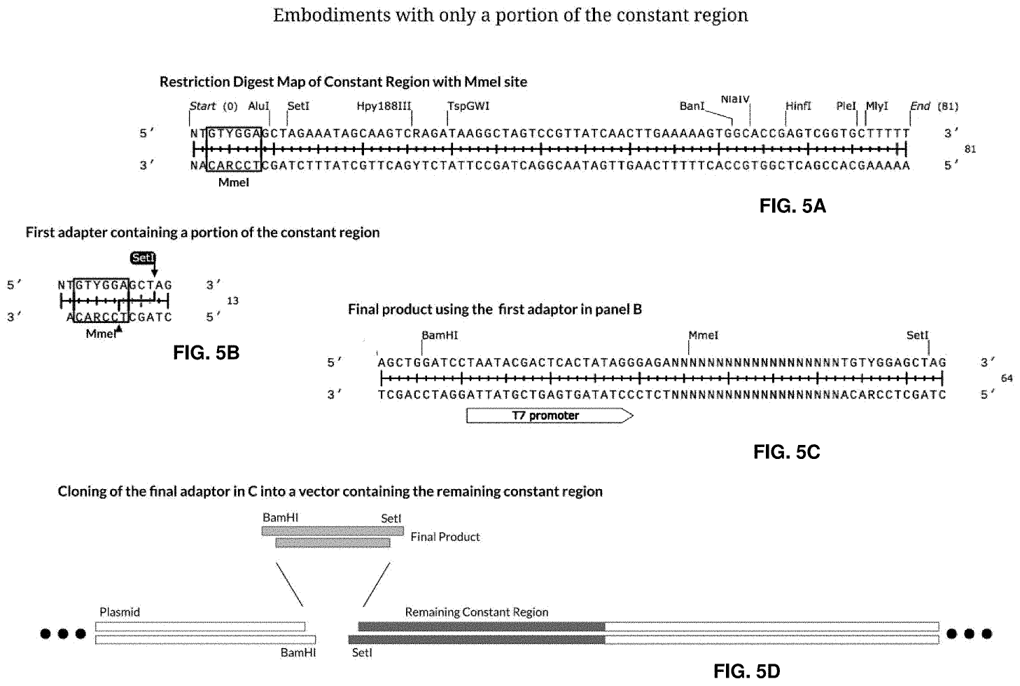

The following are examples of various polynucleotides encoding for sgRNAs or crRNAs recognized by CRISPR Cas9 proteins. SEQ ID NO: 4 to SEQ ID NO: 148 relate to Cas9 systems. See FIG. 5A.

The following examples highlight MmeI site TCCRAC at an end, 1 base pair from an end, or 2 base pairs from an end. The variable region of the examples can be 18, 19, or 20 base pairs long.

The following are examples of various polynucleotides encoding for sgRNAs or crRNAs recognized by CRISPR Cas9 proteins. See FIG. 5A. SEQ ID NO: 4 to SEQ ID NO: 148 relate to Cas9 systems.

The following examples highlights MmeI site TCCRAC at an end, 1 base pair from an end, or 2 base pairs from an end. The variable region of the examples can be 18, 19, or 20 base pairs long.

In various embodiments with MmeI site TCCRAC as shown in FIG. 5B, the polynucleotide is double stranded having sense strand SEQ ID NO: 4 and antisense strand SEQ ID NO: 5. When DNA containing a variable region is ligated to the polynucleotide and subsequently digested with MmeI, the polynucleotide has sense strand SEQ ID NO: 6 and antisense strand SEQ ID NO: 7 that can be transcribed to an sgRNA or crRNA with sequence SEQ ID NO: 8.

In various embodiments with MmeI site TCCRAC as shown in FIG. 5B, the polynucleotide is double stranded having sense strand SEQ ID NO: 9 and antisense strand SEQ ID NO: 10. When DNA containing a variable region is ligated to the polynucleotide and subsequently digested with MmeI, the polynucleotide has sense strand SEQ ID NO: 11 and antisense strand SEQ ID NO: 12 that can be transcribed to an sgRNA or crRNA with sequence SEQ ID NO: 13.

In various embodiments with MmeI site TCCRAC as shown in FIG. 5B, the polynucleotide is double stranded having sense strand SEQ ID NO: 14 and antisense strand SEQ ID NO: 15. When DNA containing a variable region is ligated to the polynucleotide and subsequently digested with MmeI, the polynucleotide has sense strand SEQ ID NO: 16 and antisense strand SEQ ID NO: 17 that can be transcribed to an sgRNA or crRNA with sequence SEQ ID NO: 18.

In various embodiments with MmeI site TCCRAC and HpaII site C/CGG or MspI site C/CGG as shown in FIG. 5B, the polynucleotide is double stranded having sense strand SEQ ID NO: 19 and antisense strand SEQ ID NO: 20. The polynucleotide can be digested with HpaII or MspI to form a compatible end. When DNA containing a variable region is ligated to the polynucleotide and subsequently digested with MmeI, the polynucleotide has sense strand SEQ ID NO: 21 and antisense strand SEQ ID NO: 22 that can be transcribed to an sgRNA or crRNA with sequence SEQ ID NO: 23.

In various embodiments with MmeI site TCCRAC and ScrFI site CC/NGG as shown in FIG. 5B, the polynucleotide is double stranded having sense strand SEQ ID NO: 24 and antisense strand SEQ ID NO: 25. The polynucleotide can be digested with ScrFI to form a compatible end. When DNA containing a variable region is ligated to the polynucleotide and subsequently digested with MmeI, the polynucleotide has sense strand SEQ ID NO: 26 and antisense strand SEQ ID NO: 27 that can be transcribed to an sgRNA or crRNA with sequence SEQ ID NO: 28.

In various embodiments with MmeI site TCCRAC and BfaI site C/TAG as shown in FIG. 5B, the polynucleotide is double stranded having sense strand SEQ ID NO: 29 and antisense strand SEQ ID NO: 30. The polynucleotide can be digested with BfaI to form a compatible end. When DNA containing a variable region is ligated to the polynucleotide and subsequently digested with MmeI, the polynucleotide has sense strand SEQ ID NO: 31 and antisense strand SEQ ID NO: 32 that can be transcribed to an sgRNA or crRNA with sequence SEQ ID NO: 33.

The following examples highlights NmeAIII site GCCGAG at an end, 1 base pair from an end, or 2 base pairs from an end. The variable region of the examples can be 18, 19, or 20 base pairs long.

In various embodiments with NmeAIII site GCCGAG as shown in FIG. 5B, the polynucleotide is double stranded having sense strand SEQ ID NO: 34 and antisense strand SEQ ID NO: 35. When DNA containing a variable region is ligated to the polynucleotide and subsequently digested with NmeAIII, the polynucleotide has sense strand SEQ ID NO: 36 and antisense strand SEQ ID NO: 37 that can be transcribed to an sgRNA or crRNA with sequence SEQ ID NO: 38.

In various embodiments with NmeAIII site GCCGAG as shown in FIG. 5B, the polynucleotide is double stranded having sense strand SEQ ID NO: 39 and antisense strand SEQ ID NO: 40. When DNA containing a variable region is ligated to the polynucleotide and subsequently digested with NmeAIII, the polynucleotide has sense strand SEQ ID NO: 41 and antisense strand SEQ ID NO: 42 that can be transcribed to an sgRNA or crRNA with sequence SEQ ID NO: 43.

In various embodiments with NmeAIII site GCCGAG as shown in FIG. 5B, the polynucleotide is double stranded having sense strand SEQ ID NO: 44 and antisense strand SEQ ID NO: 45. When DNA containing a variable region is ligated to the polynucleotide and subsequently digested with NmeAIII, the polynucleotide has sense strand SEQ ID NO: 46 and antisense strand SEQ ID NO: 47 that can be transcribed to an sgRNA or crRNA with sequence SEQ ID NO: 48.

The following examples highlight different restriction enzymes sites at an end. The variable regions of the following examples can be 18 base pairs, 19 base pairs, or 20 base pairs long.

In various embodiments with ApyPI site ATCGAC, the polynucleotide is double stranded having sense strand SEQ ID NO: 49 and antisense strand SEQ ID NO: 50. When DNA containing a variable region is ligated to the polynucleotide and subsequently digested with ApyPI, the polynucleotide has sense strand SEQ ID NO: 51 and antisense strand SEQ ID NO: 52 that can be transcribed to an sgRNA or crRNA with sequence SEQ ID NO: 53.

In various embodiments with AquII site GCCGNAC, the polynucleotide is double stranded having sense strand SEQ ID NO: 54 and antisense strand SEQ ID NO: 55. When DNA containing a variable region is ligated to the polynucleotide and subsequently digested with AquII, the polynucleotide has sense strand SEQ ID NO: 56 and antisense strand SEQ ID NO: 57 that can be transcribed to an sgRNA or crRNA with sequence SEQ ID NO: 58.

In various embodiments with AquIII site GAGGAG, the polynucleotide is double stranded having sense strand SEQ ID NO: 59 and antisense strand SEQ ID NO: 60. When DNA containing a variable region is ligated to the polynucleotide and subsequently digested with AquIII, the polynucleotide has sense strand SEQ ID NO: 61 and antisense strand SEQ ID NO: 62 that can be transcribed to an sgRNA or crRNA with sequence SEQ ID NO: 63.

In various embodiments with AquIV site GRGGAAG, the polynucleotide is double stranded having sense strand SEQ ID NO: 64 and antisense strand SEQ ID NO: 65. When DNA containing a variable region is ligated to the polynucleotide and subsequently digested with AquIV, the polynucleotide has sense strand SEQ ID NO: 66 and antisense strand SEQ ID NO: 67 that can be transcribed to an sgRNA or crRNA with sequence SEQ ID NO: 68.

In various embodiments with CdpI site GCGGAG, the polynucleotide is double stranded having sense strand SEQ ID NO: 69 and antisense strand SEQ ID NO: 70. When DNA containing a variable region is ligated to the polynucleotide and subsequently digested with CdpI, the polynucleotide has sense strand SEQ ID NO: 71 and antisense strand SEQ ID NO: 72 that can be transcribed to an sgRNA or crRNA with sequence SEQ ID NO: 73.

In various embodiments with CstMI site AAGGAG, the polynucleotide is double stranded having sense strand SEQ ID NO: 74 and antisense strand SEQ ID NO: 75. When DNA containing a variable region is ligated to the polynucleotide and subsequently digested with CstMI, the polynucleotide has sense strand SEQ ID NO: 76 and antisense strand SEQ ID NO: 77 that can be transcribed to an sgRNA or crRNA with sequence SEQ ID NO: 78.

In various embodiments with DraRI site CAAGNAC, the polynucleotide is double stranded having sense strand SEQ ID NO: 79 and antisense strand SEQ ID NO: 80. When DNA containing a variable region is ligated to the polynucleotide and subsequently digested with DraRI, the polynucleotide has sense strand SEQ ID NO: 81 and antisense strand SEQ ID NO: 82 that can be transcribed to an sgRNA or crRNA with sequence SEQ ID NO: 83.

In various embodiments with DrdIV site TACGAC, the polynucleotide is double stranded having sense strand SEQ ID NO: 84 and antisense strand SEQ ID NO: 85. When DNA containing a variable region is ligated to the polynucleotide and subsequently digested with DrdIV, the polynucleotide has sense strand SEQ ID NO: 86 and antisense strand SEQ ID NO: 87 that can be transcribed to an sgRNA or crRNA with sequence SEQ ID NO: 88.

In various embodiments with EsaSSI site GACCAC, the polynucleotide is double stranded having sense strand SEQ ID NO: 89 and antisense strand SEQ ID NO: 90. When DNA containing a variable region is ligated to the polynucleotide and subsequently digested with EsaSSI, the polynucleotide has sense strand SEQ ID NO: 91 and anti sense strand SEQ ID NO: 92 that can be transcribed to an sgRNA or crRNA with sequence SEQ ID NO: 93.

In various embodiments with MaqI site CRTTGAC, the polynucleotide is double stranded having sense strand SEQ ID NO: 94 and antisense strand SEQ ID NO: 95. When DNA containing a variable region is ligated to the polynucleotide and subsequently digested with MaqI, the polynucleotide has sense strand SEQ ID NO: 96 and antisense strand SEQ ID NO: 97 that can be transcribed to an sgRNA or crRNA with sequence SEQ ID NO: 98.

In various embodiments with NhaXI site CAAGRAG, the polynucleotide is double stranded having sense strand SEQ ID NO: 99 and antisense strand SEQ ID NO: 100. When DNA containing a variable region is ligated to the polynucleotide and subsequently digested with NhaXI, the polynucleotide has sense strand SEQ ID NO: 101 and antisense strand SEQ ID NO: 102 that can be transcribed to an sgRNA or crRNA with sequence SEQ ID NO: 103.

In various embodiments with NlaCI site CATCAC, the polynucleotide is double stranded having sense strand SEQ ID NO: 104 and antisense strand SEQ ID NO: 105. When DNA containing a variable region is ligated to the polynucleotide and subsequently digested with NlaCI, the polynucleotide has sense strand SEQ ID NO: 106 and antisense strand SEQ ID NO: 107 that can be transcribed to an sgRNA or crRNA with sequence SEQ ID NO: 108.

In various embodiments with PlaDI site CATCAG, the polynucleotide is double stranded having sense strand SEQ ID NO: 109 and antisense strand SEQ ID NO: 110. When DNA containing a variable region is ligated to the polynucleotide and subsequently digested with PlaDI, the polynucleotide has sense strand SEQ ID NO: 111 and antisense strand SEQ ID NO: 112 that can be transcribed to an sgRNA or crRNA with sequence SEQ ID NO: 113.

In various embodiments with PspOMII site CGCCCAR, the polynucleotide is double stranded having sense strand SEQ ID NO: 114 and antisense strand SEQ ID NO: 115. When DNA containing a variable region is ligated to the polynucleotide and subsequently digested with PspOMII, the polynucleotide has sense strand SEQ ID NO: 116 and antisense strand SEQ ID NO: 117 that can be transcribed to an sgRNA or crRNA with sequence SEQ ID NO: 118.

In various embodiments with PspPRI site CCYCAG, the polynucleotide is double stranded having sense strand SEQ ID NO: 119 and antisense strand SEQ ID NO: 120. When DNA containing a variable region is ligated to the polynucleotide and subsequently digested with PspPRI, the polynucleotide has sense strand SEQ ID NO: 121 and antisense strand SEQ ID NO: 122 that can be transcribed to an sgRNA or crRNA with sequence SEQ ID NO: 123.

In various embodiments with Rcel site CATCGAC, the polynucleotide is double stranded having sense strand SEQ ID NO: 124 and antisense strand SEQ ID NO: 125. When DNA containing a variable region is ligated to the polynucleotide and subsequently digested with Rcel, the polynucleotide has sense strand SEQ ID NO: 126 and antisense strand SEQ ID NO: 127 that can be transcribed to an sgRNA or crRNA with sequence SEQ ID NO: 128.

In various embodiments with RpaB5I site CGRGGAC, the polynucleotide is double stranded having sense strand SEQ ID NO: 129 and antisense strand SEQ ID NO: 130. When DNA containing a variable region is ligated to the polynucleotide and subsequently digested with RpaB5I, the polynucleotide has sense strand SEQ ID NO: 131 and antisense strand SEQ ID NO: 132 that can be transcribed to an sgRNA or crRNA with sequence SEQ ID NO: 133.

In various embodiments with SdeAI site CAGRAG, the polynucleotide is double stranded having sense strand SEQ ID NO: 134 and antisense strand SEQ ID NO: 135. When DNA containing a variable region is ligated to the polynucleotide and subsequently digested with SdeAI, the polynucleotide has sense strand SEQ ID NO: 136 and antisense strand SEQ ID NO: 137 that can be transcribed to an sgRNA or crRNA with sequence SEQ ID NO: 138.

In various embodiments with SpoDI site GCGGRAG, the polynucleotide is double stranded having sense strand SEQ ID NO: 139 and antisense strand SEQ ID NO: 140. When DNA containing a variable region is ligated to the polynucleotide and subsequently digested with SpoDI, the polynucleotide has sense strand SEQ ID NO: 141 and antisense strand SEQ ID NO: 142 that can be transcribed to an sgRNA or crRNA with sequence SEQ ID NO: 143.

In various embodiments with BsbI site CAACAC, the polynucleotide is double stranded having sense strand SEQ ID NO: 144 and antisense strand SEQ ID NO: 145. When DNA containing a variable region is ligated to the polynucleotide and subsequently digested with BsbI, the polynucleotide has sense strand SEQ ID NO: 146 and antisense strand SEQ ID NO: 147 that can be transcribed to an sgRNA or crRNA with sequence SEQ ID NO: 148.

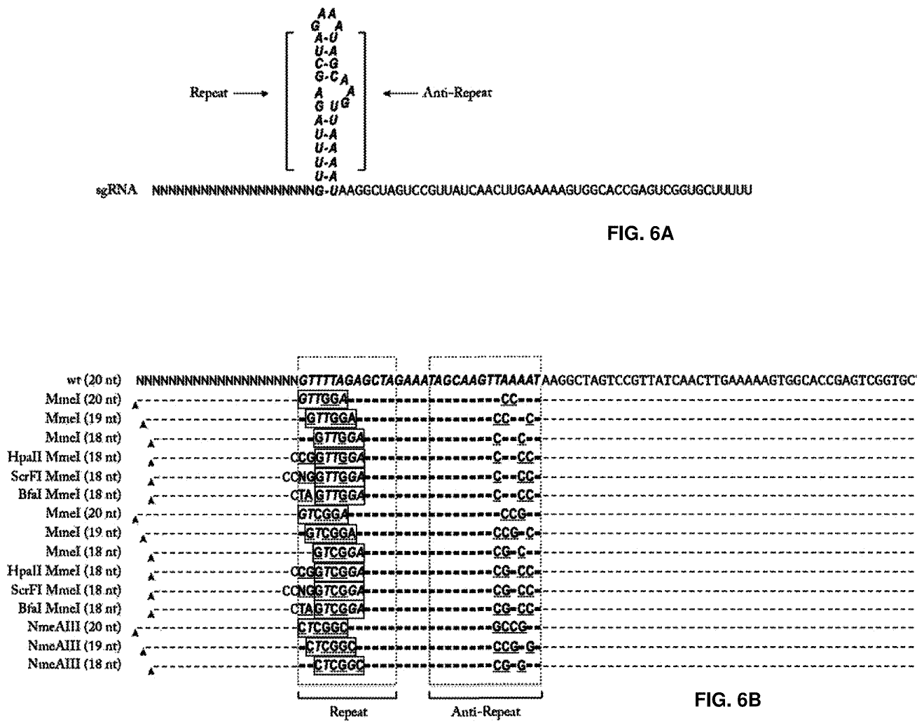

The following are examples of various polynucleotides encoding for sgRNAs or crRNAs recognized by CRISPR Cpf1 proteins. SEQ ID NO: 152 to SEQ ID NO: 406 relate to Cpf1 systems. FIG. 6A shows the hairpin structure of a wildtype cRNA for CRISPR Cpf1.

The following examples highlights MmeI site TCCRAC at an end, 1 base pair from an end, or 2 base pairs from an end. The variable region of the examples can be 18, 19, or 20 base pairs long.

In various embodiments with MmeI site TCCRAC as shown in FIG. 6B, the polynucleotide is double stranded having sense strand SEQ ID NO: 152 and antisense strand SEQ ID NO: 153. When DNA containing a variable region is ligated to the polynucleotide and subsequently digested with MmeI, the polynucleotide has sense strand SEQ ID NO: 154 and antisense strand SEQ ID NO: 155 that can be transcribed to an sgRNA or crRNA with sequence SEQ ID NO: 156.

In various embodiments with MmeI site TCCRAC as shown in FIG. 6B, the polynucleotide is double stranded having sense strand SEQ ID NO: 157 and antisense strand SEQ ID NO: 158. When DNA containing a variable region is ligated to the polynucleotide and subsequently digested with MmeI, the polynucleotide has sense strand SEQ ID NO: 159 and antisense strand SEQ ID NO: 160 that can be transcribed to an sgRNA or crRNA with sequence SEQ ID NO: 161.

In various embodiments with MmeI site TCCRAC as shown in FIG. 6B, the polynucleotide is double stranded having sense strand SEQ ID NO: 162 and antisense strand SEQ ID NO: 163. The polynucleotide can be prepared to have an AT overhang allowing the nucleotide to be compatible with DNA digested with PacI. When DNA containing a variable region is ligated to the polynucleotide and subsequently digested with MmeI, the polynucleotide has sense strand SEQ ID NO: 164 and antisense strand SEQ ID NO: 165 that can be transcribed to an sgRNA or crRNA with sequence SEQ ID NO: 166.

In various embodiments with MmeI site TCCRAC as shown in FIG. 6B, the polynucleotide is double stranded having sense strand SEQ ID NO: 167 and antisense strand SEQ ID NO: 168. The polynucleotide can be prepared to have an AT overhang allowing the nucleotide to be compatible with DNA digested with PacI. When DNA containing a variable region is ligated to the polynucleotide and subsequently digested with MmeI, the polynucleotide has sense strand SEQ ID NO: 169 and antisense strand SEQ ID NO: 170 that can be transcribed to an sgRNA or crRNA with sequence SEQ ID NO: 171.

The following examples highlights NmeAIII site GCCGAG at an end, 1 base pair from an end, or 2 base pair from an end. The variable region of the examples can be 18, 19, or 20 base pairs long.

In various embodiments with NmeAIII site GCCGAG as shown in FIG. 6B, the polynucleotide is double stranded having sense strand SEQ ID NO: 172 and antisense strand SEQ ID NO: 173. When DNA containing a variable region is ligated to the polynucleotide and subsequently digested with NmeAIII, the polynucleotide has sense strand SEQ ID NO: 174 and antisense strand SEQ ID NO: 175 that can be transcribed to an sgRNA or crRNA with sequence SEQ ID NO: 176.

In various embodiments with NmeAIII site GCCGAG as shown in FIG. 6B, the polynucleotide is double stranded having sense strand SEQ ID NO: 177 and antisense strand SEQ ID NO: 178. When DNA containing a variable region is ligated to the polynucleotide and subsequently digested with NmeAIII, the polynucleotide has sense strand SEQ ID NO: 179 and antisense strand SEQ ID NO: 180 that can be transcribed to an sgRNA or crRNA with sequence SEQ ID NO: 181.

In various embodiments with NmeAIII site GCCGAG as shown in FIG. 6B, the polynucleotide is double stranded having sense strand SEQ ID NO: 182 and antisense strand SEQ ID NO: 183. The polynucleotide can be prepared to have an AT overhang allowing the nucleotide to be compatible with DNA digested with Pad. When DNA containing a variable region is ligated to the polynucleotide and subsequently digested with NmeAIII, the polynucleotide has sense strand SEQ ID NO: 184 and antisense strand SEQ ID NO: 185 that can be transcribed to an sgRNA or crRNA with sequence SEQ ID NO: 186.

In various embodiments with NmeAIII site GCCGAG as shown in FIG. 6B, the polynucleotide is double stranded having sense strand SEQ ID NO: 187 and antisense strand SEQ ID NO: 188. The polynucleotide can be prepared to have an AT overhang allowing the nucleotide to be compatible with DNA digested with Pad. When DNA containing a variable region is ligated to the polynucleotide and subsequently digested with NmeAIII, the polynucleotide has sense strand SEQ ID NO: 189 and antisense strand SEQ ID NO: 190 that can be transcribed to an sgRNA or crRNA with sequence SEQ ID NO: 191.

The following examples highlight different restriction enzymes sites 1 base pair from an end. The variable regions of the following examples can be 19 base pairs or 20 base pairs long.

In various embodiments with ApyPI site ATCGAC, the polynucleotide is double stranded having sense strand SEQ ID NO: 192 and antisense strand SEQ ID NO: 193. When DNA containing a variable region is ligated to the polynucleotide and subsequently digested with ApyPI, the polynucleotide has sense strand SEQ ID NO: 194 and antisense strand SEQ ID NO: 195 that can be transcribed to an sgRNA or crRNA with sequence SEQ ID NO: 196.

In various embodiments with AquII site GCCGNAC, the polynucleotide is double stranded having sense strand SEQ ID NO: 197 and antisense strand SEQ ID NO: 198. When DNA containing a variable region is ligated to the polynucleotide and subsequently digested with AquII, the polynucleotide has sense strand SEQ ID NO: 199 and antisense strand SEQ ID NO: 200 that can be transcribed to an sgRNA or crRNA with sequence SEQ ID NO: 201.

In various embodiments with AquIII site GAGGAG, the polynucleotide is double stranded having sense strand SEQ ID NO: 202 and antisense strand SEQ ID NO: 203. When DNA containing a variable region is ligated to the polynucleotide and subsequently digested with AquIII, the polynucleotide has sense strand SEQ ID NO: 204 and antisense strand SEQ ID NO: 205 that can be transcribed to an sgRNA or crRNA with sequence SEQ ID NO: 206.

In various embodiments with AquIV site GRGGAAG, the polynucleotide is double stranded having sense strand SEQ ID NO: 207 and antisense strand SEQ ID NO: 208. When DNA containing a variable region is ligated to the polynucleotide and subsequently digested with AquIV, the polynucleotide has sense strand SEQ ID NO: 209 and antisense strand SEQ ID NO: 210 that can be transcribed to an sgRNA or crRNA with sequence SEQ ID NO: 211.

In various embodiments with CdpI site GCGGAG, the polynucleotide is double stranded having sense strand SEQ ID NO: 212 and antisense strand SEQ ID NO: 213. When DNA containing a variable region is ligated to the polynucleotide and subsequently digested with CdpI, the polynucleotide has sense strand SEQ ID NO: 214 and antisense strand SEQ ID NO: 215 that can be transcribed to an sgRNA or crRNA with sequence SEQ ID NO: 216.

In various embodiments with CstMI site AAGGAG, the polynucleotide is double stranded having sense strand SEQ ID NO: 217 and antisense strand SEQ ID NO: 218. When DNA containing a variable region is ligated to the polynucleotide and subsequently digested with CstMI, the polynucleotide has sense strand SEQ ID NO: 219 and antisense strand SEQ ID NO: 220 that can be transcribed to an sgRNA or crRNA with sequence SEQ ID NO: 221.

In various embodiments with DraRI site CAAGNAC, the polynucleotide is double stranded having sense strand SEQ ID NO: 222 and antisense strand SEQ ID NO: 223. When DNA containing a variable region is ligated to the polynucleotide and subsequently digested with DraRI, the polynucleotide has sense strand SEQ ID NO: 224 and antisense strand SEQ ID NO: 225 that can be transcribed to an sgRNA or crRNA with sequence SEQ ID NO: 226.

In various embodiments with DrdIV site TACGAC, the polynucleotide is double stranded having sense strand SEQ ID NO: 227 and antisense strand SEQ ID NO: 228. When DNA containing a variable region is ligated to the polynucleotide and subsequently digested with DrdIV, the polynucleotide has sense strand SEQ ID NO: 229 and antisense strand SEQ ID NO: 230 that can be transcribed to an sgRNA or crRNA with sequence SEQ ID NO: 231.

In various embodiments with EsaSSI site GACCAC, the polynucleotide is double stranded having sense strand SEQ ID NO: 232 and antisense strand SEQ ID NO: 233. When DNA containing a variable region is ligated to the polynucleotide and subsequently digested with EsaSSI, the polynucleotide has sense strand SEQ ID NO: 234 and antisense strand SEQ ID NO: 235 that can be transcribed to an sgRNA or crRNA with sequence SEQ ID NO: 236.

In various embodiments with MaqI site CRTTGAC, the polynucleotide is double stranded having sense strand SEQ ID NO: 237 and antisense strand SEQ ID NO: 238. When DNA containing a variable region is ligated to the polynucleotide and subsequently digested with MaqI, the polynucleotide has sense strand SEQ ID NO: 239 and antisense strand SEQ ID NO: 240 that can be transcribed to an sgRNA or crRNA with sequence SEQ ID NO: 241.

In various embodiments with NhaXI site CAAGRAG, the polynucleotide is double stranded having sense strand SEQ ID NO: 242 and antisense strand SEQ ID NO: 243. When DNA containing a variable region is ligated to the polynucleotide and subsequently digested with NhaXI, the polynucleotide has sense strand SEQ ID NO: 244 and antisense strand SEQ ID NO: 245 that can be transcribed to an sgRNA or crRNA with sequence SEQ ID NO: 246.

In various embodiments with NlaCI site CATCAC, the polynucleotide is double stranded having sense strand SEQ ID NO: 247 and antisense strand SEQ ID NO: 248. When DNA containing a variable region is ligated to the polynucleotide and subsequently digested with NlaCI, the polynucleotide has sense strand SEQ ID NO: 249 and antisense strand SEQ ID NO: 250 that can be transcribed to an sgRNA or crRNA with sequence SEQ ID NO: 251.

In various embodiments with PlaDI site CATCAG, the polynucleotide is double stranded having sense strand SEQ ID NO: 252 and antisense strand SEQ ID NO: 253. When DNA containing a variable region is ligated to the polynucleotide and subsequently digested with PlaDI, the polynucleotide has sense strand SEQ ID NO: 254 and antisense strand SEQ ID NO: 255 that can be transcribed to an sgRNA or crRNA with sequence SEQ ID NO: 256.

In various embodiments with PspOMII site CGCCCAR, the polynucleotide is double stranded having sense strand SEQ ID NO: 257 and antisense strand SEQ ID NO: 258. When DNA containing a variable region is ligated to the polynucleotide and subsequently digested with PspOMII, the polynucleotide has sense strand SEQ ID NO: 259 and antisense strand SEQ ID NO: 260 that can be transcribed to an sgRNA or crRNA with sequence SEQ ID NO: 261.

In various embodiments with PspPRI site CCYCAG, the polynucleotide is double stranded having sense strand SEQ ID NO: 262 and antisense strand SEQ ID NO: 263. When DNA containing a variable region is ligated to the polynucleotide and subsequently digested with PspPRI, the polynucleotide has sense strand SEQ ID NO: 264 and antisense strand SEQ ID NO: 265 that can be transcribed to an sgRNA or crRNA with sequence SEQ ID NO: 266.

In various embodiments with Rcel site CATCGAC, the polynucleotide is double stranded having sense strand SEQ ID NO: 267 and antisense strand SEQ ID NO: 268. When DNA containing a variable region is ligated to the polynucleotide and subsequently digested with Rcel, the polynucleotide has sense strand SEQ ID NO: 269 and antisense strand SEQ ID NO: 270 that can be transcribed to an sgRNA or crRNA with sequence SEQ ID NO: 271.

In various embodiments with RpaB5I site CGRGGAC, the polynucleotide is double stranded having sense strand SEQ ID NO: 272 and antisense strand SEQ ID NO: 273. When DNA containing a variable region is ligated to the polynucleotide and subsequently digested with RpaB5I, the polynucleotide has sense strand SEQ ID NO: 274 and antisense strand SEQ ID NO: 275 that can be transcribed to an sgRNA or crRNA with sequence SEQ ID NO: 276.

In various embodiments with SdeAI site CAGRAG, the polynucleotide is double stranded having sense strand SEQ ID NO: 277 and antisense strand SEQ ID NO: 278. When DNA containing a variable region is ligated to the polynucleotide and subsequently digested with SdeAI, the polynucleotide has sense strand SEQ ID NO: 279 and antisense strand SEQ ID NO: 280 that can be transcribed to an sgRNA or crRNA with sequence SEQ ID NO: 281.

In various embodiments with SpoDI site GCGGRAG, the polynucleotide is double stranded having sense strand SEQ ID NO: 282 and antisense strand SEQ ID NO: 283. When DNA containing a variable region is ligated to the polynucleotide and subsequently digested with SpoDI, the polynucleotide has sense strand SEQ ID NO: 284 and antisense strand SEQ ID NO: 285 that can be transcribed to an sgRNA or crRNA with sequence SEQ ID NO: 286.

In various embodiments with BsbI site CAACAC, the polynucleotide is double stranded having sense strand SEQ ID NO: 287 and antisense strand SEQ ID NO: 288. When DNA containing a variable region is ligated to the polynucleotide and subsequently digested with BsbI, the polynucleotide has sense strand SEQ ID NO: 289 and antisense strand SEQ ID NO: 290 that can be transcribed to an sgRNA or crRNA with sequence SEQ ID NO: 291.

The following examples highlight different restriction enzymes sites 2 base pair from an end. The variable regions of the following examples can be 18 base pairs or 19 base pairs long.

In various embodiments with ApyPI site ATCGAC, the polynucleotide is double stranded having sense strand SEQ ID NO: 292 and antisense strand SEQ ID NO: 293. When DNA containing a variable region is ligated to the polynucleotide and subsequently digested with ApyPI, the polynucleotide has sense strand SEQ ID NO: 294 and antisense strand SEQ ID NO: 295 that can be transcribed to an sgRNA or crRNA with sequence SEQ ID NO: 296.

In various embodiments with AquII site GCCGNAC, the polynucleotide is double stranded having sense strand SEQ ID NO:297 and antisense strand SEQ ID NO: 298. When DNA containing a variable region is ligated to the polynucleotide and subsequently digested with AquII, the polynucleotide has sense strand SEQ ID NO: 299 and antisense strand SEQ ID NO: 300 that can be transcribed to an sgRNA or crRNA with sequence SEQ ID NO: 301.

In various embodiments with AquIII site GAGGAG, the polynucleotide is double stranded having sense strand SEQ ID NO:302 and antisense strand SEQ ID NO: 303. When DNA containing a variable region is ligated to the polynucleotide and subsequently digested with AquIII, the polynucleotide has sense strand SEQ ID NO: 304 and antisense strand SEQ ID NO: 305 that can be transcribed to an sgRNA or crRNA with sequence SEQ ID NO: 306.

In various embodiments with AquIV site GRGGAAG, the polynucleotide is double stranded having sense strand SEQ ID NO:307 and antisense strand SEQ ID NO: 308. When DNA containing a variable region is ligated to the polynucleotide and subsequently digested with AquIV, the polynucleotide has sense strand SEQ ID NO: 309 and antisense strand SEQ ID NO: 310 that can be transcribed to an sgRNA or crRNA with sequence SEQ ID NO: 311.

In various embodiments with CdpI site GCGGAG, the polynucleotide is double stranded having sense strand SEQ ID NO:312 and antisense strand SEQ ID NO: 313. When DNA containing a variable region is ligated to the polynucleotide and subsequently digested with CdpI, the polynucleotide has sense strand SEQ ID NO: 314 and antisense strand SEQ ID NO: 315 that can be transcribed to an sgRNA or crRNA with sequence SEQ ID NO: 316.

In various embodiments with CstMI site AAGGAG, the polynucleotide is double stranded having sense strand SEQ ID NO:317 and antisense strand SEQ ID NO: 318. When DNA containing a variable region is ligated to the polynucleotide and subsequently digested with CstMI, the polynucleotide has sense strand SEQ ID NO: 319 and antisense strand SEQ ID NO: 320 that can be transcribed to an sgRNA or crRNA with sequence SEQ ID NO: 321.

In various embodiments with DraRI site CAAGNAC, the polynucleotide is double stranded having sense strand SEQ ID NO:322 and antisense strand SEQ ID NO: 323. When DNA containing a variable region is ligated to the polynucleotide and subsequently digested with DraRI, the polynucleotide has sense strand SEQ ID NO: 324 and antisense strand SEQ ID NO: 325 that can be transcribed to an sgRNA or crRNA with sequence SEQ ID NO: 326.

In various embodiments with DrdIV site TACGAC, the polynucleotide is double stranded having sense strand SEQ ID NO:327 and antisense strand SEQ ID NO: 328. When DNA containing a variable region is ligated to the polynucleotide and subsequently digested with DrdIV, the polynucleotide has sense strand SEQ ID NO: 329 and antisense strand SEQ ID NO: 330 that can be transcribed to an sgRNA or crRNA with sequence SEQ ID NO: 331.

In various embodiments with EsaSSI site GACCAC, the polynucleotide is double stranded having sense strand SEQ ID NO:332 and antisense strand SEQ ID NO: 333. When DNA containing a variable region is ligated to the polynucleotide and subsequently digested with EsaSSI, the polynucleotide has sense strand SEQ ID NO: 334 and antisense strand SEQ ID NO: 335 that can be transcribed to an sgRNA or crRNA with sequence SEQ ID NO: 336.

In various embodiments with MaqI site CRTTGAC, the polynucleotide is double stranded having sense strand SEQ ID NO:337 and antisense strand SEQ ID NO: 338. When DNA containing a variable region is ligated to the polynucleotide and subsequently digested with MaqI, the polynucleotide has sense strand SEQ ID NO: 339 and antisense strand SEQ ID NO: 340 that can be transcribed to an sgRNA or crRNA with sequence SEQ ID NO: 341.

In various embodiments with NhaXI site CAAGRAG, the polynucleotide is double stranded having sense strand SEQ ID NO:342 and antisense strand SEQ ID NO: 343. When DNA containing a variable region is ligated to the polynucleotide and subsequently digested with NhaXI, the polynucleotide has sense strand SEQ ID NO: 344 and antisense strand SEQ ID NO: 345 that can be transcribed to an sgRNA or crRNA with sequence SEQ ID NO: 346.

In various embodiments with PlaDI site CATCAG, the polynucleotide is double stranded having sense strand SEQ ID NO:347 and antisense strand SEQ ID NO: 348. When DNA containing a variable region is ligated to the polynucleotide and subsequently digested with PlaDI, the polynucleotide has sense strand SEQ ID NO: 349 and antisense strand SEQ ID NO: 350 that can be transcribed to an sgRNA or crRNA with sequence SEQ ID NO: 351.

In various embodiments with PspOMII site CGCCCAR, the polynucleotide is double stranded having sense strand SEQ ID NO:352 and antisense strand SEQ ID NO: 353. When DNA containing a variable region is ligated to the polynucleotide and subsequently digested with PspOMII, the polynucleotide has sense strand SEQ ID NO: 354 and antisense strand SEQ ID NO: 355 that can be transcribed to an sgRNA or crRNA with sequence SEQ ID NO: 356.

In various embodiments with PspPRI site CCYCAG, the polynucleotide is double stranded having sense strand SEQ ID NO:357 and antisense strand SEQ ID NO: 358. When DNA containing a variable region is ligated to the polynucleotide and subsequently digested with PspPRI, the polynucleotide has sense strand SEQ ID NO: 359 and antisense strand SEQ ID NO: 360 that can be transcribed to an sgRNA or crRNA with sequence SEQ ID NO: 361.

In various embodiments with Rcel site CATCGAC, the polynucleotide is double stranded having sense strand SEQ ID NO:362 and antisense strand SEQ ID NO: 363. When DNA containing a variable region is ligated to the polynucleotide and subsequently digested with Rcel, the polynucleotide has sense strand SEQ ID NO: 364 and antisense strand SEQ ID NO: 365 that can be transcribed to an sgRNA or crRNA with sequence SEQ ID NO: 366.

In various embodiments with RpaB5I site CGRGGAC, the polynucleotide is double stranded having sense strand SEQ ID NO:367 and antisense strand SEQ ID NO: 368. When DNA containing a variable region is ligated to the polynucleotide and subsequently digested with RpaB5I, the polynucleotide has sense strand SEQ ID NO: 369 and antisense strand SEQ ID NO: 370 that can be transcribed to an sgRNA or crRNA with sequence SEQ ID NO: 371.

In various embodiments with SdeAI site CAGRAG, the polynucleotide is double stranded having sense strand SEQ ID NO:372 and antisense strand SEQ ID NO: 373. When DNA containing a variable region is ligated to the polynucleotide and subsequently digested with SdeAI, the polynucleotide has sense strand SEQ ID NO: 374 and antisense strand SEQ ID NO: 375 that can be transcribed to an sgRNA or crRNA with sequence SEQ ID NO: 376.

In various embodiments with SpoDI site GCGGRAG, the polynucleotide is double stranded having sense strand SEQ ID NO:377 and antisense strand SEQ ID NO: 378. When DNA containing a variable region is ligated to the polynucleotide and subsequently digested with SpoDI, the polynucleotide has sense strand SEQ ID NO: 379 and antisense strand SEQ ID NO: 380 that can be transcribed to an sgRNA or crRNA with sequence SEQ ID NO: 381.

In various embodiments with, the polynucleotide is double stranded having sense strand SEQ ID NO:382 and antisense strand SEQ ID NO: 383. When DNA containing a variable region is ligated to the polynucleotide and subsequently digested with BsbI, the polynucleotide has sense strand SEQ ID NO: 384 and antisense strand SEQ ID NO: 385 that can be transcribed to an sgRNA or crRNA with sequence SEQ ID NO: 386.

The following examples highlight different restriction enzymes sites 3 base pair from an end. The variable regions of the following examples can be 18 base pairs long.

In various embodiments with PlaDI site CATCAG, the polynucleotide is double stranded having sense strand SEQ ID NO:387 and antisense strand SEQ ID NO: 388. When DNA containing a variable region is ligated to the polynucleotide and subsequently digested with PlaDI, the polynucleotide has sense strand SEQ ID NO: 389 and antisense strand SEQ ID NO: 390 that can be transcribed to an sgRNA or crRNA with sequence SEQ ID NO: 391.

In various embodiments with PspPRI site CCYCAG, the polynucleotide is double stranded having sense strand SEQ ID NO:392 and antisense strand SEQ ID NO: 393. When DNA containing a variable region is ligated to the polynucleotide and subsequently digested with PspPRI, the polynucleotide has sense strand SEQ ID NO: 394 and antisense strand SEQ ID NO: 395 that can be transcribed to an sgRNA or crRNA with sequence SEQ ID NO: 396.

In various embodiments with SdeAI site CAGRAG, the polynucleotide is double stranded having sense strand SEQ ID NO:397 and antisense strand SEQ ID NO: 398. When DNA containing a variable region is ligated to the polynucleotide and subsequently digested with SdeAI, the polynucleotide has sense strand SEQ ID NO: 399 and antisense strand SEQ ID NO: 400 that can be transcribed to an sgRNA or crRNA with sequence SEQ ID NO: 401.

In various embodiments with BsbI site CAACAC, the polynucleotide is double stranded having sense strand SEQ ID NO:402 and antisense strand SEQ ID NO: 403. When DNA containing a variable region is ligated to the polynucleotide and subsequently digested with BsbI, the polynucleotide has sense strand SEQ ID NO: 404 and antisense strand SEQ ID NO: 405 that can be transcribed to an sgRNA or crRNA with sequence SEQ ID NO: 406.

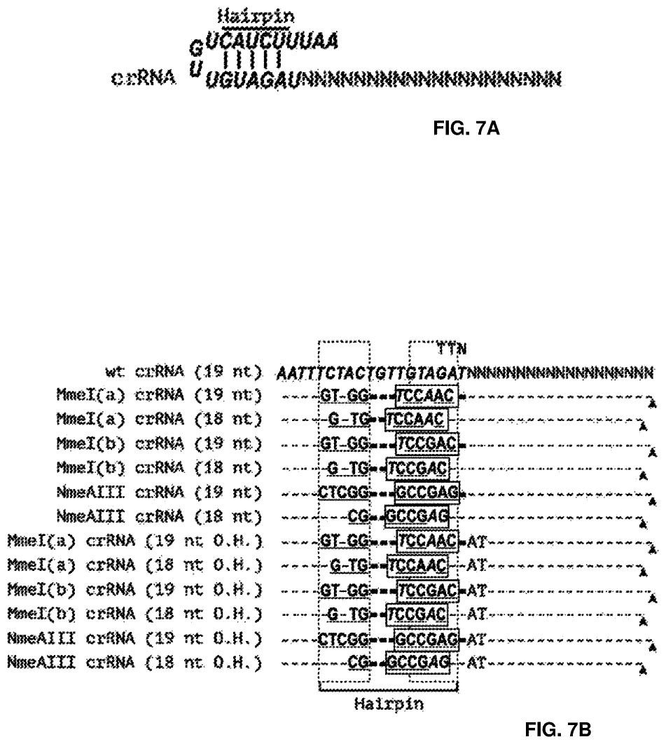

SEQ ID NOS: 424-439 and 441 to 453 to are shown in FIGS. 6 and 7, respectively. The sequences in the Tables below list a number additional sequences that are useful in the methods of the present invention. Each sequence satisfies the following criteria:

1. The re-creation of existing restriction enzyme sites upon ligation is prevented.
2. The Type IIS sequence is situated such that the enzyme cuts at least 17 bp and not more than 25 bp upstream or downstream of the scaffold/constant region as cas9 has the variable region upstream, whereas in a Cpf1 system, the variable region is downstream
3. Base pairing interactions in the stem loop are maintained without introducing a second Type II RS enzyme site (e.g., a second MmeI site).
4. Optional overhangs can be included to aid ligation which could be generated using Mung Bean Nuclease (or similar enzyme) during digestion and ligation to blunt the products for ligation.

A representative embodiment entails use of the combination of ScrFI with MmeI (SEQ ID NOs: 454/455. This sequence contains the normal MmeI site where it will cut 18 bp upstream, most often, and generate a separate two-basepair N overhang.

This basic embodiment can be modified with combinations of the following:
1. In certain embodiments different overhangs can be utilized for ligation (SEQ ID NOs: 458/459, 460/461, 462/463, 464/465, 466/467, 472/473, 474/475, 476/477, 478/479, 480/481, 484/485, 486/487, 488/489, 492/493, 494/495, 498/499, 500/501)
2. As mentioned above, different Type IIS enzymes are useful in the methods of the invention (SEQ ID NOs: 480/481, 482/483, 484/485, 486/487, 488/489, 490/491, 492/493, 494/495, 496/497, 498/499, 500/501)
3. In other embodiments, engineered Type IIS nucleases that recognize different motifs are employed (SEQ ID NOs: 456/457, 458/459, 460/461, 462/463, 468/469, 472/473, 476/477) 4. In other aspects, overlapping overhang and Type IIS recognition motifs are utilized (SEQ ID NOs: 466/467)
5. In other approaches the position of the Type IIS motif can be adjusted as exemplified in (SEQ ID NOs: 466/467, 468/469, 470/471, 472/473, 474/475, 476/477, 478/479, 480/481, 482/483, 484/485, 486/487, 488/489, 490/491, 492/493, 494/495, 496/497, 498/499, 500/501)
6. Other embodiments include two-part guide systems as described below.
7. Cpf1 scaffolds are also provided.

The sequence(s) listed and characterized below in Table II, or any combination thereof, provide examples which may be used in the compositions, methods, or kits described herein.

TABLE II

| SEQ | | | | | |
|---|---|---|---|---|---|
| ID | | Type IIS | Type IIS site | Restriction | |
| NO: | Strand | enzyme | position | Enzyme | Overhang |
| 454 | Forward | MmeI | 2 | ScrFI | N |
| 455 | Reverse | | 2 | | |
| 456 | Forward | MmeI | 2 | ScrFI | N |
| | | (Engineered) | | | |
| 457 | Reverse | | 2 | | |
| 458 | Forward | MmeI | 2 | MspI or HpaII | CG |
| | | (Engineered) | | | |
| 459 | Reverse | | 2 | | |
| 460 | Forward | MmeI | 2 | BfaI | TA |
| | | (Engineered) | | | |
| 461 | Reverse | | 2 | | |
| 462 | Forward | MmeI | 2 | PacI | |
| | | (Engineered) | | | |
| 463 | Reverse | | 2 | | AT |
| 464 | Forward | MmeI | 2 | PacI | |
| 465 | Reverse | | 2 | | AT |
| 466 | Forward | MmeI | 1 | MspI or HpaII | CG |
| 467 | Reverse | | 1 | | |
| 468 | Forward | MmeI | 1 | ScrFI | N |
| | | (Engineered) | | | |
| 469 | Reverse | | 1 | | |
| 470 | Forward | MmeI | 1 | ScrFI | N |
| 471 | Reverse | | 1 | | |
| 472 | Forward | MmeI | 1 | PacI | |
| | | (Engineered) | | | |
| 473 | Reverse | | 1 | | AT |
| 474 | Forward | MmeI | 1 | PacI | |
| 475 | Reverse | | 1 | | AT |
| 476 | Forward | MmeI | 0 | PacI | |
| | | (Engineered) | | | |
| 477 | Reverse | | 0 | | AT |
| 478 | Forward | MmeI | 0 | PacI | |
| 479 | Reverse | | 0 | | AT |
| 480 | Forward | NmeAIII | 3 | MspI or HpaII | CG |
| 481 | Reverse | | 3 | | |
| 482 | Forward | NmeAIII | 3 | ScrFI | N |
| 483 | Reverse | | 3 | | |
| 484 | Forward | NmeAIII | 3 | Bfal | TA |
| 485 | Reverse | | 3 | | |
| 486 | Forward | NmeAIII | 3 | PacI | |
| 487 | Reverse | | 3 | | AT |
| 488 | Forward | NmeAIII | 2 | MspI or HpaII | CG |
| 489 | Reverse | | 2 | | |
| 490 | Forward | NmeAIII | 2 | ScrFI | N |
| 491 | Reverse | | 2 | | |
| 492 | Forward | NmeAIII | 2 | BfaI | TA |
| 493 | Reverse | | 2 | | |
| 494 | Forward | NmeAIII | 2 | PacI | |
| 495 | Reverse | | 2 | | AT |
| 496 | Forward | NmeAIII | 1 | ScrFI | N |
| 497 | Reverse | | 1 | | |
| 498 | Forward | NmeAIII | 1 | PacI | |
| 499 | Reverse | | 1 | | AT |
| 500 | Forward | NmeAIII | 0 | PacI | |
| 501 | Reverse | | 0 | | AT |

In a preferred embodiment, the sequence(s) listed and characterized below in Table III, or any combination thereof, may be used in the compositions, methods, or kits described herein.

TABLE III

| | | Type | Type | | | | |
|---|---|---|---|---|---|---|---|
| | | IIS | IIS site | | | Sequence1 (Used | Sequence2 |
| Type | Strand | enzyme | position | RE | Overhang | in Kit) | (In Plasmid) |
| DNA | Forward | MmeI | 2 | ScrFI | N | nt 1 to nt 11 of | nt 12 to nt 81 |
| | | | | | | SEQ ID NO: 454 | of |
| | | | | | | | SEQ ID NO: |
| | | | | | | | 454 |
| DNA | Reverse | | 2 | | | nt 75 to nt 80 of | nt 1 to nt 74 |
| | | | | | | SEQ ID NO: 455 | of |
| | | | | | | | SEQ ID NO: |
| | | | | | | | 455 |
| DNA | Forward | MmeI | 2 | ScrFI | N | nt 1 to nt 11 of | nt 12 to nt 81 |
| | | (Engine- | | | | SEQ ID NO: 456 | of SEQ ID |
| | | ered) | | | | | NO: 456 |
| DNA | Reverse | | 2 | | | nt 75 to nt 80 of | nt 1 to nt 74 |
| | | | | | | SEQ ID NO: 457 | of SEQ ID |
| | | | | | | | NO: 457 |
| DNA | Forward | MmeI | 1 | MspI or | CG | nt 1 to nt 11 of | nt 12 to nt 81 |
| | | | | HpaII | | SEQ ID NO: 466 | of SEQ ID |
| | | | | | | | NO: 466 |
| DNA | Reverse | | 1 | | | nt 75 to nt 79 of | nt 1 to nt 74 |
| | | | | | | SEQ ID NO: 467 | of SEQ ID |
| | | | | | | | NO: 467 |
| DNA | Forward | MmeI | 1 | ScrFI | N | nt 1 to nt 11 of | nt 12 to nt 81 |
| | | (Engine- | | | | SEQ ID NO: 468 | of SEQ ID |
| | | ered) | | | | | NO: 468 |
| DNA | Reverse | | 1 | | | nt 75 to nt 80 of | nt 1 to nt 74 |
| | | | | | | SEQ ID NO: 469 | of SEQ ID |
| | | | | | | | NO: 469 |
| DNA | Forward | MmeI | 1 | ScrFI | N | nt 1 to nt 11 of | nt 12 to nt 81 |
| | | | | | | SEQ ID NO: 470 | of SEQ ID |
| | | | | | | | NO: 470 |
| DNA | Reverse | | 1 | | | nt 75 to nt 80 of | nt 1 to nt 74 |
| | | | | | | SEQ ID NO: 471 | of SEQ ID |
| | | | | | | | NO: 471 |
| DNA | Forward | MmeI | 1 | PacI | | nt 1 to nt 11 of | nt 12 to nt 81 |
| | | (Engine- | | | | SEQ ID NO: 472 | of SEQ ID |
| | | ered) | | | | | NO: 472 |
| DNA | Reverse | | 1 | | AT | nt 75 to nt 83 of | nt 1 to nt 74 |
| | | | | | | SEQ ID NO: 473 | of SEQ ID |
| | | | | | | | NO: 473 |
| DNA | Forward | MmeI | 1 | PacI | | nt 1 to nt 11 of | nt 12 to nt 81 |
| | | | | | | SEQ ID NO: 474 | of SEQ ID |
| | | | | | | | NO: 474 |
| DNA | Reverse | | 1 | | AT | nt 75 to nt 83 of | |
| | | | | | | SEQ ID NO: 475 | |
| DNA | Forward | MmeI | 0 | PacI | | nt 1 to nt 11 of | nt 12 to nt 81 |
| | | (Engine | | | | SEQ ID NO: 476 | of SEQ ID |
| | | ered) | | | | | NO: 476 |
| DNA | Reverse | | 0 | | AT | nt 75 to nt 81 of | nt 1 to nt 74 |
| | | | | | | SEQ ID NO: 477 | of SEQ ID |
| | | | | | | | NO: 477 |
| DNA | Forward | MmeI | 0 | PacI | | nt 1 to nt 11 of | nt 12 to nt 81 |
| | | | | | | SEQ ID NO: 478 | of SEQ ID |
| | | | | | | | NO: 478 |
| DNA | Reverse | | 0 | | AT | nt 75 to nt 81 of | nt 1 to nt 74 |
| | | | | | | SEQ ID NO: 479 | of SEQ ID |
| | | | | | | | NO: 479 |
| DNA | Forward | NmeAIII | 2 | MspI or | CG | nt 1 to nt 30 of | nt 31 to nt 81 |
| | | | | HpaII | | SEQ ID NO: 488 | of SEQ ID |
| | | | | | | | NO: 488 |
| DNA | Reverse | | 2 | | | nt 56 to nt 79 of | nt 1 to nt 55 |
| | | | | | | SEQ ID NO: 489 | of SEQ ID |
| | | | | | | | NO: 489 |
| DNA | Forward | NmeAIII | 2 | ScrFI | N | nt 1 to nt 30 of | nt 31 to nt 81 |
| | | | | | | SEQ ID NO: 490 | of SEQ ID |
| | | | | | | | NO: 490 |
| DNA | Reverse | | 2 | | | nt 56 to nt 80 of | nt 1 to nt 55 |
| | | | | | | SEQ ID NO: 491 | of SEQ ID |
| | | | | | | | NO: 491 |
| DNA | Forward | NmeAIII | 2 | BfaI | TA | nt 1 to nt 30 of | nt 31 to nt 81 |
| | | | | | | SEQ ID NO: 492 | of SEQ ID |
| | | | | | | | NO: 492 |
| DNA | Reverse | | 2 | | | nt 56 to nt 79 of | nt 1 to nt 55 |
| | | | | | | SEQ ID NO: 493 | of SEQ ID |
| | | | | | | | NO: 493 |
| DNA | Forward | NmeAIII | 2 | PacI | | nt 1 to nt 30 of | nt 31 to nt 81 |
| | | | | | | SEQ ID NO: 494 | of SEQ ID |
| | | | | | | | NO: 494 |
| DNA | Reverse | | 2 | | AT | nt 56 to nt 83 of | nt 1 to nt 55 |
| | | | | | | SEQ ID NO: 495 | of SEQ ID |
| | | | | | | | NO: 495 |
| DNA | Forward | NmeAIII | 1 | ScrFI | N | nt 1 to nt 11 of | nt 12 to nt 81 |
| | | | | | | SEQ ID NO: 496 | of SEQ ID |
| | | | | | | | NO: 496 |
| DNA | Reverse | | 1 | | | nt 75 to nt 80 of | nt 1 to nt 74 |
| | | | | | | SEQ ID NO: 497 | of SEQ ID |
| | | | | | | | NO: 497 |
| DNA | Forward | NmeAIII | 1 | PacI | | nt 1 to nt 11 of | nt 12 to nt 81 |
| | | | | | | SEQ ID NO: 498 | of SEQ ID |
| | | | | | | | NO: 498 |
| DNA | Reverse | | 1 | | TA | nt 75 to nt 83 of | nt 1 to nt 74 |
| | | | | | | SEQ ID NO: 499 | of SEQ ID |
| | | | | | | | NO: 499 |
| DNA | Forward | NmeAIII | 0 | PacI | | nt 1 to nt 11 of | nt 12 to nt 81 |
| | | | | | | SEQ ID NO: 500 | of SEQ ID |
| | | | | | | | NO: 500 |
| DNA | Reverse | | 0 | | AT | nt 75 to nt 83 of | nt 1 to nt 74 |
| | | | | | | SEQ ID NO: 501 | of SEQ ID |
| | | | | | | | NO: 501 |

In certain embodiments, any sequence(s) listed and characterized in Table IV, or any combinations thereof, may be used in the compositions, methods or kits described herein.

TABLE IV

| | | | | | | crRNA | trascrRNA |
|---|---|---|---|---|---|---|---|
| | | Type IIS | Type IIS | Restriction | | (SEQ | (SEQ ID |
| Type | Strand | enzyme | site position | Enzyme | Overhang | ID NO) | NO) |
| DNA | Forward | MmeI | 2 | ScrFI | N | 502 | 550 |
| DNA | Reverse | | 2 | | | 503 | 551 |
| DNA | Forward | MmeI | 2 | ScrFI | N | 504 | 552 |
| | | (Engine- | | | | | |
| | | ered) | | | | | |
| DNA | Reverse | | 2 | | | 505 | 553 |
| DNA | Forward | MmeI | 2 | MspI or | CG | 506 | 554 |
| | | (Engine- | | HpaII | | | |
| | | ered) | | | | | |
| DNA | Reverse | | 2 | | | 507 | 555 |
| DNA | Forward | MmeI | 2 | BfaI | TA | 508 | 556 |
| | | (Engine- | | | | | |
| | | ered) | | | | | |
| DNA | Reverse | | 2 | | | 509 | 557 |
| DNA | Forward | MmeI | 2 | PacI | | 510 | 558 |
| | | (Engine- | | | | | |
| | | ered) | | | | | |
| DNA | Reverse | | 2 | | AT | 511 | 559 |
| DNA | Forward | MmeI | 2 | PacI | | 512 | 560 |
| DNA | Reverse | | 2 | | AT | 513 | 561 |
| DNA | Forward | MmeI | 1 | MspI or | CG | 514 | 562 |
| | | | | HpaII | | | |
| DNA | Reverse | | 1 | | | 515 | 563 |
| DNA | Forward | MmeI | 1 | ScrFI | N | 516 | 564 |
| | | (Engine- | | | | | |
| | | ered) | | | | | |
| DNA | Reverse | | 1 | | | 517 | 565 |
| DNA | Forward | MmeI | 1 | ScrFI | N | 518 | 566 |
| DNA | Reverse | | 1 | | | 519 | 567 |
| DNA | Forward | MmeI | 1 | PacI | | 520 | 568 |
| | | (Engine- | | | | | |
| | | ered) | | | | | |
| DNA | Reverse | | 1 | | AT | 521 | 569 |
| DNA | Forward | MmeI | 1 | PacI | | 522 | 570 |
| DNA | Reverse | | 1 | | AT | 523 | 571 |
| DNA | Forward | MmeI | 0 | PacI | | 524 | 572 |
| | | (Engine- | | | | | |
| | | ered) | | | | | |
| DNA | Reverse | | 0 | | AT | 525 | 573 |
| DNA | Forward | MmeI | 0 | PacI | | 526 | 574 |
| DNA | Reverse | | 0 | | AT | 527 | 575 |
| DNA | Forward | NmeAIII | 3 | MspI or | CG | 528 | 576 |
| | | | | HpaII | | | |
| DNA | Reverse | | 3 | | | 529 | 577 |
| DNA | Forward | NmeAIII | 3 | ScrFI | N | 530 | 578 |
| DNA | Reverse | | 3 | | | 531 | 579 |
| DNA | Forward | NmeAIII | 3 | BfaI | TA | 532 | 580 |
| DNA | Reverse | | 3 | | | 533 | 581 |
| DNA | Forward | NmeAIII | 3 | PacI | | 534 | 582 |
| DNA | Reverse | | 3 | | AT | 535 | 583 |
| DNA | Forward | NmeAIII | 2 | MspI or | CG | 536 | 584 |
| | | | | HpaII | | | |
| DNA | Reverse | | 2 | | | 537 | 585 |
| DNA | Forward | NmeAIII | 2 | ScrFI | N | 538 | 586 |
| DNA | Reverse | | 2 | | | 539 | 587 |
| DNA | Forward | NmeAIII | 2 | BfaI | TA | 540 | 588 |
| DNA | Reverse | | 2 | | | 541 | 589 |
| DNA | Forward | NmeAIII | 2 | PacI | | 542 | 590 |
| DNA | Reverse | | 2 | | AT | 543 | 591 |
| DNA | Forward | NmeAIII | 1 | ScrFI | N | 544 | 592 |
| DNA | Reverse | | 1 | | | 545 | 593 |
| DNA | Forward | NmeAIII | 1 | PacI | | 546 | 594 |
| DNA | Reverse | | 1 | | AT | 547 | 595 |
| DNA | Forward | NmeAIII | 0 | PacI | | 548 | 596 |
| DNA | Reverse | | 0 | | AT | 549 | 597 |

In various embodiments, the polynucleotide(s) further include(s) a modification at least one modified sugar moiety, at least one modified internucleotide linkage, at least one modified nucleotide, or combinations thereof. The modification of various embodiments can be located at or adjacent to the end of the polynucleotide. In various embodiments, the internucleotide linkage includes phosphorothioate, alkylphosphonate, phosphorodithioate, alkylphosphonothioate, phosphoramidate, carbamate, carbonate, phosphate triester, acetamidate, carboxymethyl ester, or combinations thereof. In various embodiments, the modified nucleotide is selected from a peptide nucleic acid, a locked nucleic acid (LNA), or combination thereof. In various embodiments, the modified sugar moiety is selected from: a 2′-O-methoxyethyl modified sugar moiety, a 2′-methoxy modified sugar moiety, a 2′-O-alkyl modified sugar moiety, a bicyclic sugar moiety, or combinations thereof. For example, the last two nucleotides from an end of the polynucleotide of various embodiments can have phosphate backbones that have been modified to include phosphorothioate. In this example, phosphorothioate is resistant to nuclease degradation and allows for the polynucleotide of various embodiments to be ligated with various types of DNA when during the ligation is occurring in the presence of nucleases.

In various embodiments, the polynucleotide(s) is/are attached, affixed, or immobilized on a support such as a solid support. The support of various embodiments can include two-dimensional surfaces such as microarray slides or three-dimensional surfaces such as beads or micro-spheres including polystyrene micro-spheres, magnetic micro spheres, silica micro-spheres, or fluorescent micro-spheres.

In various embodiments are disclosed expression cassettes, plasmid, or vectors including the polynucleotide encoding for the CRISPR sgRNA or CRISPR crRNA. In other embodiments are disclosed expression cassettes, plasmid, or vectors including the polynucleotide encoding for the CRISPR sgRNA or CRISPR crRNA of various embodiments and a promoter polynucleotide operably linked to the polynucleotide of various embodiments, wherein the promoter polynucleotide is recognized by an RNA polymerase and is capable of directing the RNA polymerase to transcribe the CRISPR sgRNA or CRISPR crRNA from the polynucleotide of various embodiments. For example, the polynucleotide could be oriented within a plasmid including a topoisomerase as described in U.S. Pat. No. 5,766,891, which is incorporated in its entirety by reference herein, or a cloning system such as a TOPO® Cloning System (Thermo Fisher Scientific).

### Enzymatic CRISPR Library Generation Kit

In some embodiments, a kit comprises one or more reagents for use in a process utilizing one or more of the elements described herein. Reagents may be provided in any suitable container. For example, a kit may provide one or more reaction or storage buffers. Reagents may be provided in a form that is usable in a particular assay, or in a form that requires addition of one or more other components before use (e.g. in concentrate or lyophilized form). A buffer can be any buffer, including but not limited to a sodium carbonate buffer, a sodium bicarbonate buffer, a borate buffer, a Tris buffer, a MOPS buffer, a HEPES buffer, and combinations thereof. In some embodiments, the buffer is alkaline. In some embodiments, the buffer has a pH from about 7 to about 10. In some embodiments, the kit comprises one or more oligonucleotides corresponding to a guide sequence for insertion into a vector so as to operably link the guide sequence and a regulatory element. In some embodiments, the kit comprises a homologous recombination template polynucleotide. In certain preferred embodiments, the buffer contains 50 mM Potassium Acetate; 20 mM Tris Acetate; 10 mM Magnesium Acetate; 100 μg/ml Bovine Serum Albumin; 1 mM ATP and 7.5% Polyethylene Glycol 6000.

Reaction mixes for an exemplary two step reaction process are set forth below.

### 1. HpaII Digestion/Cas9 Adapter Ligation (20° C. for 30 Min)

| | | |
|---|---|---|
| 27.5 | μl | Water (up to 50 μl) |
| 2 | μl | Lambda DNA 500 ng/μl (>10 pmol cut sites) |
| 5 | μl | 10x cutsmart buffer |
| 5 | μl | 10 mM ATP |
| 1 | μl | 10 μM scaffold adapter (10 pmol) |
| 1 | μl | HpaII |
| 1 | μl | T4 DNA Ligase (high concentration) |
| 7.5 | μl | 50% PEG 6000 |

Resuspend capture beads in the reaction for 15 minutes.
Wash twice and place in a new tube.

### 2. MmeI Digestion/T7 Adapter Ligation (20° C. for 30 Min), Resuspend in:

| | | |
|---|---|---|
| 26.5 | μl | Water |
| 5 | μl | 10x Cutsmart buffer |
| 5 | μl | 10 mM ATP |
| 3 | μl | 10 uM T7 adapter (30 pmol) |
| 1 | μl | 2.5 mM SAM |
| 1 | μl | MmeI (G6) |
| 1 | μl | T4 Ligase (high concentration) |
| 7.5 | μl | 50% PEG 6000 |

Wash twice and place in a new tube.

### 3. Bst Elution and Nick Repair (45° C. for 15 Minutes), Resuspend in:

| | | |
|---|---|---|
| 43 | μl | H20 |
| 5 | μl | 10x Cutsmart buffer |
| 1 | μl | 10 mM dNTPs |
| 1 | μl | Bst 3.0 polymerase |

Collect buffer and column purify
May PCR amplify if desired

Most current applications of CRISPR-Cas9 take advantage of this simplicity to target single or small sets of genetic loci. Although this results in rapid and efficient targeting of single genes, CRISPR-Cas9 is not widely used for screening mutants to discover novel genes and pathways because of the expense of synthesizing complex libraries. We have generated a novel method for creating CRISPR libraries efficiently and inexpensively from any source of DNA, including genomic DNA from any species, PCR products, double-stranded cDNA, etc. There are two versions of the kit, one using a combination of enzymes that comprehensively digests the input DNA at available PAM sites to create all possible CRISPRS and one that selects one of these enzymes to create high levels, but not comprehensive coverage. The advantage of the second is that it removes the need for blunting the digestion products. The presently described guide strand libraries can be used in any species and requires only a small amount of enzymes. The protocol can be adapted for all sources of DNA and can be completed in under four hours.

In one aspect, a kit for practicing the methods disclosed can comprise magnetic beads with attached oligos (e.g, streptavidin beads with biotinylated oligos), at least two different enzymes, optionally a mung bean nuclease, one or more enzyme digestion buffers, polyethylene glycol (PEG), ATP, and a T7 promoter (or other suitable promoter) oligo.

The following materials and methods are provided to facilitate the practice of the exemplified subject matter.

### Materials and Methods

### DNA Substrates for Library Construction

Two libraries were made. For the first library, Lambda phage DNA, commonly used as a control DNA substrate, was ordered from New England Biolabs. The phage DNA was isolated from a dam-E. coli strain, which can be digested with restriction endonucleases that are sensitive to dam methylation. The small, well-defined genome, at 48,502 base pairs in length, results in a library of 656 predicted sgRNAs when used as a substrate for library generation. For the second library, A 200 base pair fragment corresponding to the first exon of the tyrosinase gene in zebrafish was amplified by PCR. This fragment was selected due to the single HpaII cut site in the middle of the fragment. When used as a substrate for library generation, the library results in 2 sgRNAs that target the coding region of the Tyr gene, which could be used in knockout experiments.

### Adapter Preparation

Oligos were synthesised and ordered from IDT and were either purified using standard desalting purification or PAGE purification for longer oligos. Complementary oligos were combined in Cutsmart buffer from New England Biolabs, and hybridized by heat denaturation at 95° C. for two minutes and slowly cooled to room temperature using a PCR machine. The scaffold adapter contained either the standard sgRNA sequence, or a sequence with an extended first stem loop that more closely resembles the native two-part crRNA:tracrRNA system and has been shown to be more efficient in some cases. The longer adapter also contained a cloning adapter sequence that could be digested for cloning into a plasmid using Golden Gate assembly. The adapter also contained either the sequence for the standard MmeI enzyme or a rationally engineered variant that recognized G instead of C in the sixth position of the binding site. The adapter was also designed to contain a two base pair sticky end that matches the enzyme first used to digest the starting DNA substrate. The adapter also contained a long (˜20 nt) single stranded overhang that could be used to capture the library from solution using magnetic beads.

The promoter adapter was designed to contain either the T7 RNA polymerase promoter sequence for direct transcription of the library or a cloning adapter sequence that could be digested for cloning into a plasmid using Golden Gate assembly. The adapter was designed to contain a two base-pair, NN overhang compatible with the NN overhang created by MmeI digestion.

In some embodiments, a portion of the constant region is provided. In this case, the first adapter contains a non-palindromic Type II restriction site within a sequence coding for only a portion of the CRISPR guide, with the remainder added via the second adapter or by cloning the final product into a vector containing the remaining portion of the sequence. For example, the Cas9 sgRNA sequence, e.g., SEQ ID NO: 1 contains restriction sites for the Type II enzymes AluI, SetI, MseI, TspGWI, BanI, NlaIV, HinfI, PleI, and MlyI. An adapter containing a modified sgRNA sequence containing a non-palindromic Type II restriction site and the guide RNA sequence up to and including the restriction enzyme binding site of choice could be synthesized, used in our method, and then cloned into a vector containing the remaining sgRNA template using said restriction enzyme. The resulting vector would then contain a complete sgRNA or crRNA constant region capable of coding for an RNA that binds Cas9. Based on the data we presented here, the desired site can also be engineered into the constant region sequence if appropriate modifications are made to maintain the necessary stem loop structures. Finally, in alternative approaches, other cloning methods, such as Gibson, Golden Gate, blunt-ended cloning, or synthesized compatible overhangs that do not require the presence of a restriction site, can also be employed.

Enzymatic CRISPR/Cas9 sgRNA Library Generation

CRISPR sgRNA template libraries were created enzymatically using a novel method presented here. Input DNA was first digested using the HpaII enzyme, which digests at the PAM containing motif CCGG, and simultaneously ligated in the same reaction to an sgRNA scaffold template modified to contain an MmeI site and a ligation overhang. The ligation overhang is designed to be compatible with the CG overhang created by HpaII digestion without regenerating the site upon ligation to prevent redigestion of the ligation product. The ligation product was then purified from the reaction using magnetic beads containing a capture oligo for the sgRNA scaffold template. Following purification, the beads were washed and resuspended in a second reaction containing MmeI to digest 18-19 bases upstream of the scaffold, creating a protospacer, and ligase to attach a U6 promoter. The reaction was washed and the resulting sgRNA template eluted from the beads. Finally, the library was amplified using 10 cycles of PCR.

### Capture Beads Preparation

50 μL of streptavidin coated magnetic beads were resuspended in 20 μL of 1× CutSmart® Buffer containing 100 pmol of biotinylated oligo. After incubating beads at room temperature for 15 minutes, 30 μL of 1× CutSmart® Buffer was added and the beads were washed twice and placed in a new tube.

Library Displacement from Magnetic Beads

Capture oligos were designed to be complementary to the long single stranded end of the scaffold adapter. The oligos were designed to hybridize at 50° C. so that they could hybridize to the scaffold adapter at room temperature. They were also designed with a biotin moiety on the 3′ end, so that the 5′ end was protruding from the beads.

After preparation of the capture beads, the tube was placed in a magnetic rack for 1 minute or until the solution was clear. The supernatant was then removed and discarded from the tube. The beads were then resuspended in the solution that contained the final library template or an intermediate product in order to hybridize it to the capture oligo, thereby providing a method of physical separation using a magnetic field. The beads were then washed with Cutsmart buffer to remove reagents or products not attached to the beads. The beads were then suspended in a buffer that contained the strand displacing polymerase Bst 3.0 and the dNTPs required for extension and incubated at 45° C. for 15 minutes. At this temperature the polymerase has reduced activity, but the library is not likely to melt at the nicks still in the library at this stage. Because of the directionality of the oligo attached to the beads and extension direction of the polymerase, the single stranded end of the adapter can be used as a template and extension by the polymerase displaces the capture oligo and extension prevents re-hybridization. The polymerase will also repair the nicks in the library by extending the DNA strand at the nick and displacing the nicked strand. sgRNA TranscriptionEither the MEGAscript™ T7 Transcription Kit (Thermo Fisher Scientific) or the HiScribe™ T7 Quick High Yield RNA Synthesis Kit (New England Biolabs) was used to transcribe the sgRNA template libraries. In either case 210-300 ng of the template was used and incubated at 37° C. for 2 hours. DNase I was then added and the reaction was then incubated for an additional 15 minutes at 37° C. The sgRNA was then purified using either the RNA Clean & Concentrator-5 Kit (Zymo Research) or by phenol-chloroform extraction.

### Cas9 In Vitro Digestion

Cas9 Nuclease from S. pyogenes and 10×NEBuffer 3.1 were purchased from New England Biolabs. The transcribed sgRNA library was allowed to complex with Cas9 molecules in the buffer. The DNA substrate to be digested was then added to the solution.

cDNA Synthesis and Normalization

In an eukaryotic cell, the mRNA population constitutes approximately 1% of total RNA with the number of transcripts varying from several thousand to several tens of thousands. Normally, the high abundance transcripts (several thousand mRNA copies per cell) of as few as 5-10 genes account for 20% of the cellular mRNA. The intermediate abundance transcripts (several hundred copies per cell) of 500-2000 genes constitute about 40-60% of the cellular mRNA. The remaining 20-40% of mRNA is represented by rare transcripts (from one to several dozen mRNA copies per cell). Such an enormous difference in abundance complicates large-scale transcriptome analysis, which results in recurrent sequencing of more abundant cDNAs. cDNA normalization decreases the prevalence of high abundance transcripts and equalizes transcript concentrations in a cDNA sample, thereby dramatically increasing the efficiency of sequencing and rare gene discovery.

One approach for nucleic acid normalization exemplified in PANC1 Rat Beta Cells entails collection of cells by trypsinizing a 10 cm plate and placing the sample in Trizol solution. Total RNA was then extracted using the Zymo Directzol kit (Zymo Research) using standard procedures. cDNA of polyadenylated RNA species was then generated using the Mint-2 cDNA synthesis kit (Evrogen) and normalized by subtractive hybridization using the Trimmer-2 kit (Evrogen).

The examples following are provided to illustrate certain embodiments of the invention. They are not intended to limit the invention in any way.

### EXAMPLES

### Method Design and Overview

CRISPR libraries are rapidly becoming an important tool for gene discovery across a wide-range of fields and applications, including drug screening, crop improvement, molecular studies of embryonic development, and others. Current methods for these experiments synthesize large pooled libraries of individually designed CRISPR sgRNAs. While synthesis is straight-forward, it is limited by several factors. First, although prices have decreased in recent years, synthesis of large pools remains expensive and requires a turnaround time of 3-4 weeks. In addition, synthesis reactions can introduce errors, lowering the percentage of guides with the correct sequence. Cost constraints also limit the coverage of the library to a few guides per gene and severely hinder the ability to make custom libraries targeted to a specific cell-type or tissue. Finally, these libraries require significant a priori knowledge of the genome as they rely

on the genomic sequence and annotations to design and choose guides. This limits the ability to variant match the library to a particular sample and can completely prevent their use in many species with poor genomic annotation data.

One method developed in our applications. In the present example, in order to further reduce the total number of steps in our earlier method, we sought to combine each pair of restriction digestion and ligation reactions into single reactions (FIGS. 1 to 4).

The resulting process involves the collection of DNA or RNA from a source tissue and enzymatically processing the sample into guides using a series of four reactions: 1) digestion of the input DNA with a restriction enzyme targeting PAM sequences, 2) ligation of the digestion product to a modified DNA scaffold, 3) digestion with a Type IIS restriction enzyme, 4) ligation of a promoter to the Type IIS ligation product. Final products are then bead-purified using a photocleavable biotin capture oligo for use in downstream-reaction process that also provides higher yields and requires less input. The method for combining each of the steps is described in detail below.

In one aspect of the method, the initial digestion and ligation steps are combined. In one aspect, the scaffold adapter sequence is modified in the repeat:anti-repeat duplex, a preferred modification is effective to prevent digestion of the scaffold adapter.

When the scaffold adapter is ligated onto the digested DNA fragments, only the overhang must match, and the restriction enzyme binding site can be destroyed during the ligation if the double stranded region has a base pair change. Destroying the restriction enzyme binding site upon ligation of the adapter would prevent the HpaII restriction enzyme from cleaving off the adapter, and permit digestion and ligation in a single reaction, simplifying the procedure. It would also force the reaction to use all of the scaffold adapter, increasing the efficiency and yield of the reaction.

Unfortunately, the HpaII and MmeI restriction sites overlap in such a way that the single base pair change cannot be made. However, it has been shown that the binding site of MmeI can be modified (Morgan and Luyten, 2009). It has been difficult to engineer most type II restriction enzymes to cut at different sequences because the binding domain of these enzymes is physically located in the same space as the endonuclease domain, so they depend on each other for maintaining the structure and function of the enzyme. However, because the DNA binding domain and the endonuclease domain of type IIS restriction enzymes such as MmeI are separate, some of the amino acids in the binding domain have been engineered to recognize other DNA sequences. We replaced the standard MmeI enzyme with an engineered version of

MmeI that binds to guanine instead of cytosine in the sixth position, TCCRAG instead of TCCRAC (Morgan and Luyten, 2009), which allowed the corresponding change to be made in the scaffold adapter sequence.

In order to combine the first digestion and ligation step into a single step, the enzyme must work in the same buffer. To increase the ligation speed and efficiency, high concentrations of ATP and 7.5% polyethylene glycol (PEG) were used in the ligation steps as a macromolecular crowding reagent. The activity of some enzymes is enhanced if PEG is included in the reaction buffer, but the activity of others is inhibited. We tested the activity of MspI (which has the same binding site as HpaII) to determine if it is affected by having PEG in the buffer and found that the activity of MspI is not affected by PEG. Thus, the digestion of input DNA and ligation of the scaffold adapter can be carried out simultaneously. We used this shortened process to create a small library. Although it was unclear whether the MmeI site could be incorporated without disrupting important interactions between the RNA and the protein, in vitro digestion of a DNA fragment with Cas9 in complex with this sgRNA showed the specificity and endonuclease activity of Cas9 was retained.

### Combining the Second Digestion and Ligation Steps (Chemical Modification of the Promoter Adapter by a Methyltransferase to Prevent Redigestion of the Promoter Adapter)

In order to combine steps 1 and 2 of the library generation process, we needed a way to prevent the digestion of the adapter after it was ligated. We attempted to change the sequence in such a way that destroyed the first restriction enzyme binding site when the adapter was ligated. Unfortunately, this required the modification of the MmeI binding site as well. A mutant MmeI enzyme (E806K and R808D) that recognized a G instead of a C in position 6 of the recognition sequence (TCCRAG instead of TCCRAC) was obtained. Using this enzyme enabled the necessary sequence modifications to achieve our goal of preventing re-digestion by the first Type II enzyme. Modified MmeI can be synthesized or commercially outsourced from companies such as NEB

MmeI is a Type IIS restriction enzyme that simultaneously methylates its binding site while cutting the DNA approximately 20 bp upstream. In biotechnological applications, this methylation is purely an artifact of the digestion and can actually be detrimental as it potentially inhibits downstream uses of the DNA, such as blocking methylation sensitive enzymes. As MmeI itself is methylation sensitive, the change has the effect of blocking re-digestion of the same DNA by MmeI itself. This is generally not desirable, as it limits the effectiveness of the enzyme by decreasing the number of enzymes bound to the DNA and forming the dimers required for MmeI function.

However, we have developed a novel use of this methylation by using it to combine the MmeI digestion and promoter ligation steps of our protocol to eliminate the need for purification between the two steps and to consolidate the incubation times. Combining these two steps results in an enzymatic reaction containing the ligated target/scaffold DNA, the upstream promoter sequence, a Type IIS restriction enzyme with methylation activity, and a ligase. Within this reaction, the DNA is first digested and simultaneously methylated by the Type IIS enzyme. This product is then ligated to the promoter sequence in the same reaction. The resulting product will not be re-digested by the MmeI due to the methyl group attached to its binding site during the first ligation. The resulting product can then be easily purified or PCR amplified.

### Combination of all Steps

All four steps outlined above can be combined into a single reaction. The two modifications listed above force the reaction to proceed in the forward direction. In the first step, the DNA can be digested with HpaII. If the DNA is re-ligated to itself or another digested DNA fragment, it will be digested again. However, if it is ligated to an adapter, it will not be digested because the binding site is destroyed. A similar process happens during the subsequent MmeI digestion and T7 ligation. Although the binding site for MmeI cannot be destroyed, when it cleaves, it methylates the A in the second position, preventing MmeI from cutting again at that site, and therefore will not be able to cut off the T7 adapter after it has been ligated. In addition, each ligation reaction involves unique overhangs not compatible with any other product or the oligos are not phosphorylated. For example, the T7 adapter cannot ligate directly to the first adapter, even if present during the initial digestion. Therefore, the four steps are combinable, and all four steps can be carried out in a single reaction.

### Nicked Ligation Products

During the library generation process, adapters are ligated onto digested DNA fragments. Unphosphorylated adapters were synthesized, as these adapters are incapable of ligating to themselves, but this also results in a nick in the final ligation product. The data presented herein demonstrate that the ligation reaction can still take place even if a single phosphate group is present on only one of the double stranded DNA fragments.

In a later step, the nick generated is repaired using a polymerase. In addition, without modification, the second adapter can also ligate to itself, decreasing the efficiency of the library construction process. To avoid this, the second set of adapters were also synthesized without a phosphate group. In vitro digestion with the MmeI restriction enzyme demonstrated that it cuts across this nick with high efficiency. In certain instances, the ratio of the DNA input to the adapter was increased to 2:1, which reduces the formation of any aberrant shorter library products below detectable levels.

### Polyethylene Glycol (PEG) as a Molecular Crowding Agent

The speed and efficiency of the ligation steps were also an important consideration when developing the method described here. It has been shown that polyethylene glycol (PEG) can be used to speed up enzymatic reactions, but it can also decrease the enzymatic activity of some enzymes (Zimmerman and Pheiffer, 1983). T4 ligase activity is demonstrably increased when a macromolecular crowding reagent such as PEG is used. By using a buffer with a final concentration of 4%, 5%, 6%, and more preferably, 7.5% and up to about 10% PEG 6000 and using a high concentration of both T4 DNA ligase (8000-100,000 units per μL and ATP (0.1 tp 10 mM 1 mM) we were able to increase the speed and efficiency of these ligation reactions. The other enzymes we used were also tested to determine their activity in response to the molecular crowding reagent used. The enzymatic activity of MspI did not change based on visualization of digested DNA on a gel However, the activity of MmeI was somewhat reduced, but still functional.

### Construction of the Library on the Surface Magnetic Beads

To simplify the purification and handling of the sgRNA library during construction, the library was constructed on the surface of streptavidin coated magnetic beads. Magnetic beads with a poly-T oligonucleotide can be used to capture mRNA in the preparation of cDNA. Some methods for building out strands of DNA on magnetic beads have also been published (Pengpumkiat et al., 2016). Because magnetic beads have liquid-phase like reaction kinetics, they are suitable for construction of the library using the method reactions described herein. Streptavidin magnetic beads (50 μL) bind about 100 pmol of biotinylated DNA. Different biotinylated oligos were synthesized and the binding capacity of the beads was tested in both

the recommended binding buffer as well as CutSmart® buffer. There was little difference between the two buffers when attaching the oligos to the beads. Double stranded DNA adapters that include a single biotin molecule were also attached to the beads. All of the different schemes for attaching the library to the beads we tested were shown to be effective. The library was attached to the beads and the resultant product purified before digesting with MmeI to increase efficiency of cleavage. Notably, an alternative approach could entail increasing the concentration of MmeI and performing digestion prior to bead attachment. However, the present method, where the substrates were purified before cutting, allowed the buffer to be exchanged for a buffer optimized for the endonuclease activity of MmeI.
Library Displacement from the Solid Support

Magnetic beads have been used in biological reactions as a solid support to facilitate post-reaction cleanup, buffer exchange, and enzyme removal. Oligonucleotides can be attached to solid supports using several different methods, but removal from the beads is often desired for downstream applications. Because of problems encountered with removal of the library from the beads using UV light, alternative methods of detaching the library were employed.

One method employs a restriction enzyme to cleave the DNA. However, increasing the number of enzymes could potentially reduce the overall complexity of the library as they would occasionally target the genomic protospacers. This could be mitigated by using extremely rare cutting enzymes (e.g. meganucleases). Chemical methods were also considered, but would require a separate buffer and it was unclear whether the altered conditions would damage the library. It is possible to add the beads to a PCR reaction, but because the library contained nicks, heating the library would result in the library fragments separating and incorrect amplification products would be generated. Furthermore, depending on the fidelity of the polymerase, PCR could increase the number of mutations in the final library. Finally, antibody methods are available, but they can be expensive.

Another method employs a photo-cleavable biotin moiety attached to the scaffold adapter. This biotin moiety has been synthesized previously and can be attached to

the 5′ end of an oligonucleotide. Upon irradiation with ultraviolet light, the biotin is cleaved from the oligo and leaves the oligo with a phosphorylated 5′ end. We found that in a clear solution, the biotin is readily cleaved with the UV light. However, when attached to magnetic beads, only a small fraction of the biotin was actually cleaved, likely due to the opacity of the beads interfering with passage of light to the photocleavable moiety. Using agarose beads could have solved this problem, but the handling capabilities of the magnetic beads are very convenient and can easily be incorporated into automated processes.

An alternative approach entails displacement of the library from the beads using a short oligo. DNA nanotechnology applications can use strand displacement reactions to construct 3 dimensional structures (Ijas et al., 2018). Strand displacement reactions can also be used to build logical circuits and therefore carry out mathematical calculations (Jiang et al., 2019). Toehold-mediated strand displacement entails use of a toehold (e.g., a short, single stranded overhang on a DNA duplex). When a third oligonucleotide is added that is complementary not only to the duplex, but the toehold as well, this oligo can displace the shorter oligo of the duplex. We designed an adapter that could be captured by a short biotinylated oligo through hybridization and then displaced by another short oligo. However, as described below, the strand could be displaced by the Bst 3.0 polymerase used to repair nicks in the library, and thus we proceed with the Bst 3.0 based approach. The strand displacing polymerase Bst 3.0 has high displacement activity and can extend DNA at nicks, displacing the strand as it fills in the template. Because there was a nick in the DNA where the capture oligo hybridized during the capture of the library (FIG. 3), the Bst polymerase should displace the capture oligo and fill in the gap at the same time, essentially eluting the library from the beads.

An adapter was designed with a 19 bp overhang with a melting temperature of approximately 50° C. that could hybridize to the short biotinylated capture oligo attached to the beads. The adapter can then be attached to the beads by simply incubating at room temperature with the capture beads. Incubating the beads with Bst 3.0 elongates the DNA at the nick and displaces the library from the beads. Because the polymerase extends the DNA to the end of the fragment, this reaction is not reversible, and the library remains detached from the beads. Notably, other strand-displacing enzymes in addition to Bst3.0 (e.g., Phi29, Bst1.0, Bst 2.0 and large Klenow fragment) can be employed for this purpose.

In summary, polymerase dependent strand displacement can be used to irreversibly elute oligonucleotides from solid supports, creating complete, double-stranded DNA in the process. We also confirmed that the adapters would still hybridize to the beads in the presence of PEG. Our data reveal that DNA actually hybridizes better in PEG and experiments show that hybridization of the adapter to the beads takes place in 7.5% PEG when resuspended well. It is, therefore, possible to carry out ligations in PEG and subsequently hybridize the DNA to the beads.

To transcribe the sgRNA, the nicks must first be repaired. T4 polynucleotide kinase has little activity on nicked DNA substrates, so the nicks could not simply be sealed by phosphorylation and ligation. By deciding which strands would be phosphorylated during the process, using either addition or removal of a phosphate group, nicks were placed in positions that would allow nick repair using a strand displacing polymerase. As discussed above, the final library was contacted with the DNA polymerase Bst 3.0 and dNTPs and the data demonstrated that it effectively repaired the nicks in the library. The polymerase has full activity at 65° C. and 75% activity at 45° C. Because the overlap holding the library fragments at this point is only 18 bp long, we incubated the beads at 45° C. to prevent the fragments from coming apart. The strand displacing polymerase phi29 works at lower temperatures and could also be used to displace the library, although the displacement activity of this polymerase is not as high. In this approach, the single stranded fragments left after this process can then be removed using standard methods.

Phosphorothioate Protected Adapters with Endonuclease Cocktail

An alternative purification method is to selectively degrade any incomplete DNA products. This entails protecting both sides of the final library with adapters that included phosphorothioate linkages to protect from exonuclease degradation and then adding the appropriate nucleases. The presence of 6 consecutive phosphorothioates in place of phosphates at the 5′ end of an oligonucleotide can effectively block digestion by lambda exonuclease (Eckstein and Gish, 2018). Lambda Exonuclease works on double stranded and single stranded DNA substrates in the 5′ to 3′ direction but is effectively blocked by 6 consecutive phosphorothioates on the 5′ end (shown by black bold letters in FIG. 3). Exonuclease I can only act on single stranded DNA substrates and digests in the 3′ to 5′ direction. Adapters were synthesized with phosphorothioates such that digestion by exonucleases was prevented if and only if both adapters are attached at opposite ends of the product. Because the final library will be the only product with the protecting groups on both ends, it will be the only product that will not be degraded when both exonucleases are added to the solution (FIG. 4). This method can be used in place of the bead purification method.

In the present example, a rapid, efficient method for enzymatically generating CRISPR sgRNA libraries by combining restriction and ligation steps in a single reaction vessel is disclosed. Combining these steps makes the process faster and more efficient. Mixing the enzymes into a single reaction eliminates the need for a heat inactivation step and a second incubation step, reducing the amount of time and number of sample manipulations required in the process. Combining these steps also increases the efficiency of the reaction. In both steps, the elimination of additional magnetic bead separation and wash steps limits the losses of reaction product that occur during these steps. Further advantages are achieved in the first step because the reagents are designed to drive the reaction to completion. During the ligation reaction, two products can occur: the ligation with the scaffold, which is desirable, and ligation with another digestion product, which is not. The undesirable ligation of two products from the first digestion are normally removed during later purification steps as an unwanted side-product. As these fragments are not included in the final library, more input DNA must be supplied to account for these losses. Our inventive modification to the scaffold results in a ligation product without the restriction site, thereby preventing re-digestion of the desired product. Accordingly, the desired ligation products accumulate, while ligations between two DNA input fragments are re-digested by the restriction enzyme—driving the equilibrium of the reaction towards completion and reducing unwanted side products.

### Sources of Input DNA

CRISPR gRNA libraries generated using the process described herein can be used in highly customizable CRISPR library applications as they can be generated from a wide variety of DNA sources. Thus, the first step in enzymatically generating an sgRNA library is to decide on a DNA substrate to use as a starting material, as the substrate that is acquired will determine what the library can be used for in downstream applications. Below are several different, but non-limiting options of sources that can be used as a starting substrate. For example

### Option 1: Genomic DNA

PANC1 Rat Beta Cells were collected by trypsinizing a 10 cm plate and DNA extracted using the GeneCatcher gDNA (genomic DNA) Blood Kit (ThermoFisher).

Option 2: cDNA Synthesis and Normalization

PANC1 Rat Beta Cells were collected by trypsinizing a 10 cm plate and placing the sample in Trizol solution. Total RNA was then extracted using the Zymo Directzol kit (Zymo Research) using standard procedures. cDNA of polyadenylated RNA species was then generated using the Mint-2 cDNA synthesis kit (Evrogen) and normalized by subtractive hybridization using the Trimmer-2 kit (Evrogen).

### Option 3: Chromatin Immunoprecipitation

Chromatin immunoprecipitation (ChIP) was performed as previously described (Schaffer et al., 2013). Cells were first crosslinked with formaldehyde and then lysed and DNA collected. Pulldowns were conducted with rabbit anti-Nkx6.1 antiserum (1:250). Crosslinks in the chromatin were then reversed and DNA purified by Phenol:Chloroform extraction followed by ethanol precipitation.

### Option 4: PCR Amplification of the Region of Interest

The putative enhancer region driving insulin expression was amplified by PCR. As the region was 20 kb long, 10 PCR reactions were created, each producing a 2 kb fragment. The PCR products were then purified using the Zymo Research DNA Clean and Concentrate kit and pooled for library generation.

### Lentivirus Generation

The resulting Beta cell transcriptome sgRNA library was cloned into a modified lentiviral vector containing the Cas9 coding sequence under the control of the ef1alpha core promoter. Thus, a single lentivirus will produce both Cas9 and the sgRNA. Calculations based on previously published RNA-seq data showed that beta-cells express approximately 11,000 genes. Thus, based on an average cutting distance of 256 bases (with two guides made at each cut) and an average mRNA length of 2.1 kb, we estimate our library contains approximately 200,000 sgRNA sequences targeting each expressed gene an average of 16 times—providing a high coverage library targeted to beta-cell relevant genes. Lentiviral constructs were then electroporated into DH5alpha cells and grown overnight before harvesting the plasmids using the Monarch Plasmid Miniprep kit (New England Biolabs). A total of 50 electroporations were conducted to ensure at least 10× coverage of the library. The sgRNA templates in the resulting library were PCR amplified and sequenced on an Illumina 2500 sequencer in the BYU Sequencing Center to characterize the library before transfection.

An Exemplary Protocol for Enzymatically Generating sgRNAs
1. Attach gRNA scaffolds to beads (30 min, concurrent with step 2 or provided in kit)
   1.1. Place 10 μl of beads in a 1.5 ml microcentrifuge tube, place on magnet for 1 minute and discard supernatant
   1.2. Add 500 μl of wash buffer, rock for 5 minutes, place on magnet for 1 minute and discard supernatant
   1.3. Add 50 μl of scaffold (100 ng/ul biotinylated and annealed scaffold oligo) solution
   1.4. Incubate for 10 minutes with gentle rocking, place on magnet for 1 minute and discard supernatant.
   1.5. Wash with 500 μl 1× cutsmart buffer, rock for 5 minutes, place on magnet for 1 minute and discard supernatant
2. Digest source DNA (e.g. genomic DNA, a PCR product, or double-stranded cDNA) (30 min, concurrent with step 1)
   2.1. Combine 1 μg of DNA with 5 μl Cutsmart buffer, and 1 μl digestion enzyme or enzymes (e.g. MspI) and bring to 50 μl with water, incubate for 30 minutes at 37° C.
3. Ligate DNA to scaffolds (10 min)
   3.1. Add digested DNA mix from Step 1, 1 μl Ligase and 1 μl ATP+PEG to beads. Rock for 5 minutes, place on magnet for 1 minute and discard supernatant.
   3.2. Wash with 500 μl 1× cutsmart buffer, rock for 5 minutes, place on magnet for 1 minute and discard supernatant
4. Digest with MmeI (45 min)
   4.1. Add 24 μl of 1× cutsmart buffer and 1 ul of MmeI enzyme, incubate at 37° C. for 30 min with rocking, place on magnet for 1 minute and discard supernatant.
   4.2. Wash with 500 μl 1× cutsmart buffer, rock for 5 minutes, place on magnet for 1 minute and discard supernatant
5. Ligate T7 promoter (10 minutes)
   5.1. Add 50 μl T7 promoter mix, 1 μl Ligase and 1 μl ATP+PEG to beads. Rock for 5 minutes, place on magnet for 1 minute and discard supernatant.
   5.2. Wash with 500 μl 1×PCR buffer, rock for 5 minutes, place on magnet for 1 minute and discard supernatant
6. Purify library (15 minutes)
   6.1. Resuspend beads in 50 μl Bead elution buffer containing 5 μl 10× cutsmart buffer, 1 μl dNTP, and 1 μl Bst 3.0.
   6.2. Transfer to PCR tube and incubate at 45° C. for 15 minutes.
   6.3. Place beads on magnet and keep supernatant. Discard beads.

An Exemplary Protocol for Enzymatically Generating sgRNAs with a combined first digestion and ligation
1. Attach gRNA scaffolds to beads (30 min, may be provided in kit)
   1.1. Place 10 μl of beads in a 1.5 ml microcentrifuge tube, place on magnet for 1 minute and discard supernatant
   1.2. Add 500 μl of wash buffer, rock for 5 minutes, place on magnet for 1 minute and discard supernatant
   1.3. Add 50 μl of scaffold (100 ng/ul biotinylated and annealed scaffold oligo) solution
   1.4. Incubate for 10 minutes with gentle rocking, place on magnet for 1 minute and discard supernatant.
   1.5. Wash with 500 μl 1× cutsmart buffer, rock for 5 minutes, place on magnet for 1 minute and discard supernatant
2. Digest and ligate source DNA (e.g. genomic DNA, a PCR product, or double-stranded cDNA) (30 min)
   2.1. Combine 1 μg of DNA with 5 μl Cutsmart buffer, 1 μl enzyme or enzymes (e.g. MspI), 1 μl Ligase and 1 μl ATP+PEG. Bring to 50 μl with water and add to beads. Incubate for 30 minutes, place on magnet for 1 minute and discard supernatant.
   2.2. Wash with 500 μl 1× cutsmart buffer, rock for 5 minutes, place on magnet for 1 minute and discard supernatant
3. Digest with MmeI (45 min)
   3.1. Add 24 μl of 1× cutsmart buffer and 1 ul of MmeI enzyme, incubate at 37° C. for 30 min with rocking, place on magnet for 1 minute and discard supernatant.
   3.2. Wash with 500 μl 1× cutsmart buffer, rock for 5 minutes, place on magnet for 1 minute and discard supernatant
4. Ligate T7 promoter (10 minutes)
   4.1. Add 50 μl T7 promoter mix, 1 μl Ligase and 1 μl ATP+PEG to beads. Rock for 5 minutes, place on magnet for 1 minute and discard supernatant.
   4.2. Wash with 500 μl 1×PCR buffer, rock for 5 minutes, place on magnet for 1 minute and discard supernatant
5. Purify library (15 minutes)
   5.1. Resuspend beads in 50 μl Bead elution buffer containing 5 μl 10× cutsmart buffer, 1 μl dNTP, and 1 μl Bst 3.0.
   5.2. Transfer to PCR tube and incubate at 45° C. for 15 minutes.
   5.3. Place beads on magnet and keep supernatant. Discard beads.

An Exemplary Protocol for Enzymatically Generating sgRNAs with a combined second digestion and ligation
1. Attach gRNA scaffolds to beads (30 min, may be provided in kit)
   1.1. Place 10 μl of beads in a 1.5 ml microcentrifuge tube, place on magnet for 1 minute and discard supernatant
   1.2. Add 500 μl of wash buffer, rock for 5 minutes, place on magnet for 1 minute and discard supernatant
   1.3. Add 50 μl of scaffold (100 ng/ul biotinylated and annealed scaffold oligo) solution
   1.4. Incubate for 10 minutes with gentle rocking, place on magnet for 1 minute and discard supernatant.
   1.5. Wash with 500 μl 1× cutsmart buffer, rock for 5 minutes, place on magnet for 1 minute and discard supernatant
2. Digest source DNA (e.g., genomic DNA, a PCR product, or double-stranded cDNA) (30 min)
   2.1. Combine 1 μg of DNA with 5 μl Cutsmart buffer, 1 μl digestion enzyme or enzymes (e.g. MspI) and bring to 50 μl with water, incubate for 30 minutes at 37° C.
3. Ligate DNA to scaffolds (10 min)
   3.1. Add digested DNA mix from Step 1, 1 μl Ligase and 1 μl ATP+PEG to beads. Rock for 5 minutes, place on magnet for 1 minute and discard supernatant.
   3.2. Wash with 500 μl 1× cutsmart buffer, rock for 5 minutes, place on magnet for 1 minute and discard supernatant
4. Digest with MmeI and Ligate Promoter (45 min)
   4.1. Add 24 μl of 1× cutsmart buffer, 1 ul of MmeI (G6) enzyme, 1 ul 2.5 mM SAM, 22 μl T7 promoter mix, 1 μl Ligase and 1 μl ATP+PEG to beads. Incubate at 20° C. for 30 minutes, place on magnet for 1 minute and discard supernatant.
   4.2. Wash with 500 μl 1×PCR buffer, rock for 5 minutes, place on magnet for 1 minute and discard supernatant
5. Purify library (15 minutes)
   5.1. Resuspend beads in 50 μl Bead elution buffer containing 5 μl 10× cutsmart buffer, 1 μl dNTP, and 1 μl Bst 3.0.
   5.2. Transfer to PCR tube and incubate at 45° C. for 15 minutes.
   5.3. Place beads on magnet and keep supernatant. Discard beads.

An Exemplary Protocol for Enzymatically Generating sgRNAs with a combined first digestion and ligation followed by a combined second digestion and ligation
1. Attach gRNA scaffolds to beads (30 min, concurrent with step 1 or provided in kit)
   1.1. Place 10 μl of beads in a 1.5 ml microcentrifuge tube, place on magnet for 1 minute and discard supernatant
   1.2. Add 500 μl of wash buffer, rock for 5 minutes, place on magnet for 1 minute and discard supernatant
   1.3. Add 50 μl of scaffold (100 ng/ul biotinylated and annealed scaffold oligo) solution
   1.4. Incubate for 10 minutes with gentle rocking, place on magnet for 1 minute and discard supernatant.
   1.5. Wash with 500 μl 1× cutsmart buffer, rock for 5 minutes, place on magnet for 1 minute and discard supernatant
2. Digest and ligate source DNA (e.g. genomic DNA, a PCR product, or double-stranded cDNA) (30 min)
   2.1. Combine 1 μg of DNA with 5 μl Cutsmart buffer, 1 μl enzyme or enzymes (e.g. MspI), 1 μl Ligase and 1 μl ATP+PEG. Bring to 50 μl with water and add to beads. Rock for 30 minutes, place on magnet for 1 minute and discard supernatant.
   2.2. Wash with 500 μl 1× cutsmart buffer, rock for 5 minutes, place on magnet for 1 minute and discard supernatant
3. Digest with MmeI and Ligate Promoter (45 min)
   3.1. Add 24 μl of 1× cutsmart buffer, 1 ul of MmeI (G6) enzyme, 1 ul 2.5 mM SAM, 22 μl T7 promoter mix, 1 μl Ligase and 1 μl ATP+PEG to beads. Incubate at 20° C. for 30 minutes, place on magnet for 1 minute and discard supernatant.
   3.2. Wash with 500 μl 1×PCR buffer, rock for 5 minutes, place on magnet for 1 minute and discard supernatant
4. Purify library (15 minutes)
   4.1. Resuspend beads in 50 μl Bead elution buffer containing 5 μl 10× cutsmart buffer, 1 μl dNTP, and 1 μl Bst 3.0.
   4.2. Transfer to PCR tube and incubate at 45° C. for 15 minutes.
   4.3. Place beads on magnet and keep supernatant. Discard beads.

An Exemplary Protocol for Enzymatically Generating sgRNAs with a single vessel reaction for all digestion and ligation steps
1. Attach gRNA scaffolds to beads (30 min, may be provided in kit)
   1.1. Place 10 μl of beads in a 1.5 ml microcentrifuge tube, place on magnet for 1 minute and discard supernatant
   1.2. Add 500 μl of wash buffer, rock for 5 minutes, place on magnet for 1 minute and discard supernatant
   1.3. Add 50 μl of scaffold (100 ng/ul biotinylated and annealed scaffold oligo) solution
   1.4. Incubate for 10 minutes with gentle rocking, place on magnet for 1 minute and discard supernatant.
   1.5. Wash with 500 μl 1× cutsmart buffer, rock for 5 minutes, place on magnet for 1 minute and discard supernatant
2. Digest and ligate source DNA (e.g. genomic DNA, a PCR product, or double-stranded cDNA) (30 min)
   2.1. Combine 1 μg of DNA with 5 μl Cutsmart buffer, 1 μl digestion enzyme or enzymes (e.g. MspI), 1 μl Ligase, 1 ul of MmeI (G6) enzyme, 1 ul 2.5 mM SAM, 22 μl T7 promoter mix, and 1 μl ATP+PEG. Bring to 50 μl with water and add to beads. Incubate for 30 minutes, place on magnet for 1 minute and discard supernatant.
   2.2. Wash with 500 μl 1×PCR buffer, rock for 5 minutes, place on magnet for 1 minute and discard supernatant
3. Purify library (15 minutes)
   3.1. Resuspend beads in 50 μl Bead elution buffer containing 5 μl 10× cutsmart buffer, 1 μl dNTP, and 1 μl Bst 3.0.
   3.2. Transfer to PCR tube and incubate at 45° C. for 15 minutes.
   3.3. Place beads on magnet and keep supernatant. Discard beads.

The library produced as described above can be used in a variety of applications. For example, there is increasing interest in creating libraries containing thousands of different sgRNAs targeting large sets of genes (Shalem et al., 2014). Several labs have been able to create sgRNA for a few model organisms (Shalem et al., 2014, Doenchet al., 2016). One such library was designed and chemically synthesized for use in Drosophila cells. This library was composed of 40,279 different sgRNAs and targeted 13,501 different genes (Bassett et al., 2015). The library was cloned into a plasmid containing the ubiquitous U6 promoter and a separate expression construct consisting of an actin promoter followed by the Cas9 mRNA sequence. The plasmid library was then maintained in millions of bacterial colonies. After recovery of the plasmid library, it was transfected into a Drosophila cell line which was then screened for cellular phenotypes. The targeting regions of the sgRNAs in the plasmids were sequenced and mutated genes could then be inferred based on the sequence. Similar screens have been carried out in human cells (Wang et al., 2014), as well as cancer cells (Hart et al., 2014). Additionally, these sgRNA libraries can be used to study noncoding regions of the genome such as enhancer elements (Korkmaz et al., 2016). These screens show that complex sgRNA libraries can be powerful tools in genomic research.

Although most genetic screens using Cas9 are carried out in cell culture, it may be possible to use an sgRNA library on the level of a developing organism. This could be accomplished using one of several different strategies. First, an sgRNA library targeting the genome along with Cas9 protein could be delivered to the germline of an organism. After the germline is mutated, a traditional mating scheme could be set up and resultant mutants screened. Using Cas9 over ENU as a mutagen has a distinct advantage: a library could be constructed that would only target a subset of the genes, making the screen more focused on the genes of interest. A different approach would be to dilute the sgRNA library into small pools and PCR amplify the pools. Pools could then be transcribed and injected with Cas9 into a developing organism. Because Cas9 is able to mutate both alleles in a developing embryo, it could be possible to see phenotypes in the F0 population, eliminating the establishment of a mating scheme. When a phenotype is observed, the pool could be sequenced to generate a short list of candidate genes without requiring genetic mapping. These two advantages would drastically increase the speed of forward genetic screening.

Cas9 is not only a powerful tool for cutting DNA at targeted sites, as it can also be used to recruit other proteins (Gilbert et al., 2013). This is accomplished by fusing other proteins to Cas9 and deactivating its endonuclease domains. Fusing other proteins to Cas9 has led to innovative applications of sgRNA libraries. One of these applications is the ability to paint a chromosomal locus with a fluorescent marker (Lane et al., 2015). A bright version of GFP can be fused to a nuclease-deficient dCas9 and an sgRNA library was generated to target a small region of a chromosome. The dCas9-GFP molecules are complexed with the sgRNA library and the library is injected into living cells. The fluorescent Cas9 essentially tiles the chromosomal region and can be visualized under a fluorescent microscope. Cas9 has also been used to make changes to the epigenome. Proteins that act as activators or repressors have been fused to Cas9 and have been shown to efficiently upregulate or downregulate transcription in human and yeast cells (Gilbert et al., 2013). Cas9 has been fused to proteins that modify histones such as histone demethylases (Kearns et al., 2015). Large scale epigenetic changes could be made by using an sgRNA library in combination with these techniques.

Enzymatically generating customized gRNA libraries will enable these and many other CRISPR applications by allowing the researcher to focus the study on the genes of interest while broadening the use of CRISPR libraries into new species and new paradigms.

In one example, a first polynucleotide which encodes an RNA bound by a cas enzyme may include a constant region encoding sequence selected from a CRISPR single guide RNA (sgRNA) and a CRISPR targeting RNA (crRNA) having a non-palindromic recognition site recognized by a type II restriction enzyme oriented in the sequence and configured to cleave a second operably linked polynucleotide 17 to 27 base pairs from the recognition site, the type II restriction enzyme having methylase activity and cleavage activity, the recognition site may include a nucleotide which may be methylated by the type II restriction enzyme upon cleavage, methylation of the nucleotide altering the recognition site such that the type II restriction enzyme no longer binds the site. For example, a first polynucleotide may be operably linked to the second polynucleotide which encodes a variable or targeting region which hybridizes to a sequence of interest. Additionally, the non-palindromic recognition site when transcribed may be capable of being incorporated within a stem-loop structure of the CRISPR sgRNA or CRISPR crRNA without disrupting Cas9 binding at the constant region in the polynucleotide.

A first polynucleotide which encodes an RNA bound by a cas enzyme, may include a constant region encoding sequence for a CRISPR single guide RNA (sgRNA) or CRISPR targeting RNA (crRNA) having a non-palindromic recognition site recognized by a type II restriction enzyme oriented in the sequence such that a second operably linked polynucleotide may be cleaved 17 to 27 base pairs from the recognition site, when present, the type II restriction enzyme having methylase activity and cleavage activity, the recognition site may include a nucleotide which may be methylated by the type II restriction enzyme upon cleavage, methylation of the nucleotide altering the recognition site such that the type II enzyme no longer binds the site, wherein the first polynucleotide may be a plurality of first polynucleotides; at least a portion of the plurality of first polynucleotides being operably linked to a second polynucleotides to form a plurality of linked second polynucleotides; wherein the plurality of linked second polynucleotides are digested with the type II restriction enzyme having methylase activity to form a plurality of third polynucleotides encoding a plurality of CRISPR sgRNAs or CRISPR crRNAs, wherein at least one of the plurality of CRISPR sgRNAs or CRISPR crRNAs has a variable region different from the other CRISPR sgRNAs or CRISPR crRNAs.

In some examples, the polynucleotide or the plurality of third polynucleotides may be operably linked to a promoter sequence(s). The type II restriction enzyme binding site sequence may optionally be methylated. The Type II restriction enzyme site may be selected from the group consisting of one or more of NmeAIII, MmeI, CstMI, EcoP15I, ApyPI, AquII, AquIII, AquIV, CdpI, CstMI, DraRI, DrdIV, EsaSSI, MaqI, NhaXI, NlaCI, PlaDI, PspOMII, PspPRI, Rcel, RpaB5I, SdeAI, SpoDI, and Bsb. The polynucleotides above may also include one or more sequence adapters suitable for cloning. The polynucleotide above may be double stranded and/or may include a single stranded overhang of between 1 and 100 nucleotides on one or both ends. In some examples, the constant region may contain at least a portion of the stem loop sequence and the second constant region polynucleotide may encode the remaining portion of a functional stem loop structure, wherein the operable linkage reform a functional stem loop structure bound by cas enzyme. Additionally or alternatively, the constant region comprises elongated stem loops or additional sequences at a 5′ end, a 3′ end or both, while maintaining a CRISPR enzyme binding site.

In one example, a method for generating DNA templates for production of a CRISPR/Cas guide strand library may include providing a polynucleotide sample, digesting the polynucleotide sample at protospacer adjacent motifs (PAM) to form target region containing fragments with a first restriction enzyme (RE) in the presence of a ligase and a first adapter sequence, the first adapter sequence may include a constant region having a CRISPR enzyme binding site, the ligase operably linking the first adapter sequence to one or both ends of the fragments, thereby forming an intermediate product which lacks a binding site recognized by the first RE, the intermediate product may include at least one binding site for a second Type II RE; digesting the intermediate product with the second Type II RE in the presence of a ligase and a second adapter sequence, the Type II RE having methylation and nuclease activity which methylates the product at the recognition site and cleaves the targeting region between 17 to 27 base pairs away from the recognition site to form methylated fragments that may include a CRISPR constant region and targeting region methylated fragments, the presence of the methyl group preventing further digestion of the fragments by the second Type II RE, the second adapter optionally may include one or more of a cloning sequence, an adapter sequence, and a vector backbone. In some examples, the first digestion and ligation may be carried out in two steps. Additionally or alternatively, the second digestion and ligation are carried out in two steps. In some examples, the first digestion and ligation and second digestion and ligation are carried out in a single reaction vessel.

In some examples, the polynucleotide sample may be obtained from a source selected from one or more of an organism of interest, an organism at a selected stage of development, a tissue of interest, a cell at a selected stage of differentiation, a cell at a particular stage of the cell cycle, a tissue or cell having a selected pathology. The polynucleotide sample may be selected from cDNA synthesized from RNA, genomic DNA, mitochondrial DNA, human DNA, animal DNA, and plant DNA. The polynucleotide sample may contain polynucleotides isolated via a method selected from precipitation, hybridization, antibody isolation, and co-precipitation.

The second adapter may include a promoter element. The cloning site sequence may be present and the ligated product may be cloned into a vector the vector optionally includes an operably linked promoter sequence. All the steps may be performed in a single reaction vessel. The first polynucleotide may be a plurality of first polynucleotides. At least a portion of the plurality of first polynucleotides may be operably linked to DNA to form a plurality of second linked polynucleotides. The plurality of second linked polynucleotides may be digested with the type II restriction enzyme having methylase activity to form a plurality of third polynucleotides encoding a plurality of CRISPR sgRNAs or CRISPR crRNAs. At least one of the plurality of CRISPR sgRNAs or CRISPR crRNAs may have a variable region different from the other CRISPR sgRNAs or CRISPR crRNAs. The polynucleotide sample may be a cDNA sample which may be normalized to remove repeated transcripts from the cDNA sample, thereby increasing equal representation of transcripts in the library.

In some examples, the adapters may lack a 5′ phosphate. The adapters may contain at least six consecutive phosphorothioates at the 5′ end. The polynucleotide sequence may be digested with an enzyme selected from the group consisting of HpaII, MspI, ScrFI, BfaI, and PacI. The ligated CRISPR sgRNA or CRISPR crRNA product may include at least one nick. The second adapter may include a promoter sequence. In some embodiments, the promoter may be T7 RNA polymerase promoter. In some examples the ligated product does not maintain a G-U hydrogen bond at position 1 of the scaffold sequence. The method may further comprise purifying the ligated CRISPR sgRNA or CRISPR crRNA product. The first adapter sequence may include a 5′ single stranded overhang. The ligated CRISPR sgRNA or CRISPR crRNA product may be purified using a solid support operably linked to a capture oligonucleotide at a 3′ end, where the oligonucleotide hybridizing to the overhang of the adapter sequence. The solid support may be a magnetic bead and the purification step includes magnetic separation. Suspending the separated beads in a buffer may include Bst 3.0 polymerase and nucleotide triophosphates (NTPs) at about 45° C. for about 15 minutes, thereby repairing and extending the nicked CRISPR sgRNA or CRISPR crRNA product, the extension causing displacement of repaired CRISPR sgRNA or CRISPR crRNA product from the bead. The method may include at least one of the following: transcribing the sgRNA template libraries in the presence of DNase I; PCR amplification of the ligated CRISPR sgRNA or CRISPR crRNA product; and the digestion and ligation steps may be performed essentially simultaneously.

A method for generating DNA templates for production of a CRISPR/Cas guide strand library may include: a) providing a polynucleotide sample; b) digesting the polynucleotide sample at protospacer adjacent motifs (PAM) to form a target region containing fragments with a first restriction enzyme (RE) in the presence of a ligase and a first adapter sequence, the first adapter sequence may include a constant region having a CRISPR enzyme binding site, the ligase operably linking the first adapter sequence to one or both ends of the fragments, thereby forming an intermediate product which lacks a binding site recognized by the first RE, the intermediate product may include at least one binding site for a second Type II RE; c) digesting the intermediate product with the second Type II RE in the presence of a ligase and a second adapter sequence, the Type II RE having methylation and nuclease activity which methylates the product at the recognition site and cleaves the targeting region between 17 to 27 base pairs away from the recognition site to form methylated fragments may include a CRISPR constant region and targeting region methylated fragments, the presence of the methyl group preventing further digestion of the fragments by the second Type II RE, the second adapter optionally may include one or more of a cloning sequence, an adapter sequence, and a vector backbone; d) eluting polynucleotides from solid supports by immobilizing one or more first single stranded polynucleotide(s) to a solid support, the first single stranded polynucleotide having 5′ and 3′ ends and having binding affinity for at least a portion of a second polynucleotide(s), the 5′ end of the first single stranded polynucleotide protruding from the solid support; e) contacting the one or more first polynucleotide(s) with one or more second polynucleotide(s) of sufficient complementarity such that a polynucleotide duplex forms, thereby immobilizing the one or more second polynucleotide(s) on the solid support; f) contacting the polynucleotide duplex(s) of step b) with a polymerase having strand displacement activity in the presence of dNTPs, the second polynucleotide serving as a template for extension by the polymerase, wherein the first polynucleotide may be displaced by the extension of the second polynucleotide, and the extension preventing rehybridization between the first and second polynucleotides, the second polynucleotide encoding a CRISPR sgRNA or CRISPR crRNA.

A method for eluting polynucleotides from solid supports may include a) by immobilizing one or more first single stranded polynucleotide(s) to a solid support, the first single stranded polynucleotide having 5′ and 3′ ends and having binding affinity for at least a portion of a second polynucleotide(s), the 5′ end of the first single stranded polynucleotide protruding from the solid support; b) contacting the one or more first polynucleotide(s) with one or more second polynucleotide(s) of sufficient complementarity such that a polynucleotide duplex forms, thereby immobilizing the one or more second polynucleotide(s) on the solid support; c) contacting the polynucleotide duplex(s) of step b) with a polymerase having strand displacement activity in the presence of dNTPs, the second polynucleotide serving as a template for extension by the polymerase, wherein the first polynucleotide may be displaced by the extension of the second polynucleotide, and the extension preventing rehybridization between the first and second polynucleotides, the second polynucleotide encoding a CRISPR sgRN or CRISPR crRNA. In some examples, the support may be a bead. The bead may be a magnetic bead. The attached polynucleotide may be a plurality of polynucleotides and the second polynucleotide may be a plurality of polynucleotides with different sequences. The method may further comprise purifying the second polynucleotide from a mixture. The length of the hybridizing sequences may be adjusted to increase or decrease the temperature at which hybridization will occur. The polynucleotides may be purified at different temperatures and/or the polynucleotides are separated via a process selected from the group consisting of magnetic separation, precipitation, hybridization, antibody isolation, and co-precipitation. One or more second polynucleotides may encode for one or more CRISPR/Cas9 guide RNAs. The intermediate or ligated product may include at least five phosphorothioate linked polynucleotides at the 5′ end. The method may also include degrading any unwanted polynucleotides present in the reaction by contacting the reaction with first exonuclease and second exonucleases which cleave double stranded and single stranded DNA respectively, the phosphorothioate containing polynucleotide being resistant to exonuclease cleavage. For example, the polynucleotides lacking phosphorothioate linkages may be degraded. The polynucleotides with a single phosphorylated end may be degraded. The phosphorothioates may be incorporated by ligating adapters and/or incorporated by PCR.

A kit for production of an sgRNA guide strand library may include: a first polynucleotide which encodes an RNA bound by a cas enzyme, may include a constant region encoding sequence for a CRISPR single guide RNA (sgRNA) or CRISPR targeting RNA (crRNA) having a non-palindromic recognition site recognized by a type II restriction enzyme oriented in the sequence such that a second operably linked polynucleotide may be cleaved 17 to 27 base pairs from the recognition site, when present, the type II restriction enzyme having methylase activity and cleavage activity, the recognition site may include a nucleotide which may be methylated by the type II restriction enzyme upon cleavage, methylation of the nucleotide altering the recognition site such that the type II enzyme no longer binds the site. The kit may also include one or more of: a second polynucleotide encoding a variable or targeting sequence, one or more ligases for operably linking the constant region and targeting polynucleotides, one or more Type II restriction enzymes, a solid support, a strand displacing polymerase, one or more adapter sequences encoding a promoter and/or cloning site, buffers suitable for simultaneous digestion and ligation of a polynucleotide and optionally reagents suitable for PCR amplification, reagents suitable for normalization of input nucleic acid sample.

In any appropriate method described above, a buffer may include one or more of, or all of: 50 mM Potassium Acetate, 20 mM Tris Acetate, 10 mM Magnesium Acetate, 100 ug/ml Bovine Serum Albumin, 1 mM ATP and 7.5% Polyethylene Glycol 6000.

A polynucleotide encoding for a constant region of a CRISPR single guide RNA (sgRNA) or CRISPR targeting RNA (crRNA) may include a methyl moiety that prevents digestion of the polynucleotide by a restriction endonuclease. The methyl moiety may be added by an enzyme containing methyltransferase activity. The polynucleotide may further include: a non-palindromic recognition site for a Type IIS restriction enzyme binding site and a sequence that when ligated to DNA fragments digested with a second restriction enzyme does not restore the recognition sequence for the second restriction enzyme. In some examples, the modifications maintain the stem loop structure of the CRISPR guide RNA molecule in order for the specificity and endonuclease activity of CRISPR RNP complexes to function. The polynucleotide may include a non-palindromic recognition site for a type II restriction enzyme, the non-palindromic recognition site being oriented in a manner recognized by the type II restriction enzyme to cleave upstream of the sequence encoding for a constant region of a CRISPR single guide RNA (sgRNA) or CRISPR targeting RNA (crRNA).

In one example, a kit for generating CRISPR guide RNA (gRNA) libraries may include/result in a polynucleotide containing a methyl moiety and a sequence encoding for a constant region of a CRISPR sgRNA or CRISPR crRNA and having a sequence that does not restore a restriction enzyme binding site. The kit may further include one or more of the following: the type II restriction enzyme, a solid support, wherein the polynucleotide may be capable of being immobilized on the solid supports or is immobilized on the solid support, a strand displacing polymerase, a promoter polynucleotide recognized by an RNA polymerase, and buffers that allow digestion and ligation to occur in the same reaction vessel.

A method for generating double stranded DNA inputs for enzymatic CRISPR library generation may include one or more of the following steps: a) selection of a DNA source; and b) purification of polynucleotides from the source by physical or chemical separation. The DNA source may be selected from one or more of: an organism of interest, an organism at a selected stage of development, a tissue of interest, a cell at a selected stage of differentiation, a cell at a particular stage of the cell cycle, a tissue or cell having a selected pathology. The polynucleotides may be cDNA created from RNA, and/or the polynucleotides may be genomic DNA or mitochondrial DNA. The polynucleotides may be selected by at least one of: precipitation isolation, hybridization isolation, antibody isolation, and other co-precipitation isolation. The polynucleotides or one or more segments thereof may be amplified by PCR and/or normalized.

A method for generating DNA templates for production of a CRISPR/Cas9 guide RNA library may include: a) providing a polynucleotide sample; b) digesting the polynucleotide sample at protospacer adjacent motifs (PAM) to form fragments with a first restriction enzyme (RE) in the presence of a ligase and first adapter, wherein the first adapter sequence may include a constant region containing a sequence CRISPR enzyme can bind to and/or cloning site sequence, polynucleotide sequences to one or both ends of the fragments with a ligase, thereby forming an intermediate product which lacks binding sites recognized by the first RE; c) digesting the second polynucleotide with the type II restriction enzyme to form a third polynucleotide encoding a CRISPR sgRNA or CRISPR crRNA, wherein the type II restriction enzyme cuts the DNA at a site that may be 17 to 27 base pairs from the end of the first polynucleotide. The method may further include ligating a polynucleotide to an end of the third polynucleotide. The ligated polynucleotide may include a promoter and/or a cloning adapter. The first adaptor and/or second adapter may or may not contain a 5′ phosphate group.

A method for generating DNA templates for production of a CRISPR/Cas9 guide RNA library may include: a) providing a polynucleotide sample; b) digesting the polynucleotide sample at protospacer adjacent motifs (PAM) to form fragments with a first restriction enzyme (RE); c) ligation to a first adapter, wherein the first adapter sequence comprises a constant region containing a sequence CRISPR enzyme can bind to and/or cloning site sequence, polynucleotide sequences to one or both ends of the fragments with a ligase; d) contacting the intermediate product with a Type II S RE having methylase activity, in the presence of a ligase, the ligase operably linking a second adapter sequence to digested, methylated fragments formed from digestion of the intermediate product, thereby forming a ligated product having a variable or targeting region, and a scaffold region, wherein the second adapter sequence comprises at least one of a promoter element and cloning site sequence.

A method for generating DNA templates for production of a CRISPR/Cas9 guide RNA library may include one or more of the following steps: a) providing a polynucleotide sample; b) digesting the polynucleotide sample at protospacer adjacent motifs (PAM) to form fragments with a first restriction enzyme (RE) in the presence of a ligase and first adapter, wherein the first adapter sequence comprises a constant region containing at least one of: a sequence CRISPR enzyme can bind to and cloning site sequence, joining polynucleotide sequences to one or both ends of the fragments with a ligase, thereby forming an intermediate product which lacks binding sites recognized by the first RE; c) contacting the intermediate product with a Type II S RE, in the presence of a ligase, the ligase operably linking a second adapter sequence to the digested fragments formed from digestion of the intermediate product, thereby forming a ligated product having a variable or targeting region, and a scaffold region, wherein the second adapter sequence comprises at least one of a promoter element and cloning site sequence. Redigestion may be blocked by simultaneous modification of the DNA upon digestion. In some examples, there may be modification in attachment of a methylation moiety. A cloning site sequence may be present and the ligated product may be cloned into a vector, the vector may optionally include an operable promoter and/or scaffold sequence. In some examples, the promoter may be present and the ligated product may be cloned into a vector that may include at least part of a scaffold sequence. In one embodiment, steps a-c may be performed in a single reaction vessel.

The first polynucleotide may include a plurality of first polynucleotides; at least a portion of the plurality of first polynucleotides may be ligated with DNA to form a plurality of second polynucleotides; and the plurality of second polynucleotides may be digested with the type II restriction enzyme that may include methylase activity to form a plurality of third polynucleotides encoding a plurality of CRISPR sgRNAs or CRISPR crRNAs. In some examples, at least one of the plurality of CRISPR sgRNAs or CRISPR crRNAs may have a variable region different from the other CRISPR sgRNAs or CRISPR crRNAs. The method described above may include polynucleotide sample(s) in which cDNA may be normalized to remove abundant transcripts from the cDNA, thereby increasing equal representation of transcripts in the library. The method above may include an input polynucleotide sample that may be obtained from a source selected from the group consisting of: an organism of interest, an organism at a selected stage of development, a tissue of interest, a cell at a selected stage of differentiation, a cell at a particular stage of the cell cycle, and a tissue or cell having a selected pathology. The method may also include adapters have or do not have a 5′ phosphate. For example, the adapters contain at least six consecutive phophorothioates at the 5′ end. The promoter may include a T7 RNA polymerase promoter. The polynucleotide sequence of step a) may be digested with variety of appropriate enzymes, including an enzyme selected from the group consisting of HpaII, MspI, ScrFI, BfaI, and PacI. The ligated product of step c) may include at least one nick and/or may not maintain a G-U hydrogen bond at position 1 of the scaffold sequence. The method may further include purifying the ligated product. For example, the ligated product may be purified using a capture oligonucleotide that may include a biotin at a 3′ end, which hybridizes to the scaffold portion of the ligated product operably linked to a solid support. The solid support may be a magnetic bead and the purification step may include magnetic separation. The method may further or alternatively include suspending the separated beads in a reaction that includes Bst 3.0 polymerase and nucleotide triophosphates (NTPs) at about 45° C. for about 15 minutes, thereby repairing and extending the nicked strand. This extension may cause displacement of the repaired product from the bead. The method may include transcribing the sgRNA template libraries in the presence of DNase I and/or elution of the sgRNA template from the beads followed by PCR amplification of the sgRNA template. The first first RE may be one of MspI, HpaII, ScrFI, BsaJI. The digestion and ligation of step b) may be performed essentially simultaneously, and/or the digestion and ligation of step c) may be performed essentially simultaneously. A functional gRNA template may be produced by the method(s) described above, the template may include at least one or more operably linked sequences: a promoter, a protospacer, an adapter, a type II RE recognition sequence, and a modified scaffold sequence.

In one example, a genome wide library may include a plurality of unique CRISPR-Cas system guide sequences that are capable of targeting a plurality of target sequences in genomic loci, wherein the library may be created using one or more of the methods described above.

An illustrative method of eluting polynucleotides from solid supports may include one or more of the following steps, including: a) attaching a single stranded polynucleotide to a solid support such that the 5′ end of the polynucleotide may be protruding from the solid support and may be capable of hybridizing with at least a portion of a second polynucleotide; b) contacting the attached single stranded polynucleotide with the second polynucleotide such that hybridization between the two polynucleotides forms a polynucleotide duplex, thereby immobilizing the second polynucleotide; c) contacting the polynucleotide duplex with a polymerase exhibiting strand displacement activity in the presence of dNTPs, in which the second polynucleotide may be used as a template for extension by the polymerase, wherein the first polynucleotide may be displaced by the extension of the polymerase, and the extension may prevent rehybridization of the first and second polynucleotides. In one example, the support may include one or more beads, which may or may not have magnetic or paramagnetic properties. The attached single stranded polynucleotide may include a plurality of polynucleotides. The second polynucleotide may include a plurality of polynucleotides with different sequences. The method may also include the use of the support to purify the second polynucleotide from a mixture. A length of length of the hybridizing sequence can be adjusted to increase or decrease a temperature at which hybridization occurs. In some examples, hybridization occurs at room temperature. For example, the hybridization may occur at 0-50° C. The method may include a physical separation process for purification of oligonucleotides. In some embodiments, the second polynucleotide contains a sequence encoding for CRISPR/Cas9 guide RNAs.

A method for selectively degrading unwanted polynucleotide components of a mixture may include: a) incorporation of at least five nucleotides linked by phosphorothioates at 5′ ends of a double stranded DNA fragment to be protected; b) contacting the double stranded DNA fragments with a first exonuclease that operates on double stranded DNA and may be sensitive to phosphorothioate linkages and a second exonuclease that operates on single stranded DNA. In this method, polynucleotides with no protection may be degraded. In one example, polynucleotides with a single end may be degraded. In one embodiment, phosphorothioates may be incorporated by ligating adapters using any appropriate method including by PCR.

An illustrative method for manipulation of DNA substrates through enzymatic processes to produce nucleotide sequences may include: simultaneously digesting DNA by targeting a restriction enzyme to sites containing protospacer adjacent motif (PAM) sequences in the DNA to produce DNA fragments and ligating adaptors to ends of DNA fragments to produce an intermediate product containing the scaffold ligated with a DNA fragment of an arbitrary length; and simultaneously digesting and ligating the intermediate product to produce guide RNA templates. In one embodiment, simultaneously digesting DNA and ligating adaptors to ends of the DNA fragments may include digesting the DNA in presence of a ligase and a first adaptor. The digesting may include a type IIS restriction enzyme to digest the intermediate product to create a guide RNA including an 18 to 25 base pair protospacer connected to and engineered polynucleotide. In some examples, the type IIS restriction enzyme blocks redigestion of the ligation product. For example, the type IIS restriction blocks its own function after digestion by chemically modifying its own binding site. For example, the chemical modification of the binding site may include attaching a methyl group.

Ligation may include a connection of the digestion product to an upstream adapter to produce a guide RNA template containing an upstream adapter, a proto spacer and the engineered polynucleotide. The upstream adapter may include at least one of a promoter, a cloning site, or other DNA integration site. Additionally or alternatively, the upstream adapter does not contain a 5′ phosphate on the ligated end. In one example, the ligated product contains at least one nick. The method may include purifying the guide RNA templates. The step of purifying may include attaching polynucleotides in a sequence dependent hybridization to a bead, washing to remove reagents and fragments that are not attached to the beads, and eluting the guide RNA templates from the bead. Additionally or alternatively, purifying may include elution and nick repair of the guide RNA templates. In some examples, the elution and nick repair may occur in a same reaction. The bead may be a magnetic or paramagnetic bead. The elution and nick repair may be both performed by a single enzyme. The single enzyme may include a strand displacing polymerase to simultaneously elute and repair nicks in the attached polynucleotides. The strand displacing polymerase may use the previous captured polynucleotide as a template to displace and fill in a sequence, thereby permanently displacing the polynucleotide from the beads. A hybridized segment of the previously captured polynucleotide may be made double stranded by the polymerase thereby preventing rehybridization of the polynucleotide to the beads.

The method may include selecting a DNA source, wherein selecting may include choosing a species of organism, selecting at least one of: a developmental stage of the organism, a tissue maturation stage, a state of cell differentiation and a stage of a cell cycle, selecting environmental conditions the organism may be subject to, and selecting a tissue or cell type from the organism to be the DNA source.

The method may additionally or alternatively include extracting DNA or RNA from the selected DNA source, wherein extracting comprises at least one of: a chemical separation of cellular components and a physical separation of cellular components, wherein the extracting isolates a nucleic acid species of interest. The chemical separation of DNA or RNA may include amplification of the nucleic acid species. For example, the chemical separation may include enzymatic amplification of at least one region of the nucleic acid species. The enzymatic amplification may include polymerase chain reaction (PCR). The physical separation of DNA or RNA may include isolation of DNA or RNA by at least one of: precipitation isolation, hybridization isolation, antibody isolation, and other co-precipitation isolation. For example, the step of extracting may include extracting RNA from the selected DNA source and then converting the RNA into DNA. Additionally, the extracting may include normalizing the DNA by enzymatic or chemical methods applied to the DNA, wherein normalizing comprises balancing quantities of various DNA components in the mixture to ensure more equal representation in an output library.

A method for manipulation of DNA substrates through enzymatic processes to produce nucleotide sequences, may include one or more of the following steps, including selecting a DNA source by: choosing a species of organism and selecting at least one of a developmental stage of the organism, a tissue maturation stage, a state of cell differentiation and a stage of a cell cycle, selecting environmental conditions the organism may be subject to, and selecting a tissue or cell type from the organism to be the DNA source.

The method may also include extracting DNA or RNA from the selected DNA source, wherein extracting comprises at least one of: a chemical separation of cellular components and a physical separation of cellular components, wherein the extracting isolates a nucleic acid species of interest; wherein chemical separation of DNA or RNA comprises amplification of the nucleic acid species, wherein chemical separation comprises enzymatic amplification of at least one region of the nucleic acid species, wherein the enzymatic amplification comprises polymerase chain reaction (PCR), wherein physical separation of DNA or RNA comprises isolation of DNA or RNA by at least one of: precipitation isolation, hybridization isolation, antibody isolation, and other co-precipitation isolation; wherein extracting comprises extracting RNA from the selected DNA source and then converting the RNA into DNA; wherein extracting comprises normalizing the DNA, wherein normalizing comprises balancing quantities of various DNA components in the mixture to ensure more equal representation in an output library; simultaneously digesting the extracted DNA by targeting a restriction enzyme to sites containing PAM sequences to produce DNA fragments and ligating adaptors to ends of DNA fragments to produce an intermediate product containing the scaffold ligated with a DNA fragment of an arbitrary length.

The step of ligating may include introducing an engineered polynucleotide sequence that can associate with a CAS9 molecule (scaffold) such that the engineered polynucleotide sequence: prevents re-creation of the restriction enzyme binding site upon ligation, contains a type IIS binding site such that enzyme will cut between 18 and 25 base pairs upstream of the adapter, maintains essential base pair interactions needed to maintain the endonuclease activity and binding specificity of the CAS9, and allows ligation of the digested DNA fragments by having a blunt end or containing an overhang compatible with the ends produced by enzymatic digestion; wherein the engineered polynucleotide does not contain a 5′ phosphate on the ligated end; wherein the ligation product contains at least one nick; wherein the engineered polynucleotide sequence does not maintain the G-U hydrogen bond of the G at position 1 of the scaffold sequence, wherein the G may be replaced by a different base; wherein the engineered polynucleotide sequence comprises a single gRNA scaffold or crRNA sequence or an adapter sequence for cloning into a vector containing either a single gRNA scaffold or crRNA sequence. simultaneously digesting and ligating the intermediate product to produce a ligated product; wherein the digesting comprises a type IIS restriction enzyme to digest the intermediate product to create a guide RNA may include an 18 to 25 base pair protospacer connected to the engineered polynucleotide; wherein the type IIS restriction enzyme blocks redigestion of the ligation product; wherein the type IIS restriction blocks its own function after digestion by chemically modifying its own binding site; wherein the chemical modifying its own binding site comprises attaching a methyl group; wherein ligation comprises connection of the digestion product to an upstream adapter to produce a guide RNA template containing an upstream adapter, a proto spacer and the engineered polynucleotide; wherein the upstream adapter comprises at least one of a promoter, a cloning site, or other DNA integration site; wherein the upstream adapter does not contain a 5′ phosphate on the ligated end; wherein the ligated product contains at least one nick; purifying the guide RNA templates; wherein purifying comprises attaching polynucleotides in a sequence dependent hybridization to a bead, washing to remove reagents and fragments that are not attached to the beads, and eluting the guide RNA templates from the bead; wherein the bead comprises a magnetic or paramagnetic bead; wherein purifying comprises elution and nick repair of the guide RNA templates; wherein the elution and nick repair occurs in the same reaction; wherein the elution and nick repair are both performed by a single enzyme; wherein the single enzyme comprises a strand displacing polymerase to simultaneously elute and repair nicks in the attached polynucleotides; wherein the strand displacing polymerase uses the previous captured polynucleotide as a template to displace and fill in a sequence, thereby permanently displacing the polynucleotide from the beads; wherein a hybridized segment of the previously captured polynucleotide is made double stranded by the polymerase thereby preventing rehybridization to the polynucleotide to the beads.

A method for generating DNA templates for production of a CRISPR/Cas9 guide RNA library, wherein the method may include digesting a polynucleotide sample into digested products in the presence of a ligase and an adaptor, wherein the digested products are chemically prevented from reversing the digestion and are ligated to the adaptor in a single reaction vessel. In one example, the digesting and ligating include digesting the polynucleotide sample at protospacer adjacent motifs (PAM) to form fragments with a first restriction enzyme (RE) in the presence of a ligase and first adapter, wherein the first adapter sequence comprises a constant region containing a sequence that a CRISPR enzyme can bind to and/or cloning site sequence, and lacks sufficient sequence similarity to the first RE recognition sequence such that ligation of the adapter to one or both ends of the fragments produces an intermediate product which lacks binding sites recognized by the first RE. The digesting and ligating may include digesting the second polynucleotide with the type II restriction enzyme to form a third polynucleotide encoding a CRISPR sgRNA or CRISPR crRNA, wherein the type II restriction enzyme cuts the DNA at a site that is 17 to 27 base pairs from the end of the first polynucleotide. The digesting and ligating may include performing the digestion and ligation steps in a single reaction vessel. In one example, the steps of digesting and ligating may include unphosphorylated adapters resulting in nicked products. The polynucleotide sample may include normalized cDNA. The method described above may further including bead purification. In one embodiment, the strand displacing polymerase may be used to elute the library from a solid support and simultaneously repair any nicks in the library. In one example, a G-U wobble base pair in the guide RNA stem loop may be replaced by G-V. The method may further include digesting and ligating that includes the enzymatic addition of a methyl moiety.

As described herein, a first polynucleotide may include a sequence encoding for a constant region of one of a CRISPR single guide RNA (sgRNA) and CRISPR targeting RNA (crRNA). The sequence may include a non-palindromic recognition site recognized by a type II restriction enzyme, wherein the non-palindromic recognition site may be configured such that a second polynucleotide operably linked to the first polynucleotide may be cleaved 17 to 27 base pairs from the recognition site by the type II restriction enzyme, the type II restriction enzyme configured to add a methyl moiety to the recognition site such that methylation of the recognition site prevents subsequent cleavage of the second polynucleotide.

In one example, and according to principles described herein, a first polynucleotide may include a sequence encoding for a constant region of one of a CRISPR single guide RNA (sgRNA) and CRISPR targeting RNA (crRNA). The sequence may include a non-palindromic recognition site configured to be recognized by a type II restriction enzyme, wherein the non-palindromic recognition site is oriented such that a second polynucleotide operably linked an end adjacent to the non-palindromic recognition site, the first polynucleotide configured to be cleaved 17 to 27 base pairs from the recognition site by the type II restriction enzyme, and the type II restriction enzyme may be configured to add a methyl moiety to the recognition site such that methylation of the recognition site prevents subsequent cleavage of the second polynucleotide.

In one embodiment, a first polynucleotide may include a sequence encoding for a constant region of one of a CRISPR single guide RNA (sgRNA) and CRISPR targeting RNA (crRNA), and an end configured to operably link a second polynucleotide, the sequence may include: a non-palindromic recognition site recognized by a type II restriction enzyme, wherein the non-palindromic recognition site is configured to position and orient the type II restriction enzyme to cleave the second polynucleotide 17 to 27 base pairs from the recognition site; wherein the type II restriction enzyme is configured to add a methyl moiety to the recognition site such that the methyl moiety prevents subsequent cleavage of the second polynucleotide.

In one example, a polynucleotide may include a sequence encoding for at least part of a protein binding segment of an RNA component of a CRISPR complex. The sequence may include a non-palindromic recognition sequence for a type II restriction enzyme configured to cleave at least 17 nucleotides outside of the non-palindromic recognition sequence, the non-palindromic recognition sequence being oriented such that the type II restriction enzyme may be configured such it cannot cleave within the sequence encoding for at least part of a protein binding segment of an RNA component of a CRISPR complex.

In one embodiment, a polynucleotide encoding at least part of a protein binding segment of an RNA component of a CRISPR complex may include a non-palindromic recognition sequence for a type II restriction enzyme having methyltransferase activity and configured to cleave at least 17 nucleotides outside of the non-palindromic recognition sequence. The recognition sequence may include a nucleotide capable of being methylated by the type II restriction enzyme, wherein methylation of the nucleotide prevents cleavage of DNA by the restriction enzyme. The recognition sequence may be oriented such that, in the presence of the restriction enzyme, the DNA cleavage domain of the restriction enzyme may be positioned outside of the sequence encoding for at least part of the protein binding segment of the RNA component of the CRISPR complex.

An example of the invention may include a polynucleotide comprising a sequence encoding for a constant region.

The example of the invention may also include one or more steps, functions, or structures set forth above combined with the constant region comprising a protein binding segment of an RNA component of a CRISPR complex.

The example of the invention may also include one or more steps, functions, or structures set forth above combined with the sequence comprising a non-palindromic recognition sequence for a first restriction enzyme having methyltransferase activity and configured to cleave at least 17 nucleotides outside of the non-palindromic recognition sequence.

The example of the invention may also include one or more steps, functions, or structures set forth above combined with at least one base of the non-palindromic recognition sequence being configured to be methylated by the first restriction enzyme, thereby blocking endonuclease activity of the first restriction enzyme.

The example of the invention may also include one or more steps, functions, or structures set forth above combined with the RNA component of the CRISPR complex being configured to form a secondary structure comprising the non-palindromic recognition sequence.

The example of the invention may also include one or more steps, functions, or structures set forth above combined with at least a portion of the polynucleotide being double stranded.

The example of the invention may also include one or more steps, functions, or structures set forth above combined with at least one base of the non-palindromic recognition sequence having an attached methyl group.

The example of the invention may also include one or more steps, functions, or structures set forth above further comprising a sequence such that ligation of the polynucleotide to a second polynucleotide cleaved by a second restriction enzyme does not restore a recognition sequence of the second restriction enzyme.

Another example of the invention may include a method for generating a library of polynucleotides.

The example of the invention may also include one or more steps, functions, or structures set forth above combined with providing a first adapter.

The example of the invention may also include one or more steps, functions, or structures set forth above combined with the first adapter comprising a sequence encoding for a protein binding segment of an RNA component of a CRISPR complex.

The example of the invention may also include one or more steps, functions, or structures set forth above combined with the first adapter further comprising a second recognition sequence for a second restriction enzyme.

The example of the invention may also include one or more steps, functions, or structures set forth above combined with combining a polynucleotide sample with a first restriction enzyme, the first adapter, and a first ligase.

The example of the invention may also include one or more steps, functions, or structures set forth above combined with the polynucleotide sample comprising a first recognition sequence for the first restriction enzyme.

The example of the invention may also include one or more steps, functions, or structures set forth above combined with the first recognition sequence comprising a protospacer adjacent motif

(PAM).

The example of the invention may also include one or more steps, functions, or structures set forth above combined with the first restriction enzyme being configured to cleave the first recognition sequence to produce a cleaved fragment.

The example of the invention may also include one or more steps, functions, or structures set forth above combined with the first ligase being configured to operably linking the first adapter to the cleaved fragment to generate an intermediate polynucleotide.

The example of the invention may also include one or more steps, functions, or structures set forth above combined with the intermediate polynucleotide not containing the first recognition sequence.

The example of the invention may also include one or more steps, functions, or structures set forth above combined with combining the intermediate polynucleotide with the second restriction enzyme, a second adapter, and a second ligase.

The example of the invention may also include one or more steps, functions, or structures set forth above combined with the second restriction enzyme having methyltransferase activity.

The example of the invention may also include one or more steps, functions, or structures set forth above combined with the second restriction enzyme being configured to cleave the intermediate polynucleotide at least 17 nucleotides outside of the second recognition sequence, thereby generating a methylated polynucleotide comprising a methyl group.

The example of the invention may also include one or more steps, functions, or structures set forth above combined with the methyl group inhibiting activity of the second restriction enzyme.

The example of the invention may also include one or more steps, functions, or structures set forth above combined with the second ligase being configured to operably linking the methylated polynucleotide to the second adapter to generate a final polynucleotide.

The example of the invention may also include one or more steps, functions, or structures set forth above combined with the first adapter comprising a plurality of first adapters.

The example of the invention may also include one or more steps, functions, or structures set forth above combined with at least a portion of the plurality of first adapters being operably linked to a plurality of cleaved fragments to form a plurality of intermediate polynucleotides.

The example of the invention may also include one or more steps, functions, or structures set forth above combined with the plurality of intermediate polynucleotides being digested with the second restriction enzyme to form a plurality of methylated polynucleotides encoding a plurality of RNA components of CRISPR complexes.

The example of the invention may also include one or more steps, functions, or structures set forth above combined with at least one of the plurality of methylated polynucleotides encoding a variable region different from the other methylated polynucleotides.

The example of the invention may also include one or more steps, functions, or structures set forth above combined with cleavage and operably linking in step (b) being carried out in two separate steps.

The example of the invention may also include one or more steps, functions, or structures set forth above combined with cleavage and operably linking in step (c) being carried out in two separate steps.

The example of the invention may also include one or more steps, functions, or structures set forth above combined with step (b) and step (c) being combined to create a single step.

The example of the invention may also include one or more steps, functions, or structures set forth above combined with step (b) and step (c) being performed in a single reaction vessel.

The example of the invention may also include one or more steps, functions, or structures set forth above combined with the first restriction enzyme comprising at least one of HpaII, MspI, ScrFI, and BsaJI.

The example of the invention may also include one or more steps, functions, or structures set forth above combined with the first ligase and the second ligase comprising the same ligase.

The example of the invention may also include one or more steps, functions, or structures set forth above combined with the second adapter comprising at least a portion of a promoter sequence.

The example of the invention may also include one or more steps, functions, or structures set forth above combined with the methylated polynucleotide further comprising at least one sequence configured for molecular cloning.

The example of the invention may also include one or more steps, functions, or structures set forth above combined with at least one of the adapters lacking a 5′ phosphate.

The example of the invention may also include one or more steps, functions, or structures set forth above combined with the methylated polynucleotide comprising at least one nick.

The example of the invention may also include one or more steps, functions, or structures set forth above combined with the first adapter being purified using a solid support operably linked to a capture polynucleotide at a 3′ end.

The example of the invention may also include one or more steps, functions, or structures set forth above combined with the first adapter further comprising a 5′ overhang configured to hybridize to the capture polynucleotide.

The example of the invention may also include one or more steps, functions, or structures set forth above combined with the second recognition sequence comprising a non-palindromic recognition sequence.

Another example of the invention may include a kit for generating a library of polynucleotides encoding for RNA components of CRISPR complexes.

The example of the invention may also include one or more steps, functions, or structures set forth above combined with a polynucleotide comprising a sequence encoding for a constant region.

The example of the invention may also include one or more steps, functions, or structures set forth above combined with the sequence comprising a protein binding segment of an RNA component of a CRISPR complex.

The example of the invention may also include one or more steps, functions, or structures set forth above combined with the sequence further comprising a non-palindromic recognition sequence for a restriction enzyme having methyltransferase activity.

The example of the invention may also include one or more steps, functions, or structures set forth above combined with the restriction enzyme being configured to cleave at least 17 nucleotides outside of the non-palindromic recognition sequence.

The example of the invention may also include one or more steps, functions, or structures set forth above combined with at least one base of the non-palindromic recognition sequence being configured to be methylated by the restriction enzyme, thereby blocking the endonuclease activity of the restriction enzyme.

The example of the invention may also include one or more steps, functions, or structures set forth above combined with the kit further comprising at least one ligase.

The example of the invention may also include one or more steps, functions, or structures set forth above combined with the kit further comprising the restriction enzyme.

The example of the invention may also include one or more steps, functions, or structures set forth above combined with the kit further comprising a restriction enzyme configured to cleave a recognition sequence comprising a protospacer adjacent motif (PAM).

The example of the invention may also include one or more steps, functions, or structures set forth above combined with the kit further comprising a second polynucleotide configured to be ligated.

The example of the invention may also include one or more steps, functions, or structures set forth above combined with the kit further comprising at least one buffer to provide operable conditions for both the restriction enzyme and at least one ligase.

The example of the invention may also include one or more steps, functions, or structures set forth above further comprising a solid support configured to secure polynucleotides.

The example of the invention may also include one or more steps, functions, or structures set forth above further comprising a strand displacing polymerase.

Another example of the invention may include a method for removing a polynucleotide immobilized on a solid support.

The example of the invention may also include one or more steps, functions, or structures set forth above combined with providing a first polynucleotide attached to a solid support at the 3′ end.

The example of the invention may also include one or more steps, functions, or structures set forth above combined with providing a second polynucleotide hybridized to least a portion of the first polynucleotide.

The example of the invention may also include one or more steps, functions, or structures set forth above combined with the second polynucleotide extending past the 5′ end of the first polynucleotide.

The example of the invention may also include one or more steps, functions, or structures set forth above combined with providing a third polynucleotide hybridized to least a portion of the second polynucleotide.

The example of the invention may also include one or more steps, functions, or structures set forth above combined with all three polynucleotides being immobilized on the solid support.

The example of the invention may also include one or more steps, functions, or structures set forth above combined with contacting the hybridized second and third polynucleotides with a polymerase having strand displacement activity.

The example of the invention may also include one or more steps, functions, or structures set forth above combined with the second polynucleotide serving as a template for extension by the polymerase.

The example of the invention may also include one or more steps, functions, or structures set forth above combined with the 3′ end of the third nucleotide being extended and the first polynucleotide being displaced by the polymerase.

The example of the invention may also include one or more steps, functions, or structures set forth above combined with detaching the second and third polynucleotides from the solid support.

The example of the invention may also include one or more steps, functions, or structures set forth above combined with inhibiting rehybridization of the first and second polynucleotides.

The example of the invention may also include one or more steps, functions, or structures set forth above combined with providing the polymerase having 5′ to 3′ exonuclease activity.

The example of the invention may also include one or more steps, functions, or structures set forth above combined with displacing and degrading hybridized polynucleotides during strand extension.

The example of the invention may also include one or more steps, functions, or structures set forth above combined with the second polynucleotide and third polynucleotide comprising the same polynucleotide.

The example of the invention may also include one or more steps, functions, or structures set forth above combined with providing the 3′ end of the second polynucleotide being configured to connect to the 5′ end of the third polynucleotide by a phosphodiester bond.

The example of the invention may also include one or more steps, functions, or structures set forth above combined with providing the first, second, and third polynucleotides each comprise a plurality of polynucleotides.

The example of the invention may also include one or more steps, functions, or structures set forth above combined with the support being a bead.

The example of the invention may also include one or more steps, functions, or structures set forth above combined with providing the support being a magnetic bead.

The example of the invention may also include one or more steps, functions, or structures set forth above combined with the polynucleotides being separated by at least one of: magnetic separation, precipitation, hybridization, antibody isolation, and co-precipitation.

The example of the invention may also include one or more steps, functions, or structures set forth above combined with the plurality of first polynucleotides comprising a polynucleotide that includes a sequence different from the other first polynucleotides.

Another example of the invention may include a system to remove polynucleotides immobilized on a solid supports.

The example of the invention may also include one or more steps, functions, or structures set forth above combined with a solid support.

The example of the invention may also include one or more steps, functions, or structures set forth above combined with a first polynucleotide configured to attach to a solid support at the 3′ end.

The example of the invention may also include one or more steps, functions, or structures set forth above combined with a second polynucleotide configured to hybridize to least a portion of the first polynucleotide.

The example of the invention may also include one or more steps, functions, or structures set forth above combined with a second polynucleotide configured to extend past the 5′ end of the first polynucleotide.

The example of the invention may also include one or more steps, functions, or structures set forth above combined with a strand displacing polymerase.

The example of the invention may also include one or more steps, functions, or structures set forth above combined with the first polynucleotide being covalently attached to the solid support.

The example of the invention may also include one or more steps, functions, or structures set forth above combined with the first polynucleotide being attached to the solid support by affinity binding.

The example of the invention may also include one or more steps, functions, or structures set forth above combined with the solid support being a bead.

The example of the invention may also include one or more steps, functions, or structures set forth above combined with the solid support being a magnetic bead.

The example of the invention may also include one or more steps, functions, or structures set forth above combined with buffers that provide conditions for both hybridization and strand displacement to occur.

What is claimed is:

1. A polynucleotide comprising:
a sequence encoding for a constant region comprising a protein binding segment of an RNA component of a CRISPR complex, the sequence comprising a non-palindromic recognition sequence for a first restriction enzyme having methyltransferase activity and configured to cleave at least 17 nucleotides outside of the non-palindromic recognition sequence, wherein at least one base of the non-palindromic recognition sequence is configured to be methylated by the first restriction enzyme, thereby blocking endonuclease activity of the first restriction enzyme.

2. The polynucleotide of claim 1, wherein the RNA component of the CRISPR complex is configured to form a secondary structure comprising the non-palindromic recognition sequence.

3. The polynucleotide of claim 1, wherein at least a portion of the polynucleotide is double stranded.

4. The polynucleotide of claim 1, wherein at least one base of the non-palindromic recognition sequence has an attached methyl group.

5. The polynucleotide of claim 1, further comprising a sequence such that ligation of the polynucleotide to a second polynucleotide cleaved by a second restriction enzyme does not restore a recognition sequence of the second restriction enzyme.

6. A method for generating a library of polynucleotides, comprising:
(a) providing a first adapter, the first adapter comprising a sequence encoding for a protein binding segment of an RNA component of a CRISPR complex, the first adapter further comprising a second recognition sequence for a second restriction enzyme;
(b) combining a polynucleotide sample with a first restriction enzyme, the first adapter, and a first ligase;
the polynucleotide sample comprising a first recognition sequence for the first restriction enzyme, the first recognition sequence comprising a protospacer adjacent motif (PAM);
the first restriction enzyme being configured to cleave the first recognition sequence to produce a cleaved fragment;
the first ligase is configured to operably linking the first adapter to the cleaved fragment to generate an intermediate polynucleotide, wherein the intermediate polynucleotide does not contain the first recognition sequence;
(c) combining the intermediate polynucleotide with the second restriction enzyme, a second adapter, and a second ligase;
the second restriction enzyme having methyltransferase activity and being configured to cleave the intermediate polynucleotide at least 17 nucleotides outside of the second recognition sequence, thereby generating a methylated polynucleotide comprising a methyl group, wherein the methyl group inhibits activity of the second restriction enzyme;
the second ligase is configured to operably linking the methylated polynucleotide to the second adapter to generate a final polynucleotide.

7. The method of claim 6, wherein the first adapter comprises a plurality of first adapters, wherein at least a portion of the plurality of first adapters are operably linked to a plurality of cleaved fragments to form a plurality of intermediate polynucleotides; and the plurality of intermediate polynucleotides are digested with the second restriction enzyme to form a plurality of methylated polynucleotides encoding a plurality of RNA components of CRISPR complexes, wherein at least one of the plurality of methylated polynucleotides encodes a variable region different from the other methylated polynucleotides.

8. The method of claim 6, wherein cleavage and operably linking in step (b) are carried out in two separate steps.

9. The method of claim 6, wherein cleavage and operably linking in step (c) are carried out in two separate steps.

10. The method of claim 6, wherein step (b) and step (c) are combined to create a single step.

11. The method of claim 6, wherein step (b) and step (c) are performed in a single reaction vessel.

12. The method of claim 6, wherein the first restriction enzyme comprises at least one of HpaII, MspI, ScrFI, and BsaJI.

13. The method of claim 6, wherein the first ligase and the second ligase comprise the same ligase.

14. The method of claim 6, wherein the second adapter comprises at least a portion of a promoter sequence.

15. The method of claim 6, wherein the methylated polynucleotide further comprises at least one sequence configured for molecular cloning.

16. The method of claim 6, wherein at least one of the adapters lacks a 5′ phosphate.

17. The method of claim 6, wherein the methylated polynucleotide comprises at least one nick.

18. The method of claim 6, wherein the first adapter is purified using a solid support operably linked to a capture polynucleotide at a 3′ end, the first adapter further comprising a 5′ overhang configured to hybridize to the capture polynucleotide.

19. The method of claim 6, wherein the second recognition sequence comprises a non-palindromic recognition sequence.

20. A kit for generating a library of polynucleotides encoding for RNA components of CRISPR complexes, comprising:
a polynucleotide comprising a sequence encoding for a constant region, the sequence comprising:
a protein binding segment of an RNA component of a CRISPR complex, and
a non-palindromic recognition sequence for a restriction enzyme having methyltransferase activity wherein the restriction enzyme is configured to cleave at least 17 nucleotides outside of the non-palindromic recognition sequence, wherein at least one base of the non-palindromic recognition sequence is configured to be methylated by the restriction enzyme, thereby blocking the endonuclease activity of the restriction enzyme.

21. The kit of claim 20, wherein the kit further comprises at least one ligase.

22. The kit of claim 20, wherein the kit further comprises the restriction enzyme.

23. The kit of claim 20, wherein the kit further comprises a restriction enzyme configured to cleave a recognition sequence comprising a protospacer adjacent motif (PAM).

24. The kit of claim 20, wherein the kit further comprises a second polynucleotide configured to be ligated.

25. The kit of claim 20, wherein the kit further comprises at least one buffer to provide operable conditions for both the restriction enzyme and at least one ligase.

26. The kit of claim 20, further comprising a solid support configured to secure polynucleotides.

27. The kit of claim 20, further comprising a strand displacing polymerase.


SEQUENCE LISTING (597 sequences)

SEQ ID NO: 1 - DNA, 81 bp, Streptococcus pyogenes
gttttagagctagaaatagcaagttaaaataaggctagtccgttatcaacttgaaaaagtggcaccgagtcggtgcttttt

SEQ ID NO: 2 - DNA, 81 bp, Streptococcus pyogenes
aaaaagcaccgactcggtgccactttttcaagttgataacggactagccttattttaacttgctatttctagctctaaaac

SEQ ID NO: 3 - RNA, 81 nt, Streptococcus pyogenes
guuuuagagcuagaaauagcaaguuaaaauaaggcuaguccguuaucaacuugaaaaaguggcaccgagucggugcuuuuu

SEQ ID NO: 4 - DNA, 81 bp, Artificial Sequence
  Polynucleotide encoding constant region of sgRNA/crRNA with non-palindromic recognition site for restriction enzyme (sense or antisense), polynucleotide encoding for constant and variable regions of sgRNA/crRNA (sense or antisense), or sgRNA/crRNA.
gtyggagagctagaaatagcaagttccaataaggctagtccgttatcaacttgaaaaagtggcaccgagtcggtgcttttt

SEQ ID NO: 5 - DNA, 81 bp, Artificial Sequence
  Polynucleotide encoding constant region of sgRNA/crRNA with non-palindromic recognition site for restriction enzyme (sense or antisense), polynucleotide encoding for constant and variable regions of sgRNA/crRNA (sense or antisense), or sgRNA/crRNA.
aaaaagcaccgactcggtgccactttttcaagttgataacggactagccttattggaacttgctatttctagctctccrac

SEQ ID NO: 6 - DNA, 101 bp, Artificial Sequence
  Polynucleotide encoding constant region of sgRNA/crRNA with non-palindromic recognition site for restriction enzyme (sense or antisense), polynucleotide encoding for constant and variable regions of sgRNA/crRNA (sense or antisense), or sgRNA/crRNA.
  misc_feature (1)..(20): n is a, c, g, or t
nnnnnnnnnnnnnnnnnnnngtyggagagctagaaatagcaagttccaataaggctagtccgttatcaacttgaaaaagtggcaccgagtcggtgcttttt

SEQ ID NO: 7 - DNA, 101 bp, Artificial Sequence
  Polynucleotide encoding constant region of sgRNA/crRNA with non-palindromic recognition site for restriction enzyme (sense or antisense), polynucleotide encoding for constant and variable regions of sgRNA/crRNA (sense or antisense), or sgRNA/crRNA.
  misc_feature (82)..(101): n is a, c, g, or t
aaaaagcaccgactcggtgccactttttcaagttgataacggactagccttattggaacttgctatttctagctctccracnnnnnnnnnnnnnnnnnnnn

SEQ ID NO: 8 - RNA, 101 nt, Artificial Sequence
  Polynucleotide encoding constant region of sgRNA/crRNA with non-palindromic recognition site for restriction enzyme (sense or antisense), polynucleotide encoding for constant and variable regions of sgRNA/crRNA (sense or antisense), or sgRNA/crRNA.
  misc_feature (1)..(20): n is a, c, g, or u
nnnnnnnnnnnnnnnnnnnnguyggagagcuagaaauagcaaguuccaauaaggcuaguccguuaucaacuugaaaaaguggcaccgagucggugcuuuuu

SEQ ID NO: 9 - DNA, 81 bp, Artificial Sequence
  Polynucleotide encoding constant region of sgRNA/crRNA with non-palindromic recognition site for restriction enzyme (sense or antisense), polynucleotide encoding for constant and variable regions of sgRNA/crRNA (sense or antisense), or sgRNA/crRNA.
ggtyggaagctagaaatagcaagtccaactaaggctagtccgttatcaacttgaaaaagtggcaccgagtcggtgcttttt

SEQ ID NO: 10 - DNA, 81 bp, Artificial Sequence
  Polynucleotide encoding constant region of sgRNA/crRNA with non-palindromic recognition site for restriction enzyme (sense or antisense), polynucleotide encoding for constant and variable regions of sgRNA/crRNA (sense or antisense), or sgRNA/crRNA.
aaaaagcaccgactcggtgccactttttcaagttgataacggactagccttagttggacttgctatttctagcttccracc

SEQ ID NO: 11 - DNA, 100 bp, Artificial Sequence
  Polynucleotide encoding constant region of sgRNA/crRNA with non-palindromic recognition site for restriction enzyme (sense or antisense), polynucleotide encoding for constant and variable regions of sgRNA/crRNA (sense or antisense), or sgRNA/crRNA.
  misc_feature (1)..(19): n is a, c, g, or t
nnnnnnnnnnnnnnnnnnnggtyggaagctagaaatagcaagtccaactaaggctagtccgttatcaacttgaaaaagtggcaccgagtcggtgcttttt

SEQ ID NO: 12 - DNA, 100 bp, Artificial Sequence
  Polynucleotide encoding constant region of sgRNA/crRNA with non-palindromic recognition site for restriction enzyme (sense or antisense), polynucleotide encoding for constant and variable regions of sgRNA/crRNA (sense or antisense), or sgRNA/crRNA.
  misc_feature (82)..(100): n is a, c, g, or t
aaaaagcaccgactcggtgccactttttcaagttgataacggactagccttagttggacttgctatttctagcttccraccnnnnnnnnnnnnnnnnnnn

SEQ ID NO: 13 - RNA, 100 nt, Artificial Sequence
  Polynucleotide encoding constant region of sgRNA/crRNA with non-palindromic recognition site for restriction enzyme (sense or antisense), polynucleotide encoding for constant and variable regions of sgRNA/crRNA (sense or antisense), or sgRNA/crRNA.
  misc_feature (1)..(19): n is a, c, g, or u
nnnnnnnnnnnnnnnnnnngguyggaagcuagaaauagcaaguccaacuaaggcuaguccguuaucaacuugaaaaaguggcaccgagucggugcuuuuu

SEQ ID NO: 14 - DNA, 81 bp, Artificial Sequence
  Polynucleotide encoding constant region of sgRNA/crRNA with non-palindromic recognition site for restriction enzyme (sense or antisense), polynucleotide encoding for constant and variable regions of sgRNA/crRNA (sense or antisense), or sgRNA/crRNA.
gtgtyggagctagaaatagcaagtcaacataaggctagtccgttatcaacttgaaaaagtggcaccgagtcggtgcttttt

SEQ ID NO: 15 - DNA, 81 bp, Artificial Sequence
  Polynucleotide encoding constant region of sgRNA/crRNA with non-palindromic recognition site for restriction enzyme (sense or antisense), polynucleotide encoding for constant and variable regions of sgRNA/crRNA (sense or antisense), or sgRNA/crRNA.
aaaaagcaccgactcggtgccactttttcaagttgataacggactagccttatgttgacttgctatttctagctccracac

SEQ ID NO: 16 - DNA, 99 bp, Artificial Sequence
  Polynucleotide encoding constant region of sgRNA/crRNA with non-palindromic recognition site for restriction enzyme (sense or antisense), polynucleotide encoding for constant and variable regions of sgRNA/crRNA (sense or antisense), or sgRNA/crRNA.
  misc_feature (1)..(18): n is a, c, g, or t
nnnnnnnnnnnnnnnnnngtgtyggagctagaaatagcaagtcaacataaggctagtccgttatcaacttgaaaaagtggcaccgagtcggtgcttttt

SEQ ID NO: 17 - DNA, 99 bp, Artificial Sequence
  Polynucleotide encoding constant region of sgRNA/crRNA with non-palindromic recognition site for restriction enzyme (sense or antisense), polynucleotide encoding for constant and variable regions of sgRNA/crRNA (sense or antisense), or sgRNA/crRNA.
  misc_feature (82)..(99): n is a, c, g, or t
aaaaagcaccgactcggtgccactttttcaagttgataacggactagccttatgttgacttgctatttctagctccracacnnnnnnnnnnnnnnnnnn

SEQ ID NO: 18 - RNA, 99 nt, Artificial Sequence
  Polynucleotide encoding constant region of sgRNA/crRNA with non-palindromic recognition site for restriction enzyme (sense or antisense), polynucleotide encoding for constant and variable regions of sgRNA/crRNA (sense or antisense), or sgRNA/crRNA.
  misc_feature (1)..(18): n is a, c, g, or u
nnnnnnnnnnnnnnnnnnguguyggagcuagaaauagcaagucaacauaaggcuaguccguuaucaacuugaaaaaguggcaccgagucggugcuuuuu

SEQ ID NO: 19 - DNA, 82 bp, Artificial Sequence
  Polynucleotide encoding constant region of sgRNA/crRNA with non-palindromic recognition site for restriction enzyme (sense or antisense), polynucleotide encoding for constant and variable regions of sgRNA/crRNA (sense or antisense), or sgRNA/crRNA.
ccggtyggagctagaaatagcaagtcaacctaaggctagtccgttatcaacttgaaaaagtggcaccgagtcggtgcttttt

SEQ ID NO: 20 - DNA, 82 bp, Artificial Sequence
  Polynucleotide encoding constant region of sgRNA/crRNA with non-palindromic recognition site for restriction enzyme (sense or antisense), polynucleotide encoding for constant and variable regions of sgRNA/crRNA (sense or antisense), or sgRNA/crRNA.
aaaaagcaccgactcggtgccactttttcaagttgataacggactagccttaggttgacttgctatttctagctccraccgg

SEQ ID NO: 21 - DNA, 99 bp, Artificial Sequence
  Polynucleotide encoding constant region of sgRNA/crRNA with non-palindromic recognition site for restriction enzyme (sense or antisense), polynucleotide encoding for constant and variable regions of sgRNA/crRNA (sense or antisense), or sgRNA/crRNA.
  misc_feature (1)..(18): n is a, c, g, or t
nnnnnnnnnnnnnnnnnncggtyggagctagaaatagcaagtcaacctaaggctagtccgttatcaacttgaaaaagtggcaccgagtcggtgcttttt

SEQ ID NO: 22 - DNA, 99 bp, Artificial Sequence
  Polynucleotide encoding constant region of sgRNA/crRNA with non-palindromic recognition site for restriction enzyme (sense or antisense), polynucleotide encoding for constant and variable regions of sgRNA/crRNA (sense or antisense), or sgRNA/crRNA.
  misc_feature (82)..(99): n is a, c, g, or t
aaaaagcaccgactcggtgccactttttcaagttgataacggactagccttaggttgacttgctatttctagctccraccgnnnnnnnnnnnnnnnnnn

SEQ ID NO: 23 - RNA, 99 nt, Artificial Sequence
  Polynucleotide encoding constant region of sgRNA/crRNA with non-palindromic recognition site for restriction enzyme (sense or antisense), polynucleotide encoding for constant and variable regions of sgRNA/crRNA (sense or antisense), or sgRNA/crRNA.
  misc_feature (1)..(18): n is a, c, g, or u
nnnnnnnnnnnnnnnnnncgguyggagcuagaaauagcaagucaaccuaaggcuaguccguuaucaacuugaaaaaguggcaccgagucggugcuuuuu

SEQ ID NO: 24 - DNA, 83 bp, Artificial Sequence
  Polynucleotide encoding constant region of sgRNA/crRNA with non-palindromic recognition site for restriction enzyme (sense or antisense), polynucleotide encoding for constant and variable regions of sgRNA/crRNA (sense or antisense), or sgRNA/crRNA.
  misc_feature (3)..(3): n is a, c, g, or t
ccnggtyggagctagaaatagcaagtcaacctaaggctagtccgttatcaacttgaaaaagtggcaccgagtcggtgcttttt

SEQ ID NO: 25 - DNA, 83 bp, Artificial Sequence
  Polynucleotide encoding constant region of sgRNA/crRNA with non-palindromic recognition site for restriction enzyme (sense or antisense), polynucleotide encoding for constant and variable regions of sgRNA/crRNA (sense or antisense), or sgRNA/crRNA.
  misc_feature (81)..(81): n is a, c, g, or t
aaaaagcaccgactcggtgccactttttcaagttgataacggactagccttaggttgacttgctatttctagctccraccngg

SEQ ID NO: 26 - DNA, 99 bp, Artificial Sequence
  Polynucleotide encoding constant region of sgRNA/crRNA with non-palindromic recognition site for restriction enzyme (sense or antisense), polynucleotide encoding for constant and variable regions of sgRNA/crRNA (sense or antisense), or sgRNA/crRNA.
  misc_feature (1)..(19): n is a, c, g, or t
nnnnnnnnnnnnnnnnnnnggtyggagctagaaatagcaagtcaacctaaggctagtccgttatcaacttgaaaaagtggcaccgagtcggtgcttttt

SEQ ID NO: 27 - DNA, 99 bp, Artificial Sequence
  Polynucleotide encoding constant region of sgRNA/crRNA with non-palindromic recognition site for restriction enzyme (sense or antisense), polynucleotide encoding for constant and variable regions of sgRNA/crRNA (sense or antisense), or sgRNA/crRNA.
  misc_feature (81)..(99): n is a, c, g, or t
aaaaagcaccgactcggtgccactttttcaagttgataacggactagccttaggttgacttgctatttctagctccraccnnnnnnnnnnnnnnnnnnn

SEQ ID NO: 28 - RNA, 99 nt, Artificial Sequence
  Polynucleotide encoding constant region of sgRNA/crRNA with non-palindromic recognition site for restriction enzyme (sense or antisense), polynucleotide encoding for constant and variable regions of sgRNA/crRNA (sense or antisense), or sgRNA/crRNA.
  misc_feature (1)..(19): n is a, c, g, or u
nnnnnnnnnnnnnnnnnnngguyggagcuagaaauagcaagucaaccuaaggcuaguccguuaucaacuugaaaaaguggcaccgagucggugcuuuuu

SEQ ID NO: 29 - DNA, 82 bp, Artificial Sequence
  Polynucleotide encoding constant region of sgRNA/crRNA with non-palindromic recognition site for restriction enzyme (sense or antisense), polynucleotide encoding for constant and variable regions of sgRNA/crRNA (sense or antisense), or sgRNA/crRNA.
ctagtyggagctagaaatagcaagtcaacctaaggctagtccgttatcaacttgaaaaagtggcaccgagtcggtgcttttt

SEQ ID NO: 30 - DNA, 82 bp, Artificial Sequence
  Polynucleotide encoding constant region of sgRNA/crRNA with non-palindromic recognition site for restriction enzyme (sense or antisense), polynucleotide encoding for constant and variable regions of sgRNA/crRNA (sense or antisense), or sgRNA/crRNA.
aaaaagcaccgactcggtgccactttttcaagttgataacggactagccttaggttgacttgctatttctagctccractag

SEQ ID NO: 31 - DNA, 99 bp, Artificial Sequence
  Polynucleotide encoding constant region of sgRNA/crRNA with non-palindromic recognition site for restriction enzyme (sense or antisense), polynucleotide encoding for constant and variable regions of sgRNA/crRNA (sense or antisense), or sgRNA/crRNA.
  misc_feature (1)..(18): n is a, c, g, or t
nnnnnnnnnnnnnnnnnntagtyggagctagaaatagcaagtcaacctaaggctagtccgttatcaacttgaaaaagtggcaccgagtcggtgcttttt

SEQ ID NO: 32 - DNA, 99 bp, Artificial Sequence
  Polynucleotide encoding constant region of sgRNA/crRNA with non-palindromic recognition site for restriction enzyme (sense or antisense), polynucleotide encoding for constant and variable regions of sgRNA/crRNA (sense or antisense), or sgRNA/crRNA.
  misc_feature (82)..(99): n is a, c, g, or t
aaaaagcaccgactcggtgccactttttcaagttgataacggactagccttaggttgacttgctatttctagctccractannnnnnnnnnnnnnnnnn

SEQ ID NO: 33 - RNA, 99 nt, Artificial Sequence
  Polynucleotide encoding constant region of sgRNA/crRNA with non-palindromic recognition site for restriction enzyme (sense or antisense), polynucleotide encoding for constant and variable regions of sgRNA/crRNA (sense or antisense), or sgRNA/crRNA.
  misc_feature (1)..(18): n is a, c, g, or u
nnnnnnnnnnnnnnnnnnuaguyggagcuagaaauagcaagucaaccuaaggcuaguccguuaucaacuugaaaaaguggcaccgagucggugcuuuuu

SEQ ID NO: 34 - DNA, 81 bp, Artificial Sequence
  Polynucleotide encoding constant region of sgRNA/crRNA with non-palindromic recognition site for restriction enzyme (sense or antisense), polynucleotide encoding for constant and variable regions of sgRNA/crRNA (sense or antisense), or sgRNA/crRNA.
ctcggcgagctagaaatagcaagtgccgataaggctagtccgttatcaacttgaaaaagtggcaccgagtcggtgcttttt

SEQ ID NO: 35 - DNA, 81 bp, Artificial Sequence
  Polynucleotide encoding constant region of sgRNA/crRNA with non-palindromic recognition site for restriction enzyme (sense or antisense), polynucleotide encoding for constant and variable regions of sgRNA/crRNA (sense or antisense), or sgRNA/crRNA.
aaaaagcaccgactcggtgccactttttcaagttgataacggactagccttatcggcacttgctatttctagctcgccgag

SEQ ID NO: 36 - DNA, 101 bp, Artificial Sequence
  Polynucleotide encoding constant region of sgRNA/crRNA with non-palindromic recognition site for restriction enzyme (sense or antisense), polynucleotide encoding for constant and variable regions of sgRNA/crRNA (sense or antisense), or sgRNA/crRNA.
  misc_feature (1)..(20): n is a, c, g, or t
nnnnnnnnnnnnnnnnnnnnctcggcgagctagaaatagcaagtgccgataaggctagtccgttatcaacttgaaaaagtggcaccgagtcggtgcttttt

SEQ ID NO: 37 - DNA, 101 bp, Artificial Sequence
  Polynucleotide encoding constant region of sgRNA/crRNA with non-palindromic recognition site for restriction enzyme (sense or antisense), polynucleotide encoding for constant and variable regions of sgRNA/crRNA (sense or antisense), or sgRNA/crRNA.
  misc_feature (82)..(101): n is a, c, g, or t
aaaaagcaccgactcggtgccactttttcaagttgataacggactagccttatcggcacttgctatttctagctcgccgagnnnnnnnnnnnnnnnnnnnn

SEQ ID NO: 38 - RNA, 101 nt, Artificial Sequence
  Polynucleotide encoding constant region of sgRNA/crRNA with non-palindromic recognition site for restriction enzyme (sense or antisense), polynucleotide encoding for constant and variable regions of sgRNA/crRNA (sense or antisense), or sgRNA/crRNA.
  misc_feature (1)..(20): n is a, c, g, or u
nnnnnnnnnnnnnnnnnnnncucggcgagcuagaaauagcaagugccgauaaggcuaguccguuaucaacuugaaaaaguggcaccgagucggugcuuuuu

SEQ ID NO: 39 - DNA, 81 bp, Artificial Sequence
  Polynucleotide encoding constant region of sgRNA/crRNA with non-palindromic recognition site for restriction enzyme (sense or antisense), polynucleotide encoding for constant and variable regions of sgRNA/crRNA (sense or antisense), or sgRNA/crRNA.
gctcggcagctagaaatagcaagtccgagtaaggctagtccgttatcaacttgaaaaagtggcaccgagtcggtgcttttt

SEQ ID NO: 40 - DNA, 81 bp, Artificial Sequence
  Polynucleotide encoding constant region of sgRNA/crRNA with non-palindromic recognition site for restriction enzyme (sense or antisense), polynucleotide encoding for constant and variable regions of sgRNA/crRNA (sense or antisense), or sgRNA/crRNA.
aaaaagcaccgactcggtgccactttttcaagttgataacggactagccttactcggacttgctatttctagctgccgagc

SEQ ID NO: 41 - DNA, 100 bp, Artificial Sequence
  Polynucleotide encoding constant region of sgRNA/crRNA with non-palindromic recognition site for restriction enzyme (sense or antisense), polynucleotide encoding for constant and variable regions of sgRNA/crRNA (sense or antisense), or sgRNA/crRNA.
  misc_feature (1)..(19): n is a, c, g, or t
nnnnnnnnnnnnnnnnnnngctcggcagctagaaatagcaagtccgagtaaggctagtccgttatcaacttgaaaaagtggcaccgagtcggtgcttttt

SEQ ID NO: 42 - DNA, 100 bp, Artificial Sequence
  Polynucleotide encoding constant region of sgRNA/crRNA with non-palindromic recognition site for restriction enzyme (sense or antisense), polynucleotide encoding for constant and variable regions of sgRNA/crRNA (sense or antisense), or sgRNA/crRNA.
  misc_feature (82)..(100): n is a, c, g, or t
aaaaagcaccgactcggtgccactttttcaagttgataacggactagccttactcggacttgctatttctagctgccgagcnnnnnnnnnnnnnnnnnnn

SEQ ID NO: 43 - RNA, 100 nt, Artificial Sequence
  Polynucleotide encoding constant region of sgRNA/crRNA with non-palindromic recognition site for restriction enzyme (sense or antisense), polynucleotide encoding for constant and variable regions of sgRNA/crRNA (sense or antisense), or sgRNA/crRNA.
  misc_feature (1)..(19): n is a, c, g, or u
nnnnnnnnnnnnnnnnnnngcucggcagcuagaaauagcaaguccgaguaaggcuaguccguuaucaacuugaaaaaguggcaccgagucggugcuuuuu

SEQ ID NO: 44 - DNA, 81 bp, Artificial Sequence
  Polynucleotide encoding constant region of sgRNA/crRNA with non-palindromic recognition site for restriction enzyme (sense or antisense), polynucleotide encoding for constant and variable regions of sgRNA/crRNA (sense or antisense), or sgRNA/crRNA.
gtctcggcgctagaaatagcaagtcgagataaggctagtccgttatcaacttgaaaaagtggcaccgagtcggtgcttttt

SEQ ID NO: 45 - DNA, 81 bp, Artificial Sequence
  Polynucleotide encoding constant region of sgRNA/crRNA with non-palindromic recognition site for restriction enzyme (sense or antisense), polynucleotide encoding for constant and variable regions of sgRNA/crRNA (sense or antisense), or sgRNA/crRNA.
aaaaagcaccgactcggtgccactttttcaagttgataacggactagccttatctcgacttgctatttctagcgccgagac

SEQ ID NO: 46 - DNA, 99 bp, Artificial Sequence
  Polynucleotide encoding constant region of sgRNA/crRNA with non-palindromic recognition site for restriction enzyme (sense or antisense), polynucleotide encoding for constant and variable regions of sgRNA/crRNA (sense or antisense), or sgRNA/crRNA.
  misc_feature (1)..(18): n is a, c, g, or t
nnnnnnnnnnnnnnnnnngtctcggcgctagaaatagcaagtcgagataaggctagtccgttatcaacttgaaaaagtggcaccgagtcggtgcttttt

SEQ ID NO: 47 - DNA, 99 bp, Artificial Sequence
  Polynucleotide encoding constant region of sgRNA/crRNA with non-palindromic recognition site for restriction enzyme (sense or antisense), polynucleotide encoding for constant and variable regions of sgRNA/crRNA (sense or antisense), or sgRNA/crRNA.
  misc_feature (82)..(99): n is a, c, g, or t
aaaaagcaccgactcggtgccactttttcaagttgataacggactagccttatctcgacttgctatttctagcgccgagacnnnnnnnnnnnnnnnnnn

SEQ ID NO: 48 - RNA, 99 nt, Artificial Sequence
  Polynucleotide encoding constant region of sgRNA/crRNA with non-palindromic recognition site for restriction enzyme (sense or antisense), polynucleotide encoding for constant and variable regions of sgRNA/crRNA (sense or antisense), or sgRNA/crRNA.
  misc_feature (1)..(18): n is a, c, g, or u
nnnnnnnnnnnnnnnnnngucucggcgcuagaaauagcaagucgagauaaggcuaguccguuaucaacuugaaaaaguggcaccgagucggugcuuuuu

SEQ ID NO: 49 - DNA, 81 bp, Artificial Sequence
  Polynucleotide encoding constant region of sgRNA/crRNA with non-palindromic recognition site for restriction enzyme (sense or antisense), polynucleotide encoding for constant and variable regions of sgRNA/crRNA (sense or antisense), or sgRNA/crRNA.
gtcgatgagctagaaatagcaagtatcgataaggctagtccgttatcaacttgaaaaagtggcaccgagtcggtgcttttt

SEQ ID NO: 50 - DNA, 81 bp, Artificial Sequence
  Polynucleotide encoding constant region of sgRNA/crRNA with non-palindromic recognition site for restriction enzyme (sense or antisense), polynucleotide encoding for constant and variable regions of sgRNA/crRNA (sense or antisense), or sgRNA/crRNA.
aaaaagcaccgactcggtgccactttttcaagttgataacggactagccttatcgatacttgctatttctagctcatcgac

SEQ ID NO: 51 - DNA, 101 bp, Artificial Sequence
  Polynucleotide encoding constant region of sgRNA/crRNA with non-palindromic recognition site for restriction enzyme (sense or antisense), polynucleotide encoding for constant and variable regions of sgRNA/crRNA (sense or antisense), or sgRNA/crRNA.
  misc_feature (1)..(20): n is a, c, g, or t
nnnnnnnnnnnnnnnnnnnngtcgatgagctagaaatagcaagtatcgataaggctagtccgttatcaacttgaaaaagtggcaccgagtcggtgcttttt

SEQ ID NO: 52 - DNA, 101 bp, Artificial Sequence
  Polynucleotide encoding constant region of sgRNA/crRNA with non-palindromic recognition site for restriction enzyme (sense or antisense), polynucleotide encoding for constant and variable regions of sgRNA/crRNA (sense or antisense), or sgRNA/crRNA.
  misc_feature (82)..(101): n is a, c, g, or t
aaaaagcaccgactcggtgccactttttcaagttgataacggactagccttatcgatacttgctatttctagctcatcgacnnnnnnnnnnnnnnnnnnnn

SEQ ID NO: 53 - RNA, 101 nt, Artificial Sequence
  Polynucleotide encoding constant region of sgRNA/crRNA with non-palindromic recognition site for restriction enzyme (sense or antisense), polynucleotide encoding for constant and variable regions of sgRNA/crRNA (sense or antisense), or sgRNA/crRNA.
  misc_feature (1)..(20): n is a, c, g, or u
nnnnnnnnnnnnnnnnnnnngucgaugagcuagaaauagcaaguaucgauaaggcuaguccguuaucaacuugaaaaaguggcaccgagucggugcuuuuu

SEQ ID NO: 54 - DNA, 81 bp, Artificial Sequence
  Polynucleotide encoding constant region of sgRNA/crRNA with non-palindromic recognition site for restriction enzyme (sense or antisense), polynucleotide encoding for constant and variable regions of sgRNA/crRNA (sense or antisense), or sgRNA/crRNA.
  misc_feature (3)..(3): n is a, c, g, or t
  misc_feature (28)..(28): n is a, c, g, or t
gtncggcagctagaaatagcaaggccgnataaggctagtccgttatcaacttgaaaaagtggcaccgagtcggtgcttttt

SEQ ID NO: 55 - DNA, 81 bp, Artificial Sequence
  Polynucleotide encoding constant region of sgRNA/crRNA with non-palindromic recognition site for restriction enzyme (sense or antisense), polynucleotide encoding for constant and variable regions of sgRNA/crRNA (sense or antisense), or sgRNA/crRNA.
  misc_feature (54)..(54): n is a, c, g, or t
  misc_feature (79)..(79): n is a, c, g, or t
aaaaagcaccgactcggtgccactttttcaagttgataacggactagccttatncggccttgctatttctagctgccgnac

SEQ ID NO: 56 - DNA, 101 bp, Artificial Sequence
  Polynucleotide encoding constant region of sgRNA/crRNA with non-palindromic recognition site for restriction enzyme (sense or antisense), polynucleotide encoding for constant and variable regions of sgRNA/crRNA (sense or antisense), or sgRNA/crRNA.
  misc_feature (1)..(20): n is a, c, g, or t
  misc_feature (23)..(23): n is a, c, g, or t
  misc_feature (48)..(48): n is a, c, g, or t
nnnnnnnnnnnnnnnnnnnngtncggcagctagaaatagcaaggccgnataaggctagtccgttatcaacttgaaaaagtggcaccgagtcggtgcttttt

SEQ ID NO: 57 - DNA, 101 bp, Artificial Sequence
  Polynucleotide encoding constant region of sgRNA/crRNA with non-palindromic recognition site for restriction enzyme (sense or antisense), polynucleotide encoding for constant and variable regions of sgRNA/crRNA (sense or antisense), or sgRNA/crRNA.
  misc_feature (54)..(54): n is a, c, g, or t
  misc_feature (79)..(79): n is a, c, g, or t
  misc_feature (82)..(101): n is a, c, g, or t
aaaaagcaccgactcggtgccactttttcaagttgataacggactagccttatncggccttgctatttctagctgccgnacnnnnnnnnnnnnnnnnnnnn

SEQ ID NO: 58 - RNA, 101 nt, Artificial Sequence
  Polynucleotide encoding constant region of sgRNA/crRNA with non-palindromic recognition site for restriction enzyme (sense or antisense), polynucleotide encoding for constant and variable regions of sgRNA/crRNA (sense or antisense), or sgRNA/crRNA.
  misc_feature (1)..(20): n is a, c, g, or u
  misc_feature (23)..(23): n is a, c, g, or u
  misc_feature (48)..(48): n is a, c, g, or u
nnnnnnnnnnnnnnnnnnnnguncggcagcuagaaauagcaaggccgnauaaggcuaguccguuaucaacuugaaaaaguggcaccgagucggugcuuuuu

SEQ ID NO: 59 - DNA, 81 bp, Artificial Sequence
  Polynucleotide encoding constant region of sgRNA/crRNA with non-palindromic recognition site for restriction enzyme (sense or antisense), polynucleotide encoding for constant and variable regions of sgRNA/crRNA (sense or antisense), or sgRNA/crRNA.
ctcctcgagctagaaatagcaagtgaggataaggctagtccgttatcaacttgaaaaagtggcaccgagtcggtgcttttt

SEQ ID NO: 60 - DNA, 81 bp, Artificial Sequence
  Polynucleotide encoding constant region of sgRNA/crRNA with non-palindromic recognition site for restriction enzyme (sense or antisense), polynucleotide encoding for constant and variable regions of sgRNA/crRNA (sense or antisense), or sgRNA/crRNA.
aaaaagcaccgactcggtgccactttttcaagttgataacggactagccttatcctcacttgctatttctagctcgaggag

SEQ ID NO: 61 - DNA, 101 bp, Artificial Sequence
  Polynucleotide encoding constant region of sgRNA/crRNA with non-palindromic recognition site for restriction enzyme (sense or antisense), polynucleotide encoding for constant and variable regions of sgRNA/crRNA (sense or antisense), or sgRNA/crRNA.
  misc_feature (1)..(20): n is a, c, g, or t
nnnnnnnnnnnnnnnnnnnnctcctcgagctagaaatagcaagtgaggataaggctagtccgttatcaacttgaaaaagtggcaccgagtcggtgcttttt

SEQ ID NO: 62 - DNA, 101 bp, Artificial Sequence
  Polynucleotide encoding constant region of sgRNA/crRNA with non-palindromic recognition site for restriction enzyme (sense or antisense), polynucleotide encoding for constant and variable regions of sgRNA/crRNA (sense or antisense), or sgRNA/crRNA.
  misc_feature (82)..(101): n is a, c, g, or t
aaaaagcaccgactcggtgccactttttcaagttgataacggactagccttatcctcacttgctatttctagctcgaggagnnnnnnnnnnnnnnnnnnnn

SEQ ID NO: 63 - RNA, 101 nt, Artificial Sequence
  Polynucleotide encoding constant region of sgRNA/crRNA with non-palindromic recognition site for restriction enzyme (sense or antisense), polynucleotide encoding for constant and variable regions of sgRNA/crRNA (sense or antisense), or sgRNA/crRNA.
  misc_feature (1)..(20): n is a, c, g, or u
nnnnnnnnnnnnnnnnnnnncuccucgagcuagaaauagcaagugaggauaaggcuaguccguuaucaacuugaaaaaguggcaccgagucggugcuuuuu

SEQ ID NO: 64 - DNA, 81 bp, Artificial Sequence
  Polynucleotide encoding constant region of sgRNA/crRNA with non-palindromic recognition site for restriction enzyme (sense or antisense), polynucleotide encoding for constant and variable regions of sgRNA/crRNA (sense or antisense), or sgRNA/crRNA.
cttccycagctagaaatagcaaggrggaataaggctagtccgttatcaacttgaaaaagtggcaccgagtcggtgcttttt

SEQ ID NO: 65 - DNA, 81 bp, Artificial Sequence
  Polynucleotide encoding constant region of sgRNA/crRNA with non-palindromic recognition site for restriction enzyme (sense or antisense), polynucleotide encoding for constant and variable regions of sgRNA/crRNA (sense or antisense), or sgRNA/crRNA.
aaaaagcaccgactcggtgccactttttcaagttgataacggactagccttattccyccttgctatttctagctgrggaag

SEQ ID NO: 66 - DNA, 101 bp, Artificial Sequence
  Polynucleotide encoding constant region of sgRNA/crRNA with non-palindromic recognition site for restriction enzyme (sense or antisense), polynucleotide encoding for constant and variable regions of sgRNA/crRNA (sense or antisense), or sgRNA/crRNA.
  misc_feature (1)..(20): n is a, c, g, or t
nnnnnnnnnnnnnnnnnnnncttccycagctagaaatagcaaggrggaataaggctagtccgttatcaacttgaaaaagtggcaccgagtcggtgcttttt

SEQ ID NO: 67 - DNA, 101 bp, Artificial Sequence
  Polynucleotide encoding constant region of sgRNA/crRNA with non-palindromic recognition site for restriction enzyme (sense or antisense), polynucleotide encoding for constant and variable regions of sgRNA/crRNA (sense or antisense), or sgRNA/crRNA.
  misc_feature (82)..(101): n is a, c, g, or t
aaaaagcaccgactcggtgccactttttcaagttgataacggactagccttattccyccttgctatttctagctgrggaagnnnnnnnnnnnnnnnnnnnn

SEQ ID NO: 68 - RNA, 101 nt, Artificial Sequence
  Polynucleotide encoding constant region of sgRNA/crRNA with non-palindromic recognition site for restriction enzyme (sense or antisense), polynucleotide encoding for constant and variable regions of sgRNA/crRNA (sense or antisense), or sgRNA/crRNA.
  misc_feature (1)..(20): n is a, c, g, or u
nnnnnnnnnnnnnnnnnnnncuuccycagcuagaaauagcaaggrggaauaaggcuaguccguuaucaacuugaaaaaguggcaccgagucggugcuuuuu

SEQ ID NO: 69 - DNA, 81 bp, Artificial Sequence
  Polynucleotide encoding constant region of sgRNA/crRNA with non-palindromic recognition site for restriction enzyme (sense or antisense), polynucleotide encoding for constant and variable regions of sgRNA/crRNA (sense or antisense), or sgRNA/crRNA.
ctccgcgagctagaaatagcaagtgcggataaggctagtccgttatcaacttgaaaaagtggcaccgagtcggtgcttttt

SEQ ID NO: 70 - DNA, 81 bp, Artificial Sequence
  Polynucleotide encoding constant region of sgRNA/crRNA with non-palindromic recognition site for restriction enzyme (sense or antisense), polynucleotide encoding for constant and variable regions of sgRNA/crRNA (sense or antisense), or sgRNA/crRNA.
aaaaagcaccgactcggtgccactttttcaagttgataacggactagccttatccgcacttgctatttctagctcgcggag

SEQ ID NO: 71 - DNA, 101 bp, Artificial Sequence
  Polynucleotide encoding constant region of sgRNA/crRNA with non-palindromic recognition site for restriction enzyme (sense or antisense), polynucleotide encoding for constant and variable regions of sgRNA/crRNA (sense or antisense), or sgRNA/crRNA.
  misc_feature (1)..(20): n is a, c, g, or t
nnnnnnnnnnnnnnnnnnnnctccgcgagctagaaatagcaagtgcggataaggctagtccgttatcaacttgaaaaagtggcaccgagtcggtgcttttt

SEQ ID NO: 72 - DNA, 101 bp, Artificial Sequence
  Polynucleotide encoding constant region of sgRNA/crRNA with non-palindromic recognition site for restriction enzyme (sense or antisense), polynucleotide encoding for constant and variable regions of sgRNA/crRNA (sense or antisense), or sgRNA/crRNA.
  misc_feature (82)..(101): n is a, c, g, or t
aaaaagcaccgactcggtgccactttttcaagttgataacggactagccttatccgcacttgctatttctagctcgcggagnnnnnnnnnnnnnnnnnnnn

SEQ ID NO: 73 - RNA, 101 nt, Artificial Sequence
  Polynucleotide encoding constant region of sgRNA/crRNA with non-palindromic recognition site for restriction enzyme (sense or antisense), polynucleotide encoding for constant and variable regions of sgRNA/crRNA (sense or antisense), or sgRNA/crRNA.
  misc_feature (1)..(20): n is a, c, g, or u
nnnnnnnnnnnnnnnnnnnncuccgcgagcuagaaauagcaagugcggauaaggcuaguccguuaucaacuugaaaaaguggcaccgagucggugcuuuuu

SEQ ID NO: 74 - DNA, 81 bp, Artificial Sequence
  Polynucleotide encoding constant region of sgRNA/crRNA with non-palindromic recognition site for restriction enzyme (sense or antisense), polynucleotide encoding for constant and variable regions of sgRNA/crRNA (sense or antisense), or sgRNA/crRNA.
ctccttgagctagaaatagcaagtaaggataaggctagtccgttatcaacttgaaaaagtggcaccgagtcggtgcttttt

SEQ ID NO: 75 - DNA, 81 bp, Artificial Sequence
  Polynucleotide encoding constant region of sgRNA/crRNA with non-palindromic recognition site for restriction enzyme (sense or antisense), polynucleotide encoding for constant and variable regions of sgRNA/crRNA (sense or antisense), or sgRNA/crRNA.
aaaaagcaccgactcggtgccactttttcaagttgataacggactagccttatccttacttgctatttctagctcaaggag

SEQ ID NO: 76 - DNA, 101 bp, Artificial Sequence
  Polynucleotide encoding constant region of sgRNA/crRNA with non-palindromic recognition site for restriction enzyme (sense or antisense), polynucleotide encoding for constant and variable regions of sgRNA/crRNA (sense or antisense), or sgRNA/crRNA.
  misc_feature (1)..(20): n is a, c, g, or t
nnnnnnnnnnnnnnnnnnnnctccttgagctagaaatagcaagtaaggataaggctagtccgttatcaacttgaaaaagtggcaccgagtcggtgcttttt

SEQ ID NO: 77 - DNA, 101 bp, Artificial Sequence
  Polynucleotide encoding constant region of sgRNA/crRNA with non-palindromic recognition site for restriction enzyme (sense or antisense), polynucleotide encoding for constant and variable regions of sgRNA/crRNA (sense or antisense), or sgRNA/crRNA.
  misc_feature (82)..(101): n is a, c, g, or t
aaaaagcaccgactcggtgccactttttcaagttgataacggactagccttatccttacttgctatttctagctcaaggagnnnnnnnnnnnnnnnnnnnn

SEQ ID NO: 78 - RNA, 101 nt, Artificial Sequence
  Polynucleotide encoding constant region of sgRNA/crRNA with non-palindromic recognition site for restriction enzyme (sense or antisense), polynucleotide encoding for constant and variable regions of sgRNA/crRNA (sense or antisense), or sgRNA/crRNA.
  misc_feature (1)..(20): n is a, c, g, or u
nnnnnnnnnnnnnnnnnnnncuccuugagcuagaaauagcaaguaaggauaaggcuaguccguuaucaacuugaaaaaguggcaccgagucggugcuuuuu

SEQ ID NO: 79 - DNA, 81 bp, Artificial Sequence
  Polynucleotide encoding constant region of sgRNA/crRNA with non-palindromic recognition site for restriction enzyme (sense or antisense), polynucleotide encoding for constant and variable regions of sgRNA/crRNA (sense or antisense), or sgRNA/crRNA.
  misc_feature (3)..(3): n is a, c, g, or t
  misc_feature (28)..(28): n is a, c, g, or t
gtncttgagctagaaatagcaagcaagnataaggctagtccgttatcaacttgaaaaagtggcaccgagtcggtgcttttt

SEQ ID NO: 80 - DNA, 81 bp, Artificial Sequence
  Polynucleotide encoding constant region of sgRNA/crRNA with non-palindromic recognition site for restriction enzyme (sense or antisense), polynucleotide encoding for constant and variable regions of sgRNA/crRNA (sense or antisense), or sgRNA/crRNA.
  misc_feature (54)..(54): n is a, c, g, or t
  misc_feature (79)..(79): n is a, c, g, or t
aaaaagcaccgactcggtgccactttttcaagttgataacggactagccttatncttgcttgctatttctagctcaagnac

SEQ ID NO: 81 - DNA, 101 bp, Artificial Sequence
  Polynucleotide encoding constant region of sgRNA/crRNA with non-palindromic recognition site for restriction enzyme (sense or antisense), polynucleotide encoding for constant and variable regions of sgRNA/crRNA (sense or antisense), or sgRNA/crRNA.
  misc_feature (1)..(20): n is a, c, g, or t
  misc_feature (23)..(23): n is a, c, g, or t
  misc_feature (48)..(48): n is a, c, g, or t
nnnnnnnnnnnnnnnnnnnngtncttgagctagaaatagcaagcaagnataaggctagtccgttatcaacttgaaaaagtggcaccgagtcggtgcttttt

SEQ ID NO: 82 - DNA, 101 bp, Artificial Sequence
  Polynucleotide encoding constant region of sgRNA/crRNA with non-palindromic recognition site for restriction enzyme (sense or antisense), polynucleotide encoding for constant and variable regions of sgRNA/crRNA (sense or antisense), or sgRNA/crRNA.
  misc_feature (54)..(54): n is a, c, g, or t
  misc_feature (79)..(79): n is a, c, g, or t
  misc_feature (82)..(101): n is a, c, g, or t
aaaaagcaccgactcggtgccactttttcaagttgataacggactagccttatncttgcttgctatttctagctcaagnacnnnnnnnnnnnnnnnnnnnn

SEQ ID NO: 83 - RNA, 101 nt, Artificial Sequence
  Polynucleotide encoding constant region of sgRNA/crRNA with non-palindromic recognition site for restriction enzyme (sense or antisense), polynucleotide encoding for constant and variable regions of sgRNA/crRNA (sense or antisense), or sgRNA/crRNA.
  misc_feature (1)..(20): n is a, c, g, or u
  misc_feature (23)..(23): n is a, c, g, or u
  misc_feature (48)..(48): n is a, c, g, or u
nnnnnnnnnnnnnnnnnnnnguncuugagcuagaaauagcaagcaagnauaaggcuaguccguuaucaacuugaaaaaguggcaccgagucggugcuuuuu

SEQ ID NO: 84 - DNA, 81 bp, Artificial Sequence
  Polynucleotide encoding constant region of sgRNA/crRNA with non-palindromic recognition site for restriction enzyme (sense or antisense), polynucleotide encoding for constant and variable regions of sgRNA/crRNA (sense or antisense), or sgRNA/crRNA.
gtcgtagagctagaaatagcaagttacgataaggctagtccgttatcaacttgaaaaagtggcaccgagtcggtgcttttt

SEQ ID NO: 85 - DNA, 81 bp, Artificial Sequence
  Polynucleotide encoding constant region of sgRNA/crRNA with non-palindromic recognition site for restriction enzyme (sense or antisense), polynucleotide encoding for constant and variable regions of sgRNA/crRNA (sense or antisense), or sgRNA/crRNA.
aaaaagcaccgactcggtgccactttttcaagttgataacggactagccttatcgtaacttgctatttctagctctacgac

SEQ ID NO: 86 - DNA, 101 bp, Artificial Sequence
  Polynucleotide encoding constant region of sgRNA/crRNA with non-palindromic recognition site for restriction enzyme (sense or antisense), polynucleotide encoding for constant and variable regions of sgRNA/crRNA (sense or antisense), or sgRNA/crRNA.
  misc_feature (1)..(20): n is a, c, g, or t
nnnnnnnnnnnnnnnnnnnngtcgtagagctagaaatagcaagttacgataaggctagtccgttatcaacttgaaaaagtggcaccgagtcggtgcttttt

SEQ ID NO: 87 - DNA, 101 bp, Artificial Sequence
  Polynucleotide encoding constant region of sgRNA/crRNA with non-palindromic recognition site for restriction enzyme (sense or antisense), polynucleotide encoding for constant and variable regions of sgRNA/crRNA (sense or antisense), or sgRNA/crRNA.
  misc_feature (82)..(101): n is a, c, g, or t
aaaaagcaccgactcggtgccactttttcaagttgataacggactagccttatcgtaacttgctatttctagctctacgacnnnnnnnnnnnnnnnnnnnn

SEQ ID NO: 88 - RNA, 101 nt, Artificial Sequence
  Polynucleotide encoding constant region of sgRNA/crRNA with non-palindromic recognition site for restriction enzyme (sense or antisense), polynucleotide encoding for constant and variable regions of sgRNA/crRNA (sense or antisense), or sgRNA/crRNA.
  misc_feature (1)..(20): n is a, c, g, or u
nnnnnnnnnnnnnnnnnnnngucguagagcuagaaauagcaaguuacgauaaggcuaguccguuaucaacuugaaaaaguggcaccgagucggugcuuuuu

SEQ ID NO: 89 - DNA, 81 bp, Artificial Sequence
  Polynucleotide encoding constant region of sgRNA/crRNA with non-palindromic recognition site for restriction enzyme (sense or antisense), polynucleotide encoding for constant and variable regions of sgRNA/crRNA (sense or antisense), or sgRNA/crRNA.
gtggtcgagctagaaatagcaagtgaccataaggctagtccgttatcaacttgaaaaagtggcaccgagtcggtgcttttt

SEQ ID NO: 90 - DNA, 81 bp, Artificial Sequence
  Polynucleotide encoding constant region of sgRNA/crRNA with non-palindromic recognition site for restriction enzyme (sense or antisense), polynucleotide encoding for constant and variable regions of sgRNA/crRNA (sense or antisense), or sgRNA/crRNA.
aaaaagcaccgactcggtgccactttttcaagttgataacggactagccttatggtcacttgctatttctagctcgaccac

SEQ ID NO: 91 - DNA, 101 bp, Artificial Sequence
  Polynucleotide encoding constant region of sgRNA/crRNA with non-palindromic recognition site for restriction enzyme (sense or antisense), polynucleotide encoding for constant and variable regions of sgRNA/crRNA (sense or antisense), or sgRNA/crRNA.
  misc_feature (1)..(20): n is a, c, g, or t
nnnnnnnnnnnnnnnnnnnngtggtcgagctagaaatagcaagtgaccataaggctagtccgttatcaacttgaaaaagtggcaccgagtcggtgcttttt

SEQ ID NO: 92 - DNA, 101 bp, Artificial Sequence
  Polynucleotide encoding constant region of sgRNA/crRNA with non-palindromic recognition site for restriction enzyme (sense or antisense), polynucleotide encoding for constant and variable regions of sgRNA/crRNA (sense or antisense), or sgRNA/crRNA.
  misc_feature (82)..(101): n is a, c, g, or t
aaaaagcaccgactcggtgccactttttcaagttgataacggactagccttatggtcacttgctatttctagctcgaccacnnnnnnnnnnnnnnnnnnnn

SEQ ID NO: 93 - RNA, 101 nt, Artificial Sequence
  Polynucleotide encoding constant region of sgRNA/crRNA with non-palindromic recognition site for restriction enzyme (sense or antisense), polynucleotide encoding for constant and variable regions of sgRNA/crRNA (sense or antisense), or sgRNA/crRNA.
  misc_feature (1)..(20): n is a, c, g, or u
nnnnnnnnnnnnnnnnnnnnguggucgagcuagaaauagcaagugaccauaaggcuaguccguuaucaacuugaaaaaguggcaccgagucggugcuuuuu

SEQ ID NO: 94 - DNA, 81 bp, Artificial Sequence
  Polynucleotide encoding constant region of sgRNA/crRNA with non-palindromic recognition site for restriction enzyme (sense or antisense), polynucleotide encoding for constant and variable regions of sgRNA/crRNA (sense or antisense), or sgRNA/crRNA.
gtcaaygagctagaaatagcaagcrttgataaggctagtccgttatcaacttgaaaaagtggcaccgagtcggtgcttttt

SEQ ID NO: 95 - DNA, 81 bp, Artificial Sequence
  Polynucleotide encoding constant region of sgRNA/crRNA with non-palindromic recognition site for restriction enzyme (sense or antisense), polynucleotide encoding for constant and variable regions of sgRNA/crRNA (sense or antisense), or sgRNA/crRNA.
aaaaagcaccgactcggtgccactttttcaagttgataacggactagccttatcaaygcttgctatttctagctcrttgac

SEQ ID NO: 96 - DNA, 101 bp, Artificial Sequence
  Polynucleotide encoding constant region of sgRNA/crRNA with non-palindromic recognition site for restriction enzyme (sense or antisense), polynucleotide encoding for constant and variable regions of sgRNA/crRNA (sense or antisense), or sgRNA/crRNA.
  misc_feature (1)..(20): n is a, c, g, or t
nnnnnnnnnnnnnnnnnnnngtcaaygagctagaaatagcaagcrttgataaggctagtccgttatcaacttgaaaaagtggcaccgagtcggtgcttttt

SEQ ID NO: 97 - DNA, 101 bp, Artificial Sequence
  Polynucleotide encoding constant region of sgRNA/crRNA with non-palindromic recognition site for restriction enzyme (sense or antisense), polynucleotide encoding for constant and variable regions of sgRNA/crRNA (sense or antisense), or sgRNA/crRNA.
  misc_feature (82)..(101): n is a, c, g, or t
aaaaagcaccgactcggtgccactttttcaagttgataacggactagccttatcaaygcttgctatttctagctcrttgacnnnnnnnnnnnnnnnnnnnn

SEQ ID NO: 98 - RNA, 101 nt, Artificial Sequence
  Polynucleotide encoding constant region of sgRNA/crRNA with non-palindromic recognition site for restriction enzyme (sense or antisense), polynucleotide encoding for constant and variable regions of sgRNA/crRNA (sense or antisense), or sgRNA/crRNA.
  misc_feature (1)..(20): n is a, c, g, or u
nnnnnnnnnnnnnnnnnnnngucaaygagcuagaaauagcaagcruugauaaggcuaguccguuaucaacuugaaaaaguggcaccgagucggugcuuuuu

SEQ ID NO: 99 - DNA, 81 bp, Artificial Sequence
  Polynucleotide encoding constant region of sgRNA/crRNA with non-palindromic recognition site for restriction enzyme (sense or antisense), polynucleotide encoding for constant and variable regions of sgRNA/crRNA (sense or antisense), or sgRNA/crRNA.
ctycttgagctagaaatagcaagcaagrataaggctagtccgttatcaacttgaaaaagtggcaccgagtcggtgcttttt

SEQ ID NO: 100 - DNA, 81 bp, Artificial Sequence
  Polynucleotide encoding constant region of sgRNA/crRNA with non-palindromic recognition site for restriction enzyme (sense or antisense), polynucleotide encoding for constant and variable regions of sgRNA/crRNA (sense or antisense), or sgRNA/crRNA.
aaaaagcaccgactcggtgccactttttcaagttgataacggactagccttatycttgcttgctatttctagctcaagrag

SEQ ID NO: 101 - DNA, 101 bp, Artificial Sequence
  Polynucleotide encoding constant region of sgRNA/crRNA with non-palindromic recognition site for restriction enzyme (sense or antisense), polynucleotide encoding for constant and variable regions of sgRNA/crRNA (sense or antisense), or sgRNA/crRNA.
  misc_feature (1)..(20): n is a, c, g, or t
nnnnnnnnnnnnnnnnnnnnctycttgagctagaaatagcaagcaagrataaggctagtccgttatcaacttgaaaaagtggcaccgagtcggtgcttttt

SEQ ID NO: 102 - DNA, 101 bp, Artificial Sequence
  Polynucleotide encoding constant region of sgRNA/crRNA with non-palindromic recognition site for restriction enzyme (sense or antisense), polynucleotide encoding for constant and variable regions of sgRNA/crRNA (sense or antisense), or sgRNA/crRNA.
  misc_feature (82)..(101): n is a, c, g, or t
aaaaagcaccgactcggtgccactttttcaagttgataacggactagccttatycttgcttgctatttctagctcaagragnnnnnnnnnnnnnnnnnnnn

SEQ ID NO: 103 - RNA, 101 nt, Artificial Sequence
  Polynucleotide encoding constant region of sgRNA/crRNA with non-palindromic recognition site for restriction enzyme (sense or antisense), polynucleotide encoding for constant and variable regions of sgRNA/crRNA (sense or antisense), or sgRNA/crRNA.
  misc_feature (1)..(20): n is a, c, g, or u
nnnnnnnnnnnnnnnnnnnncurcuugagcuagaaauagcaagcaagrauaaggcuaguccguuaucaacuugaaaaaguggcaccgagucggugcuuuuu

SEQ ID NO: 104 - DNA, 81 bp, Artificial Sequence
  Polynucleotide encoding constant region of sgRNA/crRNA with non-palindromic recognition site for restriction enzyme (sense or antisense), polynucleotide encoding for constant and variable regions of sgRNA/crRNA (sense or antisense), or sgRNA/crRNA.
gtgatggagctagaaatagcaagtcatcataaggctagtccgttatcaacttgaaaaagtggcaccgagtcggtgcttttt

SEQ ID NO: 105 - DNA, 81 bp, Artificial Sequence
  Polynucleotide encoding constant region of sgRNA/crRNA with non-palindromic recognition site for restriction enzyme (sense or antisense), polynucleotide encoding for constant and variable regions of sgRNA/crRNA (sense or antisense), or sgRNA/crRNA.
aaaaagcaccgactcggtgccactttttcaagttgataacggactagccttatgatgacttgctatttctagctccatcac

SEQ ID NO: 106 - DNA, 100 bp, Artificial Sequence
  Polynucleotide encoding constant region of sgRNA/crRNA with non-palindromic recognition site for restriction enzyme (sense or antisense), polynucleotide encoding for constant and variable regions of sgRNA/crRNA (sense or antisense), or sgRNA/crRNA.
  misc_feature (1)..(19): n is a, c, g, or t
nnnnnnnnnnnnnnnnnnngtgatggagctagaaatagcaagtcatcataaggctagtccgttatcaacttgaaaaagtggcaccgagtcggtgcttttt

SEQ ID NO: 107 - DNA, 100 bp, Artificial Sequence
  Polynucleotide encoding constant region of sgRNA/crRNA with non-palindromic recognition site for restriction enzyme (sense or antisense), polynucleotide encoding for constant and variable regions of sgRNA/crRNA (sense or antisense), or sgRNA/crRNA.
  misc_feature (82)..(100): n is a, c, g, or t
aaaaagcaccgactcggtgccactttttcaagttgataacggactagccttatgatgacttgctatttctagctccatcacnnnnnnnnnnnnnnnnnnn

SEQ ID NO: 108 - RNA, 100 nt, Artificial Sequence
  Polynucleotide encoding constant region of sgRNA/crRNA with non-palindromic recognition site for restriction enzyme (sense or antisense), polynucleotide encoding for constant and variable regions of sgRNA/crRNA (sense or antisense), or sgRNA/crRNA.
  misc_feature (1)..(19): n is a, c, g, or u
nnnnnnnnnnnnnnnnnnngugauggagcuagaaauagcaagucaucauaaggcuaguccguuaucaacuugaaaaaguggcaccgagucggugcuuuuu

SEQ ID NO: 109 - DNA, 81 bp, Artificial Sequence
  Polynucleotide encoding constant region of sgRNA/crRNA with non-palindromic recognition site for restriction enzyme (sense or antisense), polynucleotide encoding for constant and variable regions of sgRNA/crRNA (sense or antisense), or sgRNA/crRNA.
ctgatggagctagaaatagcaagtcatcataaggctagtccgttatcaacttgaaaaagtggcaccgagtcggtgcttttt

SEQ ID NO: 110 - DNA, 81 bp, Artificial Sequence
  Polynucleotide encoding constant region of sgRNA/crRNA with non-palindromic recognition site for restriction enzyme (sense or antisense), polynucleotide encoding for constant and variable regions of sgRNA/crRNA (sense or antisense), or sgRNA/crRNA.
aaaaagcaccgactcggtgccactttttcaagttgataacggactagccttatgatgacttgctatttctagctccatcag

SEQ ID NO: 111 - DNA, 102 bp, Artificial Sequence
  Polynucleotide encoding constant region of sgRNA/crRNA with non-palindromic recognition site for restriction enzyme (sense or antisense), polynucleotide encoding for constant and variable regions of sgRNA/crRNA (sense or antisense), or sgRNA/crRNA.
  misc_feature (1)..(21): n is a, c, g, or t
nnnnnnnnnnnnnnnnnnnnnctgatggagctagaaatagcaagtcatcataaggctagtccgttatcaacttgaaaaagtggcaccgagtcggtgcttttt

SEQ ID NO: 112 - DNA, 102 bp, Artificial Sequence
  Polynucleotide encoding constant region of sgRNA/crRNA with non-palindromic recognition site for restriction enzyme (sense or antisense), polynucleotide encoding for constant and variable regions of sgRNA/crRNA (sense or antisense), or sgRNA/crRNA.
  misc_feature (82)..(102): n is a, c, g, or t
aaaaagcaccgactcggtgccactttttcaagttgataacggactagccttatgatgacttgctatttctagctccatcagnnnnnnnnnnnnnnnnnnnnn

SEQ ID NO: 113 - RNA, 102 nt, Artificial Sequence
  Polynucleotide encoding constant region of sgRNA/crRNA with non-palindromic recognition site for restriction enzyme (sense or antisense), polynucleotide encoding for constant and variable regions of sgRNA/crRNA (sense or antisense), or sgRNA/crRNA.
  misc_feature (1)..(21): n is a, c, g, or u
nnnnnnnnnnnnnnnnnnnnncugauggagcuagaaauagcaagucaucauaaggcuaguccguuaucaacuugaaaaaguggcaccgagucggugcuuuuu

SEQ ID NO: 114 - DNA, 81 bp, Artificial Sequence
  Polynucleotide encoding constant region of sgRNA/crRNA with non-palindromic recognition site for restriction enzyme (sense or antisense), polynucleotide encoding for constant and variable regions of sgRNA/crRNA (sense or antisense), or sgRNA/crRNA.
ytgggcgagctagaaatagcaagcgcccataaggctagtccgttatcaacttgaaaaagtggcaccgagtcggtgcttttt

SEQ ID NO: 115 - DNA, 81 bp, Artificial Sequence
  Polynucleotide encoding constant region of sgRNA/crRNA with non-palindromic recognition site for restriction enzyme (sense or antisense), polynucleotide encoding for constant and variable regions of sgRNA/crRNA (sense or antisense), or sgRNA/crRNA.
aaaaagcaccgactcggtgccactttttcaagttgataacggactagccttatgggcgcttgctatttctagctcgcccar

SEQ ID NO: 116 - DNA, 101 bp, Artificial Sequence
  Polynucleotide encoding constant region of sgRNA/crRNA with non-palindromic recognition site for restriction enzyme (sense or antisense), polynucleotide encoding for constant and variable regions of sgRNA/crRNA (sense or antisense), or sgRNA/crRNA.
  misc_feature (1)..(20): n is a, c, g, or t
nnnnnnnnnnnnnnnnnnnnytgggcgagctagaaatagcaagcgcccataaggctagtccgttatcaacttgaaaaagtggcaccgagtcggtgcttttt

SEQ ID NO: 117 - DNA, 101 bp, Artificial Sequence
  Polynucleotide encoding constant region of sgRNA/crRNA with non-palindromic recognition site for restriction enzyme (sense or antisense), polynucleotide encoding for constant and variable regions of sgRNA/crRNA (sense or antisense), or sgRNA/crRNA.
  misc_feature (82)..(101): n is a, c, g, or t
aaaaagcaccgactcggtgccactttttcaagttgataacggactagccttatgggcgcttgctatttctagctcgcccarnnnnnnnnnnnnnnnnnnnn

SEQ ID NO: 118 - RNA, 101 nt, Artificial Sequence
  Polynucleotide encoding constant region of sgRNA/crRNA with non-palindromic recognition site for restriction enzyme (sense or antisense), polynucleotide encoding for constant and variable regions of sgRNA/crRNA (sense or antisense), or sgRNA/crRNA.
  misc_feature (1)..(20): n is a, c, g, or u
nnnnnnnnnnnnnnnnnnnnyugggcgagcuagaaauagcaagcgcccauaaggcuaguccguuaucaacuugaaaaaguggcaccgagucggugcuuuuu

SEQ ID NO: 119 - DNA, 81 bp, Artificial Sequence
  Polynucleotide encoding constant region of sgRNA/crRNA with non-palindromic recognition site for restriction enzyme (sense or antisense), polynucleotide encoding for constant and variable regions of sgRNA/crRNA (sense or antisense), or sgRNA/crRNA.
ctgrgggagctagaaatagcaagtccycataaggctagtccgttatcaacttgaaaaagtggcaccgagtcggtgcttttt

SEQ ID NO: 120 - DNA, 81 bp, Artificial Sequence
  Polynucleotide encoding constant region of sgRNA/crRNA with non-palindromic recognition site for restriction enzyme (sense or antisense), polynucleotide encoding for constant and variable regions of sgRNA/crRNA (sense or antisense), or sgRNA/crRNA.
aaaaagcaccgactcggtgccactttttcaagttgataacggactagccttatgrggacttgctatttctagctcccycag

SEQ ID NO: 121 - DNA, 102 bp, Artificial Sequence
  Polynucleotide encoding constant region of sgRNA/crRNA with non-palindromic recognition site for restriction enzyme (sense or antisense), polynucleotide encoding for constant and variable regions of sgRNA/crRNA (sense or antisense), or sgRNA/crRNA.
  misc_feature (1)..(21): n is a, c, g, or t
nnnnnnnnnnnnnnnnnnnnnctgrgggagctagaaatagcaagtccycataaggctagtccgttatcaacttgaaaaagtggcaccgagtcggtgcttttt

SEQ ID NO: 122 - DNA, 102 bp, Artificial Sequence
  Polynucleotide encoding constant region of sgRNA/crRNA with non-palindromic recognition site for restriction enzyme (sense or antisense), polynucleotide encoding for constant and variable regions of sgRNA/crRNA (sense or antisense), or sgRNA/crRNA.
  misc_feature (82)..(102): n is a, c, g, or t
aaaaagcaccgactcggtgccactttttcaagttgataacggactagccttatgrggacttgctatttctagctcccycagnnnnnnnnnnnnnnnnnnnnn

SEQ ID NO: 123 - RNA, 101 nt, Artificial Sequence
  Polynucleotide encoding constant region of sgRNA/crRNA with non-palindromic recognition site for restriction enzyme (sense or antisense), polynucleotide encoding for constant and variable regions of sgRNA/crRNA (sense or antisense), or sgRNA/crRNA.
  misc_feature (1)..(21): n is a, c, g, or u
nnnnnnnnnnnnnnnnnnnnncugrgggagcuagaaauagcaagucccauaaggcuaguccguuaucaacuugaaaaaguggcaccgagucggugcuuuuu

SEQ ID NO: 124 - DNA, 81 bp, Artificial Sequence
  Polynucleotide encoding constant region of sgRNA/crRNA with non-palindromic recognition site for restriction enzyme (sense or antisense), polynucleotide encoding for constant and variable regions of sgRNA/crRNA (sense or antisense), or sgRNA/crRNA.
gtcgatgagctagaaatagcaagcatcgataaggctagtccgttatcaacttgaaaaagtggcaccgagtcggtgcttttt

SEQ ID NO: 125 - DNA, 81 bp, Artificial Sequence
  Polynucleotide encoding constant region of sgRNA/crRNA with non-palindromic recognition site for restriction enzyme (sense or antisense), polynucleotide encoding for constant and variable regions of sgRNA/crRNA (sense or antisense), or sgRNA/crRNA.
aaaaagcaccgactcggtgccactttttcaagttgataacggactagccttatcgatgcttgctatttctagctcatcgac

SEQ ID NO: 126 - DNA, 101 bp, Artificial Sequence
  Polynucleotide encoding constant region of sgRNA/crRNA with non-palindromic recognition site for restriction enzyme (sense or antisense), polynucleotide encoding for constant and variable regions of sgRNA/crRNA (sense or antisense), or sgRNA/crRNA.
  misc_feature (1)..(20): n is a, c, g, or t
nnnnnnnnnnnnnnnnnnnngtcgatgagctagaaatagcaagcatcgataaggctagtccgttatcaacttgaaaaagtggcaccgagtcggtgcttttt

SEQ ID NO: 127 - DNA, 101 bp, Artificial Sequence
  Polynucleotide encoding constant region of sgRNA/crRNA with non-palindromic recognition site for restriction enzyme (sense or antisense), polynucleotide encoding for constant and variable regions of sgRNA/crRNA (sense or antisense), or sgRNA/crRNA.
  misc_feature (82)..(101): n is a, c, g, or t
aaaaagcaccgactcggtgccactttttcaagttgataacggactagccttatcgatgcttgctatttctagctcatcgacnnnnnnnnnnnnnnnnnnnn

SEQ ID NO: 128 - RNA, 101 nt, Artificial Sequence
  Polynucleotide encoding constant region of sgRNA/crRNA with non-palindromic recognition site for restriction enzyme (sense or antisense), polynucleotide encoding for constant and variable regions of sgRNA/crRNA (sense or antisense), or sgRNA/crRNA.
  misc_feature (82)..(101): n is a, c, g, or u
gucgaugagcuagaaauagcaagcaucgauaaggcuaguccguuaucaacuugaaaaaguggcaccgagucggugcuuuuunnnnnnnnnnnnnnnnnnnn

SEQ ID NO: 129 - DNA, 81 bp, Artificial Sequence
  Polynucleotide encoding constant region of sgRNA/crRNA with non-palindromic recognition site for restriction enzyme (sense or antisense), polynucleotide encoding for constant and variable regions of sgRNA/crRNA (sense or antisense), or sgRNA/crRNA.
gtccycgagctagaaatagcaagcgrggataaggctagtccgttatcaacttgaaaaagtggcaccgagtcggtgcttttt

SEQ ID NO: 130 - DNA, 81 bp, Artificial Sequence
  Polynucleotide encoding constant region of sgRNA/crRNA with non-palindromic recognition site for restriction enzyme (sense or antisense), polynucleotide encoding for constant and variable regions of sgRNA/crRNA (sense or antisense), or sgRNA/crRNA.
aaaaagcaccgactcggtgccactttttcaagttgataacggactagccttatccycgcttgctatttctagctcgrggac

SEQ ID NO: 131 - DNA, 101 bp, Artificial Sequence
  Polynucleotide encoding constant region of sgRNA/crRNA with non-palindromic recognition site for restriction enzyme (sense or antisense), polynucleotide encoding for constant and variable regions of sgRNA/crRNA (sense or antisense), or sgRNA/crRNA.
  misc_feature (1)..(20): n is a, c, g, or t
nnnnnnnnnnnnnnnnnnnngtccycgagctagaaatagcaagcgrggataaggctagtccgttatcaacttgaaaaagtggcaccgagtcggtgcttttt

SEQ ID NO: 132 - DNA, 101 bp, Artificial Sequence
  Polynucleotide encoding constant region of sgRNA/crRNA with non-palindromic recognition site for restriction enzyme (sense or antisense), polynucleotide encoding for constant and variable regions of sgRNA/crRNA (sense or antisense), or sgRNA/crRNA.
  misc_feature (82)..(101): n is a, c, g, or t
aaaaagcaccgactcggtgccactttttcaagttgataacggactagccttatccycgcttgctatttctagctcgrggacnnnnnnnnnnnnnnnnnnnn

SEQ ID NO: 133 - RNA, 101 nt, Artificial Sequence
  Polynucleotide encoding constant region of sgRNA/crRNA with non-palindromic recognition site for restriction enzyme (sense or antisense), polynucleotide encoding for constant and variable regions of sgRNA/crRNA (sense or antisense), or sgRNA/crRNA.
  misc_feature (1)..(20): n is a, c, g, or u
nnnnnnnnnnnnnnnnnnnnguccycgagcuagaaauagcaagcgrggauaaggcuaguccguuaucaacuugaaaaaguggcaccgagucggugcuuuuu

SEQ ID NO: 134 - DNA, 81 bp, Artificial Sequence
  Polynucleotide encoding constant region of sgRNA/crRNA with non-palindromic recognition site for restriction enzyme (sense or antisense), polynucleotide encoding for constant and variable regions of sgRNA/crRNA (sense or antisense), or sgRNA/crRNA.
ctyctggagctagaaatagcaagtcagrataaggctagtccgttatcaacttgaaaaagtggcaccgagtcggtgcttttt

SEQ ID NO: 135 - DNA, 81 bp, Artificial Sequence
  Polynucleotide encoding constant region of sgRNA/crRNA with non-palindromic recognition site for restriction enzyme (sense or antisense), polynucleotide encoding for constant and variable regions of sgRNA/crRNA (sense or antisense), or sgRNA/crRNA.
aaaaagcaccgactcggtgccactttttcaagttgataacggactagccttatyctgacttgctatttctagctccagrag

SEQ ID NO: 136 - DNA, 102 bp, Artificial Sequence
  Polynucleotide encoding constant region of sgRNA/crRNA with non-palindromic recognition site for restriction enzyme (sense or antisense), polynucleotide encoding for constant and variable regions of sgRNA/crRNA (sense or antisense), or sgRNA/crRNA.
  misc_feature (1)..(21): n is a, c, g, or t
nnnnnnnnnnnnnnnnnnnnnctyctggagctagaaatagcaagtcagrataaggctagtccgttatcaacttgaaaaagtggcaccgagtcggtgcttttt

SEQ ID NO: 137 - DNA, 102 bp, Artificial Sequence
  Polynucleotide encoding constant region of sgRNA/crRNA with non-palindromic recognition site for restriction enzyme (sense or antisense), polynucleotide encoding for constant and variable regions of sgRNA/crRNA (sense or antisense), or sgRNA/crRNA.
  misc_feature (82)..(102): n is a, c, g, or t
aaaaagcaccgactcggtgccactttttcaagttgataacggactagccttatyctgacttgctatttctagctccagragnnnnnnnnnnnnnnnnnnnnn

SEQ ID NO: 138 - RNA, 101 nt, Artificial Sequence
  Polynucleotide encoding constant region of sgRNA/crRNA with non-palindromic recognition site for restriction enzyme (sense or antisense), polynucleotide encoding for constant and variable regions of sgRNA/crRNA (sense or antisense), or sgRNA/crRNA.
  misc_feature (1)..(21): n is a, c, g, or u
nnnnnnnnnnnnnnnnnnnnncuycuggagcuagaaauagcaagucagauaaggcuaguccguuaucaacuugaaaaaguggcaccgagucggugcuuuuu

SEQ ID NO: 139 - DNA, 81 bp, Artificial Sequence
  Polynucleotide encoding constant region of sgRNA/crRNA with non-palindromic recognition site for restriction enzyme (sense or antisense), polynucleotide encoding for constant and variable regions of sgRNA/crRNA (sense or antisense), or sgRNA/crRNA.
ctyccgcagctagaaatagcaaggcggrataaggctagtccgttatcaacttgaaaaagtggcaccgagtcggtgcttttt

SEQ ID NO: 140 - DNA, 81 bp, Artificial Sequence
  Polynucleotide encoding constant region of sgRNA/crRNA with non-palindromic recognition site for restriction enzyme (sense or antisense), polynucleotide encoding for constant and variable regions of sgRNA/crRNA (sense or antisense), or sgRNA/crRNA.
aaaaagcaccgactcggtgccactttttcaagttgataacggactagccttatyccgccttgctatttctagctgcggrag

SEQ ID NO: 141 - DNA, 101 bp, Artificial Sequence
  Polynucleotide encoding constant region of sgRNA/crRNA with non-palindromic recognition site for restriction enzyme (sense or antisense), polynucleotide encoding for constant and variable regions of sgRNA/crRNA (sense or antisense), or sgRNA/crRNA.
  misc_feature (1)..(20): n is a, c, g, or t
nnnnnnnnnnnnnnnnnnnnctyccgcagctagaaatagcaaggcggrataaggctagtccgttatcaacttgaaaaagtggcaccgagtcggtgcttttt

SEQ ID NO: 142 - DNA, 101 bp, Artificial Sequence
  Polynucleotide encoding constant region of sgRNA/crRNA with non-palindromic recognition site for restriction enzyme (sense or antisense), polynucleotide encoding for constant and variable regions of sgRNA/crRNA (sense or antisense), or sgRNA/crRNA.
  misc_feature (82)..(101): n is a, c, g, or t
aaaaagcaccgactcggtgccactttttcaagttgataacggactagccttatyccgccttgctatttctagctgcggragnnnnnnnnnnnnnnnnnnnn

SEQ ID NO: 143 - RNA, 101 nt, Artificial Sequence
  Polynucleotide encoding constant region of sgRNA/crRNA with non-palindromic recognition site for restriction enzyme (sense or antisense), polynucleotide encoding for constant and variable regions of sgRNA/crRNA (sense or antisense), or sgRNA/crRNA.
  misc_feature (1)..(20): n is a, c, g, or u
nnnnnnnnnnnnnnnnnnnncuyccgcagcuagaaauagcaaggcggrauaaggcuaguccguuaucaacuugaaaaaguggcaccgagucggugcuuuuu

SEQ ID NO: 144 - DNA, 81 bp, Artificial Sequence
  Polynucleotide encoding constant region of sgRNA/crRNA with non-palindromic recognition site for restriction enzyme (sense or antisense), polynucleotide encoding for constant and variable regions of sgRNA/crRNA (sense or antisense), or sgRNA/crRNA.
gtgttggagctagaaatagcaagtcaacataaggctagtccgttatcaacttgaaaaagtggcaccgagtcggtgcttttt

SEQ ID NO: 145 - DNA, 81 bp, Artificial Sequence
  Polynucleotide encoding constant region of sgRNA/crRNA with non-palindromic recognition site for restriction enzyme (sense or antisense), polynucleotide encoding for constant and variable regions of sgRNA/crRNA (sense or antisense), or sgRNA/crRNA.
aaaaagcaccgactcggtgccactttttcaagttgataacggactagccttatgttgacttgctatttctagctccaacac

SEQ ID NO: 146 - DNA, 102 bp, Artificial Sequence
  Polynucleotide encoding constant region of sgRNA/crRNA with non-palindromic recognition site for restriction enzyme (sense or antisense), polynucleotide encoding for constant and variable regions of sgRNA/crRNA (sense or antisense), or sgRNA/crRNA.
  misc_feature (1)..(21): n is a, c, g, or t
nnnnnnnnnnnnnnnnnnnnngtgttggagctagaaatagcaagtcaacataaggctagtccgttatcaacttgaaaaagtggcaccgagtcggtgcttttt

SEQ ID NO: 147 - DNA, 102 bp, Artificial Sequence
  Polynucleotide encoding constant region of sgRNA/crRNA with non-palindromic recognition site for restriction enzyme (sense or antisense), polynucleotide encoding for constant and variable regions of sgRNA/crRNA (sense or antisense), or sgRNA/crRNA.
  misc_feature (82)..(102): n is a, c, g, or t
aaaaagcaccgactcggtgccactttttcaagttgataacggactagccttatgttgacttgctatttctagctccaacacnnnnnnnnnnnnnnnnnnnnn

SEQ ID NO: 148 - RNA, 102 nt, Artificial Sequence
  Polynucleotide encoding constant region of sgRNA/crRNA with non-palindromic recognition site for restriction enzyme (sense or antisense), polynucleotide encoding for constant and variable regions of sgRNA/crRNA (sense or antisense), or sgRNA/crRNA.
  misc_feature (1)..(21): n is a, c, g, or u
nnnnnnnnnnnnnnnnnnnnnguguuggagcuagaaauagcaagucaacauaaggcuaguccguuaucaacuugaaaaaguggcaccgagucggugcuuuuu

SEQ ID NO: 149 - DNA, 19 bp, Francisella novicida
aatttctactgttgtagat

SEQ ID NO: 150 - DNA, 19 bp, Francisella novicida
atctacaacagtagaaatt

SEQ ID NO: 151 - RNA, 19 nt, Francisella novicida
aauuucuacuguuguagau

SEQ ID NO: 152 - DNA, 19 bp, Artificial Sequence
  Polynucleotide encoding constant region of sgRNA/crRNA with non-palindromic recognition site for restriction enzyme (sense or antisense), polynucleotide encoding for constant and variable regions of sgRNA/crRNA (sense or antisense), or sgRNA/crRNA.
aattgttggtgttccract

SEQ ID NO: 153 - DNA, 19 bp, Artificial Sequence
  Polynucleotide encoding constant region of sgRNA/crRNA with non-palindromic recognition site for restriction enzyme (sense or antisense), polynucleotide encoding for constant and variable regions of sgRNA/crRNA (sense or antisense), or sgRNA/crRNA.
agtyggaacaccaacaatt

SEQ ID NO: 154 - DNA, 38 bp, Artificial Sequence
  Polynucleotide encoding constant region of sgRNA/crRNA with non-palindromic recognition site for restriction enzyme (sense or antisense), polynucleotide encoding for constant and variable regions of sgRNA/crRNA (sense or antisense), or sgRNA/crRNA.
  misc_feature (20)..(38): n is a, c, g, or t
aattgttggtgttccractnnnnnnnnnnnnnnnnnnn

SEQ ID NO: 155 - DNA, 38 bp, Artificial Sequence
  Polynucleotide encoding constant region of sgRNA/crRNA with non-palindromic recognition site for restriction enzyme (sense or antisense), polynucleotide encoding for constant and variable regions of sgRNA/crRNA (sense or antisense), or sgRNA/crRNA.
  misc_feature (1)..(19): n is a, c, g, or t
nnnnnnnnnnnnnnnnnnnagtyggaacaccaacaatt

SEQ ID NO: 156 - RNA, 38 nt, Artificial Sequence
  Polynucleotide encoding constant region of sgRNA/crRNA with non-palindromic recognition site for restriction enzyme (sense or antisense), polynucleotide encoding for constant and variable regions of sgRNA/crRNA (sense or antisense), or sgRNA/crRNA.
  misc_feature (20)..(38): n is a, c, g, or u
aauuguugguguuccracunnnnnnnnnnnnnnnnnnn

SEQ ID NO: 157 - DNA, 19 bp, Artificial Sequence
  Polynucleotide encoding constant region of sgRNA/crRNA with non-palindromic recognition site for restriction enzyme (sense or antisense), polynucleotide encoding for constant and variable regions of sgRNA/crRNA (sense or antisense), or sgRNA/crRNA.
aatttgttgtgtccaacat

SEQ ID NO: 158 - DNA, 19 bp, Artificial Sequence
  Polynucleotide encoding constant region of sgRNA/crRNA with non-palindromic recognition site for restriction enzyme (sense or antisense), polynucleotide encoding for constant and variable regions of sgRNA/crRNA (sense or antisense), or sgRNA/crRNA.
atgttggacacaacaaatt

SEQ ID NO: 159 - DNA, 37 bp, Artificial Sequence
  Polynucleotide encoding constant region of sgRNA/crRNA with non-palindromic recognition site for restriction enzyme (sense or antisense), polynucleotide encoding for constant and variable regions of sgRNA/crRNA (sense or antisense), or sgRNA/crRNA.
  misc_feature (20)..(37): n is a, c, g, or t
aatttgttgtgtccaacatnnnnnnnnnnnnnnnnnn

SEQ ID NO: 160 - DNA, 37 bp, Artificial Sequence
  Polynucleotide encoding constant region of sgRNA/crRNA with non-palindromic recognition site for restriction enzyme (sense or antisense), polynucleotide encoding for constant and variable regions of sgRNA/crRNA (sense or antisense), or sgRNA/crRNA.
  misc_feature (1)..(18): n is a, c, g, or t
nnnnnnnnnnnnnnnnnnatgttggacacaacaaatt

SEQ ID NO: 161 - RNA, 37 nt, Artificial Sequence
  Polynucleotide encoding constant region of sgRNA/crRNA with non-palindromic recognition site for restriction enzyme (sense or antisense), polynucleotide encoding for constant and variable regions of sgRNA/crRNA (sense or antisense), or sgRNA/crRNA.
  misc_feature (20)..(37): n is a, c, g, or u
aauuuguuguguccaacaunnnnnnnnnnnnnnnnnn

SEQ ID NO: 162 - DNA, 21 bp, Artificial Sequence
  Polynucleotide encoding constant region of sgRNA/crRNA with non-palindromic recognition site for restriction enzyme (sense or antisense), polynucleotide encoding for constant and variable regions of sgRNA/crRNA (sense or antisense), or sgRNA/crRNA.
aattgttggtgttccractat

SEQ ID NO: 163 - DNA, 21 bp, Artificial Sequence
  Polynucleotide encoding constant region of sgRNA/crRNA with non-palindromic recognition site for restriction enzyme (sense or antisense), polynucleotide encoding for constant and variable regions of sgRNA/crRNA (sense or antisense), or sgRNA/crRNA.
atagtyggaacaccaacaatt

SEQ ID NO: 164 - DNA, 40 bp, Artificial Sequence
  Polynucleotide encoding constant region of sgRNA/crRNA with non-palindromic recognition site for restriction enzyme (sense or antisense), polynucleotide encoding for constant and variable regions of sgRNA/crRNA (sense or antisense), or sgRNA/crRNA.
  misc_feature (22)..(40): n is a, c, g, or t
aattgttggtgttccractatnnnnnnnnnnnnnnnnnnn

SEQ ID NO: 165 - DNA, 40 bp, Artificial Sequence
  Polynucleotide encoding constant region of sgRNA/crRNA with non-palindromic recognition site for restriction enzyme (sense or antisense), polynucleotide encoding for constant and variable regions of sgRNA/crRNA (sense or antisense), or sgRNA/crRNA.
  misc_feature (1)..(19): n is a, c, g, or t
nnnnnnnnnnnnnnnnnnnatagtyggaacaccaacaatt

SEQ ID NO: 166 - RNA, 40 nt, Artificial Sequence
  Polynucleotide encoding constant region of sgRNA/crRNA with non-palindromic recognition site for restriction enzyme (sense or antisense), polynucleotide encoding for constant and variable regions of sgRNA/crRNA (sense or antisense), or sgRNA/crRNA.
  misc_feature (22)..(40): n is a, c, g, or u
aauuguugguguuccracuaunnnnnnnnnnnnnnnnnnn

SEQ ID NO: 167 - DNA, 19 bp, Artificial Sequence
  Polynucleotide encoding constant region of sgRNA/crRNA with non-palindromic recognition site for restriction enzyme (sense or antisense), polynucleotide encoding for constant and variable regions of sgRNA/crRNA (sense or antisense), or sgRNA/crRNA.
aatttgttgtgtccaacat

SEQ ID NO: 168 - DNA, 19 bp, Artificial Sequence
  Polynucleotide encoding constant region of sgRNA/crRNA with non-palindromic recognition site for restriction enzyme (sense or antisense), polynucleotide encoding for constant and variable regions of sgRNA/crRNA (sense or antisense), or sgRNA/crRNA.
atgttggacacaacaaatt

SEQ ID NO: 169 - DNA, 37 bp, Artificial Sequence; identical to SEQ ID NO: 159
  Polynucleotide encoding constant region of sgRNA/crRNA with non-palindromic recognition site for restriction enzyme (sense or antisense), polynucleotide encoding for constant and variable regions of sgRNA/crRNA (sense or antisense), or sgRNA/crRNA.
  misc_feature (20)..(37): n is a, c, g, or t

SEQ ID NO: 170 - DNA, 37 bp, Artificial Sequence; identical to SEQ ID NO: 160
  Polynucleotide encoding constant region of sgRNA/crRNA with non-palindromic recognition site for restriction enzyme (sense or antisense), polynucleotide encoding for constant and variable regions of sgRNA/crRNA (sense or antisense), or sgRNA/crRNA.
  misc_feature (1)..(18): n is a, c, g, or t

SEQ ID NO: 171 - RNA, 37 nt, Artificial Sequence; identical to SEQ ID NO: 161
  Polynucleotide encoding constant region of sgRNA/crRNA with non-palindromic recognition site for restriction enzyme (sense or antisense), polynucleotide encoding for constant and variable regions of sgRNA/crRNA (sense or antisense), or sgRNA/crRNA.
  misc_feature (20)..(37): n is a, c, g, or u

SEQ ID NO: 172 - DNA, 19 bp, Artificial Sequence
  Polynucleotide encoding constant region of sgRNA/crRNA with non-palindromic recognition site for restriction enzyme (sense or antisense), polynucleotide encoding for constant and variable regions of sgRNA/crRNA (sense or antisense), or sgRNA/crRNA.
aattctcggtgtgccgagt

SEQ ID NO: 173 - DNA, 19 bp, Artificial Sequence
  Polynucleotide encoding constant region of sgRNA/crRNA with non-palindromic recognition site for restriction enzyme (sense or antisense), polynucleotide encoding for constant and variable regions of sgRNA/crRNA (sense or antisense), or sgRNA/crRNA.
actcggcacaccgagaatt

SEQ ID NO: 174 - DNA, 38 bp, Artificial Sequence
  Polynucleotide encoding constant region of sgRNA/crRNA with non-palindromic recognition site for restriction enzyme (sense or antisense), polynucleotide encoding for constant and variable regions of sgRNA/crRNA (sense or antisense), or sgRNA/crRNA.
  misc_feature (20)..(38): n is a, c, g, or t
aattctcggtgtgccgagtnnnnnnnnnnnnnnnnnnn

SEQ ID NO: 175 - DNA, 38 bp, Artificial Sequence
  Polynucleotide encoding constant region of sgRNA/crRNA with non-palindromic recognition site for restriction enzyme (sense or antisense), polynucleotide encoding for constant and variable regions of sgRNA/crRNA (sense or antisense), or sgRNA/crRNA.
  misc_feature (1)..(19): n is a, c, g, or t
nnnnnnnnnnnnnnnnnnnactcggcacaccgagaatt

SEQ ID NO: 176 - RNA, 38 nt, Artificial Sequence
  Polynucleotide encoding constant region of sgRNA/crRNA with non-palindromic recognition site for restriction enzyme (sense or antisense), polynucleotide encoding for constant and variable regions of sgRNA/crRNA (sense or antisense), or sgRNA/crRNA.
  misc_feature (20)..(38): n is a, c, g, or u
aauucucggugugccgagunnnnnnnnnnnnnnnnnnn

SEQ ID NO: 177 - DNA, 19 bp, Artificial Sequence
  Polynucleotide encoding constant region of sgRNA/crRNA with non-palindromic recognition site for restriction enzyme (sense or antisense), polynucleotide encoding for constant and variable regions of sgRNA/crRNA (sense or antisense), or sgRNA/crRNA.
aatttctcgtggccgagat

SEQ ID NO: 178 - DNA, 19 bp, Artificial Sequence
  Polynucleotide encoding constant region of sgRNA/crRNA with non-palindromic recognition site for restriction enzyme (sense or antisense), polynucleotide encoding for constant and variable regions of sgRNA/crRNA (sense or antisense), or sgRNA/crRNA.
atctcggccacgagaaatt

SEQ ID NO: 179 - DNA, 37 bp, Artificial Sequence
  Polynucleotide encoding constant region of sgRNA/crRNA with non-palindromic recognition site for restriction enzyme (sense or antisense), polynucleotide encoding for constant and variable regions of sgRNA/crRNA (sense or antisense), or sgRNA/crRNA.
  misc_feature (20)..(37): n is a, c, g, or t
aatttctcgtggccgagatnnnnnnnnnnnnnnnnnn

SEQ ID NO: 180 - DNA, 37 bp, Artificial Sequence
  Polynucleotide encoding constant region of sgRNA/crRNA with non-palindromic recognition site for restriction enzyme (sense or antisense), polynucleotide encoding for constant and variable regions of sgRNA/crRNA (sense or antisense), or sgRNA/crRNA.
  misc_feature (1)..(18): n is a, c, g, or t
nnnnnnnnnnnnnnnnnnatctcggccacgagaaatt

SEQ ID NO: 181 - RNA, 37 nt, Artificial Sequence
  Polynucleotide encoding constant region of sgRNA/crRNA with non-palindromic recognition site for restriction enzyme (sense or antisense), polynucleotide encoding for constant and variable regions of sgRNA/crRNA (sense or antisense), or sgRNA/crRNA.
  misc_feature (20)..(37): n is a, c, g, or u
aauuucucguggccgagaunnnnnnnnnnnnnnnnnn

SEQ ID NO: 182 - DNA, 21 bp, Artificial Sequence
  Polynucleotide encoding constant region of sgRNA/crRNA with non-palindromic recognition site for restriction enzyme (sense or antisense), polynucleotide encoding for constant and variable regions of sgRNA/crRNA (sense or antisense), or sgRNA/crRNA.
aattctcggtgtgccgagtat

SEQ ID NO: 183 - DNA, 21 bp, Artificial Sequence
  Polynucleotide encoding constant region of sgRNA/crRNA with non-palindromic recognition site for restriction enzyme (sense or antisense), polynucleotide encoding for constant and variable regions of sgRNA/crRNA (sense or antisense), or sgRNA/crRNA.
atactcggcacaccgagaatt

SEQ ID NO: 184 - DNA, 40 bp, Artificial Sequence
  Polynucleotide encoding constant region of sgRNA/crRNA with non-palindromic recognition site for restriction enzyme (sense or antisense), polynucleotide encoding for constant and variable regions of sgRNA/crRNA (sense or antisense), or sgRNA/crRNA.
  misc_feature (22)..(40): n is a, c, g, or t
aattctcggtgtgccgagtatnnnnnnnnnnnnnnnnnnn

SEQ ID NO: 185 - DNA, 40 bp, Artificial Sequence
  Polynucleotide encoding constant region of sgRNA/crRNA with non-palindromic recognition site for restriction enzyme (sense or antisense), polynucleotide encoding for constant and variable regions of sgRNA/crRNA (sense or antisense), or sgRNA/crRNA.
  misc_feature (1)..(19): n is a, c, g, or t
nnnnnnnnnnnnnnnnnnnatactcggcacaccgagaatt

SEQ ID NO: 186 - RNA, 40 nt, Artificial Sequence
  Polynucleotide encoding constant region of sgRNA/crRNA with non-palindromic recognition site for restriction enzyme (sense or antisense), polynucleotide encoding for constant and variable regions of sgRNA/crRNA (sense or antisense), or sgRNA/crRNA.
  misc_feature (22)..(40): n is a, c, g, or u
aauucucggugugccgaguaunnnnnnnnnnnnnnnnnnn

SEQ ID NO: 187 - DNA, 21 bp, Artificial Sequence
  Polynucleotide encoding constant region of sgRNA/crRNA with non-palindromic recognition site for restriction enzyme (sense or antisense), polynucleotide encoding for constant and variable regions of sgRNA/crRNA (sense or antisense), or sgRNA/crRNA.
aatttctcgtggccgagatat

SEQ ID NO: 188 - DNA, 21 bp, Artificial Sequence
  Polynucleotide encoding constant region of sgRNA/crRNA with non-palindromic recognition site for restriction enzyme (sense or antisense), polynucleotide encoding for constant and variable regions of sgRNA/crRNA (sense or antisense), or sgRNA/crRNA.
atatctcggccacgagaaatt

SEQ ID NO: 189 - DNA, 39 bp, Artificial Sequence
  Polynucleotide encoding constant region of sgRNA/crRNA with non-palindromic recognition site for restriction enzyme (sense or antisense), polynucleotide encoding for constant and variable regions of sgRNA/crRNA (sense or antisense), or sgRNA/crRNA.
  misc_feature (22)..(39): n is a, c, g, or t
aatttctcgtggccgagatatnnnnnnnnnnnnnnnnnn

SEQ ID NO: 190 - DNA, 39 bp, Artificial Sequence
  Polynucleotide encoding constant region of sgRNA/crRNA with non-palindromic recognition site for restriction enzyme (sense or antisense), polynucleotide encoding for constant and variable regions of sgRNA/crRNA (sense or antisense), or sgRNA/crRNA.
  misc_feature (1)..(18): n is a, c, g, or t
nnnnnnnnnnnnnnnnnnatatctcggccacgagaaatt

SEQ ID NO: 191 - RNA, 39 nt, Artificial Sequence
  Polynucleotide encoding constant region of sgRNA/crRNA with non-palindromic recognition site for restriction enzyme (sense or antisense), polynucleotide encoding for constant and variable regions of sgRNA/crRNA (sense or antisense), or sgRNA/crRNA.
  misc_feature (22)..(39): n is a, c, g, or u
aauuucucguggccgagauaunnnnnnnnnnnnnnnnnn

SEQ ID NO: 192 - DNA, 19 bp, Artificial Sequence
  Polynucleotide encoding constant region of sgRNA/crRNA with non-palindromic recognition site for restriction enzyme (sense or antisense), polynucleotide encoding for constant and variable regions of sgRNA/crRNA (sense or antisense), or sgRNA/crRNA.
aattgtcgatgtatcgact

SEQ ID NO: 193 - DNA, 19 bp, Artificial Sequence
  Polynucleotide encoding constant region of sgRNA/crRNA with non-palindromic recognition site for restriction enzyme (sense or antisense), polynucleotide encoding for constant and variable regions of sgRNA/crRNA (sense or antisense), or sgRNA/crRNA.
agtcgatacatcgacaatt

SEQ ID NO: 194 - DNA, 38 bp, Artificial Sequence
  Polynucleotide encoding constant region of sgRNA/crRNA with non-palindromic recognition site for restriction enzyme (sense or antisense), polynucleotide encoding for constant and variable regions of sgRNA/crRNA (sense or antisense), or sgRNA/crRNA.
  misc_feature (20)..(38): n is a, c, g, or t
aattgtcgatgtatcgactnnnnnnnnnnnnnnnnnnn

SEQ ID NO: 195 - DNA, 38 bp, Artificial Sequence
  Polynucleotide encoding constant region of sgRNA/crRNA with non-palindromic recognition site for restriction enzyme (sense or antisense), polynucleotide encoding for constant and variable regions of sgRNA/crRNA (sense or antisense), or sgRNA/crRNA.
  misc_feature (1)..(19): n is a, c, g, or t
nnnnnnnnnnnnnnnnnnnagtcgatacatcgacaatt

SEQ ID NO: 196 - RNA, 38 nt, Artificial Sequence
  Polynucleotide encoding constant region of sgRNA/crRNA with non-palindromic recognition site for restriction enzyme (sense or antisense), polynucleotide encoding for constant and variable regions of sgRNA/crRNA (sense or antisense), or sgRNA/crRNA.
  misc_feature (20)..(38): n is a, c, g, or u
aauugucgauguaucgacunnnnnnnnnnnnnnnnnnn

SEQ ID NO: 197 - DNA, 19 bp, Artificial Sequence
  Polynucleotide encoding constant region of sgRNA/crRNA with non-palindromic recognition site for restriction enzyme (sense or antisense), polynucleotide encoding for constant and variable regions of sgRNA/crRNA (sense or antisense), or sgRNA/crRNA.
  misc_feature (16)..(16): n is a, c, g, or t
aattgttcgtggccgnact

SEQ ID NO: 198 - DNA, 19 bp, Artificial Sequence
  Polynucleotide encoding constant region of sgRNA/crRNA with non-palindromic recognition site for restriction enzyme (sense or antisense), polynucleotide encoding for constant and variable regions of sgRNA/crRNA (sense or antisense), or sgRNA/crRNA.
  misc_feature (4)..(4): n is a, c, g, or t
agtncggccacgaacaatt

SEQ ID NO: 199 - DNA, 38 bp, Artificial Sequence
  Polynucleotide encoding constant region of sgRNA/crRNA with non-palindromic recognition site for restriction enzyme (sense or antisense), polynucleotide encoding for constant and variable regions of sgRNA/crRNA (sense or antisense), or sgRNA/crRNA.
  misc_feature (16)..(16): n is a, c, g, or t
  misc_feature (20)..(38): n is a, c, g, or t
aattgttcgtggccgnactnnnnnnnnnnnnnnnnnnn

SEQ ID NO: 200 - DNA, 38 bp, Artificial Sequence
  Polynucleotide encoding constant region of sgRNA/crRNA with non-palindromic recognition site for restriction enzyme (sense or antisense), polynucleotide encoding for constant and variable regions of sgRNA/crRNA (sense or antisense), or sgRNA/crRNA.
  misc_feature (1)..(19): n is a, c, g, or t
  misc_feature (23)..(23): n is a, c, g, or t
nnnnnnnnnnnnnnnnnnnagtncggccacgaacaatt

SEQ ID NO: 201 - RNA, 38 nt, Artificial Sequence
  Polynucleotide encoding constant region of sgRNA/crRNA with non-palindromic recognition site for restriction enzyme (sense or antisense), polynucleotide encoding for constant and variable regions of sgRNA/crRNA (sense or antisense), or sgRNA/crRNA.
  misc_feature (16)..(16): n is a, c, g, or u
  misc_feature (20)..(38): n is a, c, g, or u
aauuguucguggccgnacunnnnnnnnnnnnnnnnnnn

SEQ ID NO: 202 - DNA, 19 bp, Artificial Sequence
  Polynucleotide encoding constant region of sgRNA/crRNA with non-palindromic recognition site for restriction enzyme (sense or antisense), polynucleotide encoding for constant and variable regions of sgRNA/crRNA (sense or antisense), or sgRNA/crRNA.
aattctccttgtgaggagt

SEQ ID NO: 203 - DNA, 19 bp, Artificial Sequence
  Polynucleotide encoding constant region of sgRNA/crRNA with non-palindromic recognition site for restriction enzyme (sense or antisense), polynucleotide encoding for constant and variable regions of sgRNA/crRNA (sense or antisense), or sgRNA/crRNA.
actcctcacaaggagaatt

SEQ ID NO: 204 - DNA, 38 bp, Artificial Sequence
  Polynucleotide encoding constant region of sgRNA/crRNA with non-palindromic recognition site for restriction enzyme (sense or antisense), polynucleotide encoding for constant and variable regions of sgRNA/crRNA (sense or antisense), or sgRNA/crRNA.
  misc_feature (20)..(38): n is a, c, g, or t
aattctccttgtgaggagtnnnnnnnnnnnnnnnnnnn

SEQ ID NO: 205 - DNA, 38 bp, Artificial Sequence
  Polynucleotide encoding constant region of sgRNA/crRNA with non-palindromic recognition site for restriction enzyme (sense or antisense), polynucleotide encoding for constant and variable regions of sgRNA/crRNA (sense or antisense), or sgRNA/crRNA.
  misc_feature (1)..(19): n is a, c, g, or t
nnnnnnnnnnnnnnnnnnnactcctcacaaggagaatt

SEQ ID NO: 206 - RNA, 38 nt, Artificial Sequence
  Polynucleotide encoding constant region of sgRNA/crRNA with non-palindromic recognition site for restriction enzyme (sense or antisense), polynucleotide encoding for constant and variable regions of sgRNA/crRNA (sense or antisense), or sgRNA/crRNA.
  misc_feature (20)..(38): n is a, c, g, or u
aauucuccuugugaggagunnnnnnnnnnnnnnnnnnn

SEQ ID NO: 207 - DNA, 18 bp, Artificial Sequence
  Polynucleotide encoding constant region of sgRNA/crRNA with non-palindromic recognition site for restriction enzyme (sense or antisense), polynucleotide encoding for constant and variable regions of sgRNA/crRNA (sense or antisense), or sgRNA/crRNA.
aattcttcctggrggaag

SEQ ID NO: 208 - DNA, 18 bp, Artificial Sequence
  Polynucleotide encoding constant region of sgRNA/crRNA with non-palindromic recognition site for restriction enzyme (sense or antisense), polynucleotide encoding for constant and variable regions of sgRNA/crRNA (sense or antisense), or sgRNA/crRNA.
cttccyccaggaagaatt

SEQ ID NO: 209 - DNA, 37 bp, Artificial Sequence
  Polynucleotide encoding constant region of sgRNA/crRNA with non-palindromic recognition site for restriction enzyme (sense or antisense), polynucleotide encoding for constant and variable regions of sgRNA/crRNA (sense or antisense), or sgRNA/crRNA.
  misc_feature (19)..(37): n is a, c, g, or t
aattcttcctggrggaagnnnnnnnnnnnnnnnnnnn

SEQ ID NO: 210 - DNA, 37 bp, Artificial Sequence
  Polynucleotide encoding constant region of sgRNA/crRNA with non-palindromic recognition site for restriction enzyme (sense or antisense), polynucleotide encoding for constant and variable regions of sgRNA/crRNA (sense or antisense), or sgRNA/crRNA.
  misc_feature (1)..(19): n is a, c, g, or t
nnnnnnnnnnnnnnnnnnncttccyccaggaagaatt

SEQ ID NO: 211 - RNA, 37 nt, Artificial Sequence
  Polynucleotide encoding constant region of sgRNA/crRNA with non-palindromic recognition site for restriction enzyme (sense or antisense), polynucleotide encoding for constant and variable regions of sgRNA/crRNA (sense or antisense), or sgRNA/crRNA.
  misc_feature (19)..(37): n is a, c, g, or u
aauucuuccuggrggaagnnnnnnnnnnnnnnnnnnn

SEQ ID NO: 212 - DNA, 19 bp, Artificial Sequence
  Polynucleotide encoding constant region of sgRNA/crRNA with non-palindromic recognition site for restriction enzyme (sense or antisense), polynucleotide encoding for constant and variable regions of sgRNA/crRNA (sense or antisense), or sgRNA/crRNA.
aattctccgtgtgcggagt

SEQ ID NO: 213 - DNA, 19 bp, Artificial Sequence
  Polynucleotide encoding constant region of sgRNA/crRNA with non-palindromic recognition site for restriction enzyme (sense or antisense), polynucleotide encoding for constant and variable regions of sgRNA/crRNA (sense or antisense), or sgRNA/crRNA.
actccgcacacggagaatt

SEQ ID NO: 214 - DNA, 38 bp, Artificial Sequence
  Polynucleotide encoding constant region of sgRNA/crRNA with non-palindromic recognition site for restriction enzyme (sense or antisense), polynucleotide encoding for constant and variable regions of sgRNA/crRNA (sense or antisense), or sgRNA/crRNA.
  misc_feature (20)..(38): n is a, c, g, or t
aattctccgtgtgcggagtnnnnnnnnnnnnnnnnnnn

SEQ ID NO: 215 - DNA, 38 bp, Artificial Sequence
  Polynucleotide encoding constant region of sgRNA/crRNA with non-palindromic recognition site for restriction enzyme (sense or antisense), polynucleotide encoding for constant and variable regions of sgRNA/crRNA (sense or antisense), or sgRNA/crRNA.
  misc_feature (1)..(19): n is a, c, g, or t
nnnnnnnnnnnnnnnnnnnactccgcacacggagaatt

SEQ ID NO: 216 - RNA, 38 nt, Artificial Sequence
  Polynucleotide encoding constant region of sgRNA/crRNA with non-palindromic recognition site for restriction enzyme (sense or antisense), polynucleotide encoding for constant and variable regions of sgRNA/crRNA (sense or antisense), or sgRNA/crRNA.
  misc_feature (20)..(38): n is a, c, g, or u
aauucuccgugugcggagunnnnnnnnnnnnnnnnnnn

SEQ ID NO: 217 - DNA, 19 bp, Artificial Sequence
  Polynucleotide encoding constant region of sgRNA/crRNA with non-palindromic recognition site for restriction enzyme (sense or antisense), polynucleotide encoding for constant and variable regions of sgRNA/crRNA (sense or antisense), or sgRNA/crRNA.
aattctccttgtaaggagt

SEQ ID NO: 218 - DNA, 19 bp, Artificial Sequence
  Polynucleotide encoding constant region of sgRNA/crRNA with non-palindromic recognition site for restriction enzyme (sense or antisense), polynucleotide encoding for constant and variable regions of sgRNA/crRNA (sense or antisense), or sgRNA/crRNA.
actccttacaaggagaatt

SEQ ID NO: 219 - DNA, 38 bp, Artificial Sequence
  Polynucleotide encoding constant region of sgRNA/crRNA with non-palindromic recognition site for restriction enzyme (sense or antisense), polynucleotide encoding for constant and variable regions of sgRNA/crRNA (sense or antisense), or sgRNA/crRNA.
  misc_feature (20)..(38): n is a, c, g, or t
aattctccttgtaaggagtnnnnnnnnnnnnnnnnnnn

SEQ ID NO: 220 - DNA, 38 bp, Artificial Sequence
  Polynucleotide encoding constant region of sgRNA/crRNA with non-palindromic recognition site for restriction enzyme (sense or antisense), polynucleotide encoding for constant and variable regions of sgRNA/crRNA (sense or antisense), or sgRNA/crRNA.
  misc_feature (1)..(19): n is a, c, g, or t
nnnnnnnnnnnnnnnnnnnactccttacaaggagaatt

SEQ ID NO: 221 - RNA, 38 nt, Artificial Sequence
  Polynucleotide encoding constant region of sgRNA/crRNA with non-palindromic recognition site for restriction enzyme (sense or antisense), polynucleotide encoding for constant and variable regions of sgRNA/crRNA (sense or antisense), or sgRNA/crRNA.
  misc_feature (20)..(38): n is a, c, g, or u
aauucuccuuguaaggagunnnnnnnnnnnnnnnnnnn

SEQ ID NO: 222 - DNA, 19 bp, Artificial Sequence
  Polynucleotide encoding constant region of sgRNA/crRNA with non-palindromic recognition site for restriction enzyme (sense or antisense), polynucleotide encoding for constant and variable regions of sgRNA/crRNA (sense or antisense), or sgRNA/crRNA.
  misc_feature (16)..(16): n is a, c, g, or t
aattgttcttgcaagnact

SEQ ID NO: 223 - DNA, 19 bp, Artificial Sequence
  Polynucleotide encoding constant region of sgRNA/crRNA with non-palindromic recognition site for restriction enzyme (sense or antisense), polynucleotide encoding for constant and variable regions of sgRNA/crRNA (sense or antisense), or sgRNA/crRNA.
  misc_feature (4)..(4): n is a, c, g, or t
agtncttgcaagaacaatt

SEQ ID NO: 224 - DNA, 38 bp, Artificial Sequence
  Polynucleotide encoding constant region of sgRNA/crRNA with non-palindromic recognition site for restriction enzyme (sense or antisense), polynucleotide encoding for constant and variable regions of sgRNA/crRNA (sense or antisense), or sgRNA/crRNA.
  misc_feature (16)..(16): n is a, c, g, or t
  misc_feature (20)..(38): n is a, c, g, or t
aattgttcttgcaagnactnnnnnnnnnnnnnnnnnnn

SEQ ID NO: 225 - DNA, 38 bp, Artificial Sequence
  Polynucleotide encoding constant region of sgRNA/crRNA with non-palindromic recognition site for restriction enzyme (sense or antisense), polynucleotide encoding for constant and variable regions of sgRNA/crRNA (sense or antisense), or sgRNA/crRNA.
  misc_feature (1)..(19): n is a, c, g, or t
  misc_feature (23)..(23): n is a, c, g, or t
nnnnnnnnnnnnnnnnnnnagtncttgcaagaacaatt

SEQ ID NO: 226 - RNA, 38 nt, Artificial Sequence
  Polynucleotide encoding constant region of sgRNA/crRNA with non-palindromic recognition site for restriction enzyme (sense or antisense), polynucleotide encoding for constant and variable regions of sgRNA/crRNA (sense or antisense), or sgRNA/crRNA.
  misc_feature (16)..(16): n is a, c, g, or u
  misc_feature (20)..(38): n is a, c, g, or u
aauuguucuugcaagnacunnnnnnnnnnnnnnnnnnn

SEQ ID NO: 227 - DNA, 19 bp, Artificial Sequence
  Polynucleotide encoding constant region of sgRNA/crRNA with non-palindromic recognition site for restriction enzyme (sense or antisense), polynucleotide encoding for constant and variable regions of sgRNA/crRNA (sense or antisense), or sgRNA/crRNA.
aattgtcgttgttacgact

SEQ ID NO: 228 - DNA, 19 bp, Artificial Sequence
  Polynucleotide encoding constant region of sgRNA/crRNA with non-palindromic recognition site for restriction enzyme (sense or antisense), polynucleotide encoding for constant and variable regions of sgRNA/crRNA (sense or antisense), or sgRNA/crRNA.
agtcgtaacaacgacaatt

SEQ ID NO: 229 - DNA, 38 bp, Artificial Sequence
  Polynucleotide encoding constant region of sgRNA/crRNA with non-palindromic recognition site for restriction enzyme (sense or antisense), polynucleotide encoding for constant and variable regions of sgRNA/crRNA (sense or antisense), or sgRNA/crRNA.
  misc_feature (20)..(38): n is a, c, g, or t
aattgtcgttgttacgactnnnnnnnnnnnnnnnnnnn

SEQ ID NO: 230 - DNA, 38 bp, Artificial Sequence
  Polynucleotide encoding constant region of sgRNA/crRNA with non-palindromic recognition site for restriction enzyme (sense or antisense), polynucleotide encoding for constant and variable regions of sgRNA/crRNA (sense or antisense), or sgRNA/crRNA.
  misc_feature (1)..(19): n is a, c, g, or t
nnnnnnnnnnnnnnnnnnnagtcgtaacaacgacaatt

SEQ ID NO: 231 - RNA, 38 nt, Artificial Sequence
  Polynucleotide encoding constant region of sgRNA/crRNA with non-palindromic recognition site for restriction enzyme (sense or antisense), polynucleotide encoding for constant and variable regions of sgRNA/crRNA (sense or antisense), or sgRNA/crRNA.
  misc_feature (20)..(38): n is a, c, g, or u
aauugucguuguuacgacunnnnnnnnnnnnnnnnnnn

SEQ ID NO: 232 - DNA, 19 bp, Artificial Sequence
  Polynucleotide encoding constant region of sgRNA/crRNA with non-palindromic recognition site for restriction enzyme (sense or antisense), polynucleotide encoding for constant and variable regions of sgRNA/crRNA (sense or antisense), or sgRNA/crRNA.
aattgtggttgtgaccact

SEQ ID NO: 233 - DNA, 19 bp, Artificial Sequence
  Polynucleotide encoding constant region of sgRNA/crRNA with non-palindromic recognition site for restriction enzyme (sense or antisense), polynucleotide encoding for constant and variable regions of sgRNA/crRNA (sense or antisense), or sgRNA/crRNA.
agtggtcacaaccacaatt

SEQ ID NO: 234 - DNA, 38 bp, Artificial Sequence
  Polynucleotide encoding constant region of sgRNA/crRNA with non-palindromic recognition site for restriction enzyme (sense or antisense), polynucleotide encoding for constant and variable regions of sgRNA/crRNA (sense or antisense), or sgRNA/crRNA.
  misc_feature (20)..(38): n is a, c, g, or t
aattgtggttgtgaccactnnnnnnnnnnnnnnnnnnn

SEQ ID NO: 235 - DNA, 38 bp, Artificial Sequence
  Polynucleotide encoding constant region of sgRNA/crRNA with non-palindromic recognition site for restriction enzyme (sense or antisense), polynucleotide encoding for constant and variable regions of sgRNA/crRNA (sense or antisense), or sgRNA/crRNA.
  misc_feature (1)..(19): n is a, c, g, or t
nnnnnnnnnnnnnnnnnnnagtggtcacaaccacaatt

SEQ ID NO: 236 - RNA, 38 nt, Artificial Sequence
  Polynucleotide encoding constant region of sgRNA/crRNA with non-palindromic recognition site for restriction enzyme (sense or antisense), polynucleotide encoding for constant and variable regions of sgRNA/crRNA (sense or antisense), or sgRNA/crRNA.
  misc_feature (20)..(38): n is a, c, g, or u
aauugugguugugaccacunnnnnnnnnnnnnnnnnnn

SEQ ID NO: 237 - DNA, 19 bp, Artificial Sequence
  Polynucleotide encoding constant region of sgRNA/crRNA with non-palindromic recognition site for restriction enzyme (sense or antisense), polynucleotide encoding for constant and variable regions of sgRNA/crRNA (sense or antisense), or sgRNA/crRNA.
aattgtcaatgcrttgact

SEQ ID NO: 238 - DNA, 19 bp, Artificial Sequence
  Polynucleotide encoding constant region of sgRNA/crRNA with non-palindromic recognition site for restriction enzyme (sense or antisense), polynucleotide encoding for constant and variable regions of sgRNA/crRNA (sense or antisense), or sgRNA/crRNA.
agtcaaygcattgacaatt

SEQ ID NO: 239 - DNA, 38 bp, Artificial Sequence
  Polynucleotide encoding constant region of sgRNA/crRNA with non-palindromic recognition site for restriction enzyme (sense or antisense), polynucleotide encoding for constant and variable regions of sgRNA/crRNA (sense or antisense), or sgRNA/crRNA.
  misc_feature (20)..(38): n is a, c, g, or t
aattgtcaatgcrttgactnnnnnnnnnnnnnnnnnnn

SEQ ID NO: 240 - DNA, 38 bp, Artificial Sequence
  Polynucleotide encoding constant region of sgRNA/crRNA with non-palindromic recognition site for restriction enzyme (sense or antisense), polynucleotide encoding for constant and variable regions of sgRNA/crRNA (sense or antisense), or sgRNA/crRNA.
  misc_feature (1)..(19): n is a, c, g, or t
nnnnnnnnnnnnnnnnnnnagtcaaygcattgacaatt

SEQ ID NO: 241 - RNA, 38 nt, Artificial Sequence
  Polynucleotide encoding constant region of sgRNA/crRNA with non-palindromic recognition site for restriction enzyme (sense or antisense), polynucleotide encoding for constant and variable regions of sgRNA/crRNA (sense or antisense), or sgRNA/crRNA.
  misc_feature (20)..(38): n is a, c, g, or u
aauugucaaugcruugacunnnnnnnnnnnnnnnnnnn

SEQ ID NO: 242 - DNA, 19 bp, Artificial Sequence
  Polynucleotide encoding constant region of sgRNA/crRNA with non-palindromic recognition site for restriction enzyme (sense or antisense), polynucleotide encoding for constant and variable regions of sgRNA/crRNA (sense or antisense), or sgRNA/crRNA.
aattcttcttgcaagragt

SEQ ID NO: 243 - DNA, 19 bp, Artificial Sequence
  Polynucleotide encoding constant region of sgRNA/crRNA with non-palindromic recognition site for restriction enzyme (sense or antisense), polynucleotide encoding for constant and variable regions of sgRNA/crRNA (sense or antisense), or sgRNA/crRNA.
actycttgcaagaagaatt

SEQ ID NO: 244 - DNA, 38 bp, Artificial Sequence
  Polynucleotide encoding constant region of sgRNA/crRNA with non-palindromic recognition site for restriction enzyme (sense or antisense), polynucleotide encoding for constant and variable regions of sgRNA/crRNA (sense or antisense), or sgRNA/crRNA.
  misc_feature (20)..(38): n is a, c, g, or t
aattcttcttgcaagragtnnnnnnnnnnnnnnnnnnn

SEQ ID NO: 245 - DNA, 38 bp, Artificial Sequence
  Polynucleotide encoding constant region of sgRNA/crRNA with non-palindromic recognition site for restriction enzyme (sense or antisense), polynucleotide encoding for constant and variable regions of sgRNA/crRNA (sense or antisense), or sgRNA/crRNA.
  misc_feature (1)..(19): n is a, c, g, or t
nnnnnnnnnnnnnnnnnnnactycttgcaagaagaatt

SEQ ID NO: 246 - RNA, 38 nt, Artificial Sequence
  Polynucleotide encoding constant region of sgRNA/crRNA with non-palindromic recognition site for restriction enzyme (sense or antisense), polynucleotide encoding for constant and variable regions of sgRNA/crRNA (sense or antisense), or sgRNA/crRNA.
  misc_feature (20)..(38): n is a, c, g, or u
aauucuucuugcaagragunnnnnnnnnnnnnnnnnnn

SEQ ID NO: 247 - DNA, 19 bp, Artificial Sequence
  Polynucleotide encoding constant region of sgRNA/crRNA with non-palindromic recognition site for restriction enzyme (sense or antisense), polynucleotide encoding for constant and variable regions of sgRNA/crRNA (sense or antisense), or sgRNA/crRNA.
aattgtgattgtcatcact

SEQ ID NO: 248 - DNA, 19 bp, Artificial Sequence
  Polynucleotide encoding constant region of sgRNA/crRNA with non-palindromic recognition site for restriction enzyme (sense or antisense), polynucleotide encoding for constant and variable regions of sgRNA/crRNA (sense or antisense), or sgRNA/crRNA.
agtgatgacaatcacaatt

SEQ ID NO: 249 - DNA, 37 bp, Artificial Sequence
  Polynucleotide encoding constant region of sgRNA/crRNA with non-palindromic recognition site for restriction enzyme (sense or antisense), polynucleotide encoding for constant and variable regions of sgRNA/crRNA (sense or antisense), or sgRNA/crRNA.
  misc_feature (20)..(37): n is a, c, g, or t
aattgtgattgtcatcactnnnnnnnnnnnnnnnnnn

SEQ ID NO: 250 - DNA, 37 bp, Artificial Sequence
  Polynucleotide encoding constant region of sgRNA/crRNA with non-palindromic recognition site for restriction enzyme (sense or antisense), polynucleotide encoding for constant and variable regions of sgRNA/crRNA (sense or antisense), or sgRNA/crRNA.
  misc_feature (1)..(18): n is a, c, g, or t
nnnnnnnnnnnnnnnnnnagtgatgacaatcacaatt

SEQ ID NO: 251 - RNA, 37 nt, Artificial Sequence
  Polynucleotide encoding constant region of sgRNA/crRNA with non-palindromic recognition site for restriction enzyme (sense or antisense), polynucleotide encoding for constant and variable regions of sgRNA/crRNA (sense or antisense), or sgRNA/crRNA.
  misc_feature (20)..(37): n is a, c, g, or u
aauugugauugucaucacunnnnnnnnnnnnnnnnnn

SEQ ID NO: 252 - DNA, 19 bp, Artificial Sequence
  Polynucleotide encoding constant region of sgRNA/crRNA with non-palindromic recognition site for restriction enzyme (sense or antisense), polynucleotide encoding for constant and variable regions of sgRNA/crRNA (sense or antisense), or sgRNA/crRNA.
aattctgattgtcatcagt

SEQ ID NO: 253 - DNA, 19 bp, Artificial Sequence
  Polynucleotide encoding constant region of sgRNA/crRNA with non-palindromic recognition site for restriction enzyme (sense or antisense), polynucleotide encoding for constant and variable regions of sgRNA/crRNA (sense or antisense), or sgRNA/crRNA.
actgatgacaatcagaatt

SEQ ID NO: 254 - DNA, 39 bp, Artificial Sequence
  Polynucleotide encoding constant region of sgRNA/crRNA with non-palindromic recognition site for restriction enzyme (sense or antisense), polynucleotide encoding for constant and variable regions of sgRNA/crRNA (sense or antisense), or sgRNA/crRNA.
  misc_feature (20)..(39): n is a, c, g, or t
aattctgattgtcatcagtnnnnnnnnnnnnnnnnnnnn

SEQ ID NO: 255 - DNA, 39 bp, Artificial Sequence
  Polynucleotide encoding constant region of sgRNA/crRNA with non-palindromic recognition site for restriction enzyme (sense or antisense), polynucleotide encoding for constant and variable regions of sgRNA/crRNA (sense or antisense), or sgRNA/crRNA.
  misc_feature (1)..(20): n is a, c, g, or t
nnnnnnnnnnnnnnnnnnnnactgatgacaatcagaatt

SEQ ID NO: 256 - RNA, 39 nt, Artificial Sequence
  Polynucleotide encoding constant region of sgRNA/crRNA with non-palindromic recognition site for restriction enzyme (sense or antisense), polynucleotide encoding for constant and variable regions of sgRNA/crRNA (sense or antisense), or sgRNA/crRNA.
  misc_feature (20)..(39): n is a, c, g, or u
aauucugauugucaucagunnnnnnnnnnnnnnnnnnnn

SEQ ID NO: 257 - DNA, 19 bp, Artificial Sequence
  Polynucleotide encoding constant region of sgRNA/crRNA with non-palindromic recognition site for restriction enzyme (sense or antisense), polynucleotide encoding for constant and variable regions of sgRNA/crRNA (sense or antisense), or sgRNA/crRNA.
aattytgggtgcgcccart

SEQ ID NO: 258 - DNA, 19 bp, Artificial Sequence
  Polynucleotide encoding constant region of sgRNA/crRNA with non-palindromic recognition site for restriction enzyme (sense or antisense), polynucleotide encoding for constant and variable regions of sgRNA/crRNA (sense or antisense), or sgRNA/crRNA.
aytgggcgcacccaraatt

SEQ ID NO: 259 - DNA, 38 bp, Artificial Sequence
  Polynucleotide encoding constant region of sgRNA/crRNA with non-palindromic recognition site for restriction enzyme (sense or antisense), polynucleotide encoding for constant and variable regions of sgRNA/crRNA (sense or antisense), or sgRNA/crRNA.
  misc_feature (20)..(38): n is a, c, g, or t
aattytgggtgcgcccartnnnnnnnnnnnnnnnnnnn

SEQ ID NO: 260 - DNA, 38 bp, Artificial Sequence
  Polynucleotide encoding constant region of sgRNA/crRNA with non-palindromic recognition site for restriction enzyme (sense or antisense), polynucleotide encoding for constant and variable regions of sgRNA/crRNA (sense or antisense), or sgRNA/crRNA.
  misc_feature (1)..(19): n is a, c, g, or t
nnnnnnnnnnnnnnnnnnnaytgggcgcacccaraatt

SEQ ID NO: 261 - RNA, 38 nt, Artificial Sequence
  Polynucleotide encoding constant region of sgRNA/crRNA with non-palindromic recognition site for restriction enzyme (sense or antisense), polynucleotide encoding for constant and variable regions of sgRNA/crRNA (sense or antisense), or sgRNA/crRNA.
  misc_feature (20)..(38): n is a, c, g, or u
aauuyugggugcgcccarunnnnnnnnnnnnnnnnnnn

SEQ ID NO: 262 - DNA, 19 bp, Artificial Sequence
  Polynucleotide encoding constant region of sgRNA/crRNA with non-palindromic recognition site for restriction enzyme (sense or antisense), polynucleotide encoding for constant and variable regions of sgRNA/crRNA (sense or antisense), or sgRNA/crRNA.
aattgtgggtgtccycagt

SEQ ID NO: 263 - DNA, 19 bp, Artificial Sequence
  Polynucleotide encoding constant region of sgRNA/crRNA with non-palindromic recognition site for restriction enzyme (sense or antisense), polynucleotide encoding for constant and variable regions of sgRNA/crRNA (sense or antisense), or sgRNA/crRNA.
actgrggacacccacaatt

SEQ ID NO: 264 - DNA, 39 bp, Artificial Sequence
  Polynucleotide encoding constant region of sgRNA/crRNA with non-palindromic recognition site for restriction enzyme (sense or antisense), polynucleotide encoding for constant and variable regions of sgRNA/crRNA (sense or antisense), or sgRNA/crRNA.
  misc_feature (20)..(39): n is a, c, g, or t
aattgtgggtgtccycagtnnnnnnnnnnnnnnnnnnnn

SEQ ID NO: 265 - DNA, 39 bp, Artificial Sequence
  Polynucleotide encoding constant region of sgRNA/crRNA with non-palindromic recognition site for restriction enzyme (sense or antisense), polynucleotide encoding for constant and variable regions of sgRNA/crRNA (sense or antisense), or sgRNA/crRNA.
  misc_feature (1)..(20): n is a, c, g, or t
nnnnnnnnnnnnnnnnnnnnactgrggacacccacaatt

SEQ ID NO: 266 - RNA, 39 nt, Artificial Sequence
  Polynucleotide encoding constant region of sgRNA/crRNA with non-palindromic recognition site for restriction enzyme (sense or antisense), polynucleotide encoding for constant and variable regions of sgRNA/crRNA (sense or antisense), or sgRNA/crRNA.
  misc_feature (20)..(39): n is a, c, g, or u
aauuguggguguccycagunnnnnnnnnnnnnnnnnnnn

SEQ ID NO: 267 - DNA, 19 bp, Artificial Sequence
  Polynucleotide encoding constant region of sgRNA/crRNA with non-palindromic recognition site for restriction enzyme (sense or antisense), polynucleotide encoding for constant and variable regions of sgRNA/crRNA (sense or antisense), or sgRNA/crRNA.
aattgtcgatgcatcgact

SEQ ID NO: 268 - DNA, 19 bp, Artificial Sequence
  Polynucleotide encoding constant region of sgRNA/crRNA with non-palindromic recognition site for restriction enzyme (sense or antisense), polynucleotide encoding for constant and variable regions of sgRNA/crRNA (sense or antisense), or sgRNA/crRNA.
agtcgatgcatcgacaatt

SEQ ID NO: 269 - DNA, 38 bp, Artificial Sequence
  Polynucleotide encoding constant region of sgRNA/crRNA with non-palindromic recognition site for restriction enzyme (sense or antisense), polynucleotide encoding for constant and variable regions of sgRNA/crRNA (sense or antisense), or sgRNA/crRNA.
  misc_feature (20)..(38): n is a, c, g, or t
aattgtcgatgcatcgactnnnnnnnnnnnnnnnnnnn

SEQ ID NO: 270 - DNA, 38 bp, Artificial Sequence
  Polynucleotide encoding constant region of sgRNA/crRNA with non-palindromic recognition site for restriction enzyme (sense or antisense), polynucleotide encoding for constant and variable regions of sgRNA/crRNA (sense or antisense), or sgRNA/crRNA.
  misc_feature (1)..(19): n is a, c, g, or t
nnnnnnnnnnnnnnnnnnnagtcgatgcatcgacaatt

SEQ ID NO: 271 - RNA, 38 nt, Artificial Sequence
  Polynucleotide encoding constant region of sgRNA/crRNA with non-palindromic recognition site for restriction enzyme (sense or antisense), polynucleotide encoding for constant and variable regions of sgRNA/crRNA (sense or antisense), or sgRNA/crRNA.
  misc_feature (20)..(38): n is a, c, g, or u
aauugucgaugcaucgacunnnnnnnnnnnnnnnnnnn

SEQ ID NO: 272 - DNA, 19 bp, Artificial Sequence
  Polynucleotide encoding constant region of sgRNA/crRNA with non-palindromic recognition site for restriction enzyme (sense or antisense), polynucleotide encoding for constant and variable regions of sgRNA/crRNA (sense or antisense), or sgRNA/crRNA.
aattgtccttgcgrggact

SEQ ID NO: 273 - DNA, 19 bp, Artificial Sequence
  Polynucleotide encoding constant region of sgRNA/crRNA with non-palindromic recognition site for restriction enzyme (sense or antisense), polynucleotide encoding for constant and variable regions of sgRNA/crRNA (sense or antisense), or sgRNA/crRNA.
agtccycgcaaggacaatt

SEQ ID NO: 274 - DNA, 38 bp, Artificial Sequence
  Polynucleotide encoding constant region of sgRNA/crRNA with non-palindromic recognition site for restriction enzyme (sense or antisense), polynucleotide encoding for constant and variable regions of sgRNA/crRNA (sense or antisense), or sgRNA/crRNA.
  misc_feature (20)..(38): n is a, c, g, or t
aattgtccttgcgrggactnnnnnnnnnnnnnnnnnnn

SEQ ID NO: 275 - DNA, 38 bp, Artificial Sequence
  Polynucleotide encoding constant region of sgRNA/crRNA with non-palindromic recognition site for restriction enzyme (sense or antisense), polynucleotide encoding for constant and variable regions of sgRNA/crRNA (sense or antisense), or sgRNA/crRNA.
  misc_feature (1)..(19): n is a, c, g, or t
nnnnnnnnnnnnnnnnnnnagtccycgcaaggacaatt

SEQ ID NO: 276 - RNA, 38 nt, Artificial Sequence
  Polynucleotide encoding constant region of sgRNA/crRNA with non-palindromic recognition site for restriction enzyme (sense or antisense), polynucleotide encoding for constant and variable regions of sgRNA/crRNA (sense or antisense), or sgRNA/crRNA.
  misc_feature (20)..(38): n is a, c, g, or u
aauuguccuugcgrggacunnnnnnnnnnnnnnnnnnn

SEQ ID NO: 277 - DNA, 19 bp, Artificial Sequence
  Polynucleotide encoding constant region of sgRNA/crRNA with non-palindromic recognition site for restriction enzyme (sense or antisense), polynucleotide encoding for constant and variable regions of sgRNA/crRNA (sense or antisense), or sgRNA/crRNA.
aattcttcttgtcagragt

SEQ ID NO: 278 - DNA, 19 bp, Artificial Sequence
  Polynucleotide encoding constant region of sgRNA/crRNA with non-palindromic recognition site for restriction enzyme (sense or antisense), polynucleotide encoding for constant and variable regions of sgRNA/crRNA (sense or antisense), or sgRNA/crRNA.
actyctgacaagaagaatt

SEQ ID NO: 279 - DNA, 38 bp, Artificial Sequence
  Polynucleotide encoding constant region of sgRNA/crRNA with non-palindromic recognition site for restriction enzyme (sense or antisense), polynucleotide encoding for constant and variable regions of sgRNA/crRNA (sense or antisense), or sgRNA/crRNA.
  misc_feature (20)..(38): n is a, c, g, or t
aattcttcttgtcagragtnnnnnnnnnnnnnnnnnnn

SEQ ID NO: 280 - DNA, 38 bp, Artificial Sequence
  Polynucleotide encoding constant region of sgRNA/crRNA with non-palindromic recognition site for restriction enzyme (sense or antisense), polynucleotide encoding for constant and variable regions of sgRNA/crRNA (sense or antisense), or sgRNA/crRNA.
  misc_feature (1)..(19): n is a, c, g, or t
nnnnnnnnnnnnnnnnnnnactyctgacaagaagaatt

SEQ ID NO: 281 - RNA, 38 nt, Artificial Sequence
  Polynucleotide encoding constant region of sgRNA/crRNA with non-palindromic recognition site for restriction enzyme (sense or antisense), polynucleotide encoding for constant and variable regions of sgRNA/crRNA (sense or antisense), or sgRNA/crRNA.
  misc_feature (20)..(38): n is a, c, g, or u
aauucuucuugucagragunnnnnnnnnnnnnnnnnnn

SEQ ID NO: 282 - DNA, 19 bp, Artificial Sequence
  Polynucleotide encoding constant region of sgRNA/crRNA with non-palindromic recognition site for restriction enzyme (sense or antisense), polynucleotide encoding for constant and variable regions of sgRNA/crRNA (sense or antisense), or sgRNA/crRNA.
aattcttcctggcggragt

SEQ ID NO: 283 - DNA, 19 bp, Artificial Sequence
  Polynucleotide encoding constant region of sgRNA/crRNA with non-palindromic recognition site for restriction enzyme (sense or antisense), polynucleotide encoding for constant and variable regions of sgRNA/crRNA (sense or antisense), or sgRNA/crRNA.
actyccgccaggaagaatt

SEQ ID NO: 284 - DNA, 38 bp, Artificial Sequence
  Polynucleotide encoding constant region of sgRNA/crRNA with non-palindromic recognition site for restriction enzyme (sense or antisense), polynucleotide encoding for constant and variable regions of sgRNA/crRNA (sense or antisense), or sgRNA/crRNA.
  misc_feature (20)..(38): n is a, c, g, or t
aattcttcctggcggragtnnnnnnnnnnnnnnnnnnn

SEQ ID NO: 285 - DNA, 38 bp, Artificial Sequence
  Polynucleotide encoding constant region of sgRNA/crRNA with non-palindromic recognition site for restriction enzyme (sense or antisense), polynucleotide encoding for constant and variable regions of sgRNA/crRNA (sense or antisense), or sgRNA/crRNA.
  misc_feature (1)..(19): n is a, c, g, or t
nnnnnnnnnnnnnnnnnnnactyccgccaggaagaatt

SEQ ID NO: 286 - RNA, 38 nt, Artificial Sequence
  Polynucleotide encoding constant region of sgRNA/crRNA with non-palindromic recognition site for restriction enzyme (sense or antisense), polynucleotide encoding for constant and variable regions of sgRNA/crRNA (sense or antisense), or sgRNA/crRNA.
  misc_feature (20)..(38): n is a, c, g, or u
aauucuuccuggcggragunnnnnnnnnnnnnnnnnnn

SEQ ID NO: 287 - DNA, 19 bp, Artificial Sequence
  Polynucleotide encoding constant region of sgRNA/crRNA with non-palindromic recognition site for restriction enzyme (sense or antisense), polynucleotide encoding for constant and variable regions of sgRNA/crRNA (sense or antisense), or sgRNA/crRNA.
aattctccctggcgggagt

SEQ ID NO: 288 - DNA, 19 bp, Artificial Sequence
  Polynucleotide encoding constant region of sgRNA/crRNA with non-palindromic recognition site for restriction enzyme (sense or antisense), polynucleotide encoding for constant and variable regions of sgRNA/crRNA (sense or antisense), or sgRNA/crRNA.
actcccgccagggagaatt

SEQ ID NO: 289 - DNA, 39 bp, Artificial Sequence
  Polynucleotide encoding constant region of sgRNA/crRNA with non-palindromic recognition site for restriction enzyme (sense or antisense), polynucleotide encoding for constant and variable regions of sgRNA/crRNA (sense or antisense), or sgRNA/crRNA.
  misc_feature (20)..(39): n is a, c, g, or t
aattctccctggcgggagtnnnnnnnnnnnnnnnnnnnn

SEQ ID NO: 290 - DNA, 39 bp, Artificial Sequence
  Polynucleotide encoding constant region of sgRNA/crRNA with non-palindromic recognition site for restriction enzyme (sense or antisense), polynucleotide encoding for constant and variable regions of sgRNA/crRNA (sense or antisense), or sgRNA/crRNA.
  misc_feature (1)..(20): n is a, c, g, or t
nnnnnnnnnnnnnnnnnnnnactcccgccagggagaatt

SEQ ID NO: 291 - RNA, 39 nt, Artificial Sequence
  Polynucleotide encoding constant region of sgRNA/crRNA with non-palindromic recognition site for restriction enzyme (sense or antisense), polynucleotide encoding for constant and variable regions of sgRNA/crRNA (sense or antisense), or sgRNA/crRNA.
  misc_feature (20)..(39): n is a, c, g, or u
aauucucccuggcgggagunnnnnnnnnnnnnnnnnnnn

SEQ ID NO: 292 - DNA, 19 bp, Artificial Sequence
  Polynucleotide encoding constant region of sgRNA/crRNA with non-palindromic recognition site for restriction enzyme (sense or antisense), polynucleotide encoding for constant and variable regions of sgRNA/crRNA (sense or antisense), or sgRNA/crRNA.
aatttgtcgtgatcgacat

SEQ ID NO: 293 - DNA, 19 bp, Artificial Sequence
  Polynucleotide encoding constant region of sgRNA/crRNA with non-palindromic recognition site for restriction enzyme (sense or antisense), polynucleotide encoding for constant and variable regions of sgRNA/crRNA (sense or antisense), or sgRNA/crRNA.
atgtcgatcacgacaaatt

SEQ ID NO: 294 - DNA, 37 bp, Artificial Sequence
  Polynucleotide encoding constant region of sgRNA/crRNA with non-palindromic recognition site for restriction enzyme (sense or antisense), polynucleotide encoding for constant and variable regions of sgRNA/crRNA (sense or antisense), or sgRNA/crRNA.
  misc_feature (20)..(37): n is a, c, g, or t
aatttgtcgtgatcgacatnnnnnnnnnnnnnnnnnn

SEQ ID NO: 295 - DNA, 37 bp, Artificial Sequence
  Polynucleotide encoding constant region of sgRNA/crRNA with non-palindromic recognition site for restriction enzyme (sense or antisense), polynucleotide encoding for constant and variable regions of sgRNA/crRNA (sense or antisense), or sgRNA/crRNA.
  misc_feature (1)..(18): n is a, c, g, or t
nnnnnnnnnnnnnnnnnnatgtcgatcacgacaaatt

SEQ ID NO: 296 - RNA, 37 nt, Artificial Sequence
  Polynucleotide encoding constant region of sgRNA/crRNA with non-palindromic recognition site for restriction enzyme (sense or antisense), polynucleotide encoding for constant and variable regions of sgRNA/crRNA (sense or antisense), or sgRNA/crRNA.
  misc_feature (20)..(37): n is a, c, g, or u
aauuugucgugaucgacaunnnnnnnnnnnnnnnnnn

SEQ ID NO: 297 - DNA, 19 bp, Artificial Sequence
  Polynucleotide encoding constant region of sgRNA/crRNA with non-palindromic recognition site for restriction enzyme (sense or antisense), polynucleotide encoding for constant and variable regions of sgRNA/crRNA (sense or antisense), or sgRNA/crRNA.
  misc_feature (15)..(15): n is a, c, g, or t
aatttgttctgccgnacat

SEQ ID NO: 298 - DNA, 19 bp, Artificial Sequence
  Polynucleotide encoding constant region of sgRNA/crRNA with non-palindromic recognition site for restriction enzyme (sense or antisense), polynucleotide encoding for constant and variable regions of sgRNA/crRNA (sense or antisense), or sgRNA/crRNA.
  misc_feature (5)..(5): n is a, c, g, or t
atgtncggcagaacaaatt

SEQ ID NO: 299 - DNA, 37 bp, Artificial Sequence
  Polynucleotide encoding constant region of sgRNA/crRNA with non-palindromic recognition site for restriction enzyme (sense or antisense), polynucleotide encoding for constant and variable regions of sgRNA/crRNA (sense or antisense), or sgRNA/crRNA.
  misc_feature (15)..(15): n is a, c, g, or t
  misc_feature (20)..(37): n is a, c, g, or t
aatttgttctgccgnacatnnnnnnnnnnnnnnnnnn

SEQ ID NO: 300 - DNA, 37 bp, Artificial Sequence
  Polynucleotide encoding constant region of sgRNA/crRNA with non-palindromic recognition site for restriction enzyme (sense or antisense), polynucleotide encoding for constant and variable regions of sgRNA/crRNA (sense or antisense), or sgRNA/crRNA.
  misc_feature (1)..(18): n is a, c, g, or t
  misc_feature (23)..(23): n is a, c, g, or t
nnnnnnnnnnnnnnnnnnatgtncggcagaacaaatt

SEQ ID NO: 301 - RNA, 37 nt, Artificial Sequence
  Polynucleotide encoding constant region of sgRNA/crRNA with non-palindromic recognition site for restriction enzyme (sense or antisense), polynucleotide encoding for constant and variable regions of sgRNA/crRNA (sense or antisense), or sgRNA/crRNA.
  misc_feature (15)..(15): n is a, c, g, or u
  misc_feature (20)..(37): n is a, c, g, or u
aauuuguucugccgnacaunnnnnnnnnnnnnnnnnn

SEQ ID NO: 302 - DNA, 19 bp, Artificial Sequence
  Polynucleotide encoding constant region of sgRNA/crRNA with non-palindromic recognition site for restriction enzyme (sense or antisense), polynucleotide encoding for constant and variable regions of sgRNA/crRNA (sense or antisense), or sgRNA/crRNA.
aatttctcctggaggagat

SEQ ID NO: 303 - DNA, 19 bp, Artificial Sequence
  Polynucleotide encoding constant region of sgRNA/crRNA with non-palindromic recognition site for restriction enzyme (sense or antisense), polynucleotide encoding for constant and variable regions of sgRNA/crRNA (sense or antisense), or sgRNA/crRNA.
atctcctccaggagaaatt

SEQ ID NO: 304 - DNA, 37 bp, Artificial Sequence
  Polynucleotide encoding constant region of sgRNA/crRNA with non-palindromic recognition site for restriction enzyme (sense or antisense), polynucleotide encoding for constant and variable regions of sgRNA/crRNA (sense or antisense), or sgRNA/crRNA.
  misc_feature (20)..(37): n is a, c, g, or t
aatttctcctggaggagatnnnnnnnnnnnnnnnnnn

SEQ ID NO: 305 - DNA, 37 bp, Artificial Sequence
  Polynucleotide encoding constant region of sgRNA/crRNA with non-palindromic recognition site for restriction enzyme (sense or antisense), polynucleotide encoding for constant and variable regions of sgRNA/crRNA (sense or antisense), or sgRNA/crRNA.
  misc_feature (1)..(18): n is a, c, g, or t
nnnnnnnnnnnnnnnnnnatctcctccaggagaaatt

SEQ ID NO: 306 - RNA, 37 nt, Artificial Sequence
  Polynucleotide encoding constant region of sgRNA/crRNA with non-palindromic recognition site for restriction enzyme (sense or antisense), polynucleotide encoding for constant and variable regions of sgRNA/crRNA (sense or antisense), or sgRNA/crRNA.
  misc_feature (20)..(37): n is a, c, g, or u
aauuucuccuggaggagaunnnnnnnnnnnnnnnnnn

SEQ ID NO: 307 - DNA, 19 bp, Artificial Sequence
  Polynucleotide encoding constant region of sgRNA/crRNA with non-palindromic recognition site for restriction enzyme (sense or antisense), polynucleotide encoding for constant and variable regions of sgRNA/crRNA (sense or antisense), or sgRNA/crRNA.
aatttcttctgrggaagat

SEQ ID NO: 308 - DNA, 19 bp, Artificial Sequence
  Polynucleotide encoding constant region of sgRNA/crRNA with non-palindromic recognition site for restriction enzyme (sense or antisense), polynucleotide encoding for constant and variable regions of sgRNA/crRNA (sense or antisense), or sgRNA/crRNA.
atcttccycagaagaaatt

SEQ ID NO: 309 - DNA, 37 bp, Artificial Sequence
  Polynucleotide encoding constant region of sgRNA/crRNA with non-palindromic recognition site for restriction enzyme (sense or antisense), polynucleotide encoding for constant and variable regions of sgRNA/crRNA (sense or antisense), or sgRNA/crRNA.
  misc_feature (20)..(37): n is a, c, g, or t
aatttcttctgrggaagatnnnnnnnnnnnnnnnnnn

SEQ ID NO: 310 - DNA, 37 bp, Artificial Sequence
  Polynucleotide encoding constant region of sgRNA/crRNA with non-palindromic recognition site for restriction enzyme (sense or antisense), polynucleotide encoding for constant and variable regions of sgRNA/crRNA (sense or antisense), or sgRNA/crRNA.
  misc_feature (1)..(18): n is a, c, g, or t
nnnnnnnnnnnnnnnnnnatcttccycagaagaaatt

SEQ ID NO: 311 - RNA, 37 nt, Artificial Sequence
  Polynucleotide encoding constant region of sgRNA/crRNA with non-palindromic recognition site for restriction enzyme (sense or antisense), polynucleotide encoding for constant and variable regions of sgRNA/crRNA (sense or antisense), or sgRNA/crRNA.
  misc_feature (20)..(37): n is a, c, g, or u
aauuucuucugrggaagaunnnnnnnnnnnnnnnnnn

SEQ ID NO: 312 - DNA, 19 bp, Artificial Sequence
  Polynucleotide encoding constant region of sgRNA/crRNA with non-palindromic recognition site for restriction enzyme (sense or antisense), polynucleotide encoding for constant and variable regions of sgRNA/crRNA (sense or antisense), or sgRNA/crRNA.
aatttctcctggcggagat

SEQ ID NO: 313 - DNA, 19 bp, Artificial Sequence
  Polynucleotide encoding constant region of sgRNA/crRNA with non-palindromic recognition site for restriction enzyme (sense or antisense), polynucleotide encoding for constant and variable regions of sgRNA/crRNA (sense or antisense), or sgRNA/crRNA.
atctccgccaggagaaatt

SEQ ID NO: 314 - DNA, 37 bp, Artificial Sequence
  Polynucleotide encoding constant region of sgRNA/crRNA with non-palindromic recognition site for restriction enzyme (sense or antisense), polynucleotide encoding for constant and variable regions of sgRNA/crRNA (sense or antisense), or sgRNA/crRNA.
  misc_feature (20)..(37): n is a, c, g, or t
aatttctcctggcggagatnnnnnnnnnnnnnnnnnn

SEQ ID NO: 315 - DNA, 37 bp, Artificial Sequence
  Polynucleotide encoding constant region of sgRNA/crRNA with non-palindromic recognition site for restriction enzyme (sense or antisense), polynucleotide encoding for constant and variable regions of sgRNA/crRNA (sense or antisense), or sgRNA/crRNA.
  misc_feature (1)..(18): n is a, c, g, or t
nnnnnnnnnnnnnnnnnnatctccgccaggagaaatt

SEQ ID NO: 316 - RNA, 37 nt, Artificial Sequence
  Polynucleotide encoding constant region of sgRNA/crRNA with non-palindromic recognition site for restriction enzyme (sense or antisense), polynucleotide encoding for constant and variable regions of sgRNA/crRNA (sense or antisense), or sgRNA/crRNA.
  misc_feature (20)..(37): n is a, c, g, or u
aauuucuccuggcggagaunnnnnnnnnnnnnnnnnn

SEQ ID NO: 317 - DNA, 19 bp, Artificial Sequence
  Polynucleotide encoding constant region of sgRNA/crRNA with non-palindromic recognition site for restriction enzyme (sense or antisense), polynucleotide encoding for constant and variable regions of sgRNA/crRNA (sense or antisense), or sgRNA/crRNA.
aatttctcctgaaggagat

SEQ ID NO: 318 - DNA, 19 bp, Artificial Sequence
  Polynucleotide encoding constant region of sgRNA/crRNA with non-palindromic recognition site for restriction enzyme (sense or antisense), polynucleotide encoding for constant and variable regions of sgRNA/crRNA (sense or antisense), or sgRNA/crRNA.
atctccttcaggagaaatt

SEQ ID NO: 319 - DNA, 37 bp, Artificial Sequence
  Polynucleotide encoding constant region of sgRNA/crRNA with non-palindromic recognition site for restriction enzyme (sense or antisense), polynucleotide encoding for constant and variable regions of sgRNA/crRNA (sense or antisense), or sgRNA/crRNA.
  misc_feature (20)..(37): n is a, c, g, or t
aatttctcctgaaggagatnnnnnnnnnnnnnnnnnn

SEQ ID NO: 320 - DNA, 37 bp, Artificial Sequence
  Polynucleotide encoding constant region of sgRNA/crRNA with non-palindromic recognition site for restriction enzyme (sense or antisense), polynucleotide encoding for constant and variable regions of sgRNA/crRNA (sense or antisense), or sgRNA/crRNA.
  misc_feature (1)..(18): n is a, c, g, or t
nnnnnnnnnnnnnnnnnnatctccttcaggagaaatt

SEQ ID NO: 321 - RNA, 37 nt, Artificial Sequence
  Polynucleotide encoding constant region of sgRNA/crRNA with non-palindromic recognition site for restriction enzyme (sense or antisense), polynucleotide encoding for constant and variable regions of sgRNA/crRNA (sense or antisense), or sgRNA/crRNA.
  misc_feature (20)..(37): n is a, c, g, or u
aauuucuccugaaggagaunnnnnnnnnnnnnnnnnn

SEQ ID NO: 322 - DNA, 19 bp, Artificial Sequence
  Polynucleotide encoding constant region of sgRNA/crRNA with non-palindromic recognition site for restriction enzyme (sense or antisense), polynucleotide encoding for constant and variable regions of sgRNA/crRNA (sense or antisense), or sgRNA/crRNA.
  misc_feature (15)..(15): n is a, c, g, or t
aatttgttctcaagnacat

SEQ ID NO: 323 - DNA, 19 bp, Artificial Sequence
  Polynucleotide encoding constant region of sgRNA/crRNA with non-palindromic recognition site for restriction enzyme (sense or antisense), polynucleotide encoding for constant and variable regions of sgRNA/crRNA (sense or antisense), or sgRNA/crRNA.
  misc_feature (5)..(5): n is a, c, g, or t
atgtncttgagaacaaatt

SEQ ID NO: 324 - DNA, 37 bp, Artificial Sequence
  Polynucleotide encoding constant region of sgRNA/crRNA with non-palindromic recognition site for restriction enzyme (sense or antisense), polynucleotide encoding for constant and variable regions of sgRNA/crRNA (sense or antisense), or sgRNA/crRNA.
  misc_feature (15)..(15): n is a, c, g, or t
  misc_feature (20)..(37): n is a, c, g, or t
aatttgttctcaagnacatnnnnnnnnnnnnnnnnnn

SEQ ID NO: 325 - DNA, 37 bp, Artificial Sequence
  Polynucleotide encoding constant region of sgRNA/crRNA with non-palindromic recognition site for restriction enzyme (sense or antisense), polynucleotide encoding for constant and variable regions of sgRNA/crRNA (sense or antisense), or sgRNA/crRNA.
  misc_feature (1)..(18): n is a, c, g, or t
  misc_feature (23)..(23): n is a, c, g, or t
nnnnnnnnnnnnnnnnnnatgtncttgagaacaaatt

SEQ ID NO: 326 - RNA, 37 nt, Artificial Sequence
  Polynucleotide encoding constant region of sgRNA/crRNA with non-palindromic recognition site for restriction enzyme (sense or antisense), polynucleotide encoding for constant and variable regions of sgRNA/crRNA (sense or antisense), or sgRNA/crRNA.
  misc_feature (15)..(15): n is a, c, g, or u
  misc_feature (20)..(37): n is a, c, g, or u
aauuuguucucaagnacaunnnnnnnnnnnnnnnnnn

SEQ ID NO: 327 - DNA, 19 bp, Artificial Sequence
  Polynucleotide encoding constant region of sgRNA/crRNA with non-palindromic recognition site for restriction enzyme (sense or antisense), polynucleotide encoding for constant and variable regions of sgRNA/crRNA (sense or antisense), or sgRNA/crRNA.
aatttgtcgtgtacgacat

SEQ ID NO: 328 - DNA, 19 bp, Artificial Sequence
  Polynucleotide encoding constant region of sgRNA/crRNA with non-palindromic recognition site for restriction enzyme (sense or antisense), polynucleotide encoding for constant and variable regions of sgRNA/crRNA (sense or antisense), or sgRNA/crRNA.
atgtcgtacacgacaaatt

SEQ ID NO: 329 - DNA, 37 bp, Artificial Sequence
  Polynucleotide encoding constant region of sgRNA/crRNA with non-palindromic recognition site for restriction enzyme (sense or antisense), polynucleotide encoding for constant and variable regions of sgRNA/crRNA (sense or antisense), or sgRNA/crRNA.
  misc_feature (20)..(37): n is a, c, g, or t
aatttgtcgtgtacgacatnnnnnnnnnnnnnnnnnn

SEQ ID NO: 330 - DNA, 37 bp, Artificial Sequence
  Polynucleotide encoding constant region of sgRNA/crRNA with non-palindromic recognition site for restriction enzyme (sense or antisense), polynucleotide encoding for constant and variable regions of sgRNA/crRNA (sense or antisense), or sgRNA/crRNA.
  misc_feature (1)..(18): n is a, c, g, or t
nnnnnnnnnnnnnnnnnnatgtcgtacacgacaaatt

SEQ ID NO: 331 - RNA, 37 nt, Artificial Sequence
  Polynucleotide encoding constant region of sgRNA/crRNA with non-palindromic recognition site for restriction enzyme (sense or antisense), polynucleotide encoding for constant and variable regions of sgRNA/crRNA (sense or antisense), or sgRNA/crRNA.
  misc_feature (20)..(37): n is a, c, g, or u
aauuugucguguacgacaunnnnnnnnnnnnnnnnnn

SEQ ID NO: 332 - DNA, 19 bp, Artificial Sequence
  Polynucleotide encoding constant region of sgRNA/crRNA with non-palindromic recognition site for restriction enzyme (sense or antisense), polynucleotide encoding for constant and variable regions of sgRNA/crRNA (sense or antisense), or sgRNA/crRNA.
aatttgtggtggaccacat

SEQ ID NO: 333 - DNA, 19 bp, Artificial Sequence
  Polynucleotide encoding constant region of sgRNA/crRNA with non-palindromic recognition site for restriction enzyme (sense or antisense), polynucleotide encoding for constant and variable regions of sgRNA/crRNA (sense or antisense), or sgRNA/crRNA.
atgtggtccaccacaaatt

SEQ ID NO: 334 - DNA, 37 bp, Artificial Sequence
  Polynucleotide encoding constant region of sgRNA/crRNA with non-palindromic recognition site for restriction enzyme (sense or antisense), polynucleotide encoding for constant and variable regions of sgRNA/crRNA (sense or antisense), or sgRNA/crRNA.
  misc_feature (20)..(37): n is a, c, g, or t
aatttgtggtggaccacatnnnnnnnnnnnnnnnnnn

SEQ ID NO: 335 - DNA, 37 bp, Artificial Sequence
  Polynucleotide encoding constant region of sgRNA/crRNA with non-palindromic recognition site for restriction enzyme (sense or antisense), polynucleotide encoding for constant and variable regions of sgRNA/crRNA (sense or antisense), or sgRNA/crRNA.
  misc_feature (1)..(18): n is a, c, g, or t
nnnnnnnnnnnnnnnnnnatgtggtccaccacaaatt

SEQ ID NO: 336 - RNA, 37 nt, Artificial Sequence
  Polynucleotide encoding constant region of sgRNA/crRNA with non-palindromic recognition site for restriction enzyme (sense or antisense), polynucleotide encoding for constant and variable regions of sgRNA/crRNA (sense or antisense), or sgRNA/crRNA.
  misc_feature (20)..(37): n is a, c, g, or u
aauuugugguggaccacaunnnnnnnnnnnnnnnnnn

SEQ ID NO: 337 - DNA, 19 bp, Artificial Sequence
  Polynucleotide encoding constant region of sgRNA/crRNA with non-palindromic recognition site for restriction enzyme (sense or antisense), polynucleotide encoding for constant and variable regions of sgRNA/crRNA (sense or antisense), or sgRNA/crRNA.
aatttgtcatcrttgacat

SEQ ID NO: 338 - DNA, 19 bp, Artificial Sequence
  Polynucleotide encoding constant region of sgRNA/crRNA with non-palindromic recognition site for restriction enzyme (sense or antisense), polynucleotide encoding for constant and variable regions of sgRNA/crRNA (sense or antisense), or sgRNA/crRNA.
atgtcaaygatgacaaatt

SEQ ID NO: 339 - DNA, 37 bp, Artificial Sequence
  Polynucleotide encoding constant region of sgRNA/crRNA with non-palindromic recognition site for restriction enzyme (sense or antisense), polynucleotide encoding for constant and variable regions of sgRNA/crRNA (sense or antisense), or sgRNA/crRNA.
  misc_feature (20)..(37): n is a, c, g, or t
aatttgtcatcrttgacatnnnnnnnnnnnnnnnnnn

SEQ ID NO: 340 - DNA, 37 bp, Artificial Sequence
  Polynucleotide encoding constant region of sgRNA/crRNA with non-palindromic recognition site for restriction enzyme (sense or antisense), polynucleotide encoding for constant and variable regions of sgRNA/crRNA (sense or antisense), or sgRNA/crRNA.
  misc_feature (1)..(18): n is a, c, g, or t
nnnnnnnnnnnnnnnnnnatgtcaaygatgacaaatt

SEQ ID NO: 341 - RNA, 37 nt, Artificial Sequence
  Polynucleotide encoding constant region of sgRNA/crRNA with non-palindromic recognition site for restriction enzyme (sense or antisense), polynucleotide encoding for constant and variable regions of sgRNA/crRNA (sense or antisense), or sgRNA/crRNA.
  misc_feature (20)..(37): n is a, c, g, or u
aauuugucaucruugacaunnnnnnnnnnnnnnnnnn

SEQ ID NO: 342 - DNA, 19 bp, Artificial Sequence
  Polynucleotide encoding constant region of sgRNA/crRNA with non-palindromic recognition site for restriction enzyme (sense or antisense), polynucleotide encoding for constant and variable regions of sgRNA/crRNA (sense or antisense), or sgRNA/crRNA.
aatttcttctcaagragat

SEQ ID NO: 343 - DNA, 19 bp, Artificial Sequence
  Polynucleotide encoding constant region of sgRNA/crRNA with non-palindromic recognition site for restriction enzyme (sense or antisense), polynucleotide encoding for constant and variable regions of sgRNA/crRNA (sense or antisense), or sgRNA/crRNA.
atctycttgagaagaaatt

SEQ ID NO: 344 - DNA, 37 bp, Artificial Sequence
  Polynucleotide encoding constant region of sgRNA/crRNA with non-palindromic recognition site for restriction enzyme (sense or antisense), polynucleotide encoding for constant and variable regions of sgRNA/crRNA (sense or antisense), or sgRNA/crRNA.
  misc_feature (20)..(37): n is a, c, g, or t
aatttcttctcaagragatnnnnnnnnnnnnnnnnnn

SEQ ID NO: 345 - DNA, 37 bp, Artificial Sequence
  Polynucleotide encoding constant region of sgRNA/crRNA with non-palindromic recognition site for restriction enzyme (sense or antisense), polynucleotide encoding for constant and variable regions of sgRNA/crRNA (sense or antisense), or sgRNA/crRNA.
  misc_feature (1)..(18): n is a, c, g, or t
nnnnnnnnnnnnnnnnnnatctycttgagaagaaatt

SEQ ID NO: 346 - RNA, 37 nt, Artificial Sequence
  Polynucleotide encoding constant region of sgRNA/crRNA with non-palindromic recognition site for restriction enzyme (sense or antisense), polynucleotide encoding for constant and variable regions of sgRNA/crRNA (sense or antisense), or sgRNA/crRNA.
  misc_feature (20)..(37): n is a, c, g, or u
aauuucuucucaagragaunnnnnnnnnnnnnnnnnn

SEQ ID NO: 347 - DNA, 19 bp, Artificial Sequence
  Polynucleotide encoding constant region of sgRNA/crRNA with non-palindromic recognition site for restriction enzyme (sense or antisense), polynucleotide encoding for constant and variable regions of sgRNA/crRNA (sense or antisense), or sgRNA/crRNA.
aatttctgatgcatcagat

SEQ ID NO: 348 - DNA, 19 bp, Artificial Sequence
  Polynucleotide encoding constant region of sgRNA/crRNA with non-palindromic recognition site for restriction enzyme (sense or antisense), polynucleotide encoding for constant and variable regions of sgRNA/crRNA (sense or antisense), or sgRNA/crRNA.
atctgatgcatcagaaatt

SEQ ID NO: 349 - DNA, 38 bp, Artificial Sequence
  Polynucleotide encoding constant region of sgRNA/crRNA with non-palindromic recognition site for restriction enzyme (sense or antisense), polynucleotide encoding for constant and variable regions of sgRNA/crRNA (sense or antisense), or sgRNA/crRNA.
  misc_feature (20)..(38): n is a, c, g, or t
aatttctgatgcatcagatnnnnnnnnnnnnnnnnnnn

SEQ ID NO: 350 - DNA, 38 bp, Artificial Sequence
  Polynucleotide encoding constant region of sgRNA/crRNA with non-palindromic recognition site for restriction enzyme (sense or antisense), polynucleotide encoding for constant and variable regions of sgRNA/crRNA (sense or antisense), or sgRNA/crRNA.
  misc_feature (1)..(19): n is a, c, g, or t
nnnnnnnnnnnnnnnnnnnatctgatgcatcagaaatt

SEQ ID NO: 351 - RNA, 38 nt, Artificial Sequence
  Polynucleotide encoding constant region of sgRNA/crRNA with non-palindromic recognition site for restriction enzyme (sense or antisense), polynucleotide encoding for constant and variable regions of sgRNA/crRNA (sense or antisense), or sgRNA/crRNA.
  misc_feature (20)..(38): n is a, c, g, or u
aauuucugaugcaucagaunnnnnnnnnnnnnnnnnnn

SEQ ID NO: 352 - DNA, 19 bp, Artificial Sequence
  Polynucleotide encoding constant region of sgRNA/crRNA with non-palindromic recognition site for restriction enzyme (sense or antisense), polynucleotide encoding for constant and variable regions of sgRNA/crRNA (sense or antisense), or sgRNA/crRNA.
aattaytggtcgcccarat

SEQ ID NO: 353 - DNA, 19 bp, Artificial Sequence
  Polynucleotide encoding constant region of sgRNA/crRNA with non-palindromic recognition site for restriction enzyme (sense or antisense), polynucleotide encoding for constant and variable regions of sgRNA/crRNA (sense or antisense), or sgRNA/crRNA.
atytgggcgaccartaatt

SEQ ID NO: 354 - DNA, 37 bp, Artificial Sequence
  Polynucleotide encoding constant region of sgRNA/crRNA with non-palindromic recognition site for restriction enzyme (sense or antisense), polynucleotide encoding for constant and variable regions of sgRNA/crRNA (sense or antisense), or sgRNA/crRNA.
  misc_feature (20)..(37): n is a, c, g, or t
aattaytggtcgcccaratnnnnnnnnnnnnnnnnnn

SEQ ID NO: 355 - DNA, 37 bp, Artificial Sequence
  Polynucleotide encoding constant region of sgRNA/crRNA with non-palindromic recognition site for restriction enzyme (sense or antisense), polynucleotide encoding for constant and variable regions of sgRNA/crRNA (sense or antisense), or sgRNA/crRNA.
  misc_feature (1)..(18): n is a, c, g, or t
nnnnnnnnnnnnnnnnnnatytgggcgaccartaatt

SEQ ID NO: 356 - RNA, 37 nt, Artificial Sequence
  Polynucleotide encoding constant region of sgRNA/crRNA with non-palindromic recognition site for restriction enzyme (sense or antisense), polynucleotide encoding for constant and variable regions of sgRNA/crRNA (sense or antisense), or sgRNA/crRNA.
  misc_feature (20)..(37): n is a, c, g, or u
aauuayuccagcgggaraunnnnnnnnnnnnnnnnnn

SEQ ID NO: 357 - DNA, 19 bp, Artificial Sequence
  Polynucleotide encoding constant region of sgRNA/crRNA with non-palindromic recognition site for restriction enzyme (sense or antisense), polynucleotide encoding for constant and variable regions of sgRNA/crRNA (sense or antisense), or sgRNA/crRNA.
aatttgtgrtgccycacat

SEQ ID NO: 358 - DNA, 19 bp, Artificial Sequence
  Polynucleotide encoding constant region of sgRNA/crRNA with non-palindromic recognition site for restriction enzyme (sense or antisense), polynucleotide encoding for constant and variable regions of sgRNA/crRNA (sense or antisense), or sgRNA/crRNA.
atgtgrggcaycacaaatt

SEQ ID NO: 359 - DNA, 38 bp, Artificial Sequence
  Polynucleotide encoding constant region of sgRNA/crRNA with non-palindromic recognition site for restriction enzyme (sense or antisense), polynucleotide encoding for constant and variable regions of sgRNA/crRNA (sense or antisense), or sgRNA/crRNA.
  misc_feature (20)..(38): n is a, c, g, or t
aatttgtgrtgccycacatnnnnnnnnnnnnnnnnnnn

SEQ ID NO: 360 - DNA, 38 bp, Artificial Sequence
  Polynucleotide encoding constant region of sgRNA/crRNA with non-palindromic recognition site for restriction enzyme (sense or antisense), polynucleotide encoding for constant and variable regions of sgRNA/crRNA (sense or antisense), or sgRNA/crRNA.
  misc_feature (1)..(19): n is a, c, g, or t
nnnnnnnnnnnnnnnnnnnatgtgrggcaycacaaatt

SEQ ID NO: 361 - RNA, 38 nt, Artificial Sequence
  Polynucleotide encoding constant region of sgRNA/crRNA with non-palindromic recognition site for restriction enzyme (sense or antisense), polynucleotide encoding for constant and variable regions of sgRNA/crRNA (sense or antisense), or sgRNA/crRNA.
  misc_feature (20)..(38): n is a, c, g, or u
aauuugugrugccycacaunnnnnnnnnnnnnnnnnnn

SEQ ID NO: 362 - DNA, 19 bp, Artificial Sequence
  Polynucleotide encoding constant region of sgRNA/crRNA with non-palindromic recognition site for restriction enzyme (sense or antisense), polynucleotide encoding for constant and variable regions of sgRNA/crRNA (sense or antisense), or sgRNA/crRNA.
aatttgtcgtcatcgacat

SEQ ID NO: 363 - DNA, 19 bp, Artificial Sequence
  Polynucleotide encoding constant region of sgRNA/crRNA with non-palindromic recognition site for restriction enzyme (sense or antisense), polynucleotide encoding for constant and variable regions of sgRNA/crRNA (sense or antisense), or sgRNA/crRNA.
atgtcgatgacgacaaatt

SEQ ID NO: 364 - DNA, 37 bp, Artificial Sequence
  Polynucleotide encoding constant region of sgRNA/crRNA with non-palindromic recognition site for restriction enzyme (sense or antisense), polynucleotide encoding for constant and variable regions of sgRNA/crRNA (sense or antisense), or sgRNA/crRNA.
  misc_feature (20)..(37): n is a, c, g, or t
aatttgtcgtcatcgacatnnnnnnnnnnnnnnnnnn

SEQ ID NO: 365 - DNA, 37 bp, Artificial Sequence
  Polynucleotide encoding constant region of sgRNA/crRNA with non-palindromic recognition site for restriction enzyme (sense or antisense), polynucleotide encoding for constant and variable regions of sgRNA/crRNA (sense or antisense), or sgRNA/crRNA.
  misc_feature (1)..(18): n is a, c, g, or t
nnnnnnnnnnnnnnnnnnatgtcgatgacgacaaatt

SEQ ID NO: 366 - RNA, 37 nt, Artificial Sequence
  Polynucleotide encoding constant region of sgRNA/crRNA with non-palindromic recognition site for restriction enzyme (sense or antisense), polynucleotide encoding for constant and variable regions of sgRNA/crRNA (sense or antisense), or sgRNA/crRNA.
  misc_feature (20)..(37): n is a, c, g, or u
aauuugucgucaucgacaunnnnnnnnnnnnnnnnnn

SEQ ID NO: 367 - DNA, 19 bp, Artificial Sequence
  Polynucleotide encoding constant region of sgRNA/crRNA with non-palindromic recognition site for restriction enzyme (sense or antisense), polynucleotide encoding for constant and variable regions of sgRNA/crRNA (sense or antisense), or sgRNA/crRNA.
aatttgtcctcgrggacat

SEQ ID NO: 368 - DNA, 19 bp, Artificial Sequence
  Polynucleotide encoding constant region of sgRNA/crRNA with non-palindromic recognition site for restriction enzyme (sense or antisense), polynucleotide encoding for constant and variable regions of sgRNA/crRNA (sense or antisense), or sgRNA/crRNA.
atgtccycgaggacaaatt

SEQ ID NO: 369 - DNA, 37 bp, Artificial Sequence
  Polynucleotide encoding constant region of sgRNA/crRNA with non-palindromic recognition site for restriction enzyme (sense or antisense), polynucleotide encoding for constant and variable regions of sgRNA/crRNA (sense or antisense), or sgRNA/crRNA.
  misc_feature (20)..(37): n is a, c, g, or t
aatttgtcctcgrggacatnnnnnnnnnnnnnnnnnn

SEQ ID NO: 370 - DNA, 37 bp, Artificial Sequence
  Polynucleotide encoding constant region of sgRNA/crRNA with non-palindromic recognition site for restriction enzyme (sense or antisense), polynucleotide encoding for constant and variable regions of sgRNA/crRNA (sense or antisense), or sgRNA/crRNA.
  misc_feature (1)..(18): n is a, c, g, or t
nnnnnnnnnnnnnnnnnnatgtccycgaggacaaatt

SEQ ID NO: 371 - RNA, 37 nt, Artificial Sequence
  Polynucleotide encoding constant region of sgRNA/crRNA with non-palindromic recognition site for restriction enzyme (sense or antisense), polynucleotide encoding for constant and variable regions of sgRNA/crRNA (sense or antisense), or sgRNA/crRNA.
  misc_feature (20)..(37): n is a, c, g, or u
aauuuguccucgrggacaunnnnnnnnnnnnnnnnnn

SEQ ID NO: 372 - DNA, 19 bp, Artificial Sequence
  Polynucleotide encoding constant region of sgRNA/crRNA with non-palindromic recognition site for restriction enzyme (sense or antisense), polynucleotide encoding for constant and variable regions of sgRNA/crRNA (sense or antisense), or sgRNA/crRNA.
aatttcttctgcagragat

SEQ ID NO: 373 - DNA, 19 bp, Artificial Sequence
  Polynucleotide encoding constant region of sgRNA/crRNA with non-palindromic recognition site for restriction enzyme (sense or antisense), polynucleotide encoding for constant and variable regions of sgRNA/crRNA (sense or antisense), or sgRNA/crRNA.
atctyctgcagaagaaatt

SEQ ID NO: 374 - DNA, 38 bp, Artificial Sequence
  Polynucleotide encoding constant region of sgRNA/crRNA with non-palindromic recognition site for restriction enzyme (sense or antisense), polynucleotide encoding for constant and variable regions of sgRNA/crRNA (sense or antisense), or sgRNA/crRNA.
  misc_feature (20)..(38): n is a, c, g, or t
aatttcttctgcagragatnnnnnnnnnnnnnnnnnnn

SEQ ID NO: 375 - DNA, 38 bp, Artificial Sequence
  Polynucleotide encoding constant region of sgRNA/crRNA with non-palindromic recognition site for restriction enzyme (sense or antisense), polynucleotide encoding for constant and variable regions of sgRNA/crRNA (sense or antisense), or sgRNA/crRNA.
  misc_feature (1)..(19): n is a, c, g, or t
nnnnnnnnnnnnnnnnnnnatctyctgcagaagaaatt

SEQ ID NO: 376 - RNA, 38 nt, Artificial Sequence
  Polynucleotide encoding constant region of sgRNA/crRNA with non-palindromic recognition site for restriction enzyme (sense or antisense), polynucleotide encoding for constant and variable regions of sgRNA/crRNA (sense or antisense), or sgRNA/crRNA.
  misc_feature (20)..(38): n is a, c, g, or u
aauuucuucugcagragaunnnnnnnnnnnnnnnnnnn

SEQ ID NO: 377 - DNA, 19 bp, Artificial Sequence
  Polynucleotide encoding constant region of sgRNA/crRNA with non-palindromic recognition site for restriction enzyme (sense or antisense), polynucleotide encoding for constant and variable regions of sgRNA/crRNA (sense or antisense), or sgRNA/crRNA.
aatttcttctgcggragat

SEQ ID NO: 378 - DNA, 19 bp, Artificial Sequence
  Polynucleotide encoding constant region of sgRNA/crRNA with non-palindromic recognition site for restriction enzyme (sense or antisense), polynucleotide encoding for constant and variable regions of sgRNA/crRNA (sense or antisense), or sgRNA/crRNA.
atctyccgcagaagaaatt

SEQ ID NO: 379 - DNA, 37 bp, Artificial Sequence
  Polynucleotide encoding constant region of sgRNA/crRNA with non-palindromic recognition site for restriction enzyme (sense or antisense), polynucleotide encoding for constant and variable regions of sgRNA/crRNA (sense or antisense), or sgRNA/crRNA.
  misc_feature (20)..(37): n is a, c, g, or t
aatttcttctgcggragatnnnnnnnnnnnnnnnnnn

SEQ ID NO: 380 - DNA, 37 bp, Artificial Sequence
  Polynucleotide encoding constant region of sgRNA/crRNA with non-palindromic recognition site for restriction enzyme (sense or antisense), polynucleotide encoding for constant and variable regions of sgRNA/crRNA (sense or antisense), or sgRNA/crRNA.
  misc_feature (1)..(18): n is a, c, g, or t
nnnnnnnnnnnnnnnnnnatctyccgcagaagaaatt

SEQ ID NO: 381 - RNA, 37 nt, Artificial Sequence
  Polynucleotide encoding constant region of sgRNA/crRNA with non-palindromic recognition site for restriction enzyme (sense or antisense), polynucleotide encoding for constant and variable regions of sgRNA/crRNA (sense or antisense), or sgRNA/crRNA.
  misc_feature (20)..(37): n is a, c, g, or u
aauuucuucugcggragaunnnnnnnnnnnnnnnnnn

SEQ ID NO: 382 - DNA, 19 bp, Artificial Sequence
  Polynucleotide encoding constant region of sgRNA/crRNA with non-palindromic recognition site for restriction enzyme (sense or antisense), polynucleotide encoding for constant and variable regions of sgRNA/crRNA (sense or antisense), or sgRNA/crRNA.
aatttgtgttgcaacacat

SEQ ID NO: 383 - DNA, 19 bp, Artificial Sequence
  Polynucleotide encoding constant region of sgRNA/crRNA with non-palindromic recognition site for restriction enzyme (sense or antisense), polynucleotide encoding for constant and variable regions of sgRNA/crRNA (sense or antisense), or sgRNA/crRNA.
atgtgttgcaacacaaatt

SEQ ID NO: 384 - DNA, 38 bp, Artificial Sequence
  Polynucleotide encoding constant region of sgRNA/crRNA with non-palindromic recognition site for restriction enzyme (sense or antisense), polynucleotide encoding for constant and variable regions of sgRNA/crRNA (sense or antisense), or sgRNA/crRNA.
  misc_feature (20)..(38): n is a, c, g, or t
aatttgtgttgcaacacatnnnnnnnnnnnnnnnnnnn

SEQ ID NO: 385 - DNA, 38 bp, Artificial Sequence
  Polynucleotide encoding constant region of sgRNA/crRNA with non-palindromic recognition site for restriction enzyme (sense or antisense), polynucleotide encoding for constant and variable regions of sgRNA/crRNA (sense or antisense), or sgRNA/crRNA.
  misc_feature (1)..(19): n is a, c, g, or t
nnnnnnnnnnnnnnnnnnnatgtgttgcaacacaaatt

SEQ ID NO: 386 - RNA, 38 nt, Artificial Sequence
  Polynucleotide encoding constant region of sgRNA/crRNA with non-palindromic recognition site for restriction enzyme (sense or antisense), polynucleotide encoding for constant and variable regions of sgRNA/crRNA (sense or antisense), or sgRNA/crRNA.
  misc_feature (20)..(38): n is a, c, g, or u
aauuuguguugcaacacaunnnnnnnnnnnnnnnnnnn

SEQ ID NO: 387 - DNA, 20 bp, Artificial Sequence
  Polynucleotide encoding constant region of sgRNA/crRNA with non-palindromic recognition site for restriction enzyme (sense or antisense), polynucleotide encoding for constant and variable regions of sgRNA/crRNA (sense or antisense), or sgRNA/crRNA.
aatttcctgtgcatcaggat

SEQ ID NO: 388 - DNA, 20 bp, Artificial Sequence
  Polynucleotide encoding constant region of sgRNA/crRNA with non-palindromic recognition site for restriction enzyme (sense or antisense), polynucleotide encoding for constant and variable regions of sgRNA/crRNA (sense or antisense), or sgRNA/crRNA.
atcctgatgcacaggaaatt

SEQ ID NO: 389 - DNA, 38 bp, Artificial Sequence
  Polynucleotide encoding constant region of sgRNA/crRNA with non-palindromic recognition site for restriction enzyme (sense or antisense), polynucleotide encoding for constant and variable regions of sgRNA/crRNA (sense or antisense), or sgRNA/crRNA.
  misc_feature (21)..(38): n is a, c, g, or t
aatttcctgtgcatcaggatnnnnnnnnnnnnnnnnnn

SEQ ID NO: 390 - DNA, 38 bp, Artificial Sequence
  Polynucleotide encoding constant region of sgRNA/crRNA with non-palindromic recognition site for restriction enzyme (sense or antisense), polynucleotide encoding for constant and variable regions of sgRNA/crRNA (sense or antisense), or sgRNA/crRNA.
  misc_feature (1)..(18): n is a, c, g, or t
nnnnnnnnnnnnnnnnnnatcctgatgcacaggaaatt

SEQ ID NO: 391 - RNA, 38 nt, Artificial Sequence
  Polynucleotide encoding constant region of sgRNA/crRNA with non-palindromic recognition site for restriction enzyme (sense or antisense), polynucleotide encoding for constant and variable regions of sgRNA/crRNA (sense or antisense), or sgRNA/crRNA.
  misc_feature (21)..(38): n is a, c, g, or u
aauuuccugugcaucaggaunnnnnnnnnnnnnnnnnn

SEQ ID NO: 392 - DNA, 20 bp, Artificial Sequence
  Polynucleotide encoding constant region of sgRNA/crRNA with non-palindromic recognition site for restriction enzyme (sense or antisense), polynucleotide encoding for constant and variable regions of sgRNA/crRNA (sense or antisense), or sgRNA/crRNA.
aatttcctgtgccycaggat

SEQ ID NO: 393 - DNA, 20 bp, Artificial Sequence
  Polynucleotide encoding constant region of sgRNA/crRNA with non-palindromic recognition site for restriction enzyme (sense or antisense), polynucleotide encoding for constant and variable regions of sgRNA/crRNA (sense or antisense), or sgRNA/crRNA.
atcctgrggcacaggaaatt

SEQ ID NO: 394 - DNA, 38 bp, Artificial Sequence
  Polynucleotide encoding constant region of sgRNA/crRNA with non-palindromic recognition site for restriction enzyme (sense or antisense), polynucleotide encoding for constant and variable regions of sgRNA/crRNA (sense or antisense), or sgRNA/crRNA.
  misc_feature (21)..(38): n is a, c, g, or t
aatttcctgtgccycaggatnnnnnnnnnnnnnnnnnn

SEQ ID NO: 395 - DNA, 38 bp, Artificial Sequence
  Polynucleotide encoding constant region of sgRNA/crRNA with non-palindromic recognition site for restriction enzyme (sense or antisense), polynucleotide encoding for constant and variable regions of sgRNA/crRNA (sense or antisense), or sgRNA/crRNA.
  misc_feature (1)..(18): n is a, c, g, or t
nnnnnnnnnnnnnnnnnnatcctgrggcacaggaaatt

SEQ ID NO: 396 - RNA, 38 nt, Artificial Sequence
  Polynucleotide encoding constant region of sgRNA/crRNA with non-palindromic recognition site for restriction enzyme (sense or antisense), polynucleotide encoding for constant and variable regions of sgRNA/crRNA (sense or antisense), or sgRNA/crRNA.
  misc_feature (21)..(38): n is a, c, g, or u
aauuuccugugccycaggaunnnnnnnnnnnnnnnnnn

SEQ ID NO: 397 - DNA, 20 bp, Artificial Sequence
  Polynucleotide encoding constant region of sgRNA/crRNA with non-palindromic recognition site for restriction enzyme (sense or antisense), polynucleotide encoding for constant and variable regions of sgRNA/crRNA (sense or antisense), or sgRNA/crRNA.
aatttcctttgcagraggat

SEQ ID NO: 398 - DNA, 20 bp, Artificial Sequence
  Polynucleotide encoding constant region of sgRNA/crRNA with non-palindromic recognition site for restriction enzyme (sense or antisense), polynucleotide encoding for constant and variable regions of sgRNA/crRNA (sense or antisense), or sgRNA/crRNA.
atcctyctgcaaaggaaatt

SEQ ID NO: 399 - DNA, 38 bp, Artificial Sequence
  Polynucleotide encoding constant region of sgRNA/crRNA with non-palindromic recognition site for restriction enzyme (sense or antisense), polynucleotide encoding for constant and variable regions of sgRNA/crRNA (sense or antisense), or sgRNA/crRNA.
  misc_feature (21)..(38): n is a, c, g, or t
aatttcctttgcagraggatnnnnnnnnnnnnnnnnnn

SEQ ID NO: 400 - DNA, 38 bp, Artificial Sequence
  Polynucleotide encoding constant region of sgRNA/crRNA with non-palindromic recognition site for restriction enzyme (sense or antisense), polynucleotide encoding for constant and variable regions of sgRNA/crRNA (sense or antisense), or sgRNA/crRNA.
  misc_feature (1)..(18): n is a, c, g, or t
nnnnnnnnnnnnnnnnnnatcctyctgcaaaggaaatt

SEQ ID NO: 401 - RNA, 38 nt, Artificial Sequence
  Polynucleotide encoding constant region of sgRNA/crRNA with non-palindromic recognition site for restriction enzyme (sense or antisense), polynucleotide encoding for constant and variable regions of sgRNA/crRNA (sense or antisense), or sgRNA/crRNA.
  misc_feature (21)..(38): n is a, c, g, or u
aauuuccuuugcagraggaunnnnnnnnnnnnnnnnnn

SEQ ID NO: 402 - DNA, 20 bp, Artificial Sequence
  Polynucleotide encoding constant region of sgRNA/crRNA with non-palindromic recognition site for restriction enzyme (sense or antisense), polynucleotide encoding for constant and variable regions of sgRNA/crRNA (sense or antisense), or sgRNA/crRNA.
aatttcgtgtgcaacacgat

SEQ ID NO: 403 - DNA, 20 bp, Artificial Sequence
  Polynucleotide encoding constant region of sgRNA/crRNA with non-palindromic recognition site for restriction enzyme (sense or antisense), polynucleotide encoding for constant and variable regions of sgRNA/crRNA (sense or antisense), or sgRNA/crRNA.
atcgtgttgcacacgaaatt

SEQ ID NO: 404 - DNA, 38 bp, Artificial Sequence
  Polynucleotide encoding constant region of sgRNA/crRNA with non-palindromic recognition site for restriction enzyme (sense or antisense), polynucleotide encoding for constant and variable regions of sgRNA/crRNA (sense or antisense), or sgRNA/crRNA.
  misc_feature (21)..(38): n is a, c, g, or t
aatttcgtgtgcaacacgatnnnnnnnnnnnnnnnnnn

SEQ ID NO: 405 - DNA, 38 bp, Artificial Sequence
  Polynucleotide encoding constant region of sgRNA/crRNA with non-palindromic recognition site for restriction enzyme (sense or antisense), polynucleotide encoding for constant and variable regions of sgRNA/crRNA (sense or antisense), or sgRNA/crRNA.
  misc_feature (1)..(18): n is a, c, g, or t
nnnnnnnnnnnnnnnnnnatcgtgttgcacacgaaatt

SEQ ID NO: 406 - RNA, 38 nt, Artificial Sequence
  Polynucleotide encoding constant region of sgRNA/crRNA with non-palindromic recognition site for restriction enzyme (sense or antisense), polynucleotide encoding for constant and variable regions of sgRNA/crRNA (sense or antisense), or sgRNA/crRNA.
  misc_feature (21)..(38): n is a, c, g, or u
aauuucgugugcaacacgaunnnnnnnnnnnnnnnnnn

SEQ ID NO: 407 - DNA, 1653 bp, Artificial Sequence
  Polynucleotide containing EGFP Transgene sequence
agctcggatccaaaagcttaaatcagttgtgttaaataagagacattcaaaataaatgtaaatgagctctccaaatcagcagacttaacattctttaaaatgattgattcaatagtgataaaaatcaggcatagccagttgtaactttagataaattacagaaaatgtcaaatacagagaaccgattcttttttatgatacatccaagcacacatttaacacaatccaggcaaaccccgaatttcacagtcacaagcactgtttgtacaagagctttgcctaaggacacacagtctctataagtccaggtcgttggtttcactcttattttaaacatgtgacatttttcctgccatcctgtcttaggctgctgtttgcttcattccatgtcacattaaattcctcagtagcaccttttacacacacagccaatcttttccagaaaattcaattgctttgaagagataatgtgtgaacaaatccatttagaaaaggaaaattaagaatttgtaaaatcatctgtaaattgttggcattcttctgtatatgaacatcacatcatttacaggtaaaggtctggtcattaattatatgacaatttactggtattattttgtgaaaggggctattttcaatgcgttcatccatccttttcatccctcaaatctctcattcacgtccccctccccatctgcacactttatctcattttccaccctgctggaatctgagcacttgtgcagttatcagggctcctgtatttaggaggctctgggtgtccatgtaggggacgaacagaaacactgcagacctttatagaagaacaattgataagagtcctcatacataaagactccattagtaagccagtgacccaggagcccagaccaacagcaaagcagacagtgaccatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagtagtaaacgttgcagcaagattaca

SEQ ID NO: 408 - DNA, 100 bp, Artificial Sequence
  PCR Product
cagcaaagcagacagtgaccatggtgagcaagggcgaggagctgttcaccggggtggtgcccatcctggtcgagctggacggcgacgtaaacggccacaa

SEQ ID NO: 409 - DNA, 20 bp, Artificial Sequence
  Forward Primer
cagcaaagcagacagtgacc

SEQ ID NO: 410 - DNA, 18 bp, Artificial Sequence
  Reverse Primer
ttgtggccgtttacgtcg

SEQ ID NO: 411 - DNA, 49 bp, Artificial Sequence
  Digested PCR Product(1)
cagcaaagcagacagtgaccatggtgagcaagggcgaggagctgttcac

SEQ ID NO: 412 - DNA, 51 bp, Artificial Sequence
  Digested PCR Product(2)
cggggtggtgcccatcctggtcgagctggacggcgacgtaaacggccacaa

SEQ ID NO: 413 - DNA, 18 bp, Artificial Sequence
  MmeI digested product(1)
gggcgaggagctgttcac

SEQ ID NO: 414 - DNA, 18 bp, Artificial Sequence
  MmeI digested product(2)
gggtggtgcccatcctgg

SEQ ID NO: 415 - DNA, 29 bp, Artificial Sequence
  T7 Promoter
  misc_feature (28)..(29): n is a, c, g, or t
cggcgaaattaatacgactcactatagnn

SEQ ID NO: 416 - RNA, 102 nt, Streptococcus pyogenes
  misc_feature (1)..(20): n is a, c, g, or u
nnnnnnnnnnnnnnnnnnnnguuuuagagcuagaaauagcaaguuaaaauaaggcuaguccguuaucaacuugaaaaaguggcaccgagucggugcuuuuuu

SEQ ID NO: 417 - DNA, 102 bp, Streptococcus pyogenes
  misc_feature (1)..(20): n is a, c, g, or t
nnnnnnnnnnnnnnnnnnnngttttagagctagaaatagcaagttaaaataaggctagtccgttatcaacttgaaaaagtggcaccgagtcggtgctttttt

SEQ ID NO: 418 - DNA, 102 bp, Streptococcus pyogenes
  misc_feature (1)..(20): n is a, c, g, or t
nnnnnnnnnnnnnnnnnnnncaaaatctcgatctttatcgttcaattttattccgatcaggcaatagttgaactttttcaccgtggctcagccacgaaaaaa

SEQ ID NO: 419 - DNA, 102 bp, Artificial Sequence
  polynucleotides encoding for a sgRNA and having non-palindromic recognition sites
  misc_feature (1)..(20): n is a, c, g, or t
nnnnnnnnnnnnnnnnnnnngttggagagctagaaatagcaagttccaataaggctagtccgttatcaacttgaaaaagtggcaccgagtcggtgctttttt

SEQ ID NO: 420 - DNA, 102 bp, Artificial Sequence
  polynucleotides encoding for a sgRNA and having non-palindromic recognition sites
  misc_feature (1)..(20): n is a, c, g, or t
nnnnnnnnnnnnnnnnnnnncaacctctcgatctttatcgttcaaggttattccgatcaggcaatagttgaactttttcaccgtggctcagccacgaaaaaa

SEQ ID NO: 421 - DNA, 102 bp, Artificial Sequence
  polynucleotides encoding for a sgRNA and having non-palindromic recognition sites
  misc_feature (1)..(20): n is a, c, g, or t
nnnnnnnnnnnnnnnnnnnncggttggagctagaaatagcaagtcaacctaaggctagtccgttatcaacttgaaaaagtggcaccgagtcggtgctttttt

SEQ ID NO: 422 - DNA, 102 bp, Artificial Sequence
  polynucleotides encoding for a sgRNA and having non-palindromic recognition sites
  misc_feature (1)..(20): n is a, c, g, or t
nnnnnnnnnnnnnnnnnnnngccaacctcgatctttatcgttcagttggattccgatcaggcaatagttgaactttttcaccgtggctcagccacgaaaaaa

SEQ ID NO: 423 - RNA, 101 nt, Streptococcus pyogenes
  misc_feature (1)..(20): n is a, c, g, or u
nnnnnnnnnnnnnnnnnnnnguuuuagagcuagaaauagcaaguuaaaauaaggcuaguccguuaucaacuugaaaaaguggcaccgagucggugcuuuuu

SEQ ID NO: 424 - DNA, 101 bp, Streptococcus pyogenes
  misc_feature (1)..(20): n is a, c, g, or t
nnnnnnnnnnnnnnnnnnnngttttagagctagaaatagcaagttaaaataaggctagtccgttatcaacttgaaaaagtggcaccgagtcggtgcttttt

SEQ ID NO: 425 - DNA, 101 bp, Artificial Sequence; identical to SEQ ID NO: 424
  polynucleotides encoding for an sgRNA or crRNA recognized by a CRIPSR Cas9 protein and having non-palindromic recognition site
  misc_feature (1)..(20): n is a, c, g, or t

SEQ ID NO: 426 - DNA, 100 bp, Artificial Sequence
  polynucleotides encoding for an sgRNA or crRNA recognized by a CRIPSR Cas9 protein and having non-palindromic recognition site
  misc_feature (1)..(19): n is a, c, g, or t
nnnnnnnnnnnnnnnnnnnggttggaagctagaaatagcaagtccaactaaggctagtccgttatcaacttgaaaaagtggcaccgagtcggtgcttttt

SEQ ID NO: 427 - DNA, 99 bp, Artificial Sequence
  polynucleotides encoding for an sgRNA or crRNA recognized by a CRIPSR Cas9 protein and having non-palindromic recognition site
  misc_feature (1)..(18): n is a, c, g, or t
nnnnnnnnnnnnnnnnnngtgttggagctagaaatagcaagtcaacataaggctagtccgttatcaacttgaaaaagtggcaccgagtcggtgcttttt

SEQ ID NO: 428 - DNA, 99 bp, Artificial Sequence
  polynucleotides encoding for an sgRNA or crRNA recognized by a CRIPSR Cas9 protein and having non-palindromic recognition site
  misc_feature (1)..(17): n is a, c, g, or t
nnnnnnnnnnnnnnnnnccggttggagctagaaatagcaagtcaacctaaggctagtccgttatcaacttgaaaaagtggcaccgagtcggtgcttttt

SEQ ID NO: 429 - DNA, 99 bp, Artificial Sequence
  polynucleotides encoding for an sgRNA or crRNA recognized by a CRIPSR Cas9 protein and having non-palindromic recognition site
  misc_feature (1)..(16): n is a, c, g, or t
  misc_feature (19)..(19): n is a, c, g, or t
nnnnnnnnnnnnnnnnccnggttggagctagaaatagcaagtcaacctaaggctagtccgttatcaacttgaaaaagtggcaccgagtcggtgcttttt

SEQ ID NO: 430 - DNA, 99 bp, Artificial Sequence
  polynucleotides encoding for an sgRNA or crRNA recognized by a CRIPSR Cas9 protein and having non-palindromic recognition site
  misc_feature (1)..(17): n is a, c, g, or t
nnnnnnnnnnnnnnnnnctagttggagctagaaatagcaagtcaacctaaggctagtccgttatcaacttgaaaaagtggcaccgagtcggtgcttttt

SEQ ID NO: 431 - DNA, 101 bp, Artificial Sequence
  polynucleotides encoding for an sgRNA or crRNA recognized by a CRIPSR Cas9 protein and having non-palindromic recognition site
  misc_feature (1)..(20): n is a, c, g, or t
nnnnnnnnnnnnnnnnnnnngtcggagagctagaaatagcaagttccgataaggctagtccgttatcaacttgaaaaagtggcaccgagtcggtgcttttt

SEQ ID NO: 432 - DNA, 100 bp, Artificial Sequence
  polynucleotides encoding for an sgRNA or crRNA recognized by a CRIPSR Cas9 protein and having non-palindromic recognition site
  misc_feature (1)..(19): n is a, c, g, or t
nnnnnnnnnnnnnnnnnnnggtcggaagctagaaatagcaagtccgactaaggctagtccgttatcaacttgaaaaagtggcaccgagtcggtgcttttt

SEQ ID NO: 433 - DNA, 99 bp, Artificial Sequence
  polynucleotides encoding for an sgRNA or crRNA recognized by a CRIPSR Cas9 protein and having non-palindromic recognition site
  misc_feature (1)..(18): n is a, c, g, or t
nnnnnnnnnnnnnnnnnngtgtcggagctagaaatagcaagtcgacataaggctagtccgttatcaacttgaaaaagtggcaccgagtcggtgcttttt

SEQ ID NO: 434 - DNA, 99 bp, Artificial Sequence
  polynucleotides encoding for an sgRNA or crRNA recognized by a CRIPSR Cas9 protein and having non-palindromic recognition site
  misc_feature (1)..(17): n is a, c, g, or t
nnnnnnnnnnnnnnnnnccggtcggagctagaaatagcaagtcgacctaaggctagtccgttatcaacttgaaaaagtggcaccgagtcggtgcttttt

SEQ ID NO: 435 - DNA, 99 bp, Artificial Sequence
  polynucleotides encoding for an sgRNA or crRNA recognized by a CRIPSR Cas9 protein and having non-palindromic recognition site
  misc_feature (1)..(16): n is a, c, g, or t
  misc_feature (19)..(19): n is a, c, g, or t
nnnnnnnnnnnnnnnnccnggtcggagctagaaatagcaagtcgacctaaggctagtccgttatcaacttgaaaaagtggcaccgagtcggtgcttttt

SEQ ID NO: 436 - DNA, 99 bp, Artificial Sequence
  polynucleotides encoding for an sgRNA or crRNA recognized by a CRIPSR Cas9 protein and having non-palindromic recognition site
  misc_feature (1)..(17): n is a, c, g, or t
nnnnnnnnnnnnnnnnnctagtcggagctagaaatagcaagtcgacctaaggctagtccgttatcaacttgaaaaagtggcaccgagtcggtgcttttt

SEQ ID NO: 437 - DNA, 101 bp, Artificial Sequence; identical to SEQ ID NO: 36
  polynucleotides encoding for an sgRNA or crRNA recognized by a CRIPSR Cas9 protein and having non-palindromic recognition site
  misc_feature (1)..(20): n is a, c, g, or t

SEQ ID NO: 438 - DNA, 100 bp, Artificial Sequence; identical to SEQ ID NO: 41
  polynucleotides encoding for an sgRNA or crRNA recognized by a CRIPSR Cas9 protein and having non-palindromic recognition site
  misc_feature (1)..(19): n is a, c, g, or t

SEQ ID NO: 439 - DNA, 99 bp, Artificial Sequence; identical to SEQ ID NO: 46
  polynucleotides encoding for an sgRNA or crRNA recognized by a CRIPSR Cas9 protein and having non-palindromic recognition site
  misc_feature (1)..(18): n is a, c, g, or t

SEQ ID NO: 440 - RNA, 39 nt, Artificial Sequence
  Francisella novicida
  misc_feature (20)..(39): n is a, c, g, or u
aauuucuacuguuguagaunnnnnnnnnnnnnnnnnnnn

SEQ ID NO: 441 - DNA, 38 bp, Artificial Sequence
  Francisella novicida cRNA for CRISPR Cpf1
  misc_feature (20)..(38): n is a, c, g, or t
aatttctactgttgtagatnnnnnnnnnnnnnnnnnnn

SEQ ID NO: 442 - DNA, 38 bp, Artificial Sequence
  polynucleotides encoding for an sgRNA or crRNA recognized by a CRISPR Cpf1 protein and having non-palindromic recognition site.
  misc_feature (20)..(38): n is a, c, g, or t
aattgttggtgttccaactnnnnnnnnnnnnnnnnnnn

SEQ ID NO: 443 - DNA, 37 bp, Artificial Sequence; identical to SEQ ID NO: 159
  polynucleotides encoding for an sgRNA or crRNA recognized by a CRISPR Cpf1 protein and having non-palindromic recognition site.
  misc_feature (20)..(37): n is a, c, g, or t

SEQ ID NO: 444 - DNA, 38 bp, Artificial Sequence
  polynucleotides encoding for an sgRNA or crRNA recognized by a CRISPR Cpf1 protein and having non-palindromic recognition site.
  misc_feature (20)..(38): n is a, c, g, or t
aattgttggtgttccgactnnnnnnnnnnnnnnnnnnn

SEQ ID NO: 445 - DNA, 37 bp, Artificial Sequence
  polynucleotides encoding for an sgRNA or crRNA recognized by a CRISPR Cpf1 protein and having non-palindromic recognition site.
  misc_feature (20)..(37): n is a, c, g, or t
aatttgttgtgtccgacatnnnnnnnnnnnnnnnnnn

SEQ ID NO: 446 - DNA, 38 bp, Artificial Sequence; identical to SEQ ID NO: 174
  polynucleotides encoding for an sgRNA or crRNA recognized by a CRISPR Cpf1 protein and having non-palindromic recognition site.
  misc_feature (20)..(38): n is a, c, g, or t

SEQ ID NO: 447 - DNA, 37 bp, Artificial Sequence; identical to SEQ ID NO: 179
  polynucleotides encoding for an sgRNA or crRNA recognized by a CRISPR Cpf1 protein and having non-palindromic recognition site.
  misc_feature (20)..(37): n is a, c, g, or t

SEQ ID NO: 448 - DNA, 38 bp, Artificial Sequence
  polynucleotides encoding for an sgRNA or crRNA recognized by a CRISPR Cpf1 protein and having non-palindromic recognition site.
  misc_feature (22)..(38): n is a, c, g, or t
aattgttggtgttccaactatnnnnnnnnnnnnnnnnn

SEQ ID NO: 449 - DNA, 37 bp, Artificial Sequence
  polynucleotides encoding for an sgRNA or crRNA recognized by a CRISPR Cpf1 protein and having non-palindromic recognition site.
  misc_feature (22)..(37): n is a, c, g, or t
aatttgttgtgtccaacatatnnnnnnnnnnnnnnnn

SEQ ID NO: 450 - DNA, 38 bp, Artificial Sequence
  polynucleotides encoding for an sgRNA or crRNA recognized by a CRISPR Cpf1 protein and having non-palindromic recognition site.
  misc_feature (22)..(38): n is a, c, g, or t
aattgttggtgttccgactatnnnnnnnnnnnnnnnnn

SEQ ID NO: 451 - DNA, 37 bp, Artificial Sequence
  polynucleotides encoding for an sgRNA or crRNA recognized by a CRISPR Cpf1 protein and having non-palindromic recognition site.
  misc_feature (22)..(37): n is a, c, g, or t
aatttgttgtgtccgacatatnnnnnnnnnnnnnnnn

SEQ ID NO: 452 - DNA, 38 bp, Artificial Sequence
  polynucleotides encoding for an sgRNA or crRNA recognized by a CRISPR Cpf1 protein and having non-palindromic recognition site.
  misc_feature (22)..(38): n is a, c, g, or t
aattctcggtgtgccgagtatnnnnnnnnnnnnnnnnn

SEQ ID NO: 453 - DNA, 37 bp, Artificial Sequence
  polynucleotides encoding for an sgRNA or crRNA recognized by a CRISPR Cpf1 protein and having non-palindromic recognition site.
  misc_feature (22)..(37): n is a, c, g, or t
aatttctcgtggccgagatatnnnnnnnnnnnnnnnn

SEQ ID NO: 454 - DNA, 81 bp, Artificial Sequence
  Forward strand of a polynucleotide which encodes an RNA bound by a cas enzyme
  misc_feature (1)..(11): Sequence1 (Used in Kit)
  misc_feature (3)..(8): Type IIS binding site
  misc_feature (12)..(81): Sequence2 (In Plasmid)
ntgtyggagctagaaatagcaagtcragataaggctagtccgttatcaacttgaaaaagtggcaccgagtcggtgcttttt

SEQ ID NO: 455 - DNA, 80 bp, Artificial Sequence
  Reverse strand of a polynucleotide which encodes an RNA bound by a cas enzyme
  misc_feature (1)..(74): Sequence1 (Used in Kit)
  misc_feature (74)..(79): Type IIS binding site
  misc_feature (75)..(80): Sequence1 (Used in Kit)
aaaaagcaccgactcggtgccactttttcaagttgataacggactagccttatctygacttgctatttctagctccraca

SEQ ID NO: 456 - DNA, 81 bp, Artificial Sequence
  Forward strand of a polynucleotide which encodes an RNA bound by a cas enzyme
  misc_feature (1)..(11): Sequence1 (Used in Kit)
  misc_feature (3)..(8): Type IIS binding site
  misc_feature (12)..(81): Sequence2 (In Plasmid)
ntctyggagctagaaatagcaagtcragataaggctagtccgttatcaacttgaaaaagtggcaccgagtcggtgcttttt

SEQ ID NO: 457 - DNA, 80 bp, Artificial Sequence
  Reverse strand of a polynucleotide which encodes an RNA bound by a cas enzyme
  misc_feature (1)..(74): Sequence2 (In Plasmid)
  misc_feature (74)..(79): Type IIS binding site
  misc_feature (75)..(80): Sequence1 (Used in Kit)
aaaaagcaccgactcggtgccactttttcaagttgataacggactagccttatctygacttgctatttctagctccraga

SEQ ID NO: 458 - DNA, 81 bp, Artificial Sequence
  Forward strand of a polynucleotide which encodes an RNA bound by a cas enzyme
  misc_feature (3)..(8): Type IIS binding site
cgctyggagctagaaatagcaagtcragctaaggctagtccgttatcaacttgaaaaagtggcaccgagtcggtgcttttt

SEQ ID NO: 459 - DNA, 79 bp, Artificial Sequence
  Reverse strand of a polynucleotide which encodes an RNA bound by a cas enzyme
  misc_feature (74)..(79): Type IIS binding site
aaaaagcaccgactcggtgccactttttcaagttgataacggactagccttagctygacttgctatttctagctccrag

SEQ ID NO: 460 - DNA, 81 bp, Artificial Sequence
  Forward strand of a polynucleotide which encodes an RNA bound by a cas enzyme
  misc_feature (3)..(8): Type IIS binding site
tactyggagctagaaatagcaagtcragctaaggctagtccgttatcaacttgaaaaagtggcaccgagtcggtgcttttt

SEQ ID NO: 461 - DNA, 79 bp, Artificial Sequence; identical to SEQ ID NO: 459
  Reverse strand of a polynucleotide which encodes an RNA bound by a cas enzyme
  misc_feature (74)..(79): Type IIS binding site

SEQ ID NO: 462 - DNA, 81 bp, Artificial Sequence
  Forward strand of a polynucleotide which encodes an RNA bound by a cas enzyme
  misc_feature (3)..(8): Type IIS binding site
gtctyggagctagaaatagcaagtcragctaaggctagtccgttatcaacttgaaaaagtggcaccgagtcggtgcttttt

SEQ ID NO: 463 - DNA, 83 bp, Artificial Sequence
  Reverse strand of a polynucleotide which encodes an RNA bound by a cas enzyme
  misc_feature (74)..(79): Type IIS binding site
aaaaagcaccgactcggtgccactttttcaagttgataacggactagccttagctygacttgctatttctagctccragacat

SEQ ID NO: 464 - DNA, 81 bp, Artificial Sequence
  Forward strand of a polynucleotide which encodes an RNA bound by a cas enzyme
  misc_feature (3)..(8): Type IIS binding site
gtgtyggagctagaaatagcaagtcragctaaggctagtccgttatcaacttgaaaaagtggcaccgagtcggtgcttttt

SEQ ID NO: 465 - DNA, 83 bp, Artificial Sequence
  Reverse strand of a polynucleotide which encodes an RNA bound by a cas enzyme
  misc_feature (74)..(79): Type IIS binding site
aaaaagcaccgactcggtgccactttttcaagttgataacggactagccttagctygacttgctatttctagctccracacat

SEQ ID NO: 466 - DNA, 81 bp, Artificial Sequence
  Forward strand of a polynucleotide which encodes an RNA bound by a cas enzyme
  misc_feature (1)..(11): Sequence1 (Used in Kit)
  misc_feature (2)..(7): Type IIS binding site
  misc_feature (12)..(81): Sequence2 (In Plasmid)
cgtyggaagctagaaatagcaagaccractaaggctagtccgttatcaacttgaaaaagtggcaccgagtcggtgcttttt

SEQ ID NO: 467 - DNA, 79 bp, Artificial Sequence
  Reverse strand of a polynucleotide which encodes an RNA bound by a cas enzyme
  misc_feature (1)..(74): Sequence2 (In Plasmid)
  misc_feature (75)..(79): Type IIS binding site
  misc_feature (75)..(79): Sequence1 (Used in Kit)
aaaaagcaccgactcggtgccactttttcaagttgataacggactagccttagtyggtcttgctatttctagcttccra

SEQ ID NO: 468 - DNA, 81 bp, Artificial Sequence
  Forward strand of a polynucleotide which encodes an RNA bound by a cas enzyme
  misc_feature (1)..(11): Sequence1 (Used in Kit)
  misc_feature (2)..(7): Type IIS binding site
  misc_feature (12)..(81): Sequence2 (In Plasmid)
nctyggaagctagaaatagcaagaccractaaggctagtccgttatcaacttgaaaaagtggcaccgagtcggtgcttttt

SEQ ID NO: 469 - DNA, 80 bp, Artificial Sequence
  Reverse strand of a polynucleotide which encodes an RNA bound by a cas enzyme
  misc_feature (1)..(74): Sequence2 (In Plasmid)
  misc_feature (75)..(80): Type IIS binding site
  misc_feature (75)..(80): Sequence1 (Used in Kit)
aaaaagcaccgactcggtgccactttttcaagttgataacggactagccttagtyggtcttgctatttctagcttccrag

SEQ ID NO: 470 - DNA, 81 bp, Artificial Sequence
  Forward strand of a polynucleotide which encodes an RNA bound by a cas enzyme
  misc_feature (1)..(11): Sequence1 (Used in Kit)
  misc_feature (2)..(7): Type IIS binding site
  misc_feature (12)..(81): Sequence2 (In Plasmid)
ngtyggaagctagaaatagcaagaccractaaggctagtccgttatcaacttgaaaaagtggcaccgagtcggtgcttttt

SEQ ID NO: 471 - DNA, 80 bp, Artificial Sequence
  Reverse strand of a polynucleotide which encodes an RNA bound by a cas enzyme
  misc_feature (1)..(74): Sequence2 (In Plasmid)
  misc_feature (75)..(80): Type IIS binding site
  misc_feature (75)..(80): Sequence1 (Used in Kit)
aaaaagcaccgactcggtgccactttttcaagttgataacggactagccttagtyggtcttgctatttctagcttccrac

SEQ ID NO: 472 - DNA, 81 bp, Artificial Sequence
  Forward strand of a polynucleotide which encodes an RNA bound by a cas enzyme
  misc_feature (1)..(11): Sequence1 (Used in Kit)
  misc_feature (2)..(7): Type IIS binding site
  misc_feature (12)..(81): Sequence2 (In Plasmid)
gctyggaagctagaaatagcaagaccractaaggctagtccgttatcaacttgaaaaagtggcaccgagtcggtgcttttt

SEQ ID NO: 473 - DNA, 83 bp, Artificial Sequence
  Reverse strand of a polynucleotide which encodes an RNA bound by a cas enzyme
  misc_feature (1)..(74): Sequence2 (In Plasmid)
  misc_feature (75)..(80): Type IIS binding site
  misc_feature (75)..(83): Sequence1 (Used in Kit)
aaaaagcaccgactcggtgccactttttcaagttgataacggactagccttagtyggtcttgctatttctagcttccragcat

SEQ ID NO: 474 - DNA, 81 bp, Artificial Sequence
  Forward strand of a polynucleotide which encodes an RNA bound by a cas enzyme
  misc_feature (1)..(11): Sequence1 (Used in Kit)
  misc_feature (2)..(7): Type IIS binding site
  misc_feature (12)..(81): Sequence2 (In Plasmid)
ggtyggaagctagaaatagcaagaccractaaggctagtccgttatcaacttgaaaaagtggcaccgagtcggtgcttttt

SEQ ID NO: 475 - DNA, 83 bp, Artificial Sequence
  Reverse strand of a polynucleotide which encodes an RNA bound by a cas enzyme
  misc_feature (1)..(74): Sequence2 (In Plasmid)
  misc_feature (75)..(80): Type IIS binding site
  misc_feature (75)..(83): Sequence1 (Used in Kit)
aaaaagcaccgactcggtgccactttttcaagttgataacggactagccttagtyggtcttgctatttctagcttccraccat

SEQ ID NO: 476 - DNA, 81 bp, Artificial Sequence
  Forward strand of a polynucleotide which encodes an RNA bound by a cas enzyme
  misc_feature (1)..(6): Type IIS binding site
  misc_feature (1)..(11): Sequence1 (Used in Kit)
  misc_feature (12)..(81): Sequence2 (In Plasmid)
ctyggagagctagaaatagcaagaccractaaggctagtccgttatcaacttgaaaaagtggcaccgagtcggtgcttttt

SEQ ID NO: 477 - DNA, 81 bp, Artificial Sequence
  Reverse strand of a polynucleotide which encodes an RNA bound by a cas enzyme
  misc_feature (1)..(74): Sequence2 (In Plasmid)
  misc_feature (75)..(81): Sequence1 (Used in Kit)
  misc_feature (76)..(81): Type IIS binding site
aaaaagcaccgactcggtgccactttttcaagttgataacggactagccttagtyggtcttgctatttctagctctccrag

SEQ ID NO: 478 - DNA, 81 bp, Artificial Sequence
  Forward strand of a polynucleotide which encodes an RNA bound by a cas enzyme
  misc_feature (1)..(6): Type IIS binding site
  misc_feature (1)..(11): Sequence1 (Used in Kit)
  misc_feature (12)..(81): Sequence2 (In Plasmid)
gtyggagagctagaaatagcaagaccractaaggctagtccgttatcaacttgaaaaagtggcaccgagtcggtgcttttt

SEQ ID NO: 479 - DNA, 81 bp, Artificial Sequence
  Reverse strand of a polynucleotide which encodes an RNA bound by a cas enzyme
  misc_feature (1)..(74): Sequence2 (In Plasmid)
  misc_feature (75)..(81): Sequence1 (Used in Kit)
  misc_feature (76)..(81): Type IIS binding site
aaaaagcaccgactcggtgccactttttcaagttgataacggactagccttagtyggtcttgctatttctagctctccrac

SEQ ID NO: 480 - DNA, 81 bp, Artificial Sequence
  Forward strand of a polynucleotide which encodes an RNA bound by a cas enzyme
  misc_feature (4)..(9): Type IIS binding site
cgtctcggcctagaaataggaagtgagaataaggctagtccgttatcaacttgaaaaagtggcaccgagtcggtgcttttt

SEQ ID NO: 481 - DNA, 79 bp, Artificial Sequence
  Reverse strand of a polynucleotide which encodes an RNA bound by a cas enzyme
  misc_feature (73)..(78): Type IIS binding site
aaaaagcaccgactcggtgccactttttcaagttgataacggactagccttattctcacttcctatttctaggccgaga

SEQ ID NO: 482 - DNA, 81 bp, Artificial Sequence
  Forward strand of a polynucleotide which encodes an RNA bound by a cas enzyme
  misc_feature (1)..(1): n is a, c, g, or t
  misc_feature (4)..(9): Type IIS binding site
nttctcggcctagaaataggaagtgagaataaggctagtccgttatcaacttgaaaaagtggcaccgagtcggtgcttttt

SEQ ID NO: 483 - DNA, 80 bp, Artificial Sequence
  Reverse strand of a polynucleotide which encodes an RNA bound by a cas enzyme
  misc_feature (73)..(78): Type IIS binding site
aaaaagcaccgactcggtgccactttttcaagttgataacggactagccttattctcacttcctatttctaggccgagaa

SEQ ID NO: 484 - DNA, 81 bp, Artificial Sequence
  Forward strand of a polynucleotide which encodes an RNA bound by a cas enzyme
  misc_feature (4)..(9): Type IIS binding site
tatctcggcctagaaataggaagtgagaataaggctagtccgttatcaacttgaaaaagtggcaccgagtcggtgcttttt

SEQ ID NO: 485 - DNA, 79 bp, Artificial Sequence; identical to SEQ ID NO: 481
  Reverse strand of a polynucleotide which encodes an RNA bound by a cas enzyme
  misc_feature (73)..(78): Type IIS binding site

SEQ ID NO: 486 - DNA, 81 bp, Artificial Sequence
  Forward strand of a polynucleotide which encodes an RNA bound by a cas enzyme
  misc_feature (4)..(9): Type IIS binding site
gttctcggcctagaaataggaagtgagaataaggctagtccgttatcaacttgaaaaagtggcaccgagtcggtgcttttt

SEQ ID NO: 487 - DNA, 83 bp, Artificial Sequence
  Reverse strand of a polynucleotide which encodes an RNA bound by a cas enzyme
  misc_feature (73)..(78): Type IIS binding site
aaaaagcaccgactcggtgccactttttcaagttgataacggactagccttattctcacttcctatttctaggccgagaacat

SEQ ID NO: 488 - DNA, 81 bp, Artificial Sequence
  Forward strand of a polynucleotide which encodes an RNA bound by a cas enzyme
  misc_feature (1)..(30): Sequence1 (Used in Kit)
  misc_feature (3)..(8): Type IIS binding site
  misc_feature (31)..(81): Sequence2 (In Plasmid)
cgctcggcgctagaaatagcaaatcgagctaaggctagtccgttatcaacttgaaaaagtggcaccgagtcggtgcttttt

SEQ ID NO: 489 - DNA, 79 bp, Artificial Sequence
  Reverse strand of a polynucleotide which encodes an RNA bound by a cas enzyme
  misc_feature (1)..(55): Sequence2 (In Plasmid)
  misc_feature (56)..(79): Sequence1 (Used in Kit)
  misc_feature (74)..(79): Type IIS binding site
aaaaagcaccgactcggtgccactttttcaagttgataacggactagccttagctcgatttgctatttctagcgccgag

SEQ ID NO: 490 - DNA, 81 bp, Artificial Sequence
  Forward strand of a polynucleotide which encodes an RNA bound by a cas enzyme
  misc_feature (1)..(30): Sequence1 (Used in Kit)
  misc_feature (3)..(8): Type IIS binding site
  misc_feature (31)..(81): Sequence2 (In Plasmid)
ntctcggcgctagaaatagcaaatcgagctaaggctagtccgttatcaacttgaaaaagtggcaccgagtcggtgcttttt

SEQ ID NO: 491 - DNA, 80 bp, Artificial Sequence
  Reverse strand of a polynucleotide which encodes an RNA bound by a cas enzyme
  misc_feature (1)..(55): Sequence2 (In Plasmid)
  misc_feature (56)..(80): Sequence1 (Used in Kit)
  misc_feature (74)..(79): Type IIS binding site
aaaaagcaccgactcggtgccactttttcaagttgataacggactagccttagctcgatttgctatttctagcgccgaga

SEQ ID NO: 492 - DNA, 81 bp, Artificial Sequence
  Forward strand of a polynucleotide which encodes an RNA bound by a cas enzyme
  misc_feature (1)..(30): Sequence1 (Used in Kit)
  misc_feature (3)..(8): Type IIS binding site
  misc_feature (31)..(81): Sequence2 (In Plasmid)
tactcggcgctagaaatagcaaatcgagctaaggctagtccgttatcaacttgaaaaagtggcaccgagtcggtgcttttt

SEQ ID NO: 493 - DNA, 79 bp, Artificial Sequence; identical to SEQ ID NO: 489
  Reverse strand of a polynucleotide which encodes an RNA bound by a cas enzyme
  misc_feature (1)..(55): Sequence2 (In Plasmid)
  misc_feature (56)..(79): Sequence1 (Used in Kit)
  misc_feature (74)..(79): Type IIS binding site

SEQ ID NO: 494 - DNA, 81 bp, Artificial Sequence
  Forward strand of a polynucleotide which encodes an RNA bound by a cas enzyme
  misc_feature (1)..(30): Sequence1 (Used in Kit)
  misc_feature (3)..(8): Type IIS binding site
  misc_feature (31)..(81): Sequence2 (In Plasmid)
gtctcggcgctagaaatagcaaatcgagctaaggctagtccgttatcaacttgaaaaagtggcaccgagtcggtgcttttt

SEQ ID NO: 495 - DNA, 83 bp, Artificial Sequence
  Reverse strand of a polynucleotide which encodes an RNA bound by a cas enzyme
  misc_feature (1)..(55): Sequence2 (In Plasmid)
  misc_feature (56)..(83): Sequence1 (Used in Kit)
  misc_feature (74)..(79): Type IIS binding site
aaaaagcaccgactcggtgccactttttcaagttgataacggactagccttagctcgatttgctatttctagcgccgagacat

SEQ ID NO: 496 - DNA, 81 bp, Artificial Sequence
  Forward strand of a polynucleotide which encodes an RNA bound by a cas enzyme
  misc_feature (1)..(11): Sequence1 (Used in Kit)
  misc_feature (2)..(7): Type IIS binding site
  misc_feature (12)..(81): Sequence2 (In Plasmid)
nctcggcagctagaaatagcaagcccgagtaaggctagtccgttatcaacttgaaaaagtggcaccgagtcggtgcttttt

SEQ ID NO: 497 - DNA, 80 bp, Artificial Sequence
  Reverse strand of a polynucleotide which encodes an RNA bound by a cas enzyme
  misc_feature (1)..(74): Sequence2 (In Plasmid)
  misc_feature (75)..(80): Type IIS binding site
  misc_feature (75)..(80): Sequence1 (Used in Kit)
aaaaagcaccgactcggtgccactttttcaagttgataacggactagccttactcgggcttgctatttctagctgccgag

SEQ ID NO: 498 - DNA, 81 bp, Artificial Sequence
  Forward strand of a polynucleotide which encodes an RNA bound by a cas enzyme
  misc_feature (1)..(11): Sequence1 (Used in Kit)
  misc_feature (2)..(7): Type IIS binding site
  misc_feature (12)..(81): Sequence2 (In Plasmid)
gctcggcagctagaaatagcaagcccgagtaaggctagtccgttatcaacttgaaaaagtggcaccgagtcggtgcttttt

SEQ ID NO: 499 - DNA, 83 bp, Artificial Sequence
  Reverse strand of a polynucleotide which encodes an RNA bound by a cas enzyme
  misc_feature (1)..(74): Sequence2 (In Plasmid)
  misc_feature (75)..(80): Type IIS binding site
  misc_feature (75)..(83): Sequence1 (Used in Kit)
aaaaagcaccgactcggtgccactttttcaagttgataacggactagccttactcgggcttgctatttctagctgccgagcat

SEQ ID NO: 500 - DNA, 81 bp, Artificial Sequence; identical to SEQ ID NO: 34
  Forward strand of a polynucleotide which encodes an RNA bound by a cas enzyme
  misc_feature (1)..(6): Type IIS binding site
  misc_feature (1)..(11): Sequence1 (Used in Kit)
  misc_feature (12)..(81): Sequence2 (In Plasmid)

SEQ ID NO: 501 - DNA, 83 bp, Artificial Sequence
  Reverse strand of a polynucleotide which encodes an RNA bound by a cas enzyme
  misc_feature (1)..(74): Sequence2 (In Plasmid)
  misc_feature (75)..(83): Sequence1 (Used in Kit)
  misc_feature (76)..(81): Type IIS binding site
aaaaagcaccgactcggtgccactttttcaagttgataacggactagccttatcggcacttgctatttctagctcgccgagat

SEQ ID NO: 502 - DNA, 22 bp, Artificial Sequence
  crRNA
  misc_feature (1)..(1): n is a, c, g, or t
ntgtyggagctatgctgttttg

SEQ ID NO: 503 - DNA, 21 bp, Artificial Sequence
  crRNA
caaaacagcatagctccraca

SEQ ID NO: 504 - DNA, 22 bp, Artificial Sequence
  crRNA
  misc_feature (1)..(1): n is a, c, g, or t
ntctyggagctatgctgttttg

SEQ ID NO: 505 - DNA, 21 bp, Artificial Sequence
  crRNA
caaaacagcatagctccraga

SEQ ID NO: 506 - DNA, 22 bp, Artificial Sequence
  crRNA
cgctyggagctatgctgttttg

SEQ ID NO: 507 - DNA, 20 bp, Artificial Sequence
  crRNA
caaaacagcatagctccrag

SEQ ID NO: 508 - DNA, 22 bp, Artificial Sequence
  crRNA
tactyggagctatgctgttttg

SEQ ID NO: 509 - DNA, 20 bp, Artificial Sequence
  crRNA
caaaacagcatagctccrag

SEQ ID NO: 510 - DNA, 22 bp, Artificial Sequence
  crRNA
gtctyggagctatgctgttttg

SEQ ID NO: 511 - DNA, 24 bp, Artificial Sequence
  crRNA
caaaacagcatagctccragacat

SEQ ID NO: 512 - DNA, 22 bp, Artificial Sequence
  crRNA
gtgtyggagctatgctgttttg

SEQ ID NO: 513 - DNA, 24 bp, Artificial Sequence
  crRNA
caaaacagcatagctccracacat

SEQ ID NO: 514 - DNA, 22 bp, Artificial Sequence
  crRNA
cgtyggaagctatgctgttttg

SEQ ID NO: 515 - DNA, 20 bp, Artificial Sequence
  crRNA
caaaacagcatagcttccra

SEQ ID NO: 516 - DNA, 22 bp, Artificial Sequence
  crRNA
  misc_feature (1)..(1): n is a, c, g, or t
nctyggaagctatgctgttttg

SEQ ID NO: 517 - DNA, 21 bp, Artificial Sequence
  crRNA
caaaacagcatagcttccrag

SEQ ID NO: 518 - DNA, 22 bp, Artificial Sequence
  crRNA
  misc_feature (1)..(1): n is a, c, g, or t
ngtyggaagctatgctgttttg

SEQ ID NO: 519 - DNA, 21 bp, Artificial Sequence
  crRNA
caaaacagcatagcttccrac

SEQ ID NO: 520 - DNA, 22 bp, Artificial Sequence
  crRNA
gctyggaagctatgctgttttg

SEQ ID NO: 521 - DNA, 24 bp, Artificial Sequence
  crRNA
caaaacagcatagcttccragcat

SEQ ID NO: 522 - DNA, 22 bp, Artificial Sequence
  crRNA
ggtyggaagctatgctgttttg

SEQ ID NO: 523 - DNA, 24 bp, Artificial Sequence
  crRNA
caaaacagcatagcttccraccat

SEQ ID NO: 524 - DNA, 22 bp, Artificial Sequence
  crRNA
ctyggagagctatgctgttttg

SEQ ID NO: 525 - DNA, 24 bp, Artificial Sequence
  crRNA
caaaacagcatagctctccragat

SEQ ID NO: 526 - DNA, 22 bp, Artificial Sequence
  crRNA
gtyggagagctatgctgttttg

SEQ ID NO: 527 - DNA, 24 bp, Artificial Sequence
  crRNA
caaaacagcatagctctccracat

SEQ ID NO: 528 - DNA, 22 bp, Artificial Sequence
  crRNA
cgtctcggcctatgctgttttg

SEQ ID NO: 529 - DNA, 20 bp, Artificial Sequence
  crRNA
caaaacagcataggccgaga

SEQ ID NO: 530 - DNA, 22 bp, Artificial Sequence
  crRNA
  misc_feature (1)..(1): n is a, c, g, or t
nttctcggcctatgctgttttg

SEQ ID NO: 531 - DNA, 21 bp, Artificial Sequence
  crRNA
caaaacagcataggccgagaa

SEQ ID NO: 532 - DNA, 22 bp, Artificial Sequence
  crRNA
tatctcggcctatgctgttttg

SEQ ID NO: 533 - DNA, 20 bp, Artificial Sequence
  crRNA
caaaacagcataggccgaga

SEQ ID NO: 534 - DNA, 22 bp, Artificial Sequence
  crRNA
gttctcggcctatgctgttttg

SEQ ID NO: 535 - DNA, 24 bp, Artificial Sequence
  crRNA
caaaacagcataggccgagaacat

SEQ ID NO: 536 - DNA, 22 bp, Artificial Sequence
  crRNA
cgctcggcgctatgctgttttg

SEQ ID NO: 537 - DNA, 20 bp, Artificial Sequence
  crRNA
caaaacagcatagcgccgag

SEQ ID NO: 538 - DNA, 22 bp, Artificial Sequence
  crRNA
  misc_feature (1)..(1): n is a, c, g, or t
ntctcggcgctatgctgttttg

SEQ ID NO: 539 - DNA, 21 bp, Artificial Sequence
  crRNA
caaaacagcatagcgccgaga

SEQ ID NO: 540 - DNA, 22 bp, Artificial Sequence
  crRNA
tactcggcgctatgctgttttg

SEQ ID NO: 541 - DNA, 20 bp, Artificial Sequence
  crRNA
caaaacagcatagcgccgag

SEQ ID NO: 542 - DNA, 22 bp, Artificial Sequence
  crRNA
gtctcggcgctatgctgttttg

SEQ ID NO: 543 - DNA, 24 bp, Artificial Sequence
  crRNA
caaaacagcatagcgccgagacat

SEQ ID NO: 544 - DNA, 22 bp, Artificial Sequence
  crRNA
  misc_feature (1)..(1): n is a, c, g, or t
nctcggcagctatgctgttttg

SEQ ID NO: 545 - DNA, 21 bp, Artificial Sequence
  crRNA
caaaacagcatagctgccgag

SEQ ID NO: 546 - DNA, 22 bp, Artificial Sequence
  crRNA
gctcggcagctatgctgttttg

SEQ ID NO: 547 - DNA, 24 bp, Artificial Sequence
  crRNA
caaaacagcatagctgccgagcat

SEQ ID NO: 548 - DNA, 22 bp, Artificial Sequence
  crRNA
ctcggcgagctatgctgttttg

SEQ ID NO: 549 - DNA, 24 bp, Artificial Sequence
  crRNA
caaaacagcatagctcgccgagat

SEQ ID NO: 550 - DNA, 84 bp, Artificial Sequence
  trascrRNA
ggaaccattcaaaacagcatagcaagtcragataaggctagtccgttatcaacttgaaaaagtggcaccgagtcggtgcttttt

SEQ ID NO: 551 - DNA, 84 bp, Artificial Sequence
  trascrRNA
aaaaagcaccgactcggtgccactttttcaagttgataacggactagccttatctygacttgctatgctgttttgaatggttcc

SEQ ID NO: 552 - DNA, 84 bp, Artificial Sequence; identical to SEQ ID NO: 550
  trascrRNA

SEQ ID NO: 553 - DNA, 84 bp, Artificial Sequence; identical to SEQ ID NO: 551
  trascrRNA

SEQ ID NO: 554 - DNA, 84 bp, Artificial Sequence
  trascrRNA
ggaaccattcaaaacagcatagcaagtcragctaaggctagtccgttatcaacttgaaaaagtggcaccgagtcggtgcttttt

SEQ ID NO: 555 - DNA, 84 bp, Artificial Sequence
  trascrRNA
aaaaagcaccgactcggtgccactttttcaagttgataacggactagccttagctygacttgctatgctgttttgaatggttcc

SEQ ID NO: 556 - DNA, 84 bp, Artificial Sequence; identical to SEQ ID NO: 554
  trascrRNA

SEQ ID NO: 557 - DNA, 84 bp, Artificial Sequence; identical to SEQ ID NO: 555
  trascrRNA

SEQ ID NO: 558 - DNA, 84 bp, Artificial Sequence; identical to SEQ ID NO: 554
  trascrRNA

SEQ ID NO: 559 - DNA, 84 bp, Artificial Sequence; identical to SEQ ID NO: 555
  trascrRNA

SEQ ID NO: 560 - DNA, 84 bp, Artificial Sequence; identical to SEQ ID NO: 554
  trascrRNA

SEQ ID NO: 561 - DNA, 84 bp, Artificial Sequence; identical to SEQ ID NO: 555
  trascrRNA

SEQ ID NO: 562 - DNA, 84 bp, Artificial Sequence
  trascrRNA
ggaaccattcaaaacagcatagcaagaccractaaggctagtccgttatcaacttgaaaaagtggcaccgagtcggtgcttttt

SEQ ID NO: 563 - DNA, 84 bp, Artificial Sequence
  trascrRNA
aaaaagcaccgactcggtgccactttttcaagttgataacggactagccttagtyggtcttgctatgctgttttgaatggttcc

SEQ ID NO: 564 - DNA, 84 bp, Artificial Sequence; identical to SEQ ID NO: 562
  trascrRNA

SEQ ID NO: 565 - DNA, 84 bp, Artificial Sequence; identical to SEQ ID NO: 563
  trascrRNA

SEQ ID NO: 566 - DNA, 84 bp, Artificial Sequence; identical to SEQ ID NO: 562
  trascrRNA

SEQ ID NO: 567 - DNA, 84 bp, Artificial Sequence; identical to SEQ ID NO: 563
  trascrRNA

SEQ ID NO: 568 - DNA, 84 bp, Artificial Sequence; identical to SEQ ID NO: 562
  trascrRNA

SEQ ID NO: 569 - DNA, 84 bp, Artificial Sequence; identical to SEQ ID NO: 563
  trascrRNA

SEQ ID NO: 570 - DNA, 84 bp, Artificial Sequence; identical to SEQ ID NO: 562
  trascrRNA

SEQ ID NO: 571 - DNA, 84 bp, Artificial Sequence; identical to SEQ ID NO: 563
  trascrRNA

SEQ ID NO: 572 - DNA, 84 bp, Artificial Sequence; identical to SEQ ID NO: 562
  trascrRNA

SEQ ID NO: 573 - DNA, 84 bp, Artificial Sequence; identical to SEQ ID NO: 563
  trascrRNA

SEQ ID NO: 574 - DNA, 84 bp, Artificial Sequence; identical to SEQ ID NO: 562
  trascrRNA

SEQ ID NO: 575 - DNA, 84 bp, Artificial Sequence; identical to SEQ ID NO: 563
  trascrRNA

SEQ ID NO: 576 - DNA, 84 bp, Artificial Sequence
  trascrRNA
ggaaccattcaaaacagcataggaagtgagaataaggctagtccgttatcaacttgaaaaagtggcaccgagtcggtgcttttt

SEQ ID NO: 577 - DNA, 84 bp, Artificial Sequence
  trascrRNA
aaaaagcaccgactcggtgccactttttcaagttgataacggactagccttattctcacttcctatgctgttttgaatggttcc

SEQ ID NO: 578 - DNA, 84 bp, Artificial Sequence; identical to SEQ ID NO: 576
  trascrRNA

SEQ ID NO: 579 - DNA, 84 bp, Artificial Sequence; identical to SEQ ID NO: 577
  trascrRNA

SEQ ID NO: 580 - DNA, 84 bp, Artificial Sequence; identical to SEQ ID NO: 576
  trascrRNA

SEQ ID NO: 581 - DNA, 84 bp, Artificial Sequence; identical to SEQ ID NO: 577
  trascrRNA

SEQ ID NO: 582 - DNA, 84 bp, Artificial Sequence; identical to SEQ ID NO: 576
  trascrRNA

SEQ ID NO: 583 - DNA, 84 bp, Artificial Sequence; identical to SEQ ID NO: 577
  trascrRNA

SEQ ID NO: 584 - DNA, 84 bp, Artificial Sequence
  trascrRNA
ggaaccattcaaaacagcatagcaaatcgagctaaggctagtccgttatcaacttgaaaaagtggcaccgagtcggtgcttttt

SEQ ID NO: 585 - DNA, 84 bp, Artificial Sequence
  trascrRNA
aaaaagcaccgactcggtgccactttttcaagttgataacggactagccttagctcgatttgctatgctgttttgaatggttcc

SEQ ID NO: 586 - DNA, 84 bp, Artificial Sequence; identical to SEQ ID NO: 584
  trascrRNA

SEQ ID NO: 587 - DNA, 84 bp, Artificial Sequence; identical to SEQ ID NO: 585
  trascrRNA

SEQ ID NO: 588 - DNA, 84 bp, Artificial Sequence; identical to SEQ ID NO: 584
  trascrRNA

SEQ ID NO: 589 - DNA, 84 bp, Artificial Sequence; identical to SEQ ID NO: 585
  trascrRNA

SEQ ID NO: 590 - DNA, 84 bp, Artificial Sequence; identical to SEQ ID NO: 584
  trascrRNA

SEQ ID NO: 591 - DNA, 84 bp, Artificial Sequence; identical to SEQ ID NO: 585
  trascrRNA

SEQ ID NO: 592 - DNA, 84 bp, Artificial Sequence
  trascrRNA
ggaaccattcaaaacagcatagcaagcccgagtaaggctagtccgttatcaacttgaaaaagtggcaccgagtcggtgcttttt

SEQ ID NO: 593 - DNA, 84 bp, Artificial Sequence
  trascrRNA
aaaaagcaccgactcggtgccactttttcaagttgataacggactagccttactcgggcttgctatgctgttttgaatggttcc

SEQ ID NO: 594 - DNA, 84 bp, Artificial Sequence; identical to SEQ ID NO: 592
  trascrRNA

SEQ ID NO: 595 - DNA, 84 bp, Artificial Sequence; identical to SEQ ID NO: 593
  trascrRNA

SEQ ID NO: 596 - DNA, 84 bp, Artificial Sequence
  trascrRNA
ggaaccattcaaaacagcatagcaagtgccgataaggctagtccgttatcaacttgaaaaagtggcaccgagtcggtgcttttt

SEQ ID NO: 597 - DNA, 84 bp, Artificial Sequence
  trascrRNA
aaaaagcaccgactcggtgccactttttcaagttgataacggactagccttatcggcacttgctatgctgttttgaatggttcc